(19) United States
(12) Patent Application Publication
(10) Pub. No.: US 2010/0204265 A1
(43) Pub. Date: Aug. 12, 2010

(54) Certain Nitrogen Containing Bicyclic Chemical Entities for Treating Viral Infections

(75) Inventors: Subramanian Baskaran, Foster City, CA (US); Jack Maung, Daly City, CA (US); Martin Leon Neitzel, Pacifica, CA (US); Roopa Rai, San Carlos, CA (US); Irina Slododov, San Mateo, CA (US); Vincent W-F Tai, San Mateo, CA (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Genelabs Technologies, Inc.

(21) Appl. No.: 12/368,249

(22) Filed: Feb. 9, 2009

(51) Int. Cl. A61K 31/437; C07D 471/04; A61P 31/12

(52) U.S. Cl. 514/300; 546/121

(57) ABSTRACT

Provided are certain chemical entities, pharmaceutical compositions, and methods of treatment of a member of the flaviviradae family of viruses such as hepacivirus (Hepatitis C or HCV).

Provided are certain chemical entities, pharmaceutical compositions and methods of treatment of a member of the flaviviradae family of viruses such as hepacivirus (Hepatitis C or HCV).

The Flaviviridae family of viruses is composed of three genera: pestivirus, flavivirus and hepacivirus (hepatitis C virus). Of these genera, flaviviruses and hepaciviruses represent important pathogens of man and are prevalent throughout the world. There are 38 flaviviruses associated with human disease, including the dengue fever viruses, yellow fever virus, and Japanese encephalitis virus. Flaviviruses cause a range of acute febrile illnesses and encephalitic and hemorrhagic diseases. Hepaciviruses currently infect approximately 2 to 3% of the world population and cause persistent infections leading to chronic liver disease, cirrhosis, hepatocellular carcinoma and liver failure. Human pestiviruses have not been as extensively characterized as the animal pestiviruses. However, serological surveys indicate considerable pestivirus exposure in humans. Pestivirus infections in man have been implicated in several diseases including, but not limited to, congenital brain injury, infantile gastroenteritis and chronic diarrhea in human immunodeficiency virus (HIV).

HCV is a major causative agent for post-transfusion and for sporadic hepatitis. Infection by HCV is insidious in a high proportion of chronically infected (and infectious) carriers who may not experience clinical symptoms for many years.

At present, the only acceptable treatment for chronic HCV is interferon (IFN-alpha) and/or ribavirin and this requires at least six (6) months of treatment, which can reduce the viral load and also improve liver function in some people.

IFN-alpha belongs to a family of naturally occurring small proteins with characteristic biological effects such as antiviral, immunoregulatory and anti-tumoral activities. IFN-alpha is an important regulator of immunological control. Treatment of HCV with interferon, however, has limited long term efficacy with a response rate about 25%. In addition, treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction.

Ribavirin (1-P-D-ribofuranosyl-1H-1,2,-4-triazole-3-carboxamide), an inhibitor of inosine 5′-monophosphate dehydrogenase (IMPDH), enhances the efficacy of IFN-alpha in the treatment of HCV. Despite the introduction of Ribavirin, up to 50% of the patients do not eliminate the virus with the current standard therapy of interferon-alpha (IFN) and Ribavirin. Ribavirin causes significant hemolysis in 10-20% of patients treated at currently recommended doses, and the drug is both teratogenic and embryotoxic. By now, standard therapy of chronic hepatitis C has been changed to the combination of PEG-IFN (pegylated interferon) plus ribavirin which leads only to small improvement.

Other approaches are being taken to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among non-structral viral proteins, NS3/4a serine protease, NS5b RNA dependent RNA polymerase are considered as prime targets for new drugs.

There is a need for the development of new compounds that combat hepacivirus. There remains a need for agents with stronger response rates and fewer side effects in terms of relief of symptoms, safety, and patient mortality, both short-term and long-term and an improved therapeutic index.

Provided is at least one chemical entity selected from compounds of the Formula I:

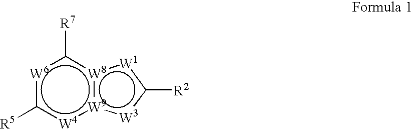

and pharmaceutically acceptable salts thereof, wherein

W¹ is selected from CR¹ and NR¹;

W³ is selected from CR³ and NR³;

W⁴ is selected from CR⁴ and N;

W⁶ is selected from CR⁶ and N;

W⁸ is selected from C and N;

W⁹ is selected from C and N;

R¹ is absent or is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR³, —CN, —NO₂, and —C(O)R¹²;

R² is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R³ is absent or is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁴ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁵ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁶ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁷ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R¹⁰ and R¹¹ are independently selected from hydrogen, optionally substituted alkyl, optionally substituted amino, optionally substituted alkoxy, optionally substituted cycloalkyl, optionally substituted heterocycloalkyl, optionally substituted aryl, and optionally substituted heteroaryl, or R¹⁰ and R¹¹, taken together with any intervening atoms, form a ring system selected from optionally substituted heterocycloalkyl, and optionally substituted heteroaryl;

R¹² is selected from hydrogen, optionally substituted alkyl, optionally substituted cycloalkyl, optionally substituted heterocycloalkyl, optionally substituted aryl, and optionally substituted heteroaryl;

R¹³ is selected from hydrogen, optionally substituted alkyl, optionally substituted cycloalkyl, optionally substituted heterocycloalkyl, optionally substituted aryl, and optionally substituted heteroaryl;

R¹⁴ is selected from optionally substituted alkyl, optionally substituted cycloalkyl, optionally substituted heterocycloalkyl, optionally substituted aryl, and optionally substituted heteroaryl;

R¹⁵ is selected from hydrogen, optionally substituted alkyl, optionally substituted cycloalkyl, optionally substituted heterocycloalkyl, optionally substituted aryl, and optionally substituted heteroaryl; and

R¹⁶ is selected from optionally substituted alkyl, optionally substituted cycloalkyl, optionally substituted heterocycloalkyl, optionally substituted aryl, and optionally substituted heteroaryl;

provided that
if W¹ is NR¹ and W³ is NR³, then R³ is absent;
if W³ is NR³ and W¹ is NR¹, then R¹ is absent;
at least one of W¹, W³, W⁸, and W⁹ is N;
no more than four of W¹, W³, W⁴, W⁶, W⁸, and W⁹ are N; and
if W¹ is N, W⁴ is N, and W⁶ is CR⁶, then W⁸ is not N;

and further provided that the compound of Formula I is not
(5-(5-chlorothiophen-2-yl)-7-(trifluoromethyl)pyrazolo[1,5-a]pyridin-2-yl)(3-(3,4-dimethoxyphenyl)-5-(2-hydroxyphenyl)-4,5-dihydro-1H-pyrazol-1-yl)methanone;
(5-(5-chlorothiophen-2-yl)-7-(trifluoromethyl)pyrazolo[1,5-a]pyridin-2-yl)(3-(2,5-dimethylphenyl)-5-(2-hydroxyphenyl)-4,5-dihydro-1H-pyrazol-1-yl)methanone; or
(5-(5-chlorothiophen-2-yl)-7-(trifluoromethyl)pyrazolo[1,5-a]pyridin-2-yl)(3-(3,4-dichlorophenyl)-5-(2-hydroxyphenyl)-4,5-dihydro-1H-pyrazol-1-yl)methanone.

Also provided is a pharmaceutical composition comprising a pharmaceutically acceptable diluent and a therapeutically effective amount of at least one chemical entity described herein.

Also provided is a pharmaceutical composition comprising a pharmaceutically acceptable diluent and a therapeutically effective amount of at least one chemical entity chosen from compounds of Formula 1a

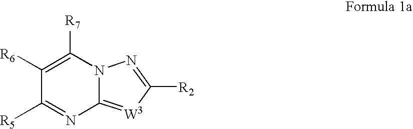

and pharmaceutically acceptable salts thereof, wherein

W³ is selected from CR³ and NR³;

R² is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, NR¹¹C(S)NR¹⁰R¹¹, —NR¹¹S(O)₂R¹⁴ —NR¹¹C(O)OR¹³, —NR¹¹C(O)R¹², —C(NR)NR¹⁰R¹¹, —C(O)NR¹⁰R¹¹, —C(O)OR¹³, —CN, —NO₂, and —C(O)R¹²;

R³ is absent or is selected from halogen, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁵ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁶ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁷ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂R¹⁰R¹¹, —NR¹⁰R¹¹, —NR¹¹C(O)NR¹⁰R¹¹, NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R¹⁰ and R¹¹ are independently selected from hydrogen, optionally substituted alkyl, optionally substituted amino, optionally substituted alkoxy, optionally substituted cycloalkyl, optionally substituted heterocycloalkyl, optionally substituted aryl, and optionally substituted heteroaryl, or R¹⁰ and R¹¹, taken together with any intervening atoms, form a ring system selected from optionally substituted heterocycloalkyl, and optionally substituted heteroaryl;

R¹² is selected from hydrogen, optionally substituted alkyl, optionally substituted cycloalkyl, optionally substituted heterocycloalkyl, optionally substituted aryl, and optionally substituted heteroaryl;

R¹³ is selected from hydrogen, optionally substituted alkyl, optionally substituted cycloalkyl, optionally substituted heterocycloalkyl, optionally substituted aryl, and optionally substituted heteroaryl;

R¹⁴ is selected from optionally substituted alkyl, optionally substituted cycloalkyl, optionally substituted heterocycloalkyl, optionally substituted aryl, and optionally substituted heteroaryl;

R¹⁵ is selected from hydrogen, optionally substituted alkyl, optionally substituted cycloalkyl, optionally substituted heterocycloalkyl, optionally substituted aryl, and optionally substituted heteroaryl; and

R¹⁶ is selected from optionally substituted alkyl, optionally substituted cycloalkyl, optionally substituted heterocycloalkyl, optionally substituted aryl, and optionally substituted heteroaryl.

Also provided is a pharmaceutical composition comprising a pharmaceutically acceptable diluent and a therapeutically effective amount of at least one chemical entity chosen from
(5-(5-chlorothiophen-2-yl)-7-(trifluoromethyl)pyrazolo[1,5-a]pyridin-2-yl)(3-(3,4-dimethoxyphenyl)-5-(2-hydroxyphenyl)-4,5-dihydro-1H-pyrazol-1-yl)methanone;
(5-(5-chlorothiophen-2-yl)-7-(trifluoromethyl)pyrazolo[1,5-a]pyridin-2-yl)(3-(2,5-dimethylphenyl)-5-(2-hydroxyphenyl)-4,5-dihydro-1H-pyrazol-1-yl)methanone; or
(5-(5-chlorothiophen-2-yl)-7-(trifluoromethyl)pyrazolo[1,5-a]pyridin-2-yl)(3-(3,4-dichlorophenyl)-5-(2-hydroxyphenyl)-4,5-dihydro-1H-pyrazol-1-yl)methanone,
   and pharmaceutically acceptable salts thereof.

Also provided are methods for treating a viral infection mediated at least in part by a virus in the flaviviridae family of viruses, such as HCV, in mammals which methods comprise administering to a mammal, that has been diagnosed with said viral infection or is at risk of developing said viral infection, a pharmaceutical composition described herein.

Other aspects and embodiments will be apparent to those skilled in the art from the following detailed description.

In a preferred embodiment, this application does not cover any specific compound disclosed in the PCT Application No.: PCT/US2008/009606 or the U.S. patent application Ser. No. 12/228,139.

As used in the present specification, the following words and phrases are generally intended to have the meanings as set forth below, except to the extent that the context in which they are used indicates otherwise.

The following abbreviations and terms have the indicated meanings throughout:

HCV: hepacivirus

HIV: human immunodeficiency virus

IFN: interferon

IMPDH: inosine 5′-monophosphate dehydrogenase

mg: milligram

kg: kilogram

MDI: metered dose inhaler

DPI: dry powder inhaler

nM: nano-Molar

wt %: weight percent

μM: micro-Molar

EC₅₀: effective concentration of compound at 50% inhibition is observed

TC₅₀: toxic concentration of compound at which 50% inhibition is observed

b: Hill's coefficient

g: gram

K: Kelvin

mL: milli-Liter

1N: 1 Normal concentration

AIDS: Acquired Immunodeficiency syndrome

It is to be understood that the terminology used herein is for the purpose of describing particular embodiments only and is not intended to limit the scope of the present specification. In this specification and in the claims that follow, reference will be made to a number of terms that shall be defined to have the following meanings:

“Alkyl” refers to monovalent saturated aliphatic hydrocarbyl groups having from 1 to carbon atoms and, in some embodiments, from 1 to 6 carbon atoms. “C_{x-y}alkyl” refers to alkyl groups having from x to y carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 and, in some embodiments, 1 to 3 or 1 or 2 substituents selected from alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio, wherein said substituents are as defined herein.

“Alkylidene” or “alkylene” refers to divalent saturated aliphatic hydrocarbyl groups having from 1 to 10 carbon atoms and, in some embodiments, from 1 to 6 carbon atoms. “(Cᵤ₋ᵥ)alkylene” refers to alkylene groups having from u to v carbon atoms. The alkylidene and alkylene groups include branched and straight chain hydrocarbyl groups. For example “(C₁₋₆)alkylene” is meant to include methylene, ethylene, propylene, 2-methypropylene, pentylene, and the like.

“Substituted alkylidene” or “substituted alkylene” refers to an alkylidene group having from 1 to 5 and, in some embodiments, 1 to 3 or 1 or 2 substituents selected from alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, SO₃H, substituted sulfonyl, sulfonyloxy, thioacyl, thiocyanate, thiol, alkylthio, and substituted alkylthio, wherein said substituents are as defined herein.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ—C_{y})alkenyl refers to alkenyl groups having from x to y carbon atoms and is meant to include for example, ethenyl, propenyl, 1,3-butadienyl, and the like.

“Substituted alkenyl” refers to alkenyl groups having from 1 to 3 substituents and, in some embodiments, 1 or 2 substituents selected from alkoxy, substituted alkoxy, acyl, acylamino, acyloxy, alkyl, substituted alkyl, alkynyl, substituted alkynyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Substituted alkynyl” refers to alkynyl groups having from 1 to 3 substituents and, in some embodiments, from 1 or 2 substituents selected from alkoxy, substituted alkoxy, acyl, acylamino, acyloxy, alkyl, substituted alkyl, alkenyl, substituted alkenyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, substituted hydrazino-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, substituted hydrazino, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR²¹R²² where R²¹ and R²² are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²³R²⁴ where R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, hydroxy, alkoxy, substituted alkoxy, amino, substituted amino, and acylamino, and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonyl” refers to the group —C(S)NR²³R²⁴ where R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonylamino” refers to the group —NR²⁰C(O)NR²³R²⁴ where R²⁰ is hydrogen or alkyl and R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonylamino” refers to the group —NR²⁰C(S)NR²³R²⁴ where R²⁰ is hydrogen or alkyl and R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonyloxy” refers to the group —O—C(O)NR²³R²⁴ where R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyl” refers to the group —SO₂NR²³R²⁴ where R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyloxy” refers to the group —O—SO₂NR²³R²⁴ where R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonylamino” refers to the group —NR²⁰—SO₂NR²³R²⁴ where R²⁰ is hydrogen or alkyl and R²³ and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amidino” refers to the group —C(═NR²⁵)NR²³R²⁴ where R²⁵, R²³, and R²⁴ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aryl” or “Ar” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“Substituted aryl” refers to aryl groups which are substituted with 1 to 8 and, in some embodiments, 1 to 5, 1 to 3, or 1 or 2 substituents selected from alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthyloxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Azido” refers to the group —N₃.

“Hydrazino” refers to the group —NHNH₂.

“Substituted hydrazino” refers to the group —NR²⁶NR²⁷R²⁸ where R²⁶, R²⁷, and R²⁸ are independently selected from hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²⁷ and R²⁸ are optionally joined, together with the nitrogen bound thereto to form a heterocyclic or substituted heterocyclic group, provided that R²⁷ and R²⁸ are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” or “carbonitrile” refers to the group —CN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR²⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)O-cycloalkyl, —NR²⁰—C(O)O-substituted cycloalkyl, —NR²⁰—C(O)O-heteroaryl, —NR²⁰—C(O)O-substituted heteroaryl, —NR²⁰—C(O)O-heterocyclic, and —NR²⁰—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “Cycloalkyl” includes cycloalkenyl groups. Examples of cycloalkyl groups include, for instance, adamantyl, cyclopropyl, cyclobutyl, cyclopentyl, cyclooctyl, and cyclohexenyl. “Cᵤ₋ᵥcycloalkyl” refers to cycloalkyl groups having u to v carbon atoms.

“Cycloalkenyl” refers to a partially saturated cycloalkyl ring having at least one site of >C═C< ring unsaturation.

“Cycloalkylene” refer to divalent cycloalkyl groups as defined herein. Examples of cycloalkyl groups include those having three to six carbon ring atoms such as cyclopropylene, cyclobutylene, cyclopentylene, and cyclohexylene.

“Substituted cycloalkyl” refers to a cycloalkyl group, as defined herein, having from 1 to 8, or 1 to 5, or in some embodiments 1 to 3 substituents selected from oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are as defined herein. The term “substituted cycloalkyl” includes substituted cycloalkenyl groups.

“Cycloalkyloxy” refers to —O-cycloalkyl wherein cycloalkyl is as defined herein.

Substituted cycloalkyloxy refers to —O-(substituted cycloalkyl) wherein substituted cycloalkyl is as defined herein.

“Cycloalkylthio” refers to —S-cycloalkyl wherein cycloalkyl is as defined herein.

“Substituted cycloalkylthio” refers to —S-(substituted cycloalkyl).

“Guanidino” refers to the group —NHC(═NH)NH₂.

“Substituted guanidino” refers to —NR²⁹C(═NR²⁹)N(R²⁹)₂ where each R²⁹ is independently selected from hydrogen, alkyl, substituted alkyl, aryl, substituted aryl, heteroaryl, substituted heteroaryl, heterocyclyl, and substituted heterocyclyl and two R²⁹ groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo, and iodo.

“Haloalkyl” refers to substitution of alkyl groups with 1 to 5 or in some embodiments 1 to 3 halo groups.

“Haloalkoxy” refers to substitution of alkoxy groups with 1 to 5 or in some embodiments 1 to 3 halo groups.

“Hydroxy” or “hydroxyl” refers to the group —OH.

“Heteroaryl” refers to an aromatic group of from 1 to 14 carbon atoms and 1 to 6 heteroatoms selected from oxygen, nitrogen, and sulfur and includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In one embodiment, the carbon, nitrogen and/or the sulfur ring atom(s) of the heteroaryl group are optionally oxidized to provide for the C═O, N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, quinazolinonyl, benzimidazolyl, benzisoxazolyl, or benzothienyl.

“Substituted heteroaryl” refers to heteroaryl groups that are substituted with from 1 to 8 or in some embodiments 1 to 5, or 1 to 3, or 1 or 2 substituents selected from the substituents defined for substituted aryl.

“Heteroaryloxy” refers to —O-heteroaryl wherein heteroaryl is as defined herein.

“Substituted heteroaryloxy refers to the group —O-(substituted heteroaryl) wherein substituted heteroaryl is as defined herein.

“Heteroarylthio” refers to the group —S-heteroaryl wherein heteroaryl is as defined herein.

“Substituted heteroarylthio” refers to the group —S-(substituted heteroaryl) wherein substituted heteroaryl is as defined herein.

“Aromatic” indicates that each of ring atoms is essentially in the same plane and has a p-orbital perpendicular to the ring plane, and in which (4n+2)π electrons, when n is 0 or a positive integer, are associated with the ring to comply with Huckel's rule. Aromatic ring systems may be depicted as a circle, which represents the (4n+2) π electrons, enclosed by an outer cyclic structure, such as, a hexagon or pentagon. For example, each of the rings in the compound of Formula I is aromatic.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from nitrogen, sulfur, phosphorus or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In one embodiment, the nitrogen, phosphorus and/or sulfur atom(s) of the heterocyclic group are optionally oxidized to provide for the N-oxide, phosphinane oxide, sulfinyl, sulfonyl moieties. More specifically the heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

“Substituted heterocyclic” or “Substituted heterocycle” or “substituted heterocycloalkyl” or “substituted heterocyclyl” refers to heterocyclic groups, as defined herein, that are substituted with from 1 to 5 or in some embodiments 1 to 3 of the substituents as defined for substituted cycloalkyl.

“Heterocyclyloxy” refers to the group —O-heterocycyl wherein heterocyclyl is as defined herein.

“Substituted heterocyclyloxy” refers to the group —O-(substituted heterocycyl) wherein substituted heterocyclyl is as defined herein.

“Heterocyclylthio” refers to the group —S-heterocycyl wherein heterocyclyl is as defined herein.

“Substituted heterocyclylthio” refers to the group —S-(substituted heterocycyl) wherein substituted heterocyclyl is as defined herein.

Examples of heterocycle and heteroaryl groups include, but are not limited to, azetidine, pyrrole, imidazole, pyrazole, pyridine, pyrazine, pyrimidine, pyridazine, pyridone, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholine, thiomorpholine (also referred to as thiamorpholine), 1,1-dioxothiomorpholine, piperidine, pyrrolidine, and tetrahydrofuran.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Spirocycloalkyl” refers to a 3 to 10 member cyclic substituent formed by replacement of two hydrogen atoms at a common carbon atom with an alkylene group having 2 to 9 carbon atoms, as exemplified by the following structure wherein the methylene group shown here attached to bonds marked with wavy lines is substituted with a spirocycloalkyl group:

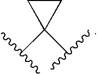

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

“Compound” and “compounds” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Racemates” refers to a mixture of enantiomers.

“Solvate” or “solvates” of a compound refer to those compounds, where compounds is as defined above, that are bound to a stoichiometric or non-stoichiometric amount of a solvent. Solvates of a compound includes solvates of all forms of the compound. In certaine mbodiments, solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Provided is at least one chemical entity selected from compounds of Formula I:

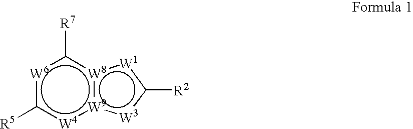

and pharmaceutically acceptable salts thereof, wherein

W¹ is selected from CR¹ and NR¹;

W³ is selected from CR³ and NR³;

W⁴ is selected from CR⁴ and N;

W⁶ is selected from CR⁶ and N;

W⁸ is selected from C and N;

W⁹ is selected from C and N;

R¹ is absent or is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²

R² is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR³, —CN, —NO₂, and —C(O)R¹²;

R³ is absent or is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR³, —NR¹¹C(O)R¹², —C(NR¹¹)NR¹⁰R¹¹, —C(O)NR¹⁰R¹¹, —C(O)OR³, —CN, —NO₂, and —C(O)R¹²;

R⁴ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁵ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁶ is selected from hydrogen, halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R⁷ is selected from halogen, optionally substituted alkyl, optionally substituted alkenyl, optionally substituted alkynyl, optionally substituted cycloalkyl, optionally substituted amino, optionally substituted heterocycloalkyl, optionally substituted aryl, optionally substituted heteroaryl, —OR¹⁵, —SR¹⁵, —S(O)R¹⁶, —S(O)₂R¹⁶, —S(O)₂NR¹⁰R¹¹, —NR¹⁰R¹¹, —NR¹¹C(O)NR¹⁰R¹¹, —NR¹¹C(S)NR¹⁰R¹¹, —NR¹¹S(O)₂R¹⁴ —NR¹¹C(O)OR¹³, —NR¹¹C(O)R¹², —C(NR¹¹)NR¹⁰R¹¹, —C(O)NR¹⁰R¹¹, —C(O)OR¹³, —CN, —NO₂, and —C(O)R¹²;

R¹⁰ and R¹¹ are independently selected from hydrogen, optionally substituted alkyl, optionally substituted amino, optionally substituted alkoxy, optionally substituted cycloalkyl, optionally substituted heterocycloalkyl, optionally substituted aryl, and optionally substituted heteroaryl, or R¹⁰ and R¹¹, taken together with any intervening atoms, form a ring system selected from optionally substituted heterocycloalkyl, and optionally substituted heteroaryl;

R¹² is selected from hydrogen, optionally substituted alkyl, optionally substituted cycloalkyl, optionally substituted heterocycloalkyl, optionally substituted aryl, and optionally substituted heteroaryl;

R¹³ is selected from hydrogen, optionally substituted alkyl, optionally substituted cycloalkyl, optionally substituted heterocycloalkyl, optionally substituted aryl, and optionally substituted heteroaryl;

R¹⁴ is selected from optionally substituted alkyl, optionally substituted cycloalkyl, optionally substituted heterocycloalkyl, optionally substituted aryl, and optionally substituted heteroaryl;

R¹⁵ is selected from hydrogen, optionally substituted alkyl, optionally substituted cycloalkyl, optionally substituted heterocycloalkyl, optionally substituted aryl, and optionally substituted heteroaryl; and

R¹⁶ is selected from optionally substituted alkyl, optionally substituted cycloalkyl, optionally substituted heterocycloalkyl, optionally substituted aryl, and optionally substituted heteroaryl;

provided that
if W¹ is NR¹ and W³ is NR³, then R³ is absent;
if W³ is NR³ and W¹ is NR¹, then R¹ is absent;
at least one of W¹, W³, W⁸, and W⁹ is N;
no more than four of W¹, W³, W⁴, W⁶, W⁸, and W⁹ are N; and
if W¹ is N, W⁴ is N, and W⁶ is CR⁶, then W⁸ is not N;

and further provided that the compound of Formula I is not
(5-(5-chlorothiophen-2-yl)-7-(trifluoromethyl)pyrazolo[1,5-a]pyridin-2-yl)(3-(3,4-dimethoxyphenyl)-5-(2-hydroxyphenyl)-4,5-dihydro-1H-pyrazol-1-yl)methanone;
(5-(5-chlorothiophen-2-yl)-7-(trifluoromethyl)pyrazolo[1,5-a]pyridin-2-yl)(3-(2,5-dimethylphenyl)-5-(2-hydroxyphenyl)-4,5-dihydro-1H-pyrazol-1-yl)methanone; or
(5-(5-chlorothiophen-2-yl)-7-(trifluoromethyl)pyrazolo[1,5-a]pyridin-2-yl)(3-(3,4-dichlorophenyl)-5-(2-hydroxyphenyl)-4,5-dihydro-1H-pyrazol-1-yl)methanone.

In some embodiments, the compound of Formula I is selected from the following compounds

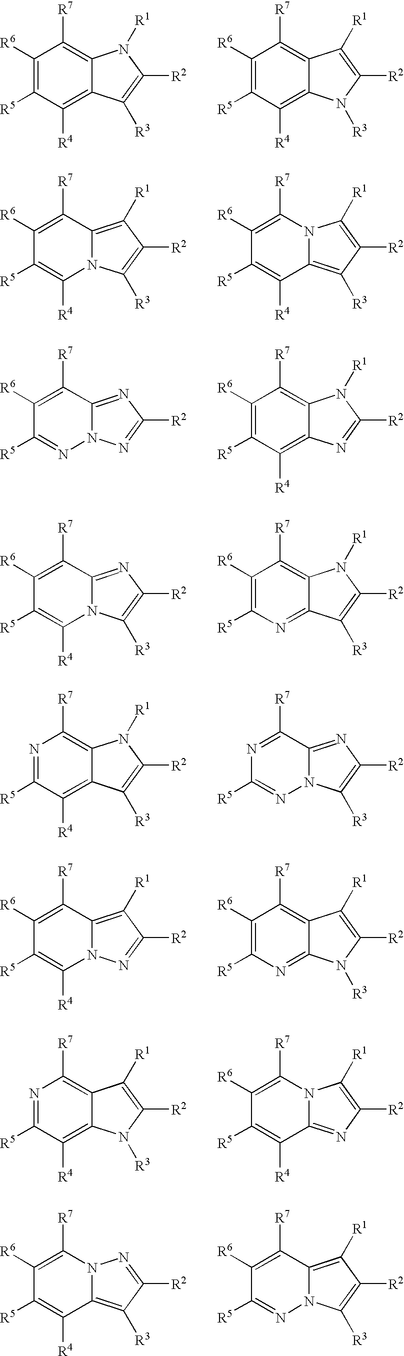

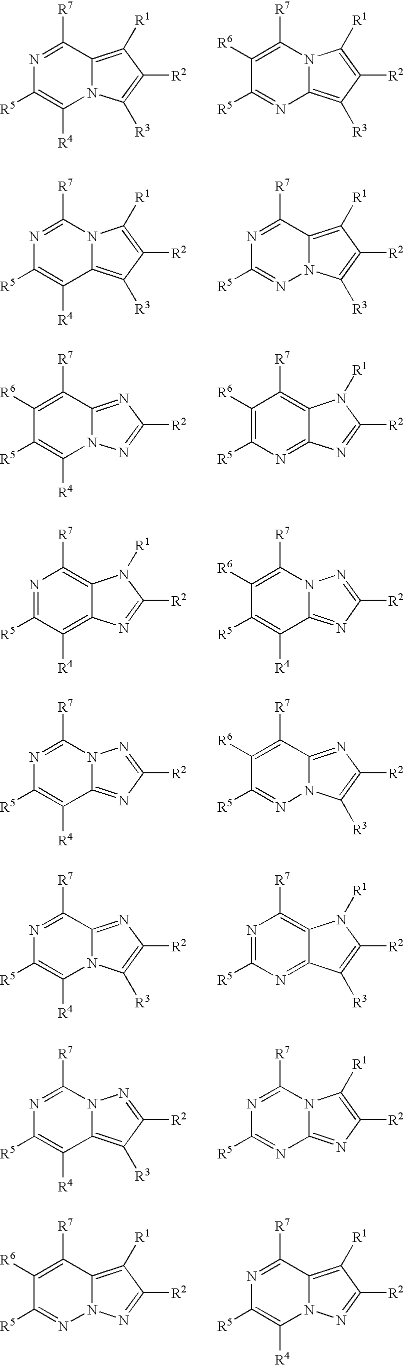

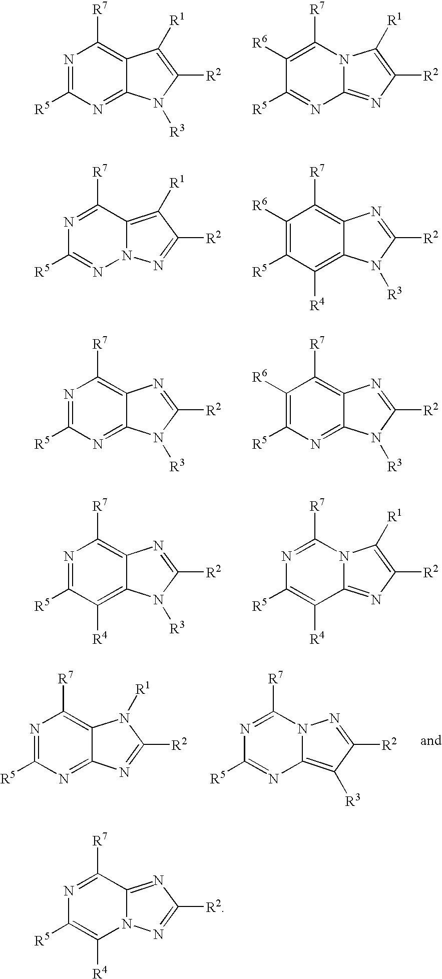

In some embodiments, the compound of Formula I is selected from the following compounds:

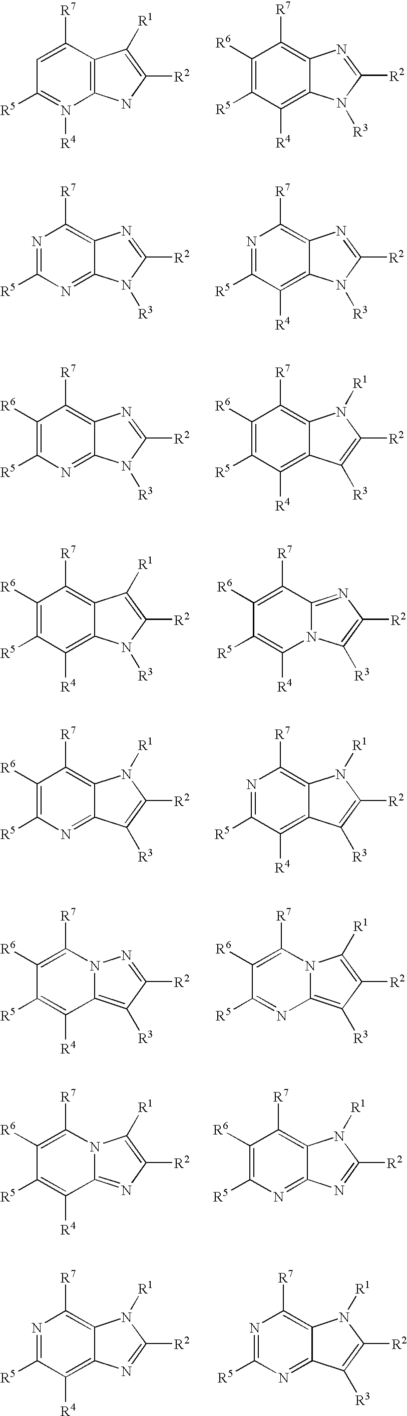

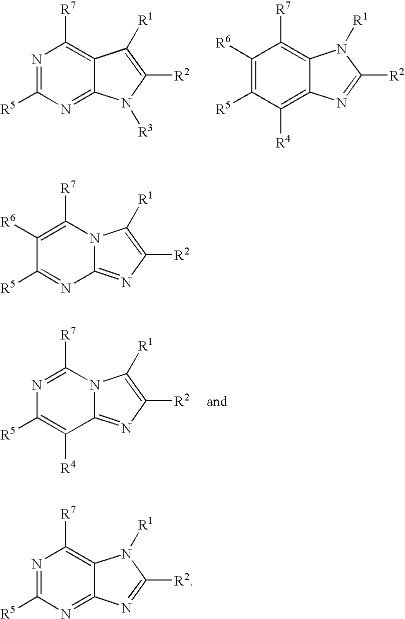

In some embodiments, the compound of Formula I is selected from the following compounds:

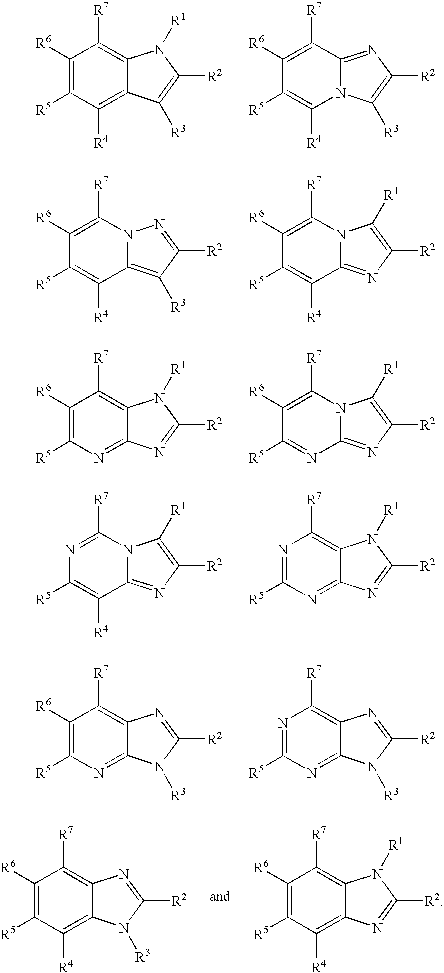

In some embodiments, the compound of Formula I is selected from the following compounds:

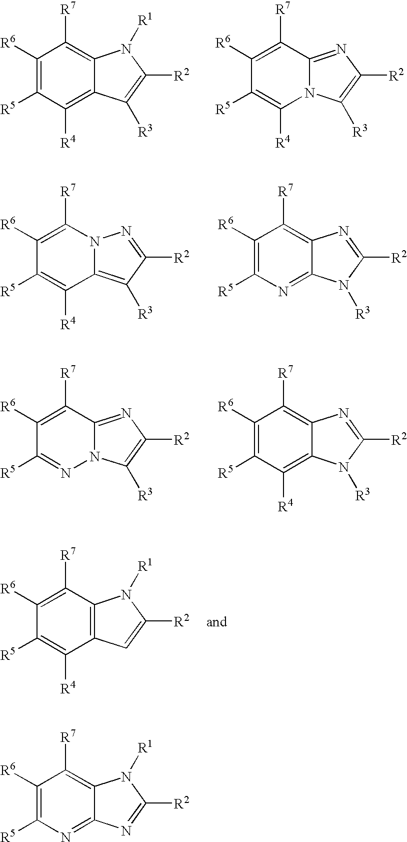

In some embodiments, the compound of Formula I is selected from the following compounds:

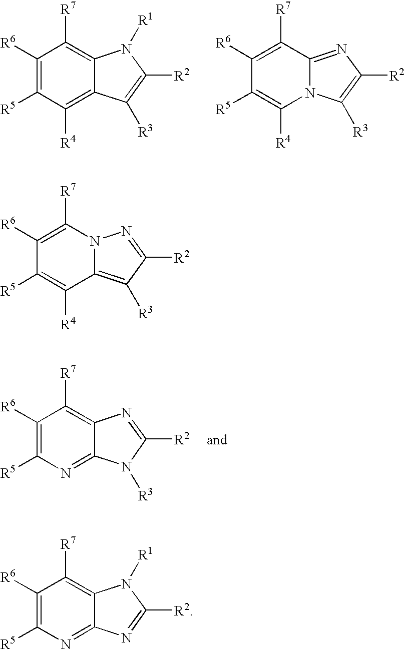

In some embodiments, the compound of Formula I is

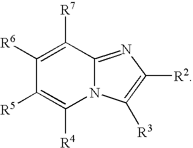

In some embodiments, R² is selected from optionally substituted alkyl, —NR¹¹S(O)₂R¹⁴, —NR¹¹C(O)NR¹⁰R¹¹, —NR¹¹C(O)OR¹³—C(O)NR¹⁰R¹¹, and —C(O)OR¹³.

In some embodiments, R² is lower alkyl substituted with —NR¹⁰R¹¹, where R¹⁰ and R¹¹ are as described herein. In some embodiments, R² is —CH₂—NR¹⁰R¹¹, where R¹⁰ and R¹¹ are as described herein.

In some embodiments, R² is lower alkyl substituted with —NR¹⁰R¹¹ and R¹⁰ and R¹¹, together with any intervening atoms, form an optionally substituted heterocycloalkyl, as described herein. In some embodiments, R² is —CH₂—NR¹⁰R¹¹ and R¹⁰ and R¹¹, together with any intervening atoms, form an optionally substituted heterocycloalkyl, as described herein.

In some embodiments, R² is lower alkyl substituted with —C(O)NR¹⁰R¹¹, where R¹⁰ and R¹¹ are as described herein. In some embodiments, R² is —CH₂—C(O)NR¹⁰R¹¹, where R¹⁰ and R¹¹ are as described herein.

In some embodiments, R² is —C(O)NR¹⁰R¹¹.

In some embodiments, R¹⁰ is selected from lower alkyl and hydrogen. In some embodiments, R¹⁰ is selected from optionally substituted alkyl, optionally substituted cycloalkyl, optionally substituted heterocycloalkyl, and optionally substituted aryl. In some embodiments, R¹⁰ is —(CR¹⁷R¹⁸)ₙR¹⁹, wherein R¹⁷ and R¹⁸ are independently selected from hydrogen, carboxy, optionally substituted aminocarbonyl, lower carboxy ester, and lower alkyl; n is 0, 1 or 2; and R¹⁹ is chosen from optionally substituted aryl and optionally substituted heteroaryl. In some embodiments, R¹⁰ is benzyl, thiophen-2-yl-ethyl, thiophen-3-yl-methyl, furan-2-yl-methyl, and furan-3-yl-methyl, each of which is optionally substituted. In some embodiments, R¹¹ is selected from lower alkyl and hydrogen.

In some embodiments, R¹⁰ and R¹¹, together with any intervening atoms, form an optionally substituted heterocycloalkyl. In some embodiments, R¹⁰ and R¹¹, together with any intervening atoms, form a substituted 3- to 7-membered nitrogen containing heterocycloalkyl which optionally further includes one or two additional heteroatoms chosen from N, O, S, S(O), S(O)₂, and P(O), wherein said 3- to 7-membered nitrogen containing heterocycloalkyl is substituted with a group —Y—R³⁰ and optionally substituted with a second group R³¹, wherein

Y is a bond or is selected from —NR¹⁰—, —NR¹¹SO₂—, —O—, —S—, —C(O)NR¹⁰—, and —S(O)₂R¹⁰—;

R³⁰ is selected from optionally substituted alkyl, optionally substituted cycloalkyl, optionally substituted heterocycloalkyl, optionally substituted aryl, and optionally substituted heteroaryl; and

R³¹ is selected from halogen, optionally substituted alkyl, optionally substituted alkenyl, optionally substituted alkynyl, optionally substituted cycloalkyl, optionally substituted heterocycloalkyl, optionally substituted aryl, optionally substituted heteroaryl, optionally substituted alkoxy, —OH, —SH, —NO₂, —NR¹⁰R¹¹, —C(O)NR¹⁰R¹¹, —C(O)OR¹³, —SO₂NR¹⁰R¹¹, —NR¹C(S)NR¹⁰R¹¹, —NR¹C(O)NR¹⁰R¹¹, —CN, —NR¹¹SO₂R¹⁴, and —NR¹¹CO₂R¹³.

In some embodiments, R¹⁰ and R¹¹, together with any intervening atoms, form a substituted 3- to 7-membered nitrogen containing heterocycloalkyl which optionally further includes one or two additional heteroatoms chosen from N, O, S, S(O), S(O)₂, and P(O), wherein said 3- to 7-membered nitrogen containing heterocycloalkyl is substituted with a group —Y—R³⁰ and optionally substituted with a second group R³¹, wherein

Y is a bond or is selected from —O—, —S—, —C(O)NR¹⁰—, and —S(O)₂R¹⁰—;

R³⁰ is selected from optionally substituted cycloalkyl, optionally substituted heterocycloalkyl, optionally substituted aryl, and optionally substituted heteroaryl; and

R³¹ is selected from halogen, optionally substituted alkyl, optionally substituted alkenyl, optionally substituted alkynyl, optionally substituted cycloalkyl, optionally substituted heterocycloalkyl, optionally substituted aryl, optionally substituted heteroaryl, optionally substituted alkoxy, —NO₂, —NR¹⁰R¹¹, —C(O)NR¹⁰R¹¹, —C(O)OR¹³, —SO₂NR¹⁰R¹¹, —NR¹¹C(S)NR¹⁰R¹¹, —NR¹¹C(O)NR¹⁰R¹¹, —CN, —NR¹¹SO₂R¹⁴, and —NR¹CO₂R³.

In some embodiments, Y is a bond or is selected from —NR¹⁰— and —O—. In some embodiments, Y is a bond or is —O—. In some embodiments, Y is a bond.

In some embodiments, R³⁰ is selected from optionally substituted aryl and optionally substituted heteroaryl. In some embodiments, R³⁰ is selected from phenyl, thiophen-2-yl, thiophen-3-yl, furan-2-yl, furan-3-yl, thiazol-2-yl, thiazol-4-yl, thiazol-5-yl, pyrazol-4-yl, imidazol-4-yl, and imidazol-2-yl. In some embodiments, R³⁰ is selected from phenyl, thiophen-2-yl, thiophen-3-yl, furan-2-yl, and furan-3-yl. In some embodiments, R³⁰ is phenyl. In some embodiments, R³⁰ is optionally substituted alkyl. In some embodiments, R³⁰ is optionally substituted lower alkyl. In some embodiments, R³⁰ is lower alkyl. In some embodiments, R³⁰ is methyl.

In some embodiments, R² is —C(O)NR¹⁰R¹¹ and R¹⁰ and R¹¹, together with any intervening atoms, form a pyrrolidinyl, piperidinyl, piperazinyl, 5,6-dihydropyridin-1(2H)-yl, 4,5-dihydro-1H-pyrazol-1-yl, 2,5-dihydro-1H-pyrrol-1-yl, or azetidinyl ring, wherein said ring is substituted with a group —Y—R³⁰ and optionally substituted with a second group R³¹ as described above.

In some embodiments, R² is lower alkyl substituted with —C(O)NR¹⁰R¹¹ and R¹⁰ and R¹¹, together with any intervening atoms, form a pyrrolidinyl, piperidinyl, piperazinyl, 5,6-dihydropyridin-1(2H)-yl, 4,5-dihydro-1H-pyrazol-1-yl, 2,5-dihydro-1H-pyrrol-1-yl, or azetidinyl ring, wherein said ring is substituted with a group —Y—R³⁰ and optionally substituted with a second group R³¹ as described above. In some embodiments, R² is —CH₂— substituted with —C(O)NR¹⁰R¹¹ and R¹⁰ and R¹¹, together with any intervening atoms, form a pyrrolidinyl, piperidinyl, piperazinyl, 5,6-dihydropyridin-1(2H)-yl, 4,5-dihydro-1H-pyrazol-1-yl, 2,5-dihydro-1H-pyrrol-1-yl, or azetidinyl ring, wherein said ring is substituted with a group —Y—R³⁰ and optionally substituted with a second group R³¹ as described above.

In some embodiments, R² is optionally substituted heteroaryl. In some embodiments, R² is isoxazol-5-yl or [1,2,4]oxadiazol-5-yl, each of which is optionally substituted. In some embodiments, R² is isoxazol-5-yl or [1,2,4]oxadiazol-5-yl, each of which is optionally substituted with a group chosen from optionally substituted aryl and optionally substituted alkyl. In some embodiments, R² is isoxazol-5-yl or [1,2,4]oxadiazol-5-yl, each of which is optionally substituted with a group chosen from optionally substituted phenyl, optionally substituted benzyl, and optionally substituted phenoxymethyl. In some embodiments, R² is isoxazol-5-yl or [1,2,4]oxadiazol-5-yl, each of which is optionally substituted with a group chosen from phenyl, benzyl, and phenoxymethyl.

In some embodiments, R³ is selected from optionally substituted alkyl and halogen. In some embodiments, R³ is selected from lower alkyl and halogen. In some embodiments, R³ is halogen. In some embodiments, R³ is selected from chlorine and bromine. In some embodiments, R³ is chlorine. In some embodiments, R³ is hydrogen.

In some embodiments, R⁴ is selected from hydrogen, optionally substituted alkyl, —NR¹¹SO₂R¹⁴, —NR¹¹C(O)NR¹⁰R¹¹, —NR¹¹CO₂R¹³ —S(O)NR¹⁰R¹¹, —NR¹¹C(O)NR¹⁰R¹¹, —CN, —NO₂, and —C(O)R¹². In some embodiments, R¹¹ is hydrogen. In some embodiments, R¹⁰ is selected from optionally substituted alkyl and optionally substituted cycloalkyl.

In some embodiments, R⁴ is selected from hydrogen and optionally substituted lower alkyl. In some embodiments, R⁴ is hydrogen.

In some embodiments, R⁴ is —CN.

In some embodiments, R⁵ is selected from optionally substituted cycloalkyl, optionally substituted aryl, optionally substituted heteroaryl, and optionally substituted heterocycloalkyl. In some embodiments, R⁵ is selected from optionally substituted cycloalkyl, optionally substituted aryl, and optionally substituted heteroaryl. In some embodiments, R⁵ is selected from optionally substituted aryl and optionally substituted heteroaryl. In some embodiments, R⁵ is selected from pyrid-3-yl, pyrazol-4-yl, phenyl, furan-2-yl, furan-3-yl, thiophen-2-yl, and thiophen-3-yl, each of which is optionally substituted. In some embodiments, R⁵ is selected from phenyl, furan-2-yl, furan-3-yl, thiophen-2-yl, and thiophen-3-yl, each of which is optionally substituted. In some embodiments, R⁵ is selected from phenyl, furan-2-yl, furan-3-yl, thiophen-2-yl, and thiophen-3-yl, each of which is optionally substituted with one or two groups chosen from lower alkyl, halogen, morpholinyl, trifluoromethyl, and lower alkoxy. In some embodiments, R⁵ is selected from phenyl, 3-fluorophenyl, furan-2-yl, furan-3-yl, thiophen-2-yl, and thiophen-3-yl.

In some embodiments, R⁶ is selected from hydrogen, halogen, optionally substituted alkyl, —OR¹⁵, —S(O)NR¹⁰R¹¹, —C(O)R¹², —NO₂, —C(O)NR¹⁰R¹¹, and —NR¹⁰R¹¹. In some embodiments, R⁶ is selected from hydrogen, halogen, optionally substituted alkyl, —S(O)NR¹⁰R¹¹, —C(O)R¹², —NO₂, —C(O)NR¹⁰R¹¹, and —NR¹⁰R¹¹. In some embodiments, R¹¹ is hydrogen. In some embodiments, R¹⁰ is selected from optionally substituted alkyl and optionally substituted cycloalkyl. In some embodiments, R¹⁰ and R¹¹, taken together with any intervening atoms, form an optionally substituted heterocycloalkyl ring.

In some embodiments, R⁶ is selected from hydrogen, halogen, and optionally substituted alkyl. In some embodiments, R⁶ is selected from hydrogen and halogen. In some embodiments, R⁶ is hydrogen.

In some embodiments, R⁷ is selected from halogen, optionally substituted alkyl, optionally substituted cycloalkyl, optionally substituted alkoxy, heterocycloalkyl, optionally substituted aryl, —SO₂NR¹⁰R¹¹, and —NR¹⁰R¹¹. In some embodiments, R⁷ is selected from halogen, optionally substituted alkyl, optionally substituted cycloalkyl, optionally substituted alkoxy, heterocycloalkyl, optionally substituted aryl, and —NR¹⁰R¹¹. In some embodiments, R⁷ is selected from optionally substituted alkyl, optionally substituted cycloalkyl, optionally substituted alkoxy, and —NR¹⁰R¹¹. In some embodiments, R⁷ is selected from optionally substituted alkyl, optionally substituted alkoxy, and —NR¹⁰R¹¹. In some embodiments, R⁷ is selected from optionally substituted lower alkoxy and optionally substituted lower alkyl.

In some embodiments, R⁷ is polyhalogenated lower alkoxy. In some embodiments, R⁷ selected from trifluoromethoxy and difluorochloromethoxy.

In some embodiments, R⁷ is polyhalogenated lower alkyl. In some embodiments, R⁷ is polyhalogenated methyl. In some embodiments, R⁷ is selected from trifluoromethyl and difluorochloromethyl. In some embodiments, R⁷ is trifluoromethyl.

In some embodiments, R⁷ is —NR¹⁰R¹¹. In some embodiments, R¹¹ is hydrogen. In some embodiments, R¹⁰ is optionally substituted lower alkyl. In some embodiments, R¹⁰ is methyl. In some embodiments, R¹⁰ is 2-hydroxyethyl.

Also provided is at least one chemical entity selected from compounds of Formula (I):

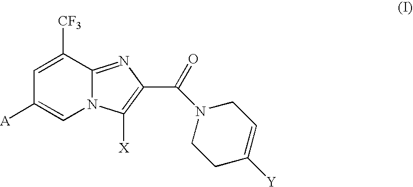

or a pharmaceutically acceptable salt thereof, wherein:
A is selected from the group consisting of furanyl, thiazolyl, imidazolyl, thienyl, dihydropyrrolyl, cyclopentenyl, phenyl, ethenyl, cyclopropylvinyl, and halo;
X is selected from the group consisting of hydrogen, halo, cyclopropyl, hydroxymethyl, hydroxyethyl, and hydroxy; and
Y is selected from the group consisting of aryl, heteroaryl, and heteroaryl substituted with 1 to 3 groups independently selected from the group consisting of halo, hydroxy, trifluoromethyl, methyl, cyano, methoxy, and ethoxy.

In some embodiments, the compound of Formula (I) is chosen from the compounds set forth in Table 1.

TABLE 1

| Compound | | |
|---|---|---|
| Number | Name | Structure |
| 111 | (3-Chloro-6-furan-3-yl-8- trifluoromethyl-imidazo[1,2- a]pyridin-2-yl)-[4-(1H-imidazol- 2-yl)-3,6-dihydro-2H-pyridin-1- yl]-methanone | 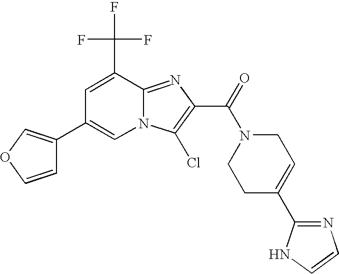 |
| 115 | (3-Chloro-6-furan-3-yl-8- trifluoromethyl-imidazo[1,2- a]pyridin-2-yl)-(3-ethoxy-3′,6′- dihydro-2′H-[2,4′]bipyridinyl-1′- yl)-methanone | 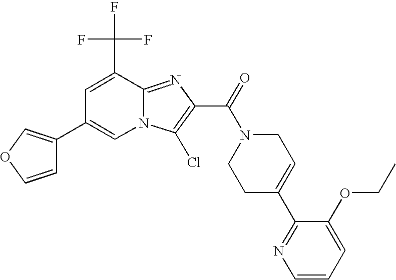 |
| 118 | (3 -Chloro-6-thiazol-2-yl-8- trifluoromethyl-imidazo[1,2- a]pyridin-2-yl)-(3-fluoro-3′,6′- dihydro-2′H-[2,4′]bipyridinyl-1′- yl)-methanone | 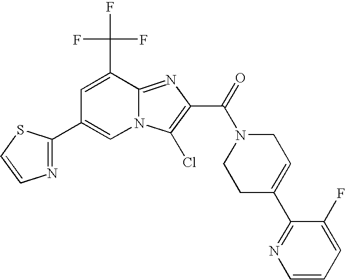 |
| 119 | (3-Chloro-6-thiazol-5-yl-8- trifluoromethyl-imidazo[1,2- a]pyridin-2-yl)-(3-fluoro-3′,6′- dihydro-2′H-[2,4′]bipyridinyl-1′- yl)-methanone | 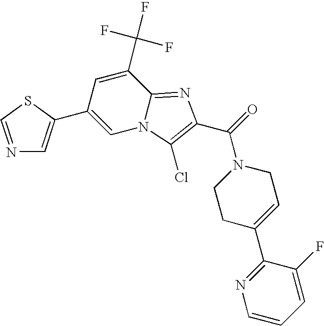 |
| 120 | (3-Chloro-6-thiazol-4-yl-8- trifluoromethyl-imidazo[1,2- a]pyridin-2-yl)-(3-fluoro-3′,6′- dihydro-2′H-[2,4′]bipyridinyl-1′- yl)-methanone | 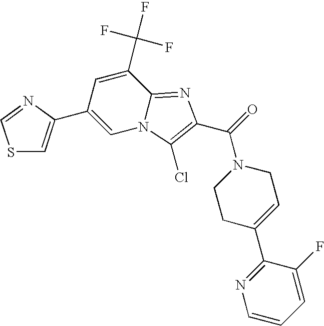 |
| 121 | [3-Chloro-6-(2,5-dihydro-1H- pyrrol-3-yl)-8-trifluoromethyl- imidazo[1,2-a]pyridin-2-yl]-(3- fluoro-3′,6′-dihydro-2′H- [2,4′]bipyridinyl-1′-yl)- methanone | 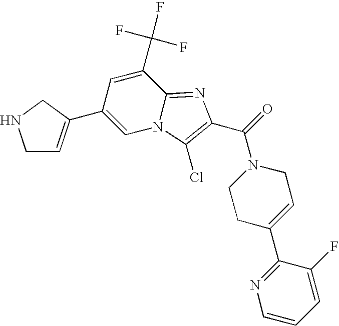 |
| 122 | [3-Chloro-6-(1H-imidazol-4-yl)- 8-trifluoromethyl-imidazo[1,2- a]pyridin-2-yl]-(3-fluoro-3′,6′- dihydro-2′H-[2,4′]bipyridinyl-1′- yl)-methanone | 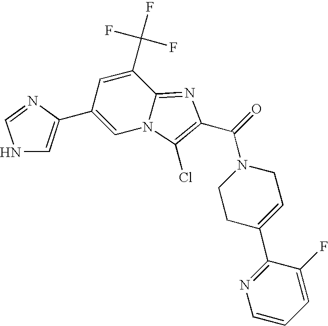 |
| 123 | (3-Chloro-6-thiophen-2-yl-8- trifluoromethyl-imidazo[1,2- a]pyridin-2-yl)-(3-fluoro-3′,6′- dihydro-2′H-[2,4′]bipyridinyl-1′- yl)-methanone | 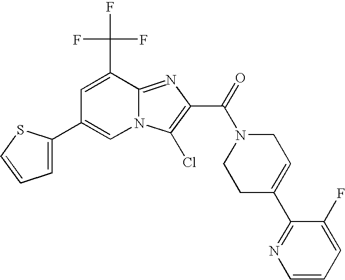 |
| 124 | (3-Chloro-6-phenyl-8- trifluoromethyl-imidazo[1,2- a]pyridin-2-yl)-(3-fluoro-3′,6′- dihydro-2′H-[2,4′]bipyridinyl-1′- yl)-methanone | 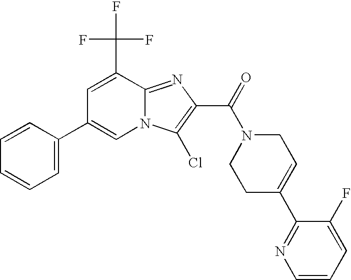 |
| 125 | (6-Bromo-3-chloro-8- trifluoromethyl-imidazo[1,2- a]pyridin-2-yl)-(3-fluoro-3′,6′- dihydro-2′H-[2,4′]bipyridinyl-1′- yl)-methanone | 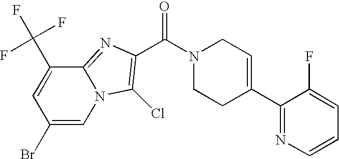 |
| 126 | (3-Chloro-8-trifluoromethyl-6- vinyl-imidazo[1,2-a]pyridin-2- yl)-(3-fluoro-3′,6′-dihydro-2′H- [2,4′]bipyridinyl-1′-yl)- methanone | 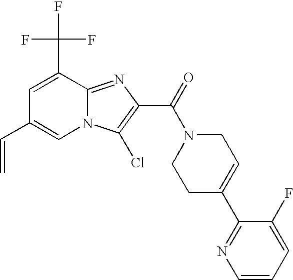 |
| 127 | [3-Chloro-6-(2-cyclopropyl- vinyl)-8-trifluoromethyl- imidazo[1,2-a]pyridin-2-yl]-(3- fluoro-3′,6′-dihydro-2′H- [2,4′]bipyridinyl-1′-yl)- methanone | 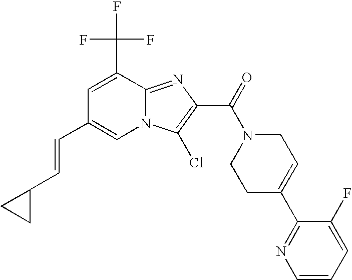 |
| 149 | (3-Cyclopropyl-6-furan-3-yl-8- trifluoromethyl-imidazo[1,2- a]pyridin-2-yl)-(3-fluoro-3′,6′- dihydro-2′H-[2,4′]bipyridinyl-1′- yl)-methanone | 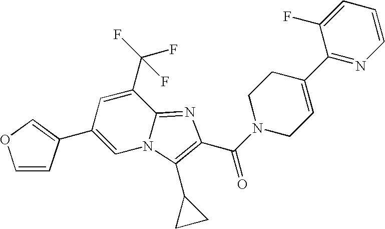 |
| 151 | (3-Chloro-6-cyclopent-1-enyl-8- trifluoromethyl-imidazo[1,2- a]pyridin-2-yl)-(3-fluoro-3′,6′- dihydro-2′H-[2,4′]bipyridinyl-1′- yl)-methanone | 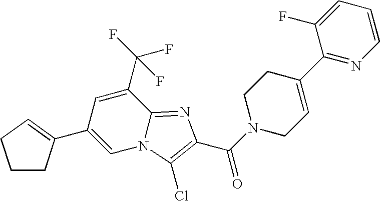 |
| 171 | (3-Fluoro-3′,6′-dihydro-2′H- [2,4′]bipyridinyl-1′-yl)-(6-furan- 3-yl-3-hydroxymethyl-8- trifluoromethyl-imidazo[1,2- a]pyridin-2-yl)-methanone | 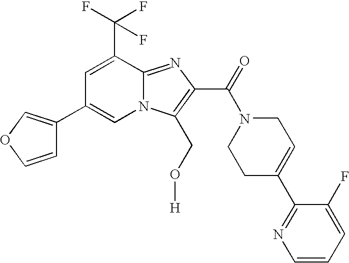 |
| 172 | (3-Fluoro-3′,6′-dihydro-2′H- [2,4′]bipyridinyl-1′-yl)-[6-furan- 3-yl-3-(1-hydroxy-ethyl)-8- trifluoromethyl-imidazo[1,2- a]pyridin-2-yl]-methanone | 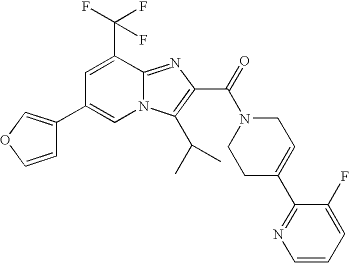 |
| 201 | (3-Chloro-6-furan-3-yl-8- trifluoromethyl-imidazo[1,2- a]pyridin-2-yl)-(3-hydroxy-3′,6′- dihydro-2′H-[2,4′]bipyridinyl-1′- yl)-methanone | 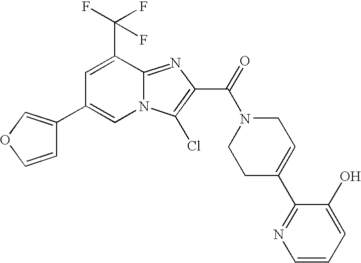 |
| 202 | (3-Chloro-3′,6′-dihydro-2′H- [2,4′]bipyridinyl-1′-yl)-(3- chloro-6-furan-3-yl-8- trifluoromethyl-imidazo[1,2- a]pyridin-2-yl)-methanone | 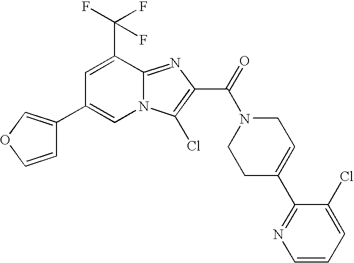 |
| 203 | (3-Chloro-6-furan-3-yl-8- trifluoromethyl-imidazo[1,2- a]pyridin-2-yl)-(5-fluoro-3′,6′- dihydro-2′H-[3,4′]bipyridinyl-1′- yl)-methanone | 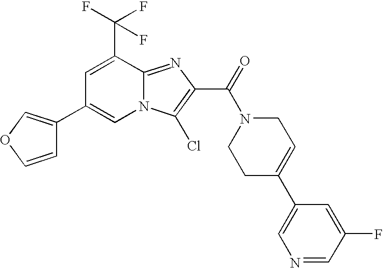 |
| 204 | (3-Chloro-6-furan-3-yl-8- trifluoromethyl-imidazo[1,2- a]pyridin-2-yl)-(6-fluoro-3′,6′- dihydro-2′H-[2,4′]bipyridinyl-1′- yl)-methanone | 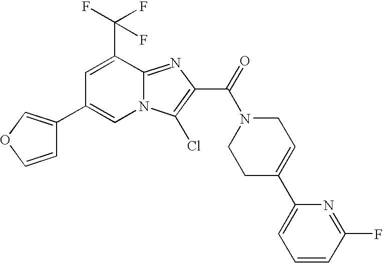 |
| 205 | (3-Chloro-6-furan-3-yl-8- trifluoromethyl-imidazo[1,2- a]pyridin-2-yl)-(3- trifluoromethyl-3′,6′-dihydro- 2′H-[2,4′]bipyridinyl-1′-yl)- methanone | 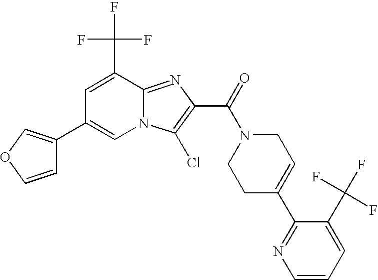 |
| 206 | (3-Chloro-6-furan-3-yl-8- trifluoromethyl-imidazo[1,2- a]pyridin-2-yl)-(3-fluoro-4- methyl-3′,6′-dihydro-2′H- [2,4′]bipyridinyl-1′-yl)- methanone | 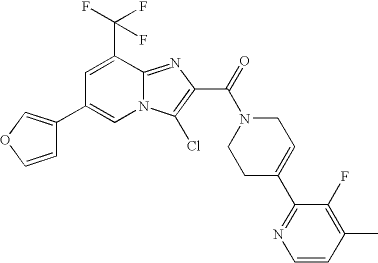 |
| 207 | (3-Chloro-6-furan-3-yl-8- trifluoromethyl-imidazo[1,2- a]pyridin-2-yl)-(3 -fluoro-6- methyl-3′,6′-dihydro-2′H- [2,4′]bipyridinyl-1′-yl)- methanone | 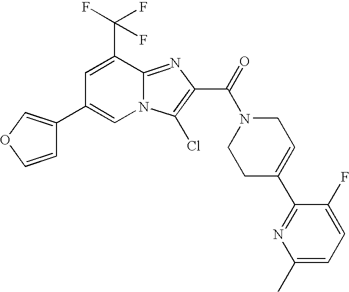 |
| 208 | (3-Chloro-6-furan-3-yl-8- trifluoromethyl-imidazo[1,2- a]pyridin-2-yl)-[4-(5-fluoro- pyrimidin-4-yl)-3,6-dihydro-2H- pyridin-1-yl]-methanone | 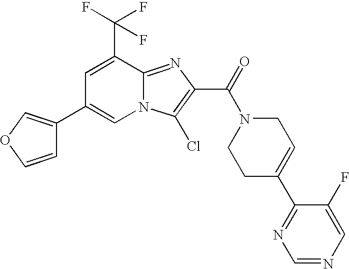 |
| 209 | (3-Chloro-6-furan-3-yl-8- trifluoromethyl-imidazo[1,2- a]pyridin-2-yl)-(4-fluoro-3′,6′- dihydro-2′H-[2,4′]bipyridinyl-1′- yl)-methanone | 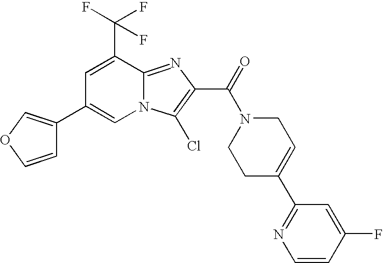 |
| 210 | (3-Chloro-6-furan-3-yl-8- trifluoromethyl-imidazo[1,2- a]pyridin-2-yl)-(3′,6′-dihydro- 2′H-[2,4′]bipyridinyl-1′-yl)- methanone | 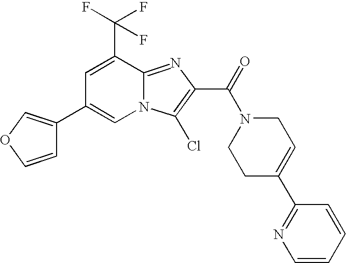 |
| 211 | 1′-(3-Chloro-6-furan-3-yl-8- trifluoromethyl-imidazo[1,2- a]pyridine-2-carbonyl)-1′,2′,3′,6′- tetrahydro-[2,4′]bipyridinyl-3- carbonitrile | 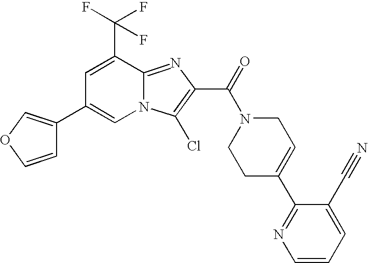 |
| 214 | (3-Chloro-6-furan-3-yl-8- trifluoromethyl-imidazo[1,2- a]pyridin-2-yl)-(3-fluoro-1- hydroxy-3′,6′-dihydro-2′H- [2,4′]bipyridinyl-1′-yl)- methanone | 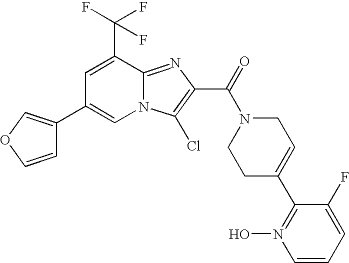 |

In a preferred embodiment, the compounds of Formula (I) do not include any of the compounds in the following table:

| Compound Name | Structure |
|---|---|
| [3-chloro-6-(furan-3-yl)-8- (trifluoromethyl)imidazo[1,2-a]pyridin-2- yl][4-(1H-imidazol-4-yl)-3,6- dihydropyridin-1(2H)-yl]methanone | 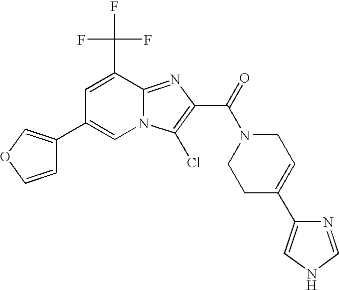 |
| (3-Chloro-6-furan-3-yl-8-trifluoromethyl- imidazo[1,2-a]pyridin-2-yl)-(4-thiazol-2- yl-3,6-dihydro-2H-pyridin-1-yl)- methanone | 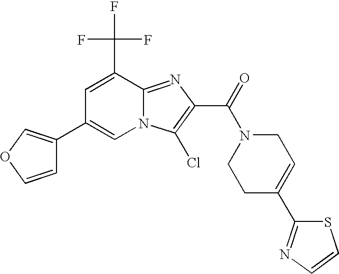 |
| (3-Chloro-6-furan-3-yl-8-trifluoromethyl- imidazo[1,2-a]pyridin-2-yl)-(4-thiazol-4- yl-3,6-dihydro-2H-pyridin-1-yl)- methanone | 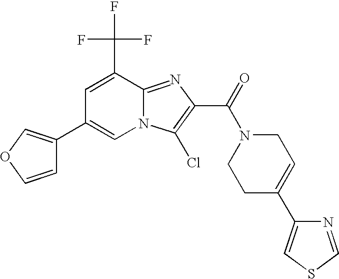 |
| (3-Chloro-6-furan-3-yl-8-trifluoromethyl- imidazo[1,2-a]pyridin-2-yl)-(4-thiazol-5- yl-3,6-dihydro-2H-pyridin-1-yl)- methanone | 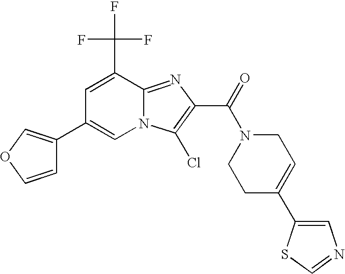 |
| [3-chloro-6-(furan-3-yl)-8- (trifluoromethyl)imidazo[1,2-a]pyridin-2- yl](3-fluoro-3′,6′-dihydro-2,4′-bipyridin- 1′(2′H)-yl)methanone | 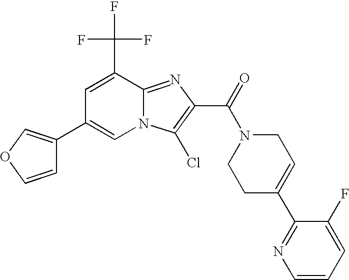 |
| [3-chloro-6-(furan-3-yl)-8- (trifluoromethyl)imidazo[1,2-a]pyridin-2- yl](3′-fluoro-3,6-dihydro-4,4′-bipyridin- 1(2H)-yl)methanone | 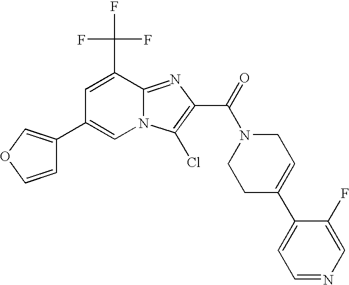 |
| (3-Chloro-6-furan-3-yl-8-trifluoromethyl- imidazo[1,2-a]pyridin-2-yl)-(4-furan-3-yl- 3,6-dihydro-2H-pyridin-1-yl)-methanone | 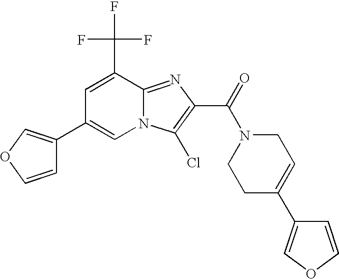 |
| (3-Chloro-6-furan-3-yl-8-trifluoromethyl- imidazo[1,2-a]pyridin-2-yl)-[4-(1-methyl- 1H-pyrazol-4-yl)-3,6-dihydro-2H-pyridin- 1-yl]-methanone | 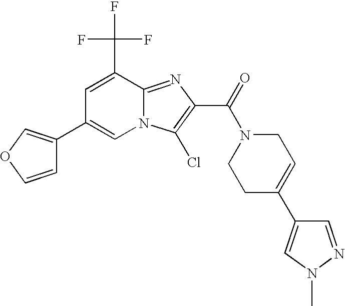 |
| (3-Chloro-6-furan-3-yl-8-trifluoromethyl- imidazo[1,2-a]pyridin-2-yl)-[4-(1H- pyrazol-4-yl)-3,6-dihydro-2H-pyridin-1- yl]-methanone | 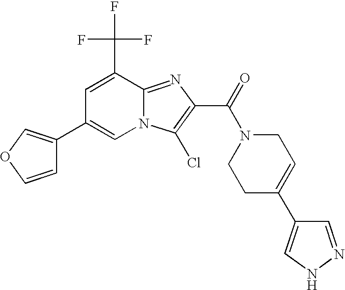 |
| (3-Chloro-6-furan-3-yl-8-trifluoromethyl- imidazo[1,2-a]pyridin-2-yl)-(2-fluoro- 3′,6′-dihydro-2′H-[3,4′]bipyridinyl-1′-yl)- methanone | 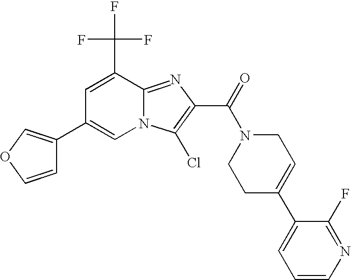 |
| (3-Chloro-6-furan-3-yl-8-trifluoromethyl- imidazo[1,2-a]pyridin-2-yl)-(4-isoxazol-4- yl-3,6-dihydro-2H-pyridin-1-yl)- methanone | 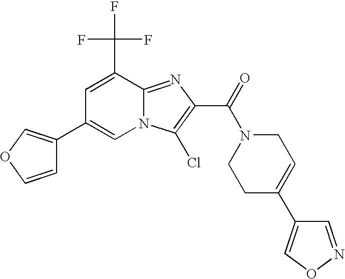 |
| [3-chloro-6-(furan-3-yl)-8- (trifluoromethyl)imidazo[1,2-a]pyridin-2- yl][4-(1H-pyrrol-3-yl)-3,6- dihydropyridin-1(2H)-yl]methanone | 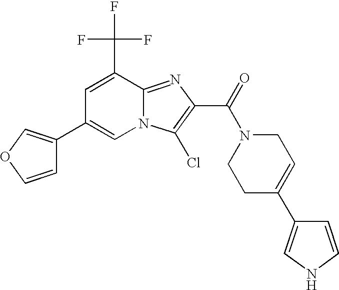 |
| [3-chloro-6-(furan-3-yl)-8- (trifluoromethyl)imidazo[1,2-a]pyridin-2- yl][4-(1H-pyrazol-5-yl)-3,6- dihydropyridin-1(2H)-yl]methanone | 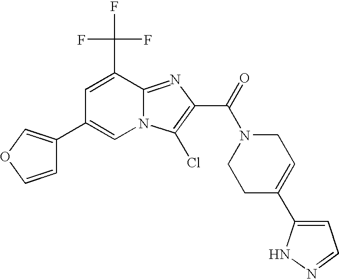 |
| [3-chloro-6-(furan-3-yl)-8- (trifluoromethyl)imidazo[1,2-a]pyridin-2- yl][4-(1-methyl-1H-pyrazol-5-yl)-3,6- dihydropyridin-1(2H)-yl]methanone | 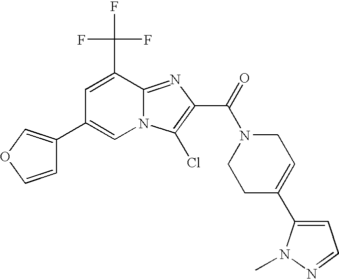 |
| [3-chloro-6-(furan-3-yl)-8- (trifluoromethyl)imidazo[1,2-a]pyridin-2- yl](2′-fluoro-3,6-dihydro-4,4′-bipyridin- 1(2H)-yl)methanone | 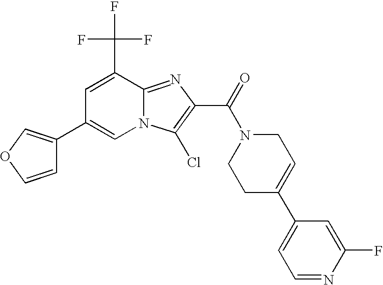 |
| (3-Chloro-6-furan-3-yl-8-trifluoromethyl- imidazo[1,2-a]pyridin-2-yl)-(4-phenyl- 3,6-dihydro-2H-pyridin-1-yl)-methanone | 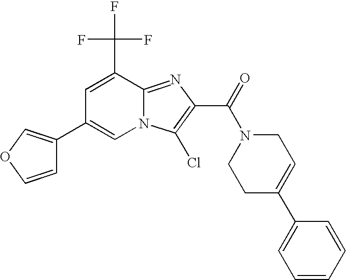 |
| (3-Chloro-6-furan-3-yl-8-trifluoromethyl- imidazo[1,2-a]pyridin-2-yl)-(4-thiophen- 2-yl-3,6-dihydro-2H-pyridin-1-yl)- methanone | 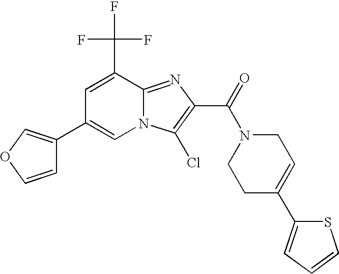 |
| (3-Chloro-6-furan-3-yl-8-trifluoromethyl- imidazo[1,2-a]pyridin-2-yl)-[4-(2-methyl- thiazol-4-yl)-3,6-dihydro-2H-pyridin-1- yl]-methanone | 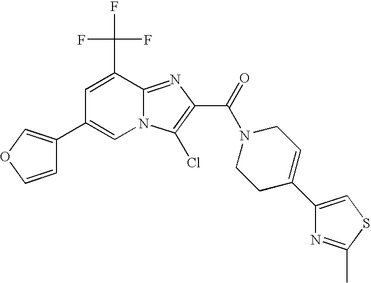 |
| (3-Chloro-6-furan-3-yl-8-trifluoromethyl- imidazo[1,2-a]pyridin-2-yl)-(2,6-difluoro- 3′,6′-dihydro-2′H-[3,4′]bipyridinyl-1′-yl)- methanone | 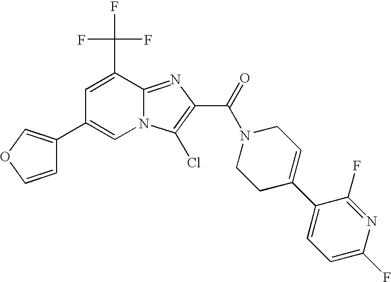 |
| [3-chloro-6-(furan-3-yl)-8- (trifluoromethyl)imidazo[1,2-a]pyridin-2- yl][4-(pyrimidin-5-yl)-3,6-dihydropyridin- 1(2H)-yl]methanone | 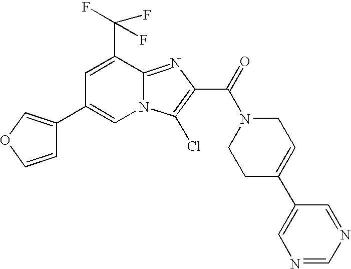 |
| [3-chloro-6-(furan-3-yl)-8- (trifluoromethyl)imidazo[1,2-a]pyridin-2- yl][4-(1,6-dihydropyrimidin-5-yl)-3,6- dihydropyridin-1(2H)-yl]methanone | 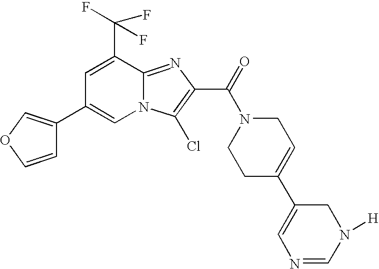 |
| [3-chloro-6-(furan-3-yl)-8- (trifluoromethyl)imidazo[1,2-a]pyridin-2- yl][4-(5-methyl-1H-pyrazol-4-yl)-3,6- dihydropyridin-1(2H)-yl]methanone | 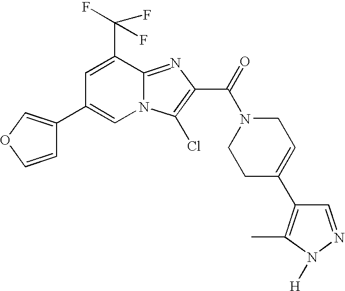 |

Also provided is at least one chemical entity selected from compounds of Formula (II):

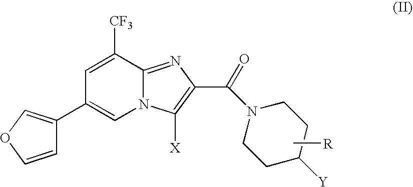

or a pharmaceutically acceptable salt thereof, wherein:
X is selected from the group consisting of hydrogen, halo, hydroxymethyl, and hydroxy;
R is selected from the group consisting of hydrogen, cyano, hydroxy, and halo; and
Y is selected from the group consisting of aryl, aryl substituted with halo, heteroaryl, and heteroaryl substituted with halo.

In some embodiments, the compounds of Formula (II) is chosen from the compounds set forth in Table 2.

TABLE 2

| Compound | | |
|---|---|---|
| Number | Name | Structure |
| 102 | (3-Chloro-6-furan-3-yl-8- trifluoromethyl-imidazo[1,2- a]pyridin-2-yl)-[4-(2-fluoro- phenyl)-3-hydroxy-piperidin-1- yl]-methanone | 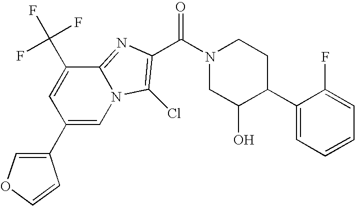 |
| 197 | (3-Chloro-6-furan-3-yl-8- trifluoromethyl-imidazo[1,2- a]pyridin-2-yl)-(4-imidazol-1-yl- piperidin-1-yl)-methanone | 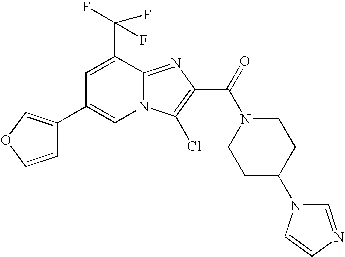 |
| 219 | (3-Chloro-6-furan-3-yl-8- trifluoromethyl-imidazo[1,2- a]pyridin-2-yl)-(3-fluoro- 3′,4′,5′,6′-tetrahydro-2′H- [2,4′]bipyridinyl-1′-yl)- methanone | 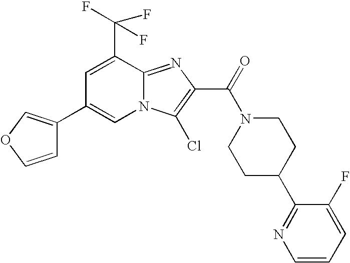 |
| 220 | 1′-(3-Chloro-6-furan-3-yl-8- trifluoromethyl-imidazo[1,2- a]pyridine-2-carbonyl)-3-fluoro- 2′,3′,5′,6′-tetrahydro-1′H- [2,4′]bipyridinyl-4′-carbonitrile | 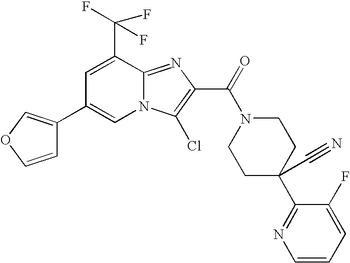 |
| 221 | 1-(3-Chloro-6-furan-3-yl-8- trifluoromethyl-imidazo[1,2- a]pyridine-2-carbonyl)-4- thiophen-2-yl-piperidine-4- carbonitrile | 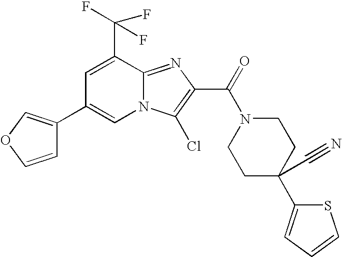 |
| 222 | (3-Chloro-6-furan-3-yl-8- trifluoromethyl-imidazo[1,2- a]pyridin-2-yl)-(3-fluoro-4′- hydroxy-3′,4′,5′,6′-tetrahydro- 2′H-[2,4′]bipyridinyl-1′-yl)- methanone | 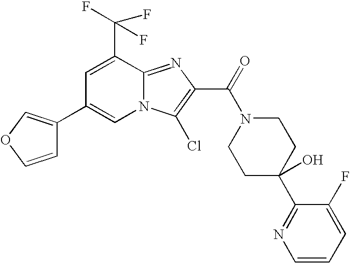 |
| 223 | (3-Chloro-6-furan-3-yl-8- trifluoromethyl-imidazo[1,2- a]pyridin-2-yl)-(3,4′-difluoro- 3′,4′,5′,6′-tetrahydro-2′H- [2,4′]bipyridinyl-1′-yl)- methanone | 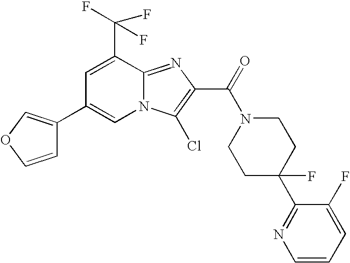 |
| 224 | (3-Chloro-6-furan-3-yl-8- trifluoromethyl-imidazo[1,2- a]pyridin-2-yl)-(4-hydroxy-4- thiazol-2-yl-piperidin-1-yl)- methanone | 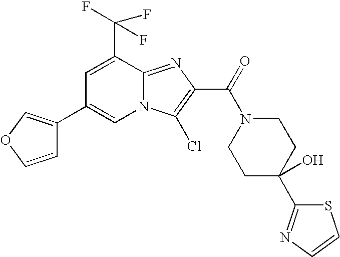 |

In a preferred embodiment, the compounds of Formula (II) do not include any of the compounds in the following table:

| Compound Name | Structure |
|---|---|
| (3-Chloro-6-furan-2-yl-8- trifluoromethyl-imidazo[1,2-a]pyridin- 2-yl)-(4-phenyl-piperidin-1-yl)- methanone | 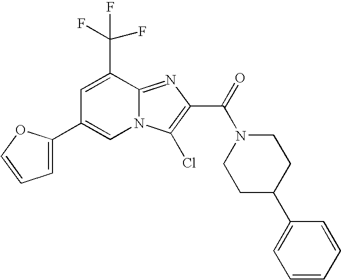 |
| (3-Chloro-6-furan-3-yl-8- trifluoromethyl-imidazo[1,2-a]pyridin- 2-yl)-[4-(2-fluoro-phenyl)-piperidin-1- yl]-methanone | 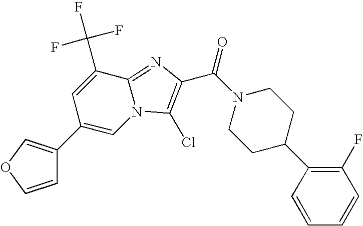 |
| (3-Chloro-6-furan-3-yl-8- trifluoromethyl-imidazo[1,2-a]pyridin- 2-yl)-[4-(3-fluoro-phenyl)-piperidin-1- yl]-methanone | 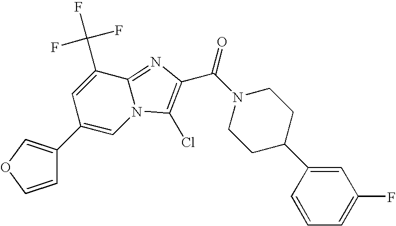 |
| (3-Chloro-6-furan-3-yl-8- trifluoromethyl-imidazo[1,2-a]pyridin- 2-yl)-[4-(4-fluoro-phenyl)-piperidin-1- yl]-methanone | 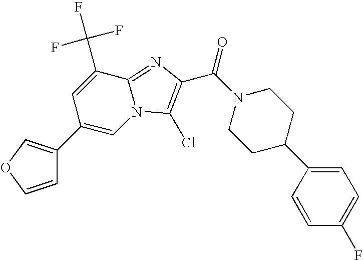 |
| (3-Chloro-6-furan-3-yl-8- trifluoromethyl-imidazo[1,2-a]pyridin- 2-yl)-(4-phenyl-piperidin-1-yl)- methanone | 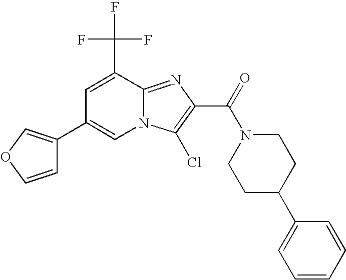 |
| (3-Chloro-6-furan-3-yl-8- trifluoromethyl-imidazo[1,2-a]pyridin- 2-yl)-[4-(2-chloro-phenyl)-piperidin-1- yl]-methanone | 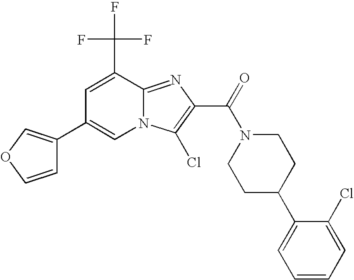 |
| (3-Chloro-6-furan-3-yl-8- trifluoromethyl-imidazo[1,2-a]pyridin- 2-yl)-(4-o-tolyl-piperidin-1-yl)- methanone | 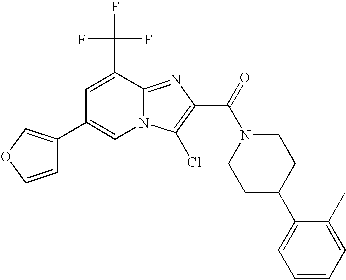 |
| (3-Chloro-6-furan-3-yl-8- trifluoromethyl-imidazo[1,2-a]pyridin- 2-yl)-(4-thiazol-2-yl-piperidin-1-yl)- methanone | 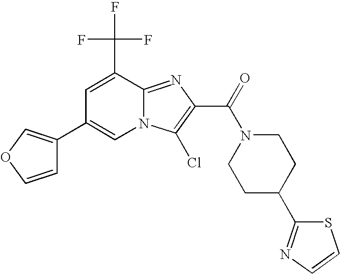 |
| (3-Chloro-6-furan-3-yl-8- trifluoromethyl-imidazo[1,2-a]pyridin- 2-yl)-(4-thiazol-4-yl-piperidin-1-yl)- methanone | 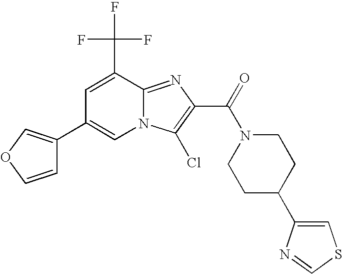 |
| [3-chloro-6-(furan-3-yl)-8- (trifluoromethyl)imidazo[1,2-a]pyridin- 2-yl][4-(thiophen-2-yl)piperidin-1- yl]methanone | 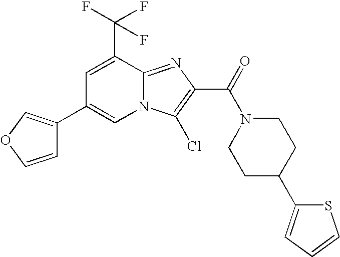 |
| 1-(3-Chloro-6-furan-3-yl-8- trifluoromethyl-imidazo[1,2- a]pyridine-2-carbonyl)-4-phenyl- piperidine-4-carbonitrile | 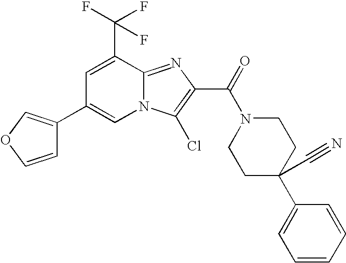 |
| (4-Benzoimidazol-1-yl-piperidin-1-yl)- (3-chloro-6-furan-3-yl-8- trifluoromethyl-imidazo[1,2-a]pyridin- 2-yl)-methanone | 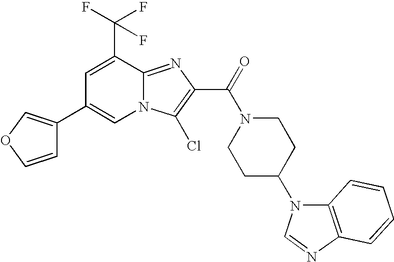 |

Also provided is at least one chemical entity selected from compounds of Formula (III):

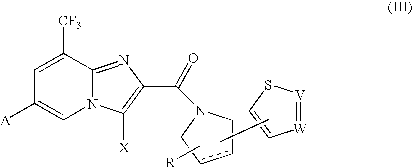

or a pharmaceutically acceptable salt thereof, wherein:
[symbol] represents a single or double bond;
X is selected from the group consisting of hydrogen, halo, cyclopropyl, hydroxymethyl, and hydroxy;
R is selected from the group consisting of hydrogen, halo, cyano, and hydroxy;
one of W and V is CH and the other is N; and
A is selected from the group consisting of furanyl, thiazolyl, imidazolyl, thienyl, pyrazolyl, pyridyl, dihydropyridyl, dihydropyrrolyl, cyclopentenyl, cyclohexenyl, phenyl, and halo.

In some embodiments, the compounds of Formula (III) is chosen from the compound set forth in Table 3.

TABLE 3

| Compound | | |
|---|---|---|
| Number | Name | Structure |
| 105 | [3-Chloro-6-(1H-pyrazol-4-yl)-8- trifluoromethyl-imidazo [1,2- a]pyridin-2-yl]-(3-thiazol-5-yl- 2,5-dihydro-pyrrol-1-yl)- methanone | 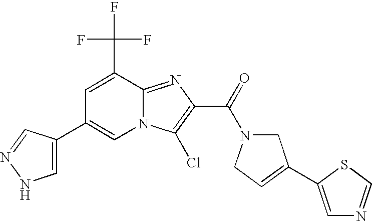 |
| 106 | (3-Chloro-6-furan-3-yl-8- trifluoromethyl-imidazo[1,2- a]pyridin-2-yl)-(3-thiazol-5-yl- 2,5-dihydro-pyrrol-1-yl)- methanone | 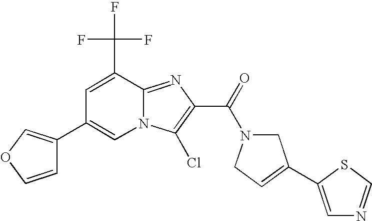 |
| 107 | [3-Chloro-6-(1H-pyrazol-4-yl)-8- trifluoromethyl-imidazo [1,2- a]pyridin-2-yl]-(3-isothiazol-4- yl-2,5-dihydro-pyrrol-1-yl)- methanone | 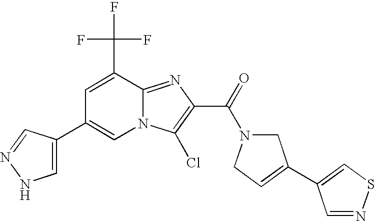 |
| 148 | (3-Cyclopropyl-6-furan-3-yl-8- trifluoromethyl-imidazo[1,2- a]pyridin-2-yl)-(3-thiazol-2-yl- 2,5-dihydro-pyrrol-1-yl)- methanone | 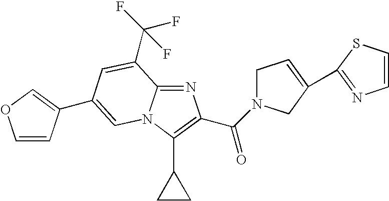 |
| 150 | (3-Chloro-6-cyclopent-1-enyl-8- trifluoromethyl-imidazo[1,2- a]pyridin-2-yl)-(3-thiazol-2-yl- 2,5-dihydro-pyrrol-1-yl)- methanone | 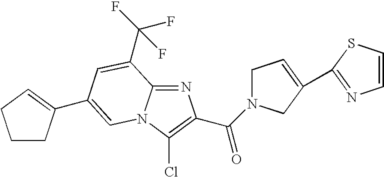 |
| 170 | (6-Furan-3-yl-3-hydroxymethyl- 8-trifluoromethyl-imidazo[1,2- a]pyridin-2-yl)-(3-thiazol-2-yl- 2,5-dihydro-pyrrol-1-yl) methanone | 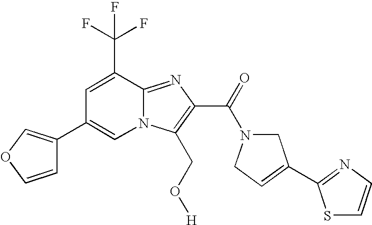 |
| 173 | [3-Hydroxymethyl-6-(1H- pyrazol-4-yl)-8-trifluoromethyl- imidazo[1,2-a]pyridin-2-yl]-(3- thiazol-2-yl-2,5-dihydro-pyrrol- 1-yl)-methanone | 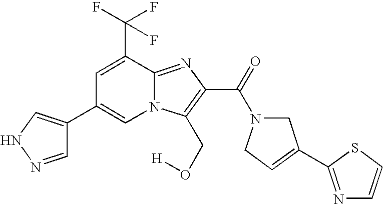 |
| 215 | (3-Chloro-6-phenyl-8- trifluoromethyl-imidazo[1,2- a]pyridin-2-yl)-(3-thiazol-2-yl- 2,5-dihydro-pyrrol-1-yl)- methanone | 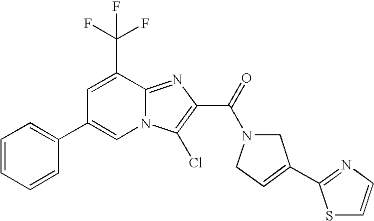 |
| 216 | (3-Chloro-6-pyridin-3-yl-8- trifluoromethyl-imidazo[1,2- a]pyridin-2-yl)-(3-thiazol-2-yl- 2,5-dihydro-pyrrol-1-yl)- methanone | 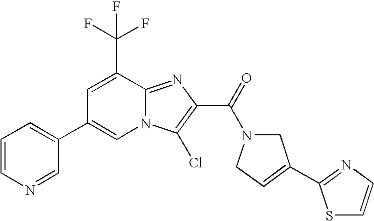 |
| 217 | (3-Chloro-6-cyclohex-1-enyl-8- trifluoromethyl-imidazo[1,2- a]pyridin-2-yl)-(3-thiazol-2-yl- 2,5-dihydro-pyrrol-1-yl)- methanone | 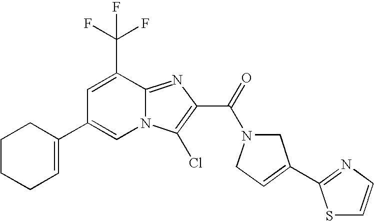 |
| 218 | [3-Chloro-6-(1,2,3,6-tetrahydro- pyridin-4-yl)-8-trifluoromethyl- imidazo[1,2-a]pyridin-2-yl]-(3- thiazol-2-yl-2,5-dihydro-pyrrol- 1-yl)-methanone | 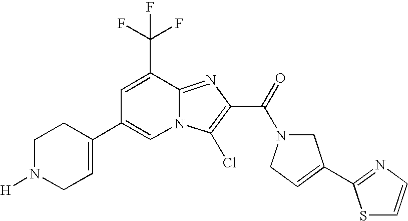 |

In a preferred embodiment, the compounds of Formula (III) do not include any of the compounds in the following table:

| Compound Name | Structure |
|---|---|
| (3-Chloro-6-furan-3-yl-8-trifluoromethyl- imidazo[1,2-a]pyridin-2-yl)-(3-thiazol-2-yl- 2,5-dihydro-pyrrol-1-yl)-methanone | 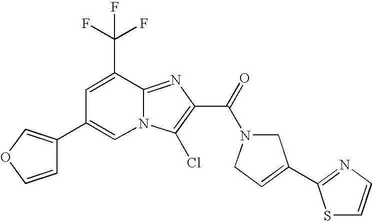 |
| (3-Chloro-6-furan-3-yl-8-trifluoromethyl- imidazo[1,2-a]pyridin-2-yl)-(3-thiazol-2-yl- pyrrolidin-1-yl)-methanone | 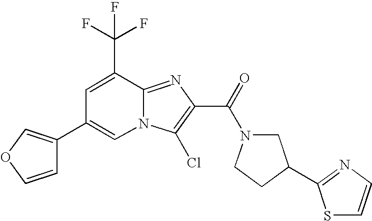 |
| [3-Bromo-6-(1H-pyrazol-4-yl)-8- trifluoromethyl-imidazo[1,2-a]pyridin-2-yl]- (3-thiazol-2-yl-pyrrolidin-1-yl)-methanone | 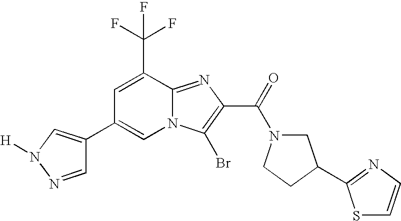 |
| [3-Bromo-6-(1H-pyrazol-4-yl)-8- trifluoromethyl-imidazo[1,2-a]pyridin-2-yl]- (3-thiazol-2-yl-2,5-dihydro-pyrrol-1-yl)- methanone | 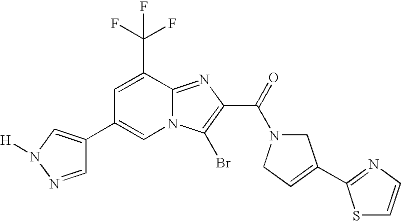 |
| [3-chloro-6-(1H-pyrazol-4-yl)-8- (trifluoromethyl)imidazo[1,2-a]pyridin-2- yl][3-(1,3-thiazol-2-yl)-2,5-dihydro-1H- pyrrol-1-yl]methanone | 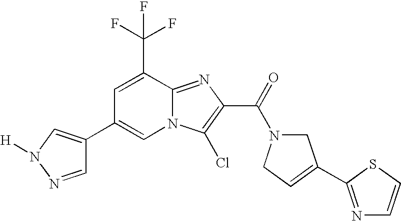 |
| [6-(1H-pyrazol-4-yl)-8- (trifluoromethyl)imidazo [1,2-a]pyridin-2- yl][3-(1,3-thiazol-2-yl)-2,5-dihydro-1H- pyrrol-1-yl]methanone | 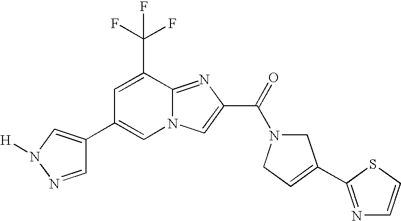 |
| [3-chloro-6-(1H-pyrazol-4-yl)-8- (trifluoromethyl)imidazo[1,2-a]pyridin-2- yl][3-(1,3-thiazol-4-yl)-2,5-dihydro-1H- pyrrol-1-yl]methanone | 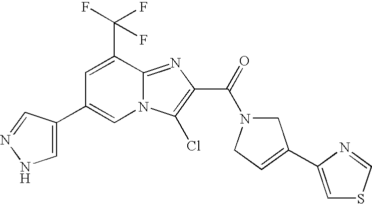 |

Also provided is at least one chemical entity selected from compounds of Formula (IV):

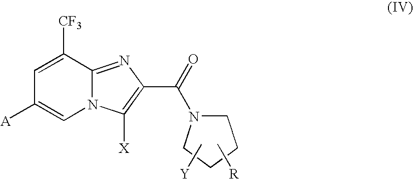

A is heteroaryl;
X is selected from the group consisting of hydrogen, halo, hydroxymethyl, and hydroxy;
R is selected from the group consisting of hydrogen, halo, cyano, and hydroxy; and
Y is selected from the group consisting of ethoxy, —O(CH₂)₂OCH₂CH₃, —NHR¹, and —NHC(O)R², wherein R¹ is selected from the group consisting of aryl, heteroaryl, and aryl substituted with alkoxy, and R² is selected from the group consisting of ethenyl, C₁-C₃ alkoxy substituted with halo, and C₁-C₃ alkyl substituted with 1 to 3 halo.

In some embodiments, the compounds of Formula (IV) is chosen from the compounds set forth in Table 4.

TABLE 4

| Compound | | |
|---|---|---|
| Number | Name | Structure |
| 162 | N-[1-(3-Chloro-6-furan-3-yl-8- trifluoromethyl-imidazo[1,2- a]pyridine-2-carbonyl)- pyrrohdin-3-yl]-2,2,2-trifluoro- acetamide | 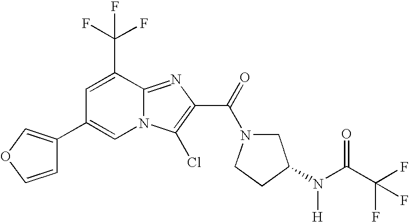 |
| 167 | N-[1-(3-Chloro-6-furan-3-yl-8- trifluoromethyl-imidazo[1,2- a]pyridine-2-carbonyl)- pyrrolidin-3-yl]-acrylamide | 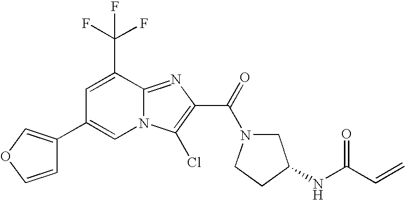 |
| 178 | (3-Chloro-6-furan-3-yl-8- trifluoromethyl-imidazo[1,2- a]pyridin-2-yl)-[3-(3-methoxy- phenylamino)-pyrrolidin-1-yl]- methanone | 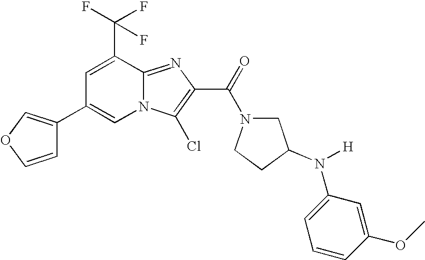 |
| 179 | [3-Chloro-6-(1H-pyrazol-4-yl)-8- trifluoromethyl-imidazo[1,2- a]pyridin-2-yl]-(3-phenylamino- pyrrolidin-1-yl)-methanone | 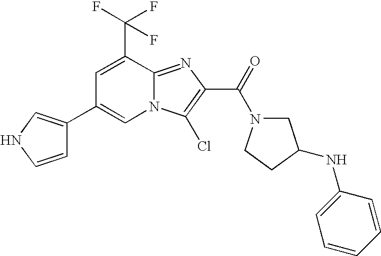 |
| 180 | (3-Chloro-6-furan-3-yl-8- trifluoromethyl-imidazo[1,2- a]pyridin-2-yl)-[3-(4-methoxy- phenylamino)-pyrrolidin-1-yl]- methanone | 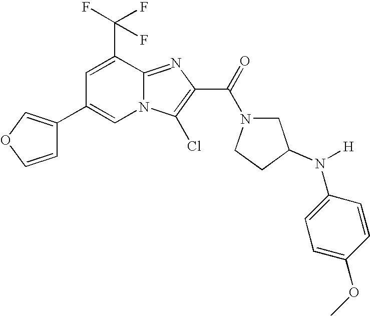 |
| 181 | [3-Chloro-6-(1H-pyrazol-4-yl)-8- trifluoromethyl-imidazo[1,2- a]pyridin-2-yl]-(3-ethoxy- pyrrolidin-1-yl)-methanone | 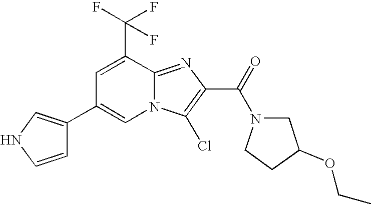 |
| 183 | (3-Chloro-6-furan-3-yl-8- trifluoromethyl-imidazo [1,2- a]pyridin-2-yl)-[3-(pyrimidin-2- ylamino)-pyrrolidin-1-yl]- methanone | 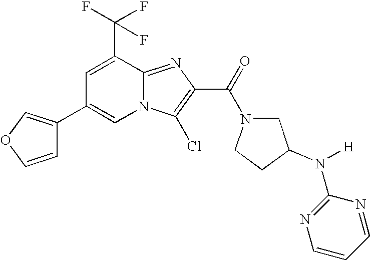 |
| 233 | [3-Chloro-6-(1H-pyrazol-4-yl)-8- trifluoromethyl-imidazo[1,2- a]pyridin-2-yl]-[3-(2-ethoxy- ethoxy)-pyrrohdin-1-yl]- methanone | 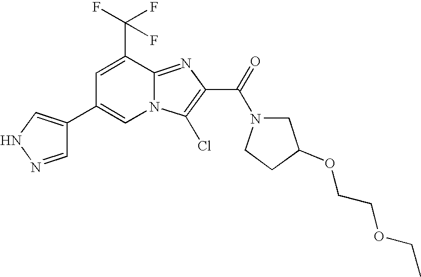 |
| 321 | [1-(3-Chloro-6-furan-3-yl-8- trifluoromethyl-imidazo[1,2- a]pyridine-2-carbonyl)- pyrrolidin-3-yl]-carbamic acid 2- chloro-ethyl ester | 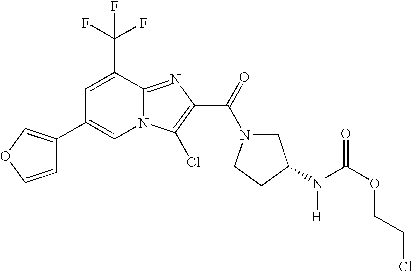 |

In a preferred embodiment, the compounds of Formula (IV) do not include the compound in the following table:

| Compound Name | Structure |
|---|---|
| (3-Chloro-6-furan-3-yl-8-trifluoromethyl- imidazo[1,2-a]pyridin-2-yl)-(3- phenylamino-pyrrolidin-1-yl)-methanone | 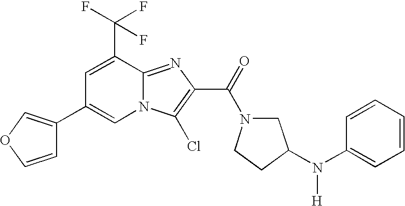 |

Also provided is at least one chemical entity selected from compounds of Formula (V):

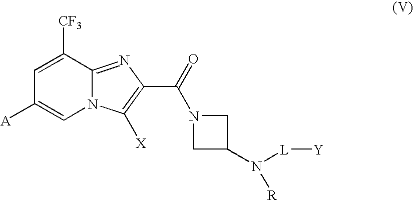

A is selected from the group consisting of halo, aryl, aryl substituted with halo, heteroaryl, and heteroaryl substituted with halo;
X is selected from the group consisting of hydrogen, halo, hydroxymethyl, and hydroxy;
R is selected from the group consisting of hydrogen, C₁-C₃ alkyl, and C₁-C₃ alkyl substituted with hydroxy;
L is —C(O)— or —S(O)₂—; and
Y is C₁-C₃ alkyl or cyclopropyl.

In some embodiments, the compounds of Formula (V) is chosen from the compounds set forth in Table 5.

TABLE 5

| Compound | | |
|---|---|---|
| Number | Name | Structure |
| 139 | N-[1-(3-Chloro-6-furan-2-yl-8- trifluoromethyl-imidazo[1,2- a]pyridine-2-carbonyl)-azetidin- 3-yl]-methanesulfonamide | 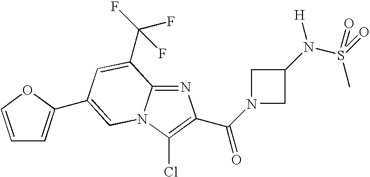 |
| 140 | N-[1-(6-Bromo-3-chloro-8- trifluoromethyl-imidazo[1,2- a]pyridine-2-carbonyl)-azetidin- 3-yl]-methanesulfonamide | 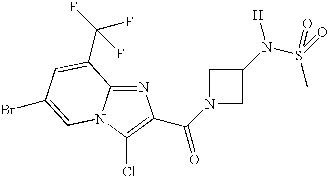 |
| 141 | N-{1-[3-Chloro-6-(3-fluoro- phenyl)-8-trifluoromethyl- imidazo[1,2-a]pyridine-2- carbonyl]-azetidin-3-yl}- methanesulfonamide | 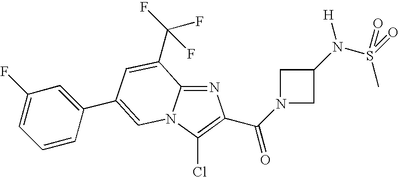 |
| 142 | N-[1-(3-Chloro-6-pyridin-3-yl-8- trifluoromethyl-imidazo[1,2- a]pyridine-2-carbonyl)-azetidin- 3-yl]-methanesulfonamide | 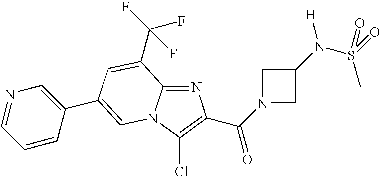 |
| 143 | N-[1-(3-Chloro-6-furan-3-yl-8- trifluoromethyl-imidazo[1,2- a]pyridine-2-carbonyl)-azetidin- 3-yl]-N-methyl- methanesulfonamide | 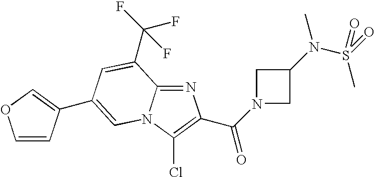 |
| 144 | N-[1-(3-Chloro-6-furan-3-yl-8- trifluoromethyl-imidazo[1,2- a]pyridine-2-carbonyl)-azetidin- 3-yl]-N-(2-hydroxy-ethyl)- methanesulfonamide | 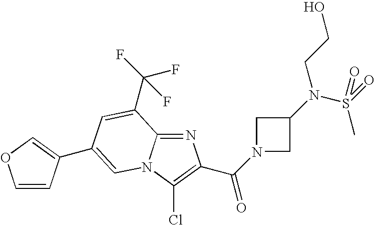 |
| 145 | N-{1-[6-(5-Bromo-furan-3-yl)-8- trifluoromethyl-imidazo[1,2- a]pyridine-2-carbonyl]-azetidin- 3-yl}-methanesulfonamide | 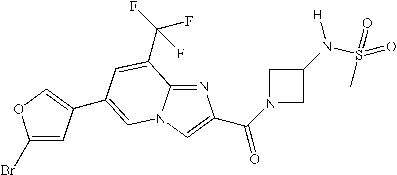 |
| 146 | Ethanesulfonic acid {1-[6-(5- bromo-furan-3-yl)-8- trifluoromethyl-imidazo[1,2- a]pyridine-2-carbonyl]-azetidin- 3-yl}-amide | 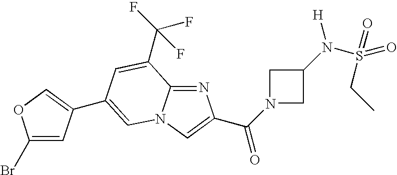 |
| 147 | Cyclopropanesulfonic acid {1- [6-(5-bromo-furan-3-yl)-8- trifluoromethyl-imidazo[1,2- a]pyridine-2-carbonyl]-azetidin- 3-yl}-amide | 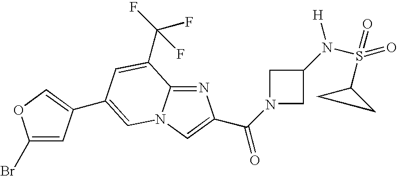 |

In a preferred embodiment, the compounds of Formula (V) do not include any of the compounds in the following table:

| Compound Name | Structure |
|---|---|
| N-[1-(3-Chloro-6-furan-3-yl-8- trifluoromethyl-imidazo[1,2-a]pyridine-2- carbonyl)-azetidin-3-yl]- methanesulfonamide | 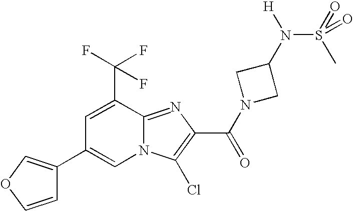 |
| N-(1-{[3-chloro-6-(furan-3-yl)-8- (trifluoromethyl)imidazo[1,2-a]pyridin-2- yl]carbonyl}azetidin-3-yl)propane-2- sulfonamide | 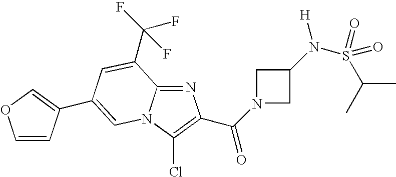 |
| N-(1-{[3-chloro-6-(furan-3-yl)-8- (trifluoromethyl)imidazo[1,2-a]pyridin-2- yl]carbonyl}azetidin-3- yl)cyclopropanesulfonamide | 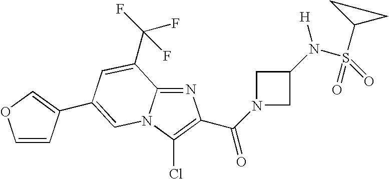 |
| N-(1-{[3-chloro-6-(furan-3-yl)-8- (trifluoromethyl)imidazo[1,2-a]pyridin-2- yl]carbonyl}azetidin-3- yl)ethanesulfonamide | 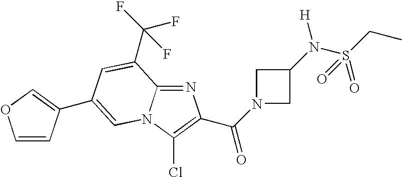 |
| N-[1-(3-Chloro-6-furan-3-yl-8- trifluoromethyl-imidazo[1,2-a]pyridine-2- carbonyl)-azetidin-3-yl]-acetamide | 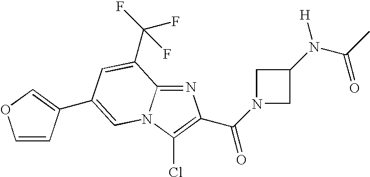 |
| N-(1-{[6-(furan-3-yl)-8- (trifluoromethyl)imidazo[1,2-a]pyridin-2- yl]carbonyl}azetidin-3- yl)methanesulfonamide | 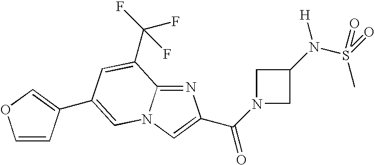 |
| N-(1-{[6-(1H-pyrazol-4-yl)-8- (trifluoromethyl)imidazo[1,2-a]pyridin-2- yl]carbonyl}azetidin-3- yl)methanesulfonamide | 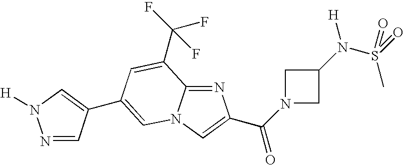 |
| N-(1-{[3-chloro-6-(1H-pyrazol-4-yl)-8- (trifluoromethyl)imidazo[1,2-a]pyridin-2- yl]carbonyl}azetidin-3- yl)methanesulfonamide | 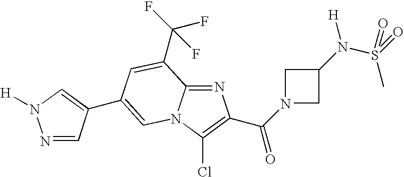 |
| N-(1-{[3-bromo-6-(1H-pyrazol-4-yl)-8- (trifluoromethyl)imidazo[1,2-a]pyridin-2- yl]carbonyl}azetidin-3- yl)methanesulfonamide | 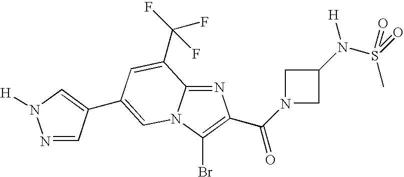 |

Also provided is at least one chemical entity selected from Table 6.

TABLE 6

| Compound | | |
|---|---|---|
| Number | Name | Structure |
| 101 | 3-Chloro-6-furan-3-yl-8- trifluoromethyl-imidazo[1,2- a]pyridine-2-carboxylic acid | 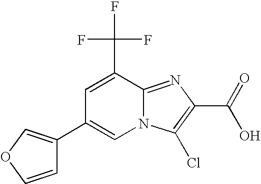 |
| 103 | [3-Chloro-6-(1H-pyrazol-4-yl)- 8-trifluoromethyl-imidazo[1,2- a]pyridin-2-yl]-[3-(2-fluoro- phenyl)-2,5-dihydro-pyrrol-1- yl]-methanone | 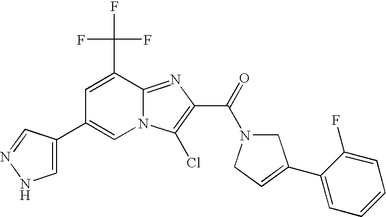 |
| 104 | [3-Chloro-6-(1H-pyrazol-4-yl)- 8-trifluoromethyl-imidazo[1,2- a]pyridin-2-yl]-[3-(1H-imidazol- 4-yl)-2,5-dihydro-pyrrol-1-yl]- methanone | 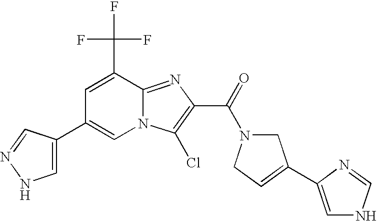 |
| 108 | (3-Chloro-6-furan-3-yl-8- trifluoromethyl-imidazo[1,2- a]pyridin-2-yl)-(4- methanesulfonyl-piperazin-1-yl)- methanone | 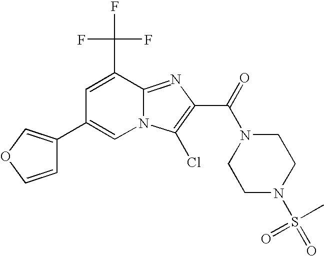 |
| 109 | 2-(6-Furan-3-yl-8- trifluoromethyl-imidazo[1,2- a]pyridin-2-yl)-1-(4-thiazol-2-yl- piperazin-1-yl)-ethanone | 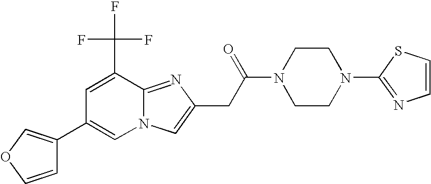 |
| 110 | 2-(6-Furan-3-yl-8- trifluoromethyl-imidazo[1,2- a]pyridin-2-yl)-1-(2-phenyl- piperidin-1-yl)-ethanone | 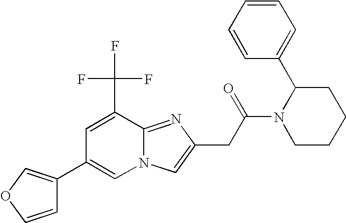 |
| 112 | [3-Chloro-6-(1H-pyrazol-4-yl)- 8-trifluoromethyl-imidazo[1,2- a]pyridin-2-yl]-[4-(1H-pyrazol- 4-yl)-3,6-dihydro-2H-pyridin-1- yl]-methanone | 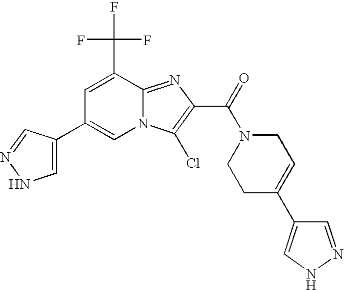 |
| 113 | 1-(1,3-Dihydro-isoindol-2-yl)-2- (6-furan-3-yl-8-trifluoromethyl- imidazo[1,2-a]pyridin-2-yl)- ethanone | 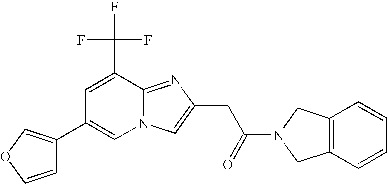 |
| 114 | 1′-(3-Chloro-6-furan-3-yl-8- trifluoromethyl-imidazo[1,2- a]pyridine-2-carbonyl)- [1,4′]bipiperidinyl-2-one | 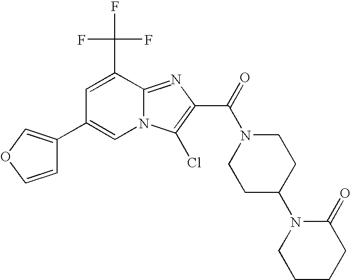 |
| 116 | (3-Fluoro-3′,6′-dihydro-2′H- [2,4′]bipyridinyl-1′-yl)-(6- thiophen-2-yl-4-trifluoromethyl- 1H-pyrrolo[2,3-b]pyridin-2-yl)- methanone | 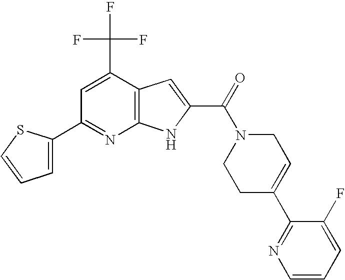 |
| 117 | (3-Fluoro-3′,6′-dihydro-2′H- [2,4′]bipyridinyl-1′-yl)-(5- thiophen-2-yl-7-trifluoromethyl- 1H-pyrrolo[3,2-b]pyridin-2-yl)- methanone | 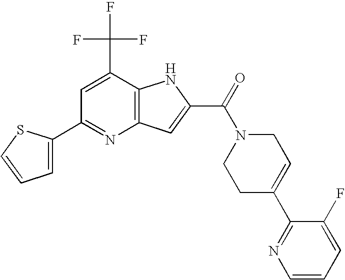 |
| 128 | 2-(3-Fluoro-phenyl)-N-(6-furan- 3-yl-8-trifluoromethyl- imidazo[1,2-a]pyridin-2- ylmethyl)-acetamide | 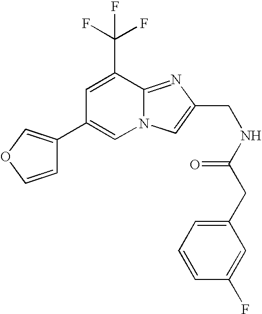 |
| 129 | 2-(2-Fluoro-phenyl)-N-(6-furan- 3-yl-8-trifluoromethyl- imidazo[1,2-a]pyridin-2- ylmethyl)-acetamide | 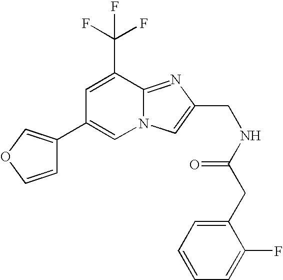 |
| 130 | N-(6-Furan-3-yl-8- trifluoromethyl-imidazo[1,2- a]pyridin-2-ylmethyl)- benzamide | 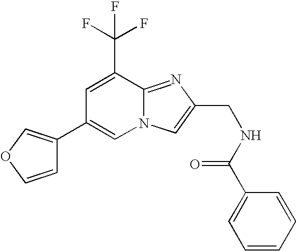 |
| 131 | [3-Chloro-6-(1H-pyrazol-4-yl)- 8-trifluoromethyl-imidazo[1,2- a]pyridin-2-yl]-(3-furan-3-yl- 2,5-dihydro-pyrrol-1-yl)- methanone | 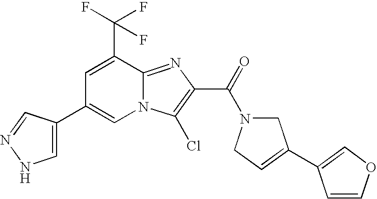 |
| 132 | [3-Chloro-6-(1H-pyrazol-4-yl)- 8-trifluoromethyl-imidazo[1,2- a]pyridin-2-yl]-(3-thiophen-3-yl- 2,5-dihydro-pyrrol-1-yl)- methanone | 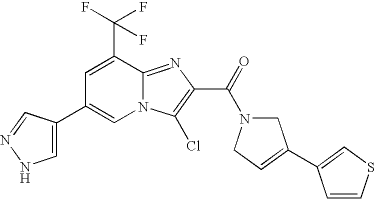 |
| 133 | [3-Chloro-6-(1H-pyrazol-4-yl)- 8-trifluoromethyl-imidazo[1,2- a]pyridin-2-yl]-(3-isoxazol-4-yl- 2,5-dihydro-pyrrol-1-yl)- methanone | 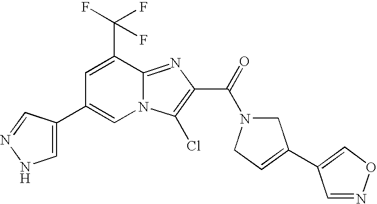 |
| 134 | [3-Chloro-6-(1H-pyrazol-4-yl)- 8-trifluoromethyl-imidazo[1,2- a]pyridin-2-yl]-(3-pyridin-3-yl- 2,5-dihydro-pyrrol-1-yl)- methanone | 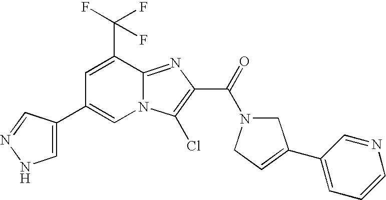 |
| 135 | [3-Chloro-6-(1H-pyrazol-4-yl)- 8-trifluoromethyl-imidazo[1,2- a]pyridin-2-yl]-[3-(1H-pyrazol- 4-yl)-2,5-dihydro-pyrrol-1-yl]- methanone | 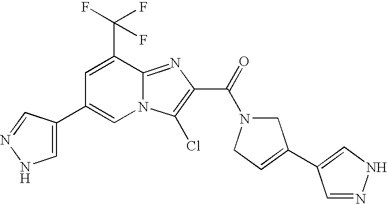 |
| 136 | [3-Chloro-6-(1H-pyrazol-4-yl)- 8-trifluoromethyl-imidazo[1,2- a]pyridin-2-yl]-[3-(1,2,3,6- tetrahydro-pyridin-4-yl)-2,5- dihydro-pyrrol-1-yl]-methanone | 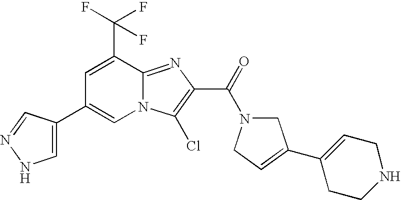 |
| 137 | [3-Chloro-6-(1H-pyrazol-4-yl)- 8-trifluoromethyl-imidazo[1,2- a]pyridin-2-yl]-[3-(1H-pyrazol- 3-yl)-2,5-dihydro-pyrrol-1-yl]- methanone | 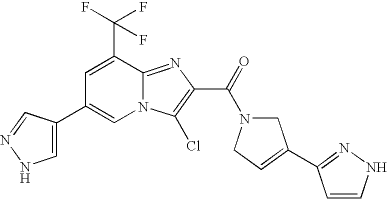 |
| 138 | N-[2-(6-Furan-3-yl-8- trifluoromethyl-imidazo[1,2- a]pyridin-2-yl)-ethyl]-benzamide | 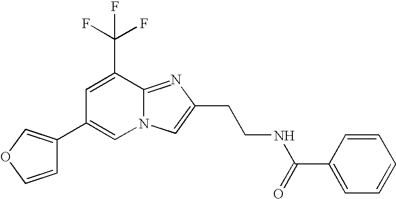 |
| 152 | (3-Chloro-6-furan-3-yl-8- trifluoromethyl-imidazo[1,2- a]pyridin-2-yl)-[4- (cyclopropylmethyl-amino)- piperidin-1-yl]-methanone | 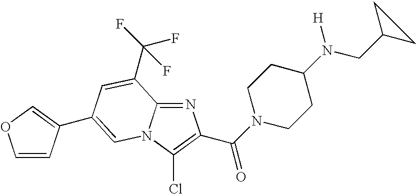 |
| 153 | 6-tert-Butoxycarbonylamino-2- [2-(6-furan-3-yl-8- trifluoromethyl-imidazo[1,2- a]pyridin-2-yl)-acetylamino]- hexanoic acid methyl ester | 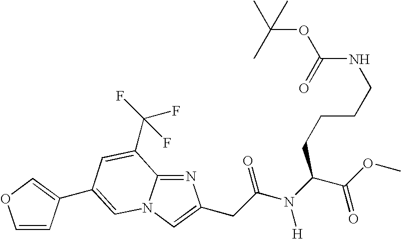 |
| 154 | 2-[(6-Furan-3-yl-8- trifluoromethyl-imidazo[1,2- a]pyridine-2-carbonyl)-amino]- 3,3-dimethyl-butyric acid methyl ester | 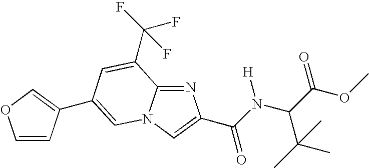 |
| 155 | 2-[(6-Furan-3-yl-8- trifluoromethyl-imidazo[1,2- a]pyridine-2-carbonyl)-amino]- 3-(3H-imidazol-4-yl)-propionic acid methyl ester | 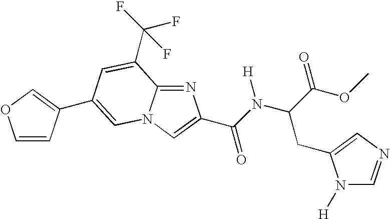 |
| 156 | 2-[(6-Furan-3-yl-8- trifluoromethyl-imidazo[1,2- a]pyridine-2-carbonyl)-amino]- 4-methyl-pentanoic acid methyl ester | 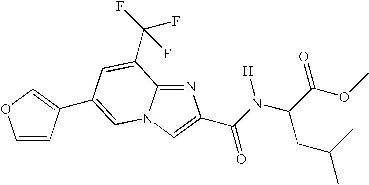 |
| 157 | 2-[(6-Furan-3-yl-8- trifluoromethyl-imidazo[1,2- a]pyridine-2-carbonyl)-amino]- 3-phenyl-propionic acid methyl ester | 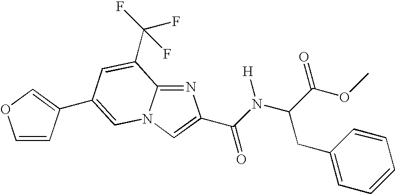 |
| 158 | (3-Chloro-6-furan-3-yl-8- trifluoromethyl-imidazo[1,2- a]pyridin-2-yl)-[4-(3-methyl- butoxy)-piperidin-1-yl]- methanone | 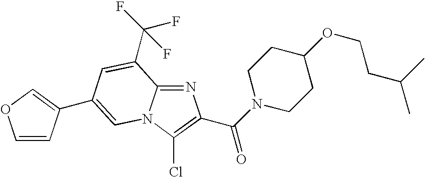 |
| 159 | 2-[1-(3-Chloro-6-furan-3-yl-8- trifluoromethyl-imidazo[1,2- a]pyridine-2-carbonyl)-1,2,3,6- tetrahydro-pyridin-4-yl]- cyclopent-2-enone | 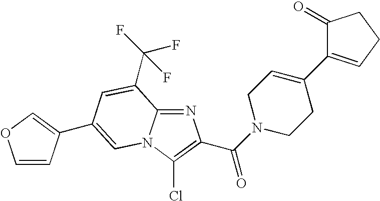 |
| 163 | 6-Furan-3-yl-2-(3-thiazol-2-yl- 2,5-dihydro-pyrrol-1-ylmethyl)- 8-trifluoromethyl-imidazo[1,2- a]pyridine | 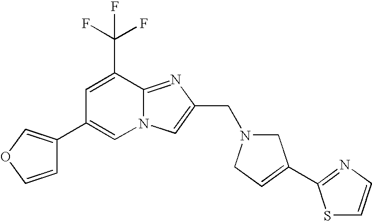 |
| 169 | 3-Chloro-8-difluoromethyl-6- furan-3-yl-2-(3-thiazol-2-yl-2,5- dihydro-pyrrole-1-carbonyl)- imidazo[1,2-a]pyridine-5- carbonitrile | 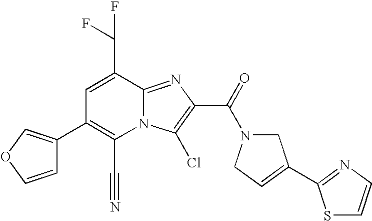 |
| 174 | [3-Chloro-6-(1H-pyrazol-4-yl)- 8-trifluoromethyl-imidazo[1,2- a]pyridin-2-yl]-(4- cyclopropylmethoxy-piperidin- 1-yl)-methanone | 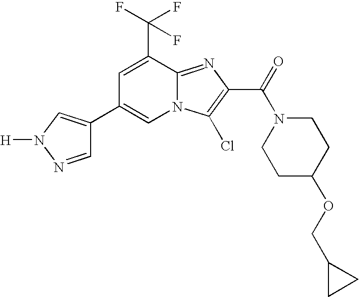 |
| 175 | (3-Chloro-6-furan-3-yl-8- trifluoromethyl-imidazo[1,2- a]pyridin-2-yl)-(4- cyclopropylmethoxy-piperidin- 1-yl)-methanone | 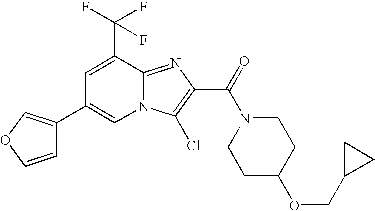 |
| 176 | [3-Chloro-6-(1H-pyrazol-4-yl)- 8-trifluoromethyl-imidazo[1,2- a]pyridin-2-yl]-(4-propoxy- piperidin-1-yl)-methanone | 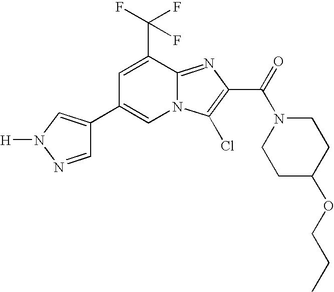 |
| 177 | [3-Chloro-6-(1H-pyrazol-4-yl)- 8-trifluoromethyl-imidazo[1,2- a]pyridin-2-yl]-(4-ethoxy- piperidin-1-yl)-methanone | 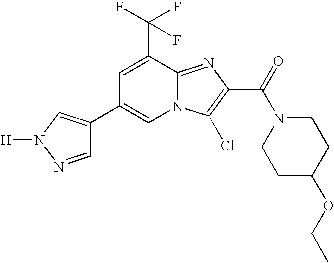 |
| 182 | 2-{1-[3-Chloro-6-(1H-pyrazol-4- yl)-8-trifluoromethyl- imidazo[1,2-a]pyridine-2- carbonyl]-piperidin-4-yloxy}- acetamide | 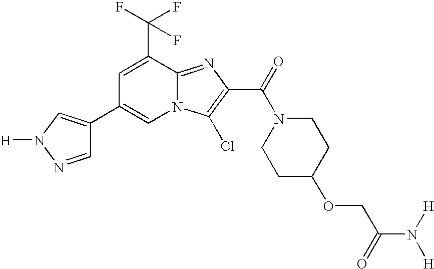 |
| 184 | 2-(6-Furan-3-yl-8- trifluoromethyl-imidazo[1,2- a]pyridin-2-yl)-N-thiazol-2-yl- acetamide | 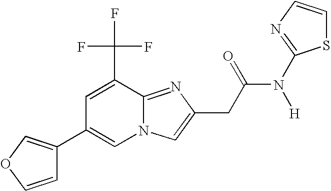 |
| 185 | N-Benzyl-2-(6-furan-3-yl-8- trifluoromethyl-imidazo[1,2- a]pyridin-2-yl)-acetamide | 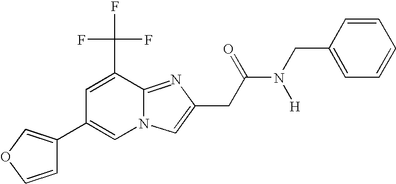 |
| 186 | 2-(6-Furan-3-yl-8- trifluoromethyl-imidazo[1,2- a]pyridin-2-yl)-1-(3-thiazol-2-yl- 2,5-dihydro-pyrrol-1-yl)- ethanone | 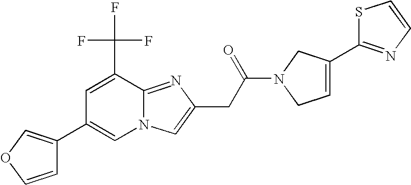 |
| 187 | 2-(6-Furan-3-yl-8- trifluoromethyl-imidazo[1,2- a]pyridin-2-yl)-1-(3-thiophen-2- yl-pyrrolidin-1-yl)-ethanone | 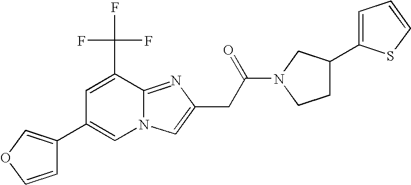 |
| 188 | 2-(6-Furan-3-yl-8- trifluoromethyl-imidazo[1,2- a]pyridin-2-yl)-1-(2-thiophen-2- yl-pyrrolidin-1-yl)-ethanone | 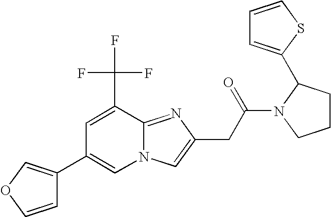 |
| 189 | Thiophene-2-sulfonic acid [2-(6- furan-3-yl-8-trifluoromethyl- imidazo[1,2-a]pyridin-2-yl)- acetyl]-amide | 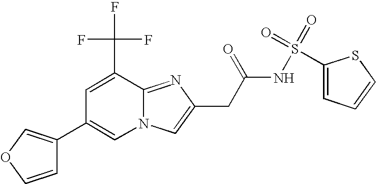 |
| 190 | N-{1-[2-(6-Furan-3-yl-8- trifluoromethyl-imidazo[1,2- a]pyridin-2-yl)-acetyl]-azetidin- 3-yl}-methanesulfonamide | 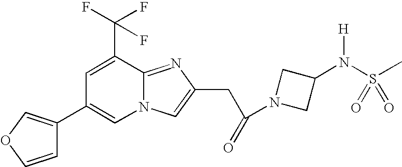 |
| 191 | 2-(6-Furan-3-yl-8- trifluoromethyl-imidazo[1,2- a]pyridin-2-yl)-1-(4-thiazol-2-yl- 3,6-dihydro-2H-pyridin-1-yl)- ethanone | 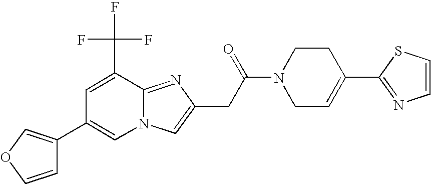 |
| 192 | 2-(6-Furan-3-yl-8- trifluoromethyl-imidazo[1,2- a]pyridin-2-yl)-N-phenethyl- acetamide | 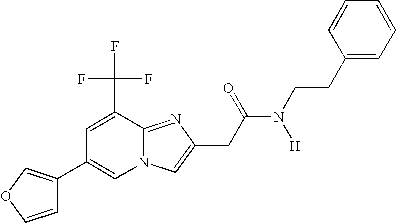 |
| 193 | 3-Chloro-6-furan-3-yl-8- trifluoromethyl-imidazo[1,2- a]pyridine-2-carboxylic acid (4- phenyl-cyclohexyl)-amide | 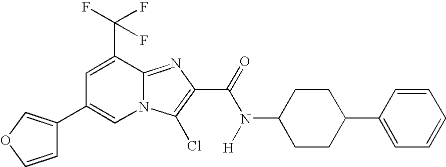 |
| 194 | (4-Benzoyl-piperidin-1-yl)-(3- chloro-6-furan-3-yl-8- trifluoromethyl-imidazo[1,2- a]pyridin-2-yl)-methanone | 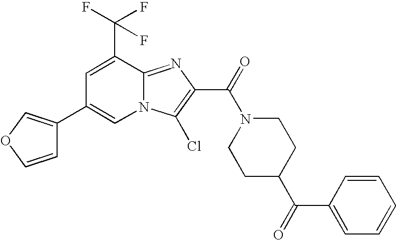 |
| 195 | (3-Chloro-6-furan-3-yl-8- trifluoromethyl-imidazo[1,2- a]pyridin-2-yl)-(4-pyrrolidin-1- yl-piperidin-1-yl)-methanone | 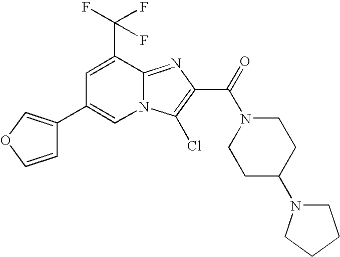 |
| 196 | [3-Chloro-6-(1H-pyrazol-4-yl)- 8-trifluoromethyl-imidazo[1,2- a]pyridin-2-yl]-(4-thiazol-2-yl- 3,6-dihydro-2H-pyridin-1-yl)- methanone | 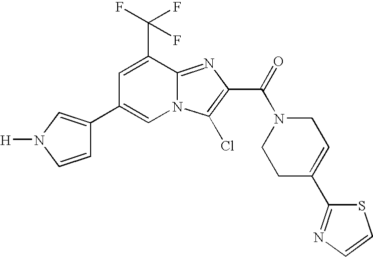 |
| 198 | (3-Chloro-6-furan-3-yl-8- trifluoromethyl-imidazo[1,2- a]pyridin-2-yl)-(4-pyridin-3- ylethynyl-3,6-dihydro-2H- pyridin-1-yl)-methanone | 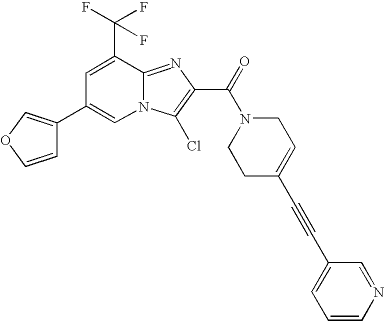 |
| 199 | (3-Chloro-6-furan-3-yl-8- trifluoromethyl-imidazo[1,2- a]pyridin-2-yl)-[4-(3,3-dimethyl- but-1-ynyl)-3,6-dihydro-2H- pyridin-1-yl]-methanone | 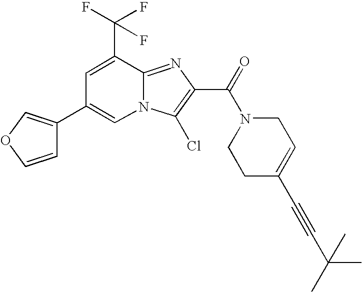 |
| 200 | (3-Chloro-6-furan-3-yl-8- trifluoromethyl-imidazo[1,2- a]pyridin-2-yl)-(4- cyclopentylethynyl-3,6-dihydro- 2H-pyridin-1-yl)-methanone | 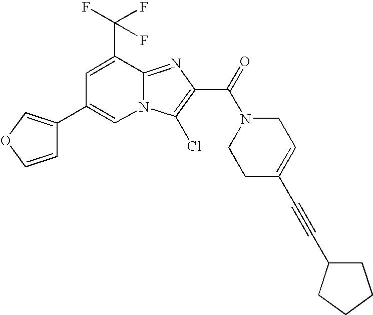 |
| 212 | (3-Chloro-6-furan-3-yl-8- trifluoromethyl-imidazo[1,2- a]pyridin-2-yl)-[4-(2- cyclopropyl-vinyl)-3,6-dihydro- 2H-pyridin-1-yl]-methanone | 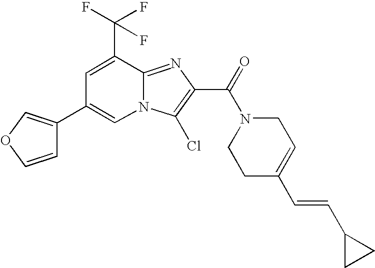 |
| 213 | (3-Chloro-6-furan-3-yl-8- trifluoromethyl-imidazo[1,2- a]pyridin-2-yl)-[4-(2,5-dihydro- 1H-pyrrol-3-yl)-3,6-dihydro-2H- pyridin-1-yl]-methanone | 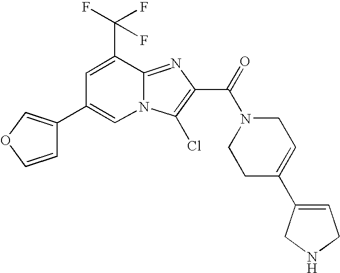 |
| 225 | (3-Chloro-6-furan-3-yl-8- trifluoromethyl-imidazo[1,2- a]pyridin-2-yl)-[4-(3-methyl- butyl)-piperidin-1-yl]-methanone | 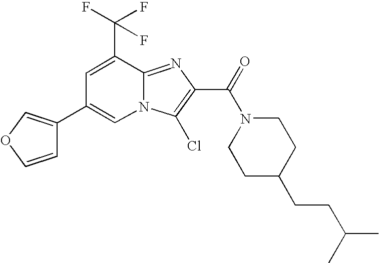 |
| 226 | Cyclopropanecarboxylic acid (6- furan-3-yl-8-trifluoromethyl- imidazo[1,2-a]pyridin-2- ylmethyl)-amide | 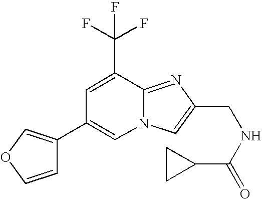 |
| 227 | Cyclopropanesulfonic acid (6- furan-3-yl-8-trifluoromethyl- imidazo[1,2-a]pyridin-2- ylmethyl)-amide | 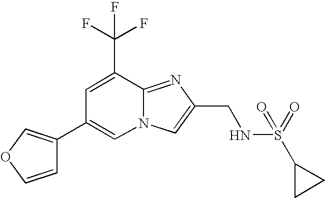 |
| 228 | 6-Furan-3-yl-2-(4-phenyl- imidazol-1-ylmethyl)-8- trifluoromethyl-imidazo[1,2- a]pyridine | 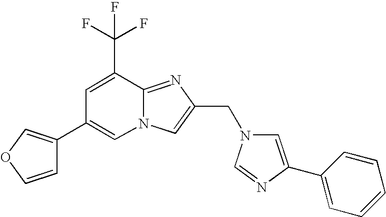 |
| 229 | 6-Furan-3-yl-2-(3-phenyl- [1,2,4,]oxadiazol-5-ylmethyl)-8- trifluoromethyl-imidazo[1,2- a]pyridine | 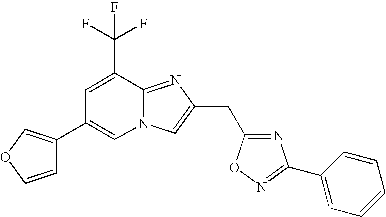 |
| 230 | 2-(3-Benzyl-[1,2,4]oxadiazol-5- ylmethyl)-6-furan-3-yl-8- trifluoromethyl-imidazo[1,2- a]pyridine | 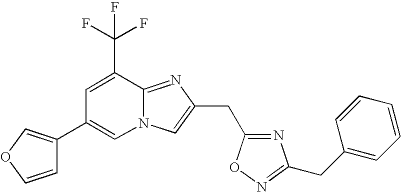 |
| 231 | 6-Furan-3-yl-2-(3- phenoxymethyl- [1,2,4]oxadiazol-5-ylmethyl)-8- trifluoromethyl-imidazo[1,2- a]pyridine | 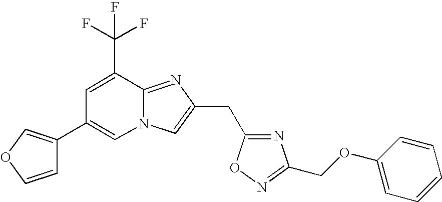 |
| 232 | 2-Methyl-propane-1-sulfonic acid (6-furan-3-yl-8- trifluoromethyl-imidazo[1,2- a]pyridin-2-ylmethyl)-amide | 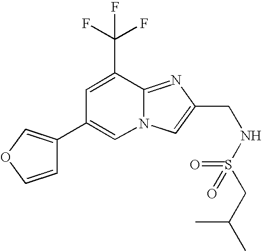 |
| 234 | 2-(6-Furan-3-yl-8- trifluoromethyl-imidazo[1,2- a]pyridin-2-ylmethyl)-isoindole- 1,3-dione | 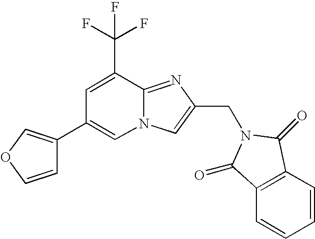 |
| 235 | 1-(4-Chloro-benzyl)-2- pyrrolidin-1-ylmethyl-1H- benzoimidazole | 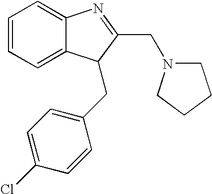 |
| 236 | (6-Furan-3-yl-8-trifluoromethyl- imidazo[1,2-a]pyridin-2-yl)- acetonitrile | 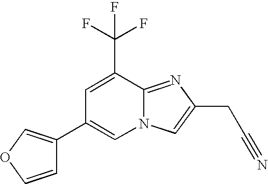 |
| 237 | 1-(3-Chloro-6-furan-3-yl-8- trifluoromethyl-imidazo[1,2- a]pyridine-2-carbonyl)- pyrrolidine-2-carboxylic acid amide | 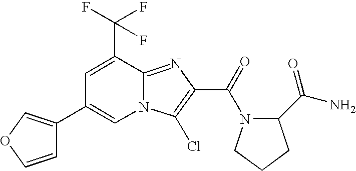 |
| 238 | [3-Chloro-6-(1H-pyrazol-4-yl)- 8-trifluoromethyl-imidazo[1,2- a]pyridin-2-yl]-(3- dimethylamino-pyrrolidin-1-yl)- methanone | 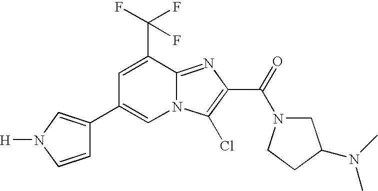 |
| 239 | 2-Dimethylamino-N-(6-furan-3- yl-8-trifluoromethyl- imidazo[1,2-a]pyridin-2- ylmethyl)-acetamide | 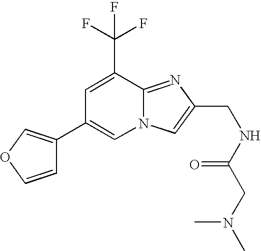 |
| 240 | 2-Acetylamino-N-(6-furan-3-yl- 8-trifluoromethyl-imidazo[1,2- a]pyridin-2-ylmethyl)-acetamide | 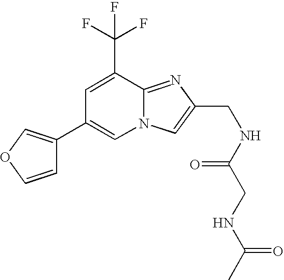 |
| 241 | 2-Amino-4-methyl-pentanoic acid (6-furan-3-yl-8- trifluoromethyl-imidazo[1,2- a]pyridin-2-ylmethyl)-amide | 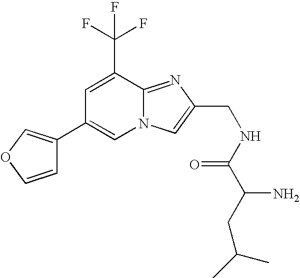 |
| 242 | [2-(6-Furan-3-yl-8- trifluoromethyl-imidazo[1,2- a]pyridin-2-yl)-ethyl]-carbamic acid tert-butyl ester | 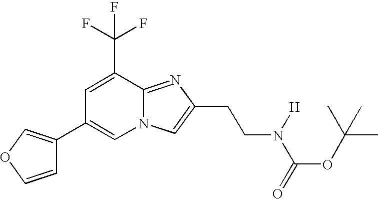 |
| 243 | 2-Acetylamino-4-methyl- pentanoic acid (6-furan-3-yl-8- trifluoromethyl-imidazo[1,2- a]pyridin-2-ylmethyl)-amide | 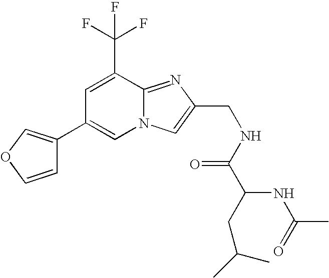 |
| 244 | 2-Acetylamino-N-(6-furan-3-yl- 8-trifluoromethyl-imidazo[1,2- a]pyridin-2-ylmethyl)- propionamide | 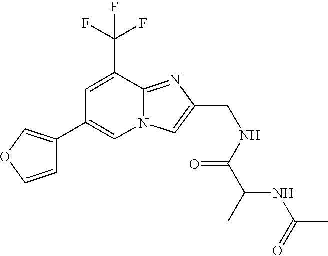 |
| 245 | 2-Acetylamino-N-(6-furan-3-yl- 8-trifluoromethyl-imidazo[1,2- a]pyridin-2-ylmethyl)-3-(1H- indol-3-yl)-propionamide | 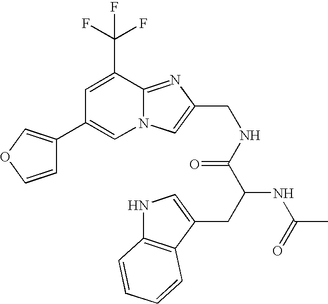 |
| 246 | [(6-Furan-3-yl-8- trifluoromethyl-imidazo[1,2- a]pyridine-2-carbonyl)-amino]- acetic acid methyl ester | 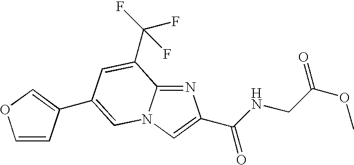 |
| 247 | 6-Furan-3-yl-8-trifluoromethyl- imidazo[1,2-a]pyridine-2- carboxylic acid carbamoylmethyl-amide | 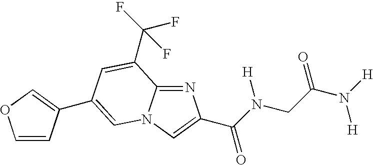 |
| 248 | 6-Furan-3-yl-8-trifluoromethyl- imidazo[1,2-a]pyridine-2- carboxylic acid methylcarbamoylmethyl-amide | 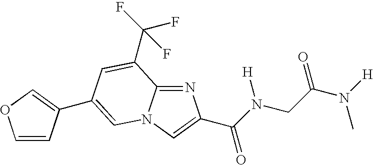 |
| 249 | 6-Furan-3-yl-8-trifluoromethyl- imidazo[1,2-a]pyridine-2- carboxylic acid dimethylcarbamoylmethyl-amide | 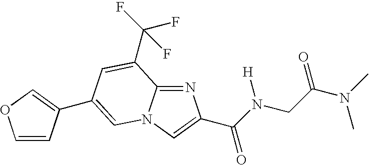 |
| 250 | 2-[(6-Furan-3-yl-8- trifluoromethyl-imidazo[1,2- a]pyridine-2-carbonyl)-amino]- propionic acid methyl ester | 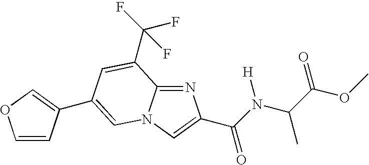 |
| 251 | 2-[(6-Furan-3-yl-8- trifluoromethyl-imidazo[1,2- a]pyridine-2-carbonyl)-amino]- 4-methylsulfanyl-butyric acid methyl ester | 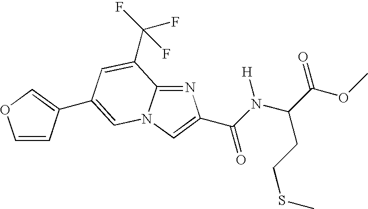 |
| 252 | 2-[(6-Furan-3-yl-8- trifluoromethyl-imidazo[1,2- a]pyridine-2-carbonyl)-amino]- 3-hydroxy-propionic acid methyl ester | 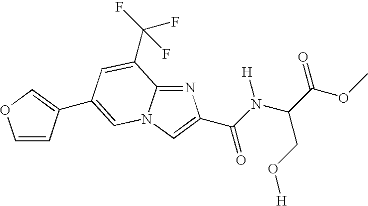 |
| 253 | 2-[(6-Furan-3-yl-8- trifluoromethyl-imidazo[1,2- a]pyridine-2-carbonyl)-amino]- 3-hydroxy-butyric acid methyl ester | 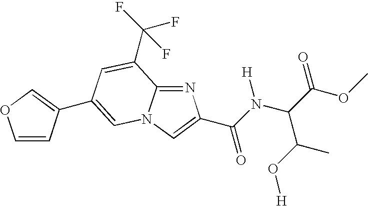 |
| 254 | 1-(6-Furan-3-yl-8- trifluoromethyl-imidazo[1,2- a]pyridine-2-carbonyl)- pyrrolidine-2-carboxylic acid methyl ester | 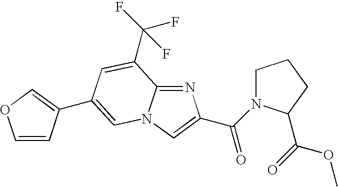 |
| 255 | [(6-Furan-3-yl-8- trifluoromethyl-imidazo[1,2- a]pyridine-2-carbonyl)-amino]- phenyl-acetic acid methyl ester | 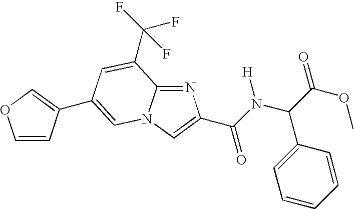 |
| 256 | 2-[(6-Furan-3-yl-8- trifluoromethyl-imidazo[1,2- a]pyridine-2-carbonyl)-amino]- 3-methyl-butyric acid methyl ester | 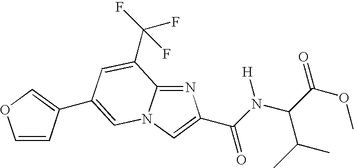 |
| 257 | 6-tert-Butoxycarbonylamino-2- [(6-furan-3-yl-8-trifluoromethyl- imidazo[1,2-a]pyridine-2- carbonyl)-amino]-hexanoic acid methyl ester | 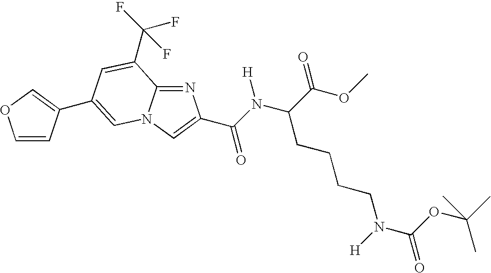 |
| 258 | 2-(6-Furan-3-yl-8- trifluoromethyl-imidazo[1,2- a]pyridin-2-yl)-ethylamine | 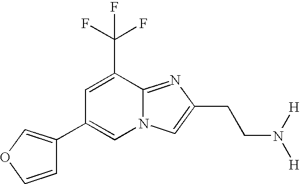 |
| 259 | 2-Acetylamino-N-(6-furan-3-yl- 8-trifluoromethyl-imidazo[1,2- a]pyridin-2-ylmethyl)-3- hydroxy-propionamide | 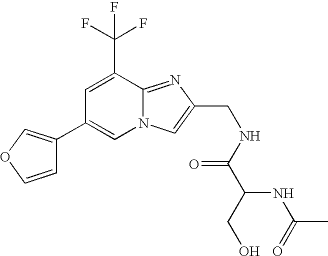 |
| 260 | 2-Acetylamino-N-(6-furan-3-yl- 8-trifluoromethyl-imidazo[1,2- a]pyridin-2-ylmethyl)-3-phenyl- propionamide | 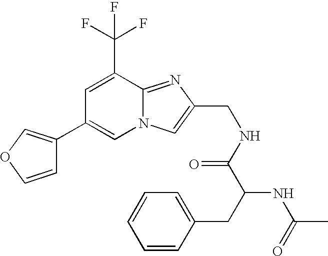 |
| 261 | 2-Acetylamino-N-(6-furan-3-yl- 8-trifluoromethyl-imidazo[1,2- a]pyridin-2-ylmethyl)-3-methyl- butyramide | 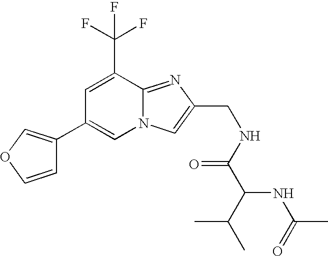 |
| 262 | 2-Dimethylamino-N-(6-furan-3- yl-8-trifluoromethyl- imidazo[1,2-a]pyridin-2- ylmethyl)-propionamide | 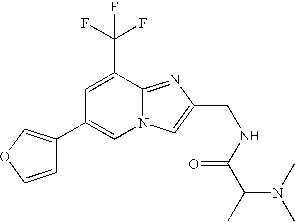 |
| 263 | Thiophene-2-carboxylic acid [2- (6-furan-3-yl-8-trifluoromethyl- imidazo[1,2-a]pyridin-2-yl)- ethyl]-amide | 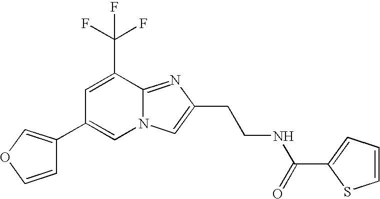 |
| 264 | [(6-Furan-3-yl-8- trifluoromethyl-imidazo[1,2- a]pyridine-2-carbonyl)-amino]- acetic acid | 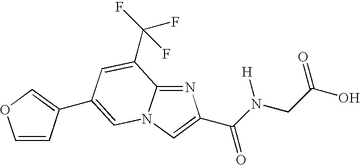 |
| 265 | 2-[(6-Furan-3-yl-8- trifluoromethyl-imidazo[1,2- a]pyridine-2-carbonyl)-amino]- propionic acid | 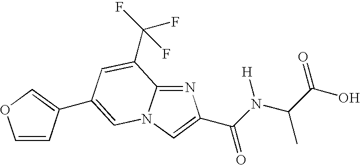 |
| 266 | 2-[(6-Furan-3-yl-8- trifluoromethyl-imidazo[1,2- a]pyridine-2-carbonyl)-amino]- 4-methylsulfanyl-butyric acid | 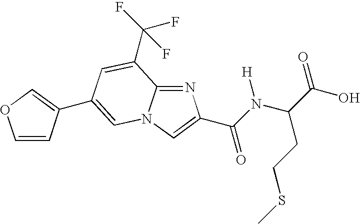 |
| 267 | 2-[(6-Furan-3-yl-8- trifluoromethyl-imidazo[1,2- a]pyridine-2-carbonyl)-amino]- 3-hydroxy-propionic acid | 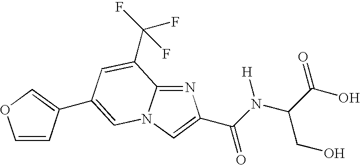 |
| 268 | (4-Benzenesulfonyl-piperidin-1- yl)-(3-chloro-6-furan-3-yl-8- trifluoromethyl-imidazo[1,2- a]pyridin-2-yl)-methanone | 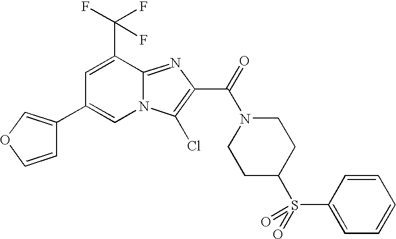 |
| 269 | 2-[(6-Furan-3-yl-8- trifluoromethyl-imidazo[1,2- a]pyridine-2-carbonyl)-amino]- 3-hydroxy-butyric acid | 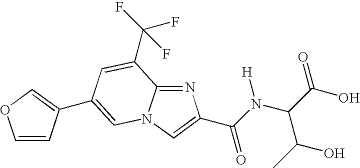 |
| 270 | 2-[(6-Furan-3-yl-8- trifluoromethyl-imidazo[1,2- a]pyridine-2-carbonyl)-amino]- 4-methyl-pentanoic acid | 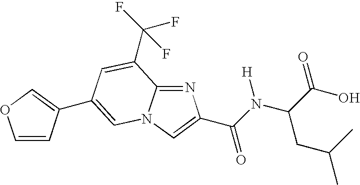 |
| 271 | [(6-Furan-3-yl-8- trifluoromethyl-imidazo[1,2- a]pyridine-2-carbonyl)-amino]- phenyl-acetic acid | 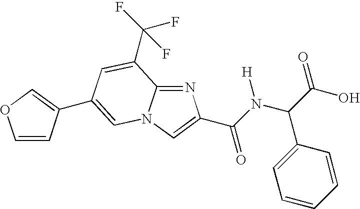 |
| 272 | 2-[(6-Furan-3-yl-8- trifluoromethyl-imidazo[1,2- a]pyridine-2-carbonyl)-amino]- 3-methyl-butyric acid | 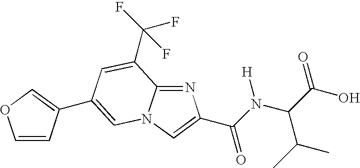 |
| 273 | 2-[(6-Furan-3-yl-8- trifluoromethyl-imidazo[1,2- a]pyridine-2-carbonyl)-amino]- 3,3-dimethyl-butyric acid | 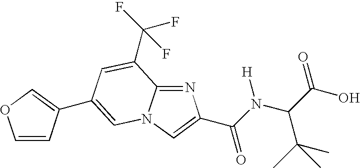 |
| 274 | N-[2-(6-Furan-3-yl-8- trifluoromethyl-imidazo[1,2- a]pyridin-2-yl)-ethyl]-2- thiophen-2-yl-acetamide | 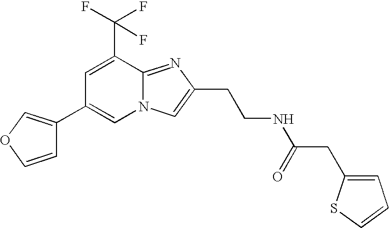 |
| 275 | Cyclopropanecarboxylic acid [2- (6-furan-3-yl-8-trifluoromethyl- imidazo[1,2-a]pyridin-2-yl)- ethyl]-amide | 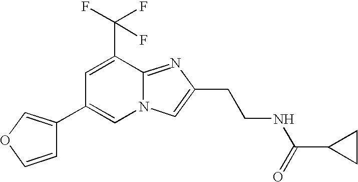 |
| 276 | 3-[(6-Furan-3-yl-8- trifluoromethyl-imidazo[1,2- a]pyridine-2-carbonyl)-amino]- propionic acid methyl ester | 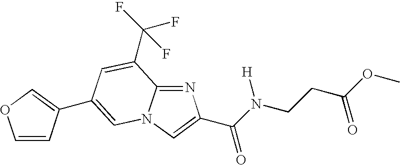 |
| 277 | 6-Furan-3-yl-8-trifluoromethyl- imidazo[1,2-a]pyridine-2- carboxylic acid (1-carbamoyl- ethyl)-amide | 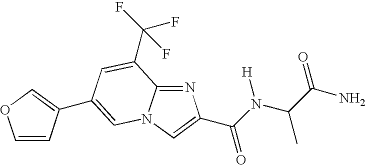 |
| 278 | 6-Furan-3-yl-8-trifluoromethyl- imidazo[1,2-a]pyridine-2- carboxylic acid (1-carbamoyl-2- methyl-propyl)-amide | 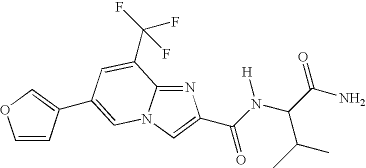 |
| 279 | 6-Furan-3-yl-8-trifluoromethyl- imidazo[1,2-a]pyridine-2- carboxylic acid (1-carbamoyl-2- hydroxy-ethyl)-amide | 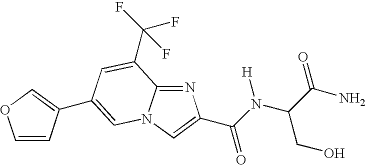 |
| 280 | 1-(6-Furan-3-yl-8- trifluoromethyl-imidazo[1,2- a]pyridine-2-carbonyl)- pyrrolidine-2-carboxylic acid amide | 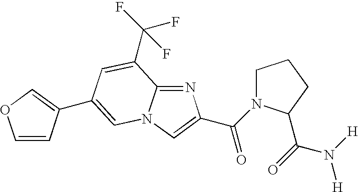 |
| 281 | 1-(6-Furan-3-yl-8- trifluoromethyl-imidazo[1,2- a]pyridine-2-carbonyl)- pyrrolidine-2-carboxylic acid dimethylamide | 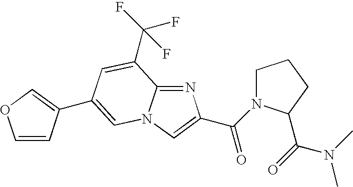 |
| 282 | 6-Furan-3-yl-8-trifluoromethyl- imidazo[1,2-a]pyridine-2- carboxylic acid (1-carbamoyl-2- hydroxy-propyl)-amide | 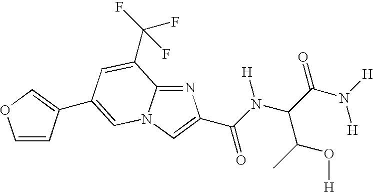 |
| 283 | 1-[2-(6-Furan-3-yl-8- trifluoromethyl-imidazo[1,2- a]pyridin-2-yl)-ethyl]-3-phenyl- urea | 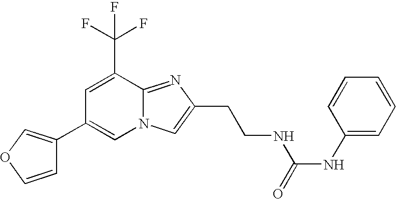 |
| 284 | 1-Acetyl-pyrrolidine-2- carboxylic acid (6-furan-3-yl-8- trifluoromethyl-imidazo[1,2- a]pyridin-2-ylmethyl)-amide | 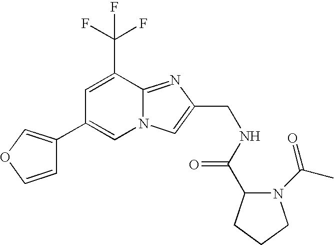 |
| 285 | 3-[(6-Furan-3-yl-8- trifluoromethyl-imidazo[1,2- a]pyridine-2-carbonyl)-amino]- propionic acid | 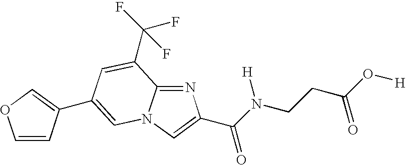 |
| 286 | 2-[(6-Furan-3-yl-8- trifluoromethyl-imidazo[1,2- a]pyridine-2-carbonyl)-amino]- 3-phenyl-propionic acid | 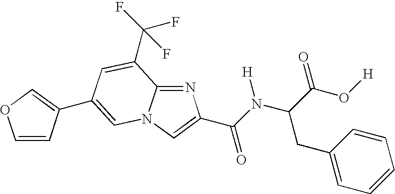 |
| 287 | 2-Acetylamino-N-(6-furan-3-yl- 8-trifluoromethyl-imidazo[1,2- a]pyridin-2-ylmethyl)-4- methylsulfanyl-butyramide | 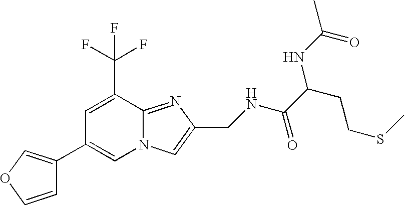 |
| 288 | 2-Acetylamino-N-(6-furan-3-yl- 8-trifluoromethyl-imidazo[1,2- a]pyridin-2-ylmethyl)-3-methyl- butyramide | 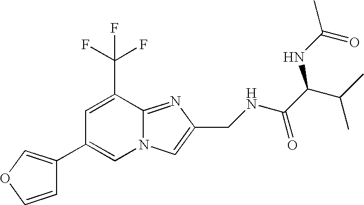 |
| 289 | 2-Acetylamino-N-(6-furan-3-yl- 8-trifluoromethyl-imidazo[1,2- a]pyridin-2-ylmethyl)-3- hydroxy-butyramide | 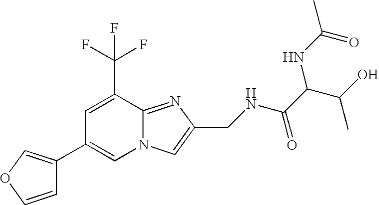 |
| 290 | [2-(6-Furan-3-yl-8- trifluoromethyl-imidazo[1,2- a]pyridin-2-yl)-acetylamino]- acetic acid methyl ester | 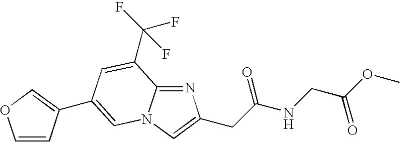 |
| 291 | 2-[2-(6-Furan-3-yl-8- trifluoromethyl-imidazo[1,2- a]pyridin-2-yl)-acetylamino]-4- methylsulfanyl-butyric acid methyl ester | 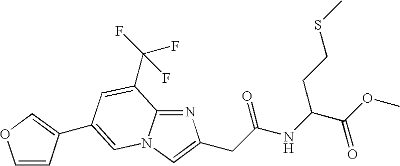 |
| 292 | 2-[2-(6-Furan-3-yl-8- trifluoromethyl-imidazo[1,2- a]pyridin-2-yl)-acetylamino]-3- hydroxy-propionic acid methyl ester | 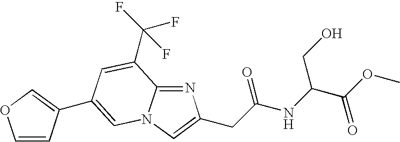 |
| 293 | 2-[2-(6-Furan-3-yl-8- trifluoromethyl-imidazo[1,2- a]pyridin-2-yl)-acetylamino]-3- (3H-imidazol-4-yl)-propionic acid methyl ester | 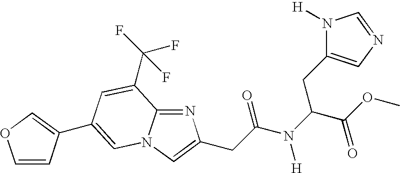 |
| 294 | 2-[2-(6-Furan-3-yl-8- trifluoromethyl-imidazo[1,2- a]pyridin-2-yl)-acetylamino]- 3,3-dimethyl-butyric acid methyl ester | 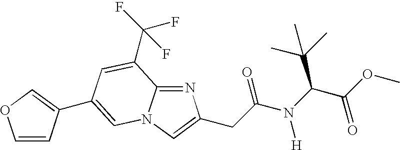 |
| 295 | 3-[2-(6-Furan-3-yl-8- trifluoromethyl-imidazo[1,2- a]pyridin-2-yl)-acetylamino]- propionic acid methyl ester | 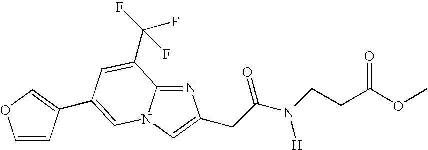 |
| 296 | 2-[2-(6-Furan-3-yl-8- trifluoromethyl-imidazo[1,2- a]pyridin-2-yl)-acetylamino]- propionamide | 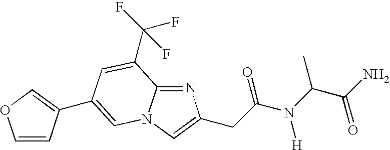 |
| 297 | 2-[2-(6-Furan-3-yl-8- trifluoromethyl-imidazol[1,2- a]pyridin-2-yl)-acetylamino]-3- methyl-butyramide | 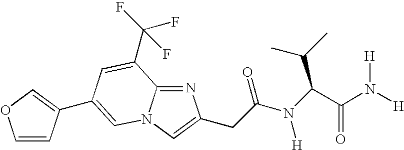 |
| 298 | 2-[2-(6-Furan-3-yl-8- trifluoromethyl-imidazo[1,2- a]pyridin-2-yl)-acetylamino]-3- hydroxy-propionamide | 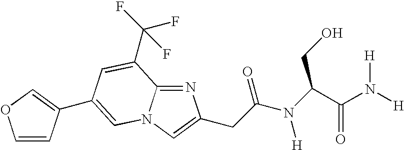 |
| 299 | 2-[2-(6-Furan-3-yl-8- trifluoromethyl-imidazo[1,2- a]pyridin-2-yl)-acetylamino]-3- hydroxy-butyramide | 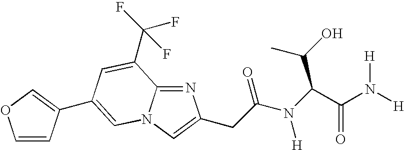 |
| 300 | N-Carbamoylmethyl-2-(6-furan- 3-yl-8-trifluoromethyl- imidazo[1,2-a]pyridin-2-yl)- acetamide | 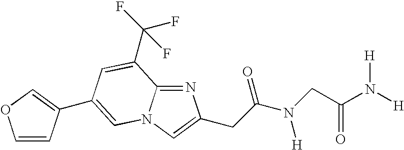 |
| 301 | 2-(6-Furan-3-yl-8- trifluoromethyl-imidazo[1,2- a]pyridin-2-yl)-N- methylcarbamoylmethyl- acetamide | 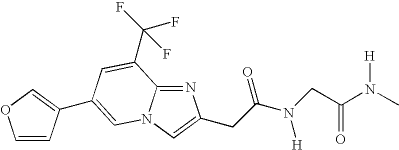 |
| 302 | N-Dimethylcarbamoylmethyl-2- (6-furan-3-yl-8-trifluoromethyl- imidazo[1,2-a]pyridin-2-yl)- acetamide | 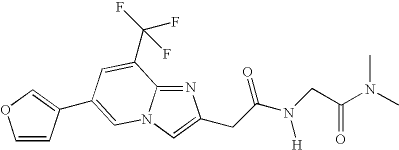 |
| 303 | 2-[2-(6-Furan-3-yl-8- trifluoromethyl-imidazo[1,2- a]pyridin-2-yl)-acetylamino]-3- (1H-indol-3-yl)-propionamide | 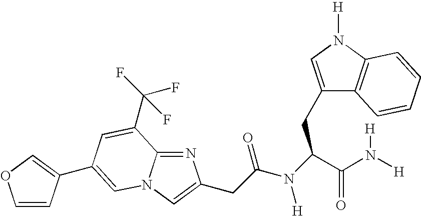 |
| 304 | 2-[2-(6-Furan-3-yl-8- trifluoromethyl-imidazo[1,2- a]pyridin-2-yl)-acetylamino]- propionic acid methyl ester | 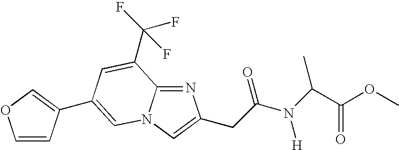 |
| 305 | 2-[2-(6-Furan-3-yl-8- trifluoromethyl-imidazo[1,2- a]pyridin-2-yl)-acetylamino]-3- hydroxy-butyric acid methyl ester | 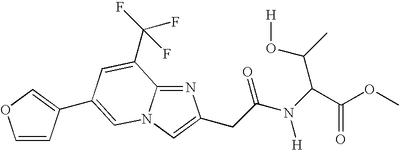 |
| 306 | 2-[2-(6-Furan-3-yl-8- trifluoromethyl-imidazo[1,2- a]pyridin-2-yl)-acetylamino]-4- methyl-pentanoic acid methyl ester | 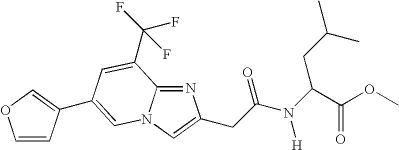 |
| 307 | 1-[2-(6-Furan-3-yl-8- trifluoromethyl-imidazo[1,2- a]pyridin-2-yl)-acetyl]- pyrrolidine-2-carboxylic acid methyl ester | 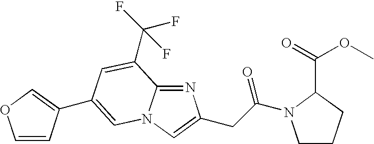 |
| 309 | [2-(6-Furan-3-yl-8- trifluoromethyl-imidazo[1,2- a]pyridin-2-yl)-acetylamino]- phenyl-acetic acid methyl ester | 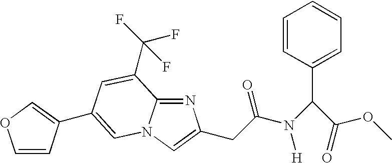 |
| 310 | 2-[2-(6-Furan-3-yl-8- trifluoromethyl-imidazo[1,2- a]pyridin-2-yl)-acetylamino]-3- methyl-butyric acid methyl ester | 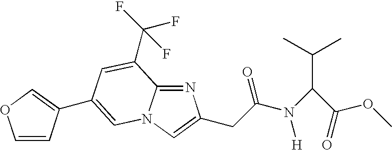 |
| 311 | 2-[2-(6-Furan-3-yl-8- trifluoromethyl-imidazol[1,2- a]pyridin-2-yl)-acetylamino]-3- (1H-indol-3-yl)-propionic acid methyl ester | 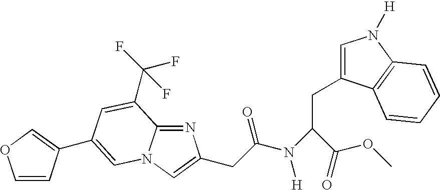 |
| 312 | 2-[2-(6-Furan-3-yl-8- trifluoromethyl-imidazo[1,2- a]pyridin-2-yl)-acetylamino]-3- phenyl-propionic acid methyl ester | 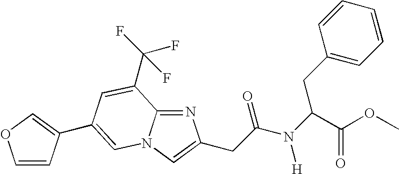 |
| 313 | 1-[2-(6-Furan-3-yl-8- trifluoromethyl-imidazo[1,2- a]pyridin-2-yl)-acetyl]- pyrrolidine-2-carboxylic acid amide | 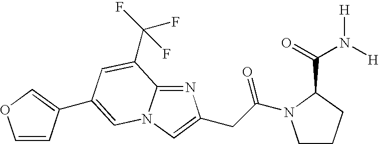 |
| 314 | 1-[2-(6-Furan-3-yl-8- trifluoromethyl-imidazo[1,2- a]pyridin-2-yl)-acetyl]- pyrrolidine-2-carboxylic acid dimethylamide | 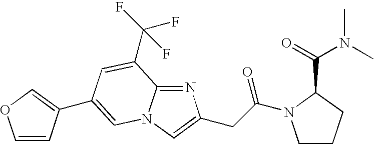 |
| 322 | N-[1-(3-Chloro-6-furan-3-yl-8- trifluoromethyl-imidazo[1,2- a]pyridine-2-carbonyl)- piperidin-4-yl]- methanesulfonamide | 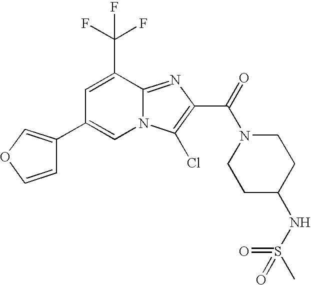 |
| 323 | N-[1-(3-Chloro-6-furan-3-yl-8- trifluoromethyl-imidazo[1,2- a]pyridine-2-carbonyl)- piperidin-4-yl]-acetamide | 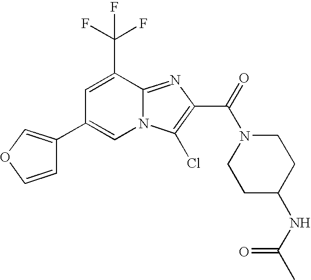 |
| 324 | 9-(3-Chloro-6-furan-3-yl-8- trifluoromethyl-imidazo[1,2- a]pyridine-2-carbonyl)-1,9- diaza-spiro[5.5]undecan-2-one | 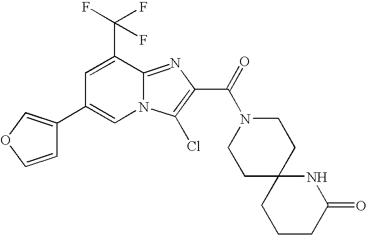 |
| 325 | 1-[4-(3-Chloro-6-furan-3-yl-8- trifluoromethyl-imidazo[1,2- a]pyridine-2-carbonyl)- [1,4]diazepan-1-yl]-ethanone | 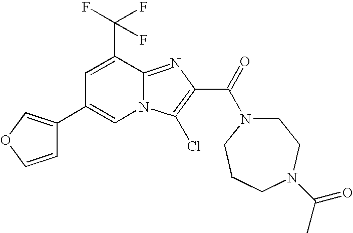 |
| 326 | (3-Chloro-6-furan-3-yl-8- trifluoromethyl-imidazo[1,2- a]pyridin-2-yl)-(4- methanesulfonyl-[1,4]diazepan- 1-yl)-methanone | 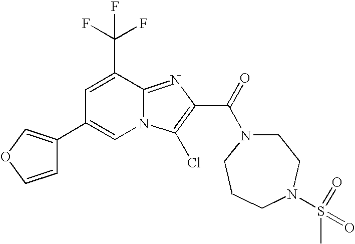 |
| 327 | 1-[8-(3-Chloro-6-furan-3-yl-8- trifluoromethyl-imidazo[1,2- a]pyridine-2-carbonyl)-1-thia- 4,8-diaza-spiro[4.5]dec-4-yl]- ethanone | 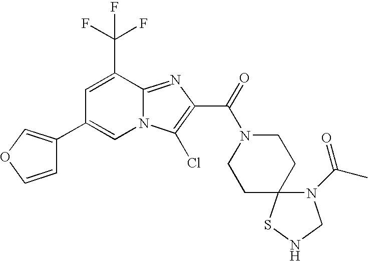 |
| 330 | 8-(3-Chloro-6-furan-3-yl-8- trifluoromethyl-imidazo[1,2- a]pyridine-2-carbonyl)-1,3,8- triaza-spiro[4.5]decane-2,4- dione | 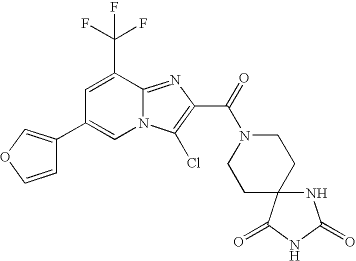 |
| 331 | 4-(3-Chloro-6-furan-3-yl-8- trifluoromethyl-imidazo[1,2- a]pyridine-2-carbonyl)- [1,4]diazepane-1-carboxylic acid methyl ester | 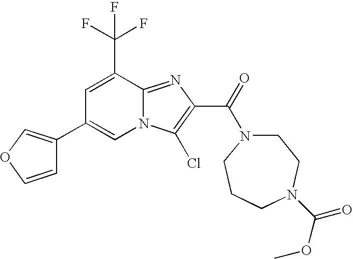 |

Also provided is a pharmaceutical composition comprising a pharmaceutically acceptable diluent and a therapeutically effective amount of at least one chemical entity described herein.

Also provided is a method for treating a viral infection in a mammal mediated at least in part by a virus in the Flaviviridae family of viruses which method comprises administering to a mammal, that has been diagnosed with said viral infection or is at risk of developing said viral infection, a compound described herein. In some embodiments, said virus is hepatitis C virus. In some embodiments, the method further comprises administration of a therapeutically effective amount of one or more agents active against hepatitis C virus. In some embodiments, said agent active against hepatitis C virus is an inhibitor of HCV proteases, HCV polymerase, HCV helicase, HCV NS4B protein, HCV entry, HCV assembly, HCV egress, HCV replicase, HCV NS5A protein, or inosine 5′-monophosphate dehydrogenase. In some embodiments, said agent active against hepatitis C virus is interferon.

The methods of synthesis for the provided chemical entities employ readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, the methods of this specification employ protecting groups which are necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the provided chemical entities may contain one or more chiral centers and such compounds can be prepared or isolated as pure stereoisomers, i.e., as individual enantiomers or diastereomers, or as stereoisomer-enriched mixtures. All such stereoisomers (and enriched mixtures) are included within the scope of this specification, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Ernka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989). The synthesis of the compounds provided generally follows either a convergent or linear synthetic pathway as described below.

Unless specified to the contrary, the reactions described herein take place at atmospheric pressure, generally within a temperature range from −10° C. to 200° C. Further, except as employed in the Examples or as otherwise specified, reaction times and conditions are intended to be approximate, e.g., taking place at about atmospheric pressure within a temperature range of about −10° C. to about 110° C. over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, N-methylpyrrolidone (“NMP”), pyridine and the like]. Unless specified to the contrary, the solvents used in the reactions described herein are inert organic solvents. Unless specified to the contrary, for each gram of the limiting reagent, one cc (or mL) of solvent constitutes a volume equivalent

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

Scheme 1 shows a method of assembling the imidazopyridine scaffold with various substituents. 2-Amino pyridine substituted with R⁷ is brominated by treatment with NBS in a solvent such as DMF. Substituted 2-aminopyridine 1.2 is cyclized to the imidazopyridine 1.3 by heating it with ethyl bromopyruvate in a solvent like DMF. Treatment of intermediate 1.3 with NCS in DMF affords the 3-chlorosubstituted imidazopyridine 1.4. Palladium mediated coupling reactions such as Suzuki couplings, Sonogashira couplings and Heck couplings can afford diversity at R⁵ in intermediates 1.5. Hydrolysis of the ester is effected by refluxing in 4N HCl and acetonitrile as co-solvent. The acid 1.6 is converted to amides 1.7 through standard amide coupling agents such as HBTU.

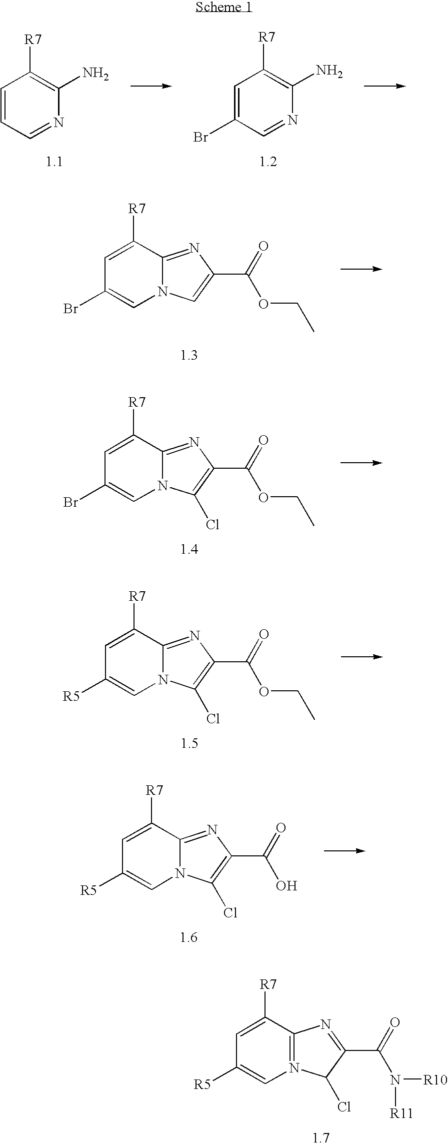

Scheme 2 shows a general scheme for the synthesis of purine analogs such as 2.5. An appropriately substituted amino dichloropyrimidine (2.1) can be converted to diaminopyrimidine such as 2.2 by stirring with an appropriately substituted primary amine (R³NH₂). Reaction with ethyl glyoxalate affords the ester intermediate 2.3. Paladium mediated coupling reactions such as Suzuki couplings, Sonogashira couplings and Heck couplings can afford diversity. Hydrolysis of the ester followed by amide coupling can afford the desired purine amide analogs such as 2.5.

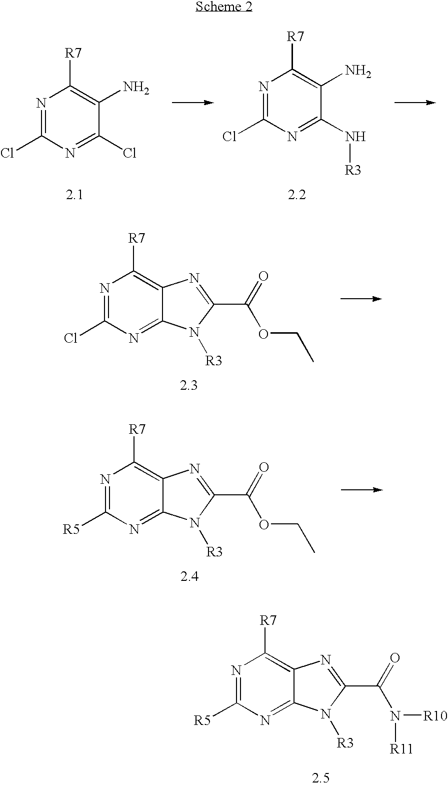

Scheme 3 shows a general scheme for the synthesis of pyrrolopyrimidines such as 3.7. The BOC protected amino bromo pyrimidine (3.2) can be prepared from the appropriately substituted amino bromo pyrimidine (3.1) using standard methods. Sonogashira coupling with ethyl propiolate would afford the alkyne 3.3. Cyclization to the 2-substituted pyrrolopyrimidine 3.4 can be done by heating with tetrabutyl ammonium fluoride. Heating 3.4 with an alkyl halide results in N-alkylation to the intermediate 3.5. Palladium mediated coupling reactions such as Suzuki couplings, Sonogashira couplings and Heck couplings can afford diversity at R⁵ in intermediates 3.6. Hydrolysis of the ester is effected by refluxing in 4N HCl and acetonitrile as co-solvent. The resulting acid is converted to amides 3.7 through standard amide coupling agents such as HBTU.

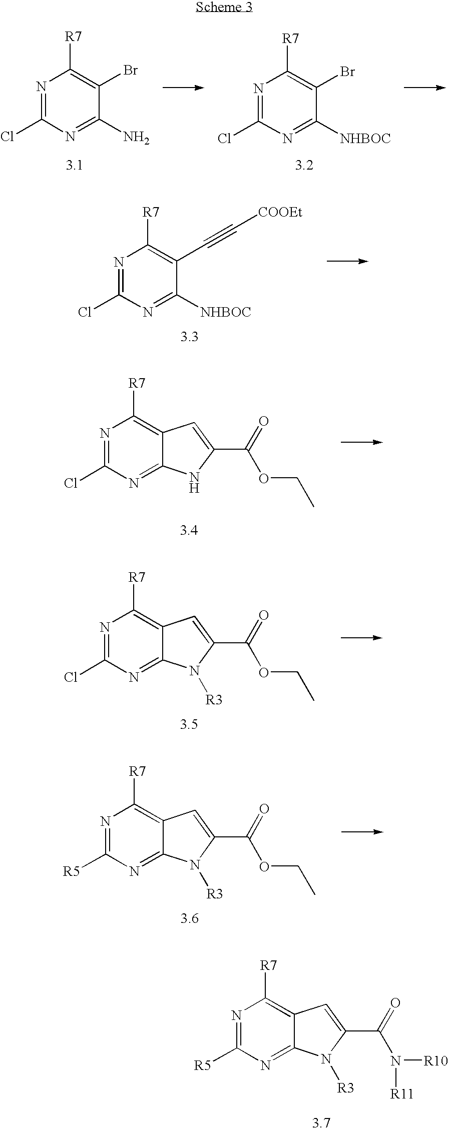

Scheme 4 describes the synthesis of imidazopyridine analogs such as 4.5. The appropriately substituted 3-amino 2-chloropyridine 4.1 when heated with a primary amine such as R3NH₂ affords the 2,3-diaminopyridine 4.2. Reaction with ethyl glyoxalate affords the ester intermediate 4.3. Hydrolysis of the ester followed by amide coupling can afford the desired imidazopyridine amide analogs such as 4.5.

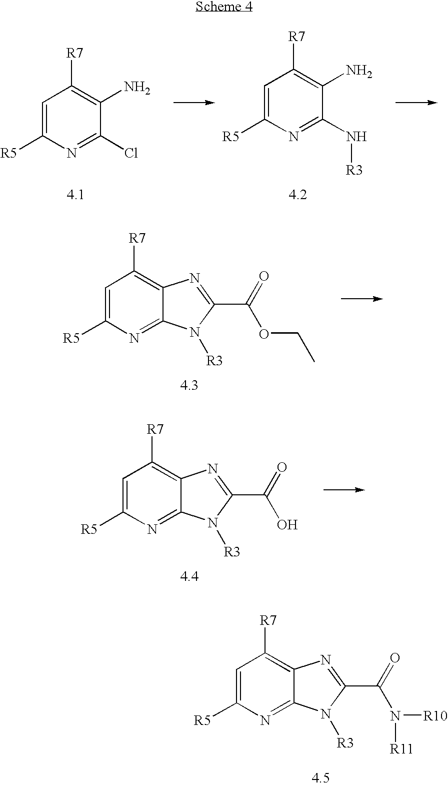

Scheme 5 describes the synthesis of pyrrolopyridine analogs such as 5.5. The appropriately substituted 3-aminopyridine such as 5.1 can be brominated at the 2-position by reaction with NBS. Sonogashira coupling with ethyl propiolate would afford the alkyne 5.3. Cyclization to the 2-substituted pyrolopyridine can be done by first protecting the amine as the Boc derivative, then heating with tetrabutyl ammonium fluoride. Hydrolysis of the ester is effected by refluxing in 4N HCl and acetonitrile as co-solvent. The resulting acid (5.4) is converted to amides 5.5 through standard amide coupling agents such as HBTU.

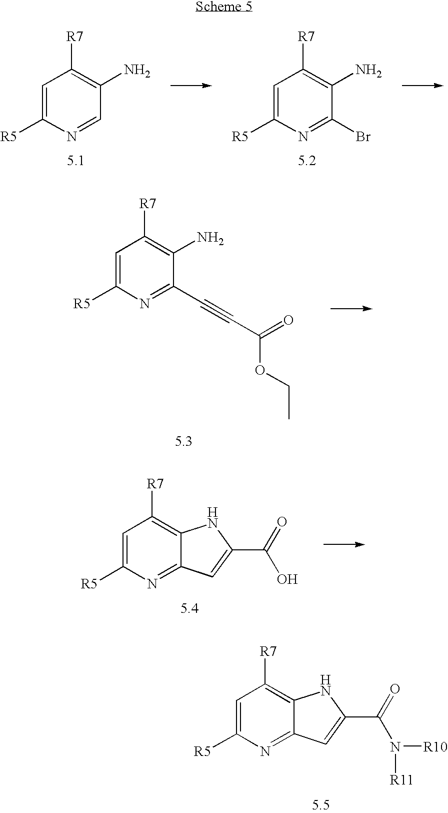

Scheme 6 shows the synthesis of pyrazolo[1,5-a]pyridines. Compounds can be prepared by 1,3-dipolar cycloaddition of substituted N-aminopyridines 6.2 with an alkyne such as methyl propiolate, dimethyl acetylenedicarboxylate or the like. N-amination of pyridines can be carried out by treating substituted pyridines 6.1 with aminating reagents such as hydroxylamine-O-sulfonic acid, O-mesitylenesulfonylhydroxylamine (MSH), O-(2,4-dinitrophenyl)hydroxylamine (Ref: C. Legault, A. B. Charette, J. Org. Chem., 2003, 68, 7119-7122; S. Löber, H. Hübner, W. Utz, P. Gmeiner, J. Med. Chem., 2001, 44, 2691-2694; also WO2006068826). Substituted pyridines can in turn be prepared by a variety of methods known in the literature such as the Chichibabin pyridine synthesis, Hantzsch pyridine synthesis, Guareschi-Thorpe pyridine synthesis, Bohlmann-Rahtz pyridine synthesis, Krohnke pyridine synthesis or Boger pyridine synthesis. Regarding the preparation of pyridines, see Comprehensive Heterocyclic Chemistry II Vol. 5, A. Katrizky, C. Rees, E. Scriven.

For example, compounds of formula 6.3, can be prepared in which dimethyl acetylenedicarboxylate is treated with optionally substituted N-aminopyridine in the presence of a suitable base such as potassium carbonate, DBU and the like, in a suitable solvent such as DMF, and the like. Compounds of formula 6.4 can be prepared by the acidic hydrolysis and chemoselective decarboxylation with a suitable acid such as concentrated sulfuric acid and the like under heating conditions.

For example, compounds of formula 6.5, in which R² is C(O)NR¹⁰R¹¹ can be prepared by reacting a deprotected carboxylic acid with a primary or secondary amine or amine salt, e.g. amine of the formula NR¹⁰R¹¹.

The reaction can be carried out with the acid in the presence of a coupling agent such as benzotriazole-1-yloxytrispyrrolidino-phosphonium hexafluorophosphate (PyBOP®), bromo-tris-pyrrolidino-phosphonium hexafluorophosphate (PyBroP®), 2-(1H-benzotriazole-1-yl)-1,1,3,3-tetramethylaminium hexafluorophosphate (HBTU), O-(7-azabenzotriazol-1-yl)-N,N,N′,N′-tetramethyluronium hexafluorophosphate (HATU), or 1,3-dicyclohexylcarbodiimide (DCC) or 1-(3-dimethylaminopropyl)-3-ethylcarbodiimide hydrochloride (EDC) optionally in the presence of 1-hydroxybenzotriazole (HOBt). As appropriate, a base such as N,N-diisopropylethylamine, triethylamine, or N-methylmorpholine can be used. The reaction is carried out in suitable organic solvents, such as DMF, THF and the like. Suitable amines and amine salts are either commercially available or they can be prepared from commercial available starting materials by methods known in the art.

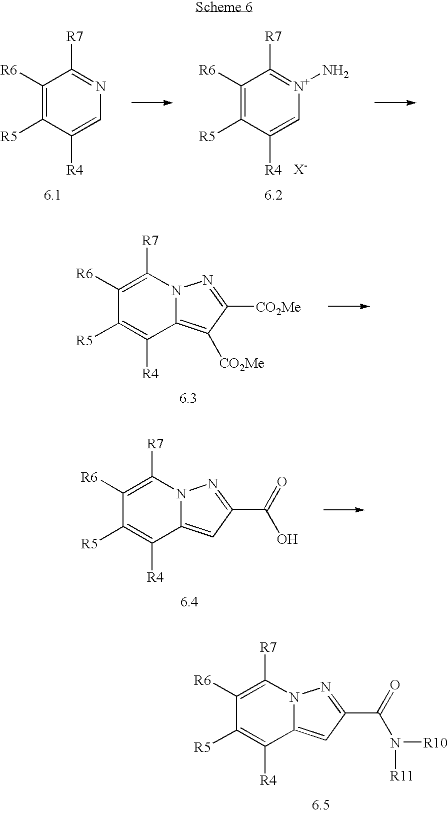

A compound of formula 7.4 or 7.5 in which R⁷ is Br, I, or alkyl can be prepared by deprotection of compound of formula 7.1 in which R⁷ is H with a base followed by addition of an electrophilic agent as shown in Scheme 7. This reaction is carried out in suitable organic solvents such as THF, ether and the like and at temperature about −78° C. Base such as n-buthyl lithium can be used for the deprotonation. Electrophilic reagents such as bromine, iodine, 1,2-dibromo-tetrachloroethane, methyl iodide can be used.

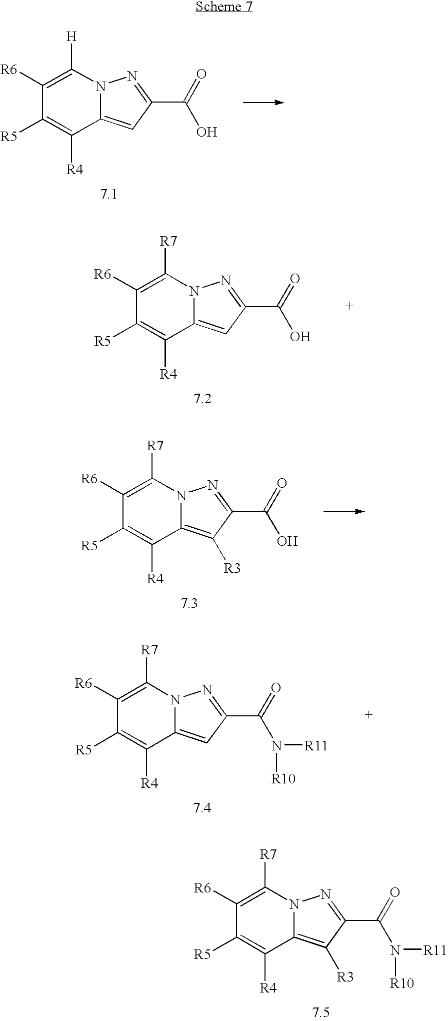

Referring to Scheme 8, a compound of formula 8.3 in which R³ is Cl, Br, or I, can be prepared by treating compounds of formula 8.1 or 8.4 in which R³ is H with electrophilic agents such as N-bromosuccinimide (NBS), N-chlorosuccinimide (NCS), N-iodosuccinimide (NIS). The reaction can be carried out in suitable solvents such as DMF, acetonitrile, chloroform, acetic acid and the like and at room temperature or heating at 40-50° C.

A compound of formula 8.3 in which R³ is NO₂ can be prepared by treating compounds of formula 8.1 in which R³ is H with nitrating agents such as fuming nitric acid, potassium nitrate or the like. The reaction can be carried out with suitable solvents such as sulfuric acid, acetic anhydride, trifluoroacetic acid and the like.

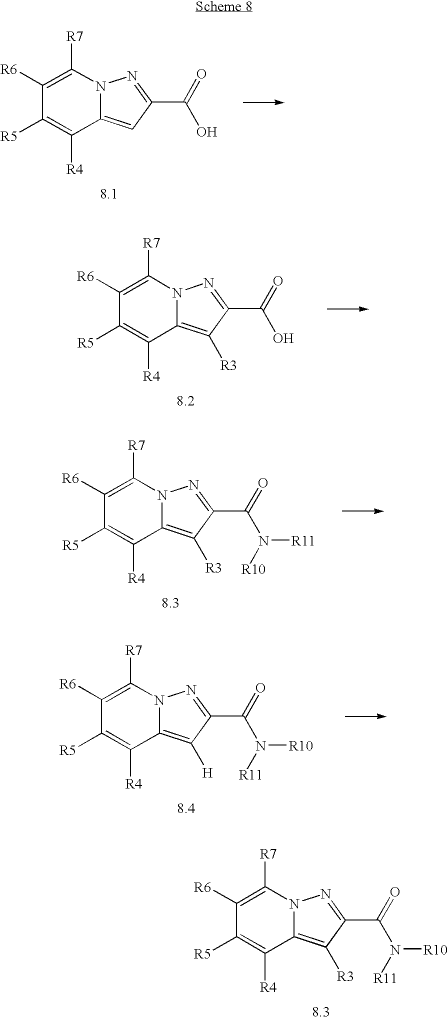

Referring to Scheme 9, a compound of formula 9.2 with R⁷ is NR¹⁰R¹¹ or OR¹⁵ can be prepared by substitution of a compound 9.1 with R⁷ is Br or Cl with an amine or alcohol in a suitable solvent such as DMF, DMA, NMP and the like. These reactions can be carried out at 120-200° C. under conventional heating or under microwave conditions.

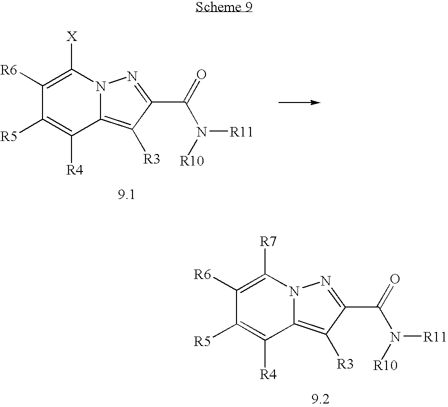

Referring to Scheme 10, a compound of formula 10.2 with R⁷ is CN, optionally substituted aryl, optionally substituted heteroaryl, or optionally substituted amino can be prepared by transition metal-mediated reactions of a compound with formula 10.1 with R⁷ is Cl, Br, or I. For example, these transition metal-mediated reactions can be one of those in the literature such as Suzuki-Miyaura reacions, Heck reactions, Stille reactions, Sonogashira reactions, and Buchwald aminations.

Similarly, a compound of formula 10.4 with R⁵ is CN, optionally substituted aryl, optionally substituted heteroaromatic rings, or optionally substituted amino can be prepared by transition metal-mediated reactions of a compound with formula 10.3 with R⁵ is Cl, Br, or I. For example, these transition metal-mediated reactions can be one of those in the literature such as Suzuki-Miyaura reacions, Heck reactions, Stille reactions, Sonogashira reactions, and Buchwal-Hartwig aminations.

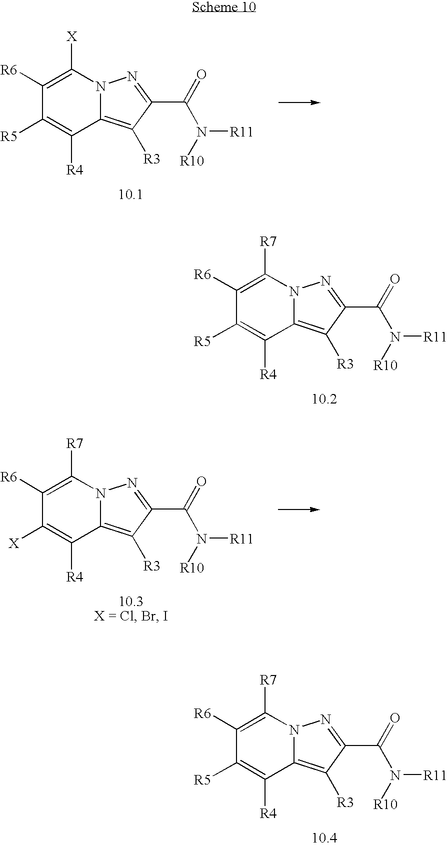

Referring to Scheme 11, compounds of formula 11.10 in which R⁷ is polyhalogenated alkyl, such as CF₂Cl or CF₃, can be prepared. Pyrazolo[1,5-a]pyridines may be prepared by Hemetsberger-Knittel synthesis by thermolysis of substituted 2-azido-2-pyridine acrylate of formula 11.8. (K. L. Stevens, et'al, Org. Lett., 2005, 7, 4653-4756; P. J. Roy, et al., Synthesis, 2005, 16, 2751-2757.)

Substituted pyridines of formula 11.5 with R⁷ is polyhalogenated alkyl, such as CF₃, or CF₂Cl, can be prepared using the Krohnke pyridine synthesis (F. Kröhnke, Synthesis, 1976, 1-24) by reacting a pyridinium salt of formula 11.4 and 4-substituted-2-oxo-but-3-enoic acid or its acid salt in the presence of ammonium acetate. The reaction can be carried out in suitable solvents such as methanol, acetic acid, water and the like and heating at 80-100° C. maybe used.

Pyridinium salt of formula 11.4 in which R⁷ is CF₂Cl or CF₃ can be prepared by reacting 1-carboxymethylpyridinium chloride 11.1 (T. Thorsteinsson, et al, J. Med. Chem. 2003, 46, 4173-4181) with anhydrides such as trifluoroacetic anhydride, dichlorofluoroacetic anhydride in the presence of a base. As appropriate, a base such as N,N-diisopropylethylamine, or triethylamine can be used. The reaction is carried out in suitable organic solvents, such as ether, THF or the like and at temperature around 0° C. The betaeine of formula 11.3 can be hydrolyzed under acidic conditions to give Pyridinium salt of formula 11.4. Acids such as hydrochloric acid can be used and heating at 40-80° C. may be used.

4-Substituted-2-oxo-but-3-enoic acid can be obtained from commercial sources or can be prepared as known in the art. Compounds with R⁵ is furan-2-yl can be prepared by reacting 2-furaldehyde with pyruvic acid in the presence of base. Suitable bases such as aqueous sodium hydroxide or aqueous potassium hydroxide can be used and temperature around 0° C. may be used.

Substituted pyridine 2-carboxyaldehyde 11.6 can be prepared by conversion of pyridine 2-carboxylic acid 11.5 to an ester followed by reduction with hydride reagents such as lithium aluminum hydride (LAH), di-isobutylaluminum hydride (DIBAL-H) and the like. The reaction can be carried out in suitable solvents such Et₂O, THF and the like and temperatures of from about −78 to 0° C. may be used. Alternatively, substituted pyridine 2-carboxyaldehyde 11.6 can be prepared by conversion of pyridine 2-carboxylic acid 11.5 to a Weinreb amide followed by reduction with hydride reagents such as lithium aluminum hydride (LAH), di-isobutylaluminum hydride (DIBAL-H) and the like. The reaction can be carried out in suitable solvents such Et₂O, THF and the like and temperatures of from about −78 to 0° C. may be used.

Substituted pyridine 2-carboxyaldehyde 11.6 can react with an alkyl azido acetate 11.7 under basic condition to give substituted 2-azido-2-pyridine acrylate of formula 11.8. Suitable bases such as sodium methoxide, sodium ethoxide, sodium tert-butoxide and the like can be used. The reaction can be carried out in suitable solvents such as methanol, ethanol, iso-propanol, tert-butanol and the like and the temperatures of from about −50 to 0° C. may be used.

Pyrazolo[1,5-a]pyridines of formula 11.9 can be prepared by heating substituted 2-azido-2-pyridine acrylate of formula 11.8. The reaction can be carried out in suitable solvents such as toluene, xylene, DMF, DMA, NMP and the like. These reactions can be carried out at 120-200° C. under conventional heating or under microwave conditions.

Esters of pyrazolo[1,5-a]pyridines of formula 11.9 can be saponified under basic conditions such as lithium hydroxide, sodium hydroxide, potassium hydroxide and the like. The reaction can be carried out in suitable solvents such as THF, methanol and the like with the addition of water. These reactions can be carried out at room temperature or optionally with heating. Similarly, the acids obtained can be coupled with an amine NHR¹⁰R¹¹ or amine salt to give compounds of formula 11.10 under standard amide coupling conditions described above.

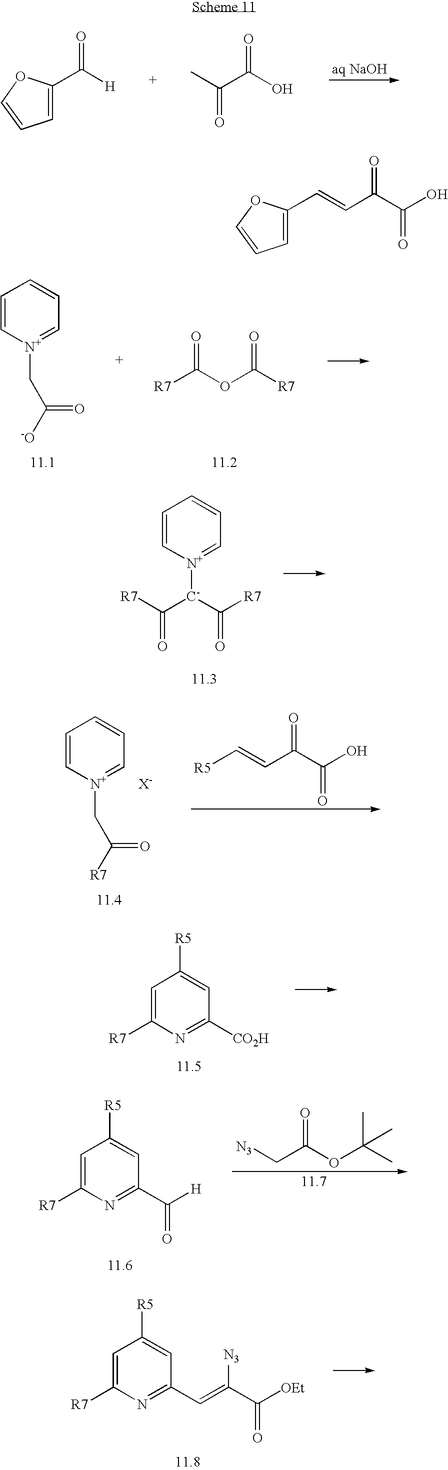

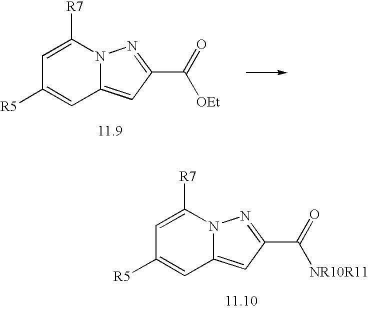

Scheme 12 describes the synthesis of imidazo[1,2-b]pyridazine analogs such as 12.6. The appropriately substituted 2-chloropyridazine 12.1 can be aminated with ammonia in solvents such as iso-propanol to give 2-aminopyridazine 12.2 and the reaction is usually carried out under heating in a sealed tube. 2-Chloropyridazine can in turn be prepared from chlorination of 2H-pyridazin-3-one with phosphoryl chloride and the like. Substituted 2-aminopyridazine can be cyclized with substituted methyl bromopyruvate in solvents such as DMF and the like and at temperatures 50-80° C. to give substituted imidazo[1,2-b]pyridazine 12.3. Halogenation at the 3-position can be carried out by reacting imidazo[1,2-b]pyridazine 12.3 with N-chlorosuccinimide, N-bromosuccinimide, N-iodosuccinimide and the like. The methyl ester of substituted imidazo[1,2-b]pyridazine 12.4 can be saponified with bases such as lithium hydroxide, sodium hydroxide, and the like and in solvents such as tetrahydrofuran, alcohol, and water. Substituted imidazo[1,2-b]pyridazine-2-carboxylic acids 12.5 can be converted to the amides 12.6 in the presence of a coupling agent such as benzotriazole-1-yloxytrispyrrolidino-phosphonium hexafluorophosphate (PyBOP®), bromo-tris-pyrrolidino-phosphonium hexafluorophosphate (PyBroP®), 2-(1H-benzotriazole-1-yl)-1,1,3,3-tetramethylaminium hexafluorophosphate (HBTU), O-(7-azabenzotriazol-1-yl)-N,N,N′,N′-tetramethyluronium hexafluorophosphate (HATU), or 1,3-dicyclohexylcarbodiimide (DCC) or 1-(3-dimethylaminopropyl)-3-ethylcarbodiimide hydrochloride (EDC) optionally in the presence of 1-hydroxybenzotriazole (HOBt). As appropriate, a base such as N,N-diisopropylethylamine, triethylamine, or N-methylmorpholine can be used. The reaction is carried out in suitable organic solvents, such as DMF, THF and the like. Suitable amines and amine salts are either commercially available or they can be prepared from commercial available starting materials by methods known in the art.

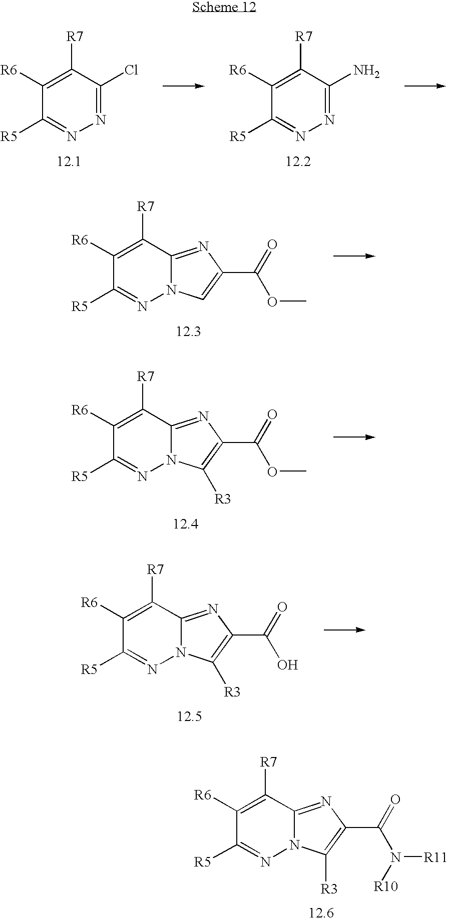

Scheme 13 describes the synthesis of benzimidazole analogs such as 13.7 and 13.8. Benzimidazole scaffold can be assembled by cyclization of substituted 2-acyl-1,2-diaminophenediamine. Substituted aniline 13.1 can be acylated with ethyl oxalyl chloride to give substituted N-phenyl-oxalamic acid ethyl ester 13.2 which in turn can be nitrated using nitric acid/sulfuric acid to give substituted N-(2-nitro-phenyl)-oxalamic acid ethyl ester 13.3. Reduction of nitro group can be carried out using sodium dithionite or other reducing reagents. Addition of aromatic or heteroaromatic groups with concomitant cyclization to benimidazole and saponification of ethyl ester can be achieved under Suzuki coupling conditions. The resultant substituted benzimidazole-2-carboxylic acids 13.5 can be converted to the amides 13.6 using standard coupling conditions as described above. Alkylation of benzimidazole can be carried out using alkyl halides, alkyl mesylate, alkyl triflates or the like and with suitable bases such as sodium hydride in solvents such as DMF, THF and the like, to give benzimidazole analogs 13.7 and 13.8

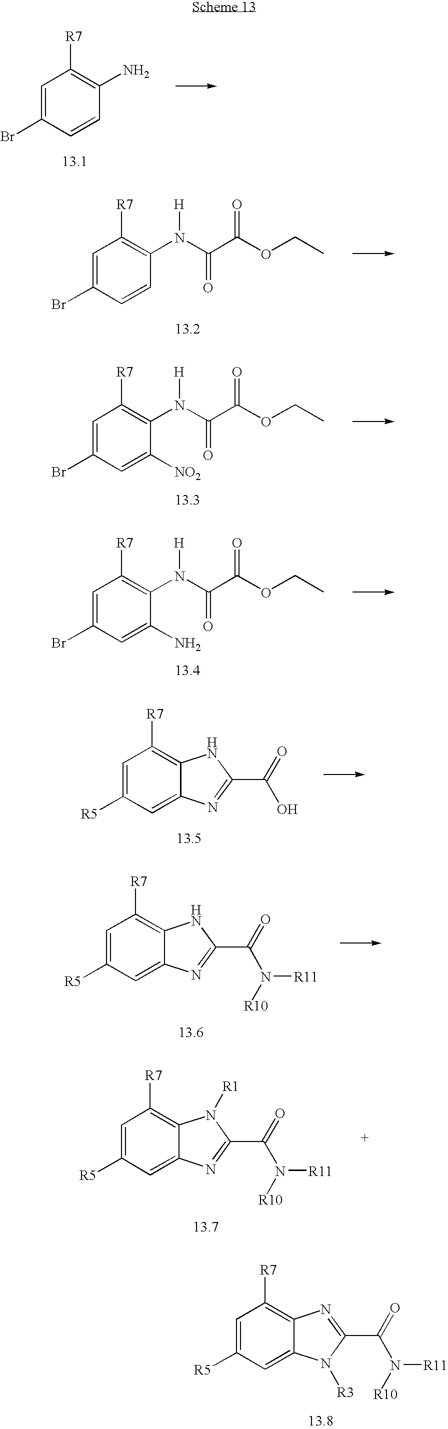

Alternatively, 1-alkyl-1H-benzimidazole derivatives can be prepared in Scheme 14. N-alkylation of substituted N-(2-nitro-phenyl)-oxalamic acid ethyl ester 14.1 can be prepared with alkyl halides, alkyl mesylates, alkyl triflates or the like with suitable bases such as sodium hydride in solvents such as DMF, THF and the like. Reduction of nitro group can be carried out using sodium dithionite or other reducing reagents. Addition of aromatic or heteroaromatic groups with concomitant cyclication to benzimidazole and saponification of ethyl ester can be achieved under Suzuki coupling conditions. The resultant substituted 1-alkyl-1H-benzoimidazole-2-carboxylic acids 14.4 can be converted to the amides 14.5 using standard coupling conditions as described above.

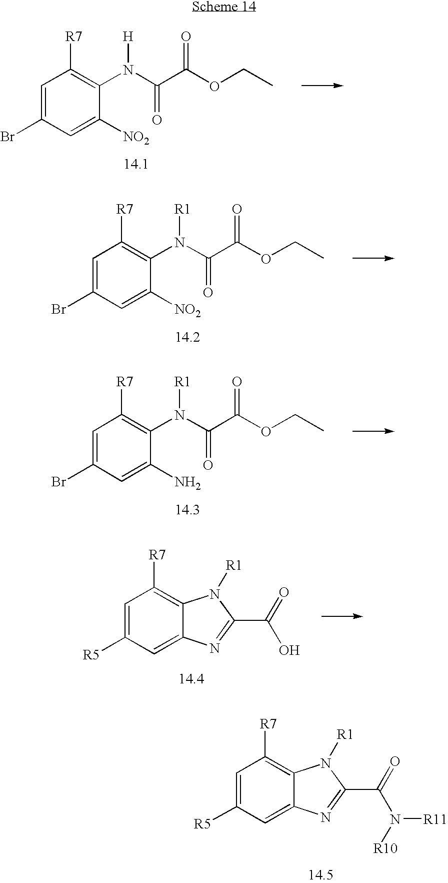

Provided are chemical entities possessing antiviral activity, including against hepatitis C virus. The chemical entities provided herein may inhibit viral replication by inhibiting the enzymes involved in replication, including RNA dependent RNA polymerase. They may also inhibit other enzymes utilized in the activity or proliferation of viruses in the flaviviridae family, such as HCV.

The chemical entities described herein are administered at a therapeutically effective dosage, e.g., a dosage sufficient to provide treatment for the disease states previously described. While human dosage levels have yet to be optimized for the chemical entities described herein, generally, a daily dose ranges from about 0.05 to 100 mg/kg of body weight; in certain embodiments, from about 0.10 to 10.0 mg/kg of body weight, and in certain embodiments, from about 0.15 to 1.0 mg/kg of body weight. Thus, for administration to a 70 kg person, in certain embodiments, the dosage range would be about from 3.5 to 7000 mg per day; in certain embodiments, about from 7.0 to 700.0 mg per day, and in certain embodiments, about from 10.0 to 100.0 mg per day. The amount of the chemical entity administered will, of course, be dependent on the subject and disease state being treated, the severity of the affliction, the manner and schedule of administration and the judgment of the prescribing physician; for example, a likely dose range for oral administration would be from about 70 to 700 mg per day, whereas for intravenous administration a likely dose range would be from about 70 to 700 mg per day depending on compound pharmacokinetics.

Administration of the chemical entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used.

Pharmaceutical compositions or formulations include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In addition, the chemical entities described herein can be co-administered with, and the pharmaceutical compositions can include, other medicinal agents, pharmaceutical agents, adjuvants, and the like. Suitable medicinal and pharmaceutical agents include therapeutically effective amounts of one or more agents active against HCV. In some embodiments, the agent active against HCV is an inhibitor of HCV proteases, HCV polymerase, HCV helicase, HCV NS4B protein, HCV entry, HCV assembly, HCV egress, HCV replicase, HCV NS5A protein, or inosine 5′-monophosphate dehydrogenase. In some embodiments, the agent active against HCV is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

Active agents against HCV include ribavirin, levovirin, viramidine, thymosin alpha-1, an inhibitor of NS3 serine protease, and inhibitor of inosine monophosphate dehydrogenase, interferon-alpha, either alone or in combination with ribavirin or levovirin. In some embodiments, the additional agent active against HCV is interferon-alpha or pegylated interferon-alpha alone or in combination with ribavirin or levovirin. In some embodiments, the agent active against hepatitis C virus is interferon.

Other suitable medicinal and pharmaceutical agents include TRH, diethylstilbesterol, theophylline, enkephalins, E series prostaglandins, compounds disclosed in U.S. Pat. No. 3,239,345 (e.g., zeranol), compounds disclosed in U.S. Pat. No. 4,036,979 (e.g., sulbenox), peptides disclosed in U.S. Pat. No. 4,411,890 growth hormone secretagogues such as GHRP-6, GHRP-1 (disclosed in U.S. Pat. No. 4,411,890 and publications WO 89/07110 and WO 89/07111), GHRP-2 (disclosed in WO 93/04081), NN7O3 (Novo Nordisk), LY444711 (Lilly), MK-677 (Merck), CP424391 (Pfizer) and B-HT920, growth hormone releasing factor and its analogs, growth hormone and its analogs and somatomedins including IGF-1 and IGF-2, alpha-adrenergic agonists, such as clonidine or serotonin 5-HT_{D} agonists, such as sumatriptan, agents which inhibit somatostatin or its release, such as physostigmine, pyridostigmine, parathyroid hormone, PTH(1-34), and bisphosphonates, such as MK-217 (alendronate).

Still other suitable medicinal and pharmaceutical agents include estrogen, testosterone, selective estrogen receptor modulators, such as tamoxifen or raloxifene, other androgen receptor modulators, such as those disclosed in Edwards, J. P. et. al., Bio. Med. Chem. Let., 9, 1003-1008 (1999) and Hamann, L. G. et. al., J. Med. Chem., 42, 210-212 (1999), and progesterone receptor agonists (“PRA”), such as levonorgestrel, medroxyprogesterone acetate (MPA).

Still other suitable medicinal and pharmaceutical agents include HIV and AIDS therapies, such as indinavir sulfate, saquinavir, saquinavir mesylate, ritonavir, lamivudine, zidovudine, lamivudine/zidovudine combinations, zalcitabine, didanosine, stavudine, and megestrol acetate.

Still other suitable medicinal and pharmaceutical agents include antiresorptive agents, hormone replacement therapies, vitamin D analogues, elemental calcium and calcium supplements, cathepsin K inhibitors, MMP inhibitors, vitronectin receptor antagonists, Src SH.sub.2 antagonists, vacular—H⁺-ATPase inhibitors, ipriflavone, fluoride, Tibo lone, pro stanoids, 17-beta hydroxysteroid dehydrogenase inhibitors and Src kinase inhibitors.

The above other therapeutic agents, when employed in combination with the chemical entities described herein, may be used, for example, in those amounts indicated in the Physicians' Desk Reference (PDR) or as otherwise determined by one of ordinary skill in the art.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition will comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

The following examples serve to more fully describe the manner of using the above-described invention. It is understood that these examples in no way serve to limit the true scope of this invention, but rather are presented for illustrative purposes.

In general, the chemical entities provided will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used, the route and form of administration, and other factors. The drug can be administered more than once a day, such as once or twice a day.

Therapeutically effective amounts of the chemical entities described herein may range from approximately 0.05 to 50 mg per kilogram body weight of the recipient per day; such as about 0.0 1-25 mg/kg/day, for example, from about 0.5 to 10 mg/kg/day. Thus, for administration to a 70 kg person, the dosage range may be about 35-70 mg per day.

In general, the chemical entities will be administered as pharmaceutical compositions by any one of the following routes: oral, systemic (e.g., transdermal, intranasal or by suppository), or parenteral (e.g., intramuscular, intravenous or subcutaneous) administration. In certain embodiments, oral administration with a convenient daily dosage regimen that can be adjusted according to the degree of affliction may be used. Compositions can take the form of tablets, pills, capsules, semisolids, powders, sustained release formulations, solutions, suspensions, elixirs, aerosols, or any other appropriate compositions. Another manner for administering the provided chemical entities is inhalation.

The choice of formulation depends on various factors such as the mode of drug administration and bioavailability of the drug substance. For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical compositions have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nM in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, at least one chemical entity described herein in combination with at least one pharmaceutically acceptable excipient. Acceptable excipients are non-toxic, aid administration, and do not adversely affect the therapeutic benefit of the at least one chemical entity described herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, for injectable solutions, include water, saline, aqueous dextrose, and glycols.

Compressed gases may be used to disperse a chemical entity described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the chemical entity in a composition can vary within the full range employed by those skilled in the art. Typically, the composition will contain, on a weight percent (wt %) basis, from about 0.01-99.99 wt % of at least one chemical entity described herein based on the total composition, with the balance being one or more suitable pharmaceutical excipients. In certain embodiments, the at least one chemical entity described herein is present at a level of about 1-80 wt %. Representative pharmaceutical compositions containing at least one chemical entity described herein are described below.

Additionally, the present specification is directed to a pharmaceutical composition comprising a therapeutically effective amount of at least one chemical entity described herein in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrognease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as ROFERON interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O, Saunders and S. A. Raybuck, “Inosine Monophosphate Dehydrogenase Consideration of Structure, Kinetics and Therapeutic Potential,” Ann. Rep. Med. Chem., 2:201-210 (2000).

The following examples serve to more fully describe the manner of using the above-described invention. It is understood that these examples in no way serve to limit the true scope of the invention, but rather are presented for illustrative purposes.

### EXAMPLE 1

### 3-Chloro-6-furan-3-yl-8-trifluoromethyl-imidazo[1,2-a]pyridine-2-carboxylic acid (Compound 101)

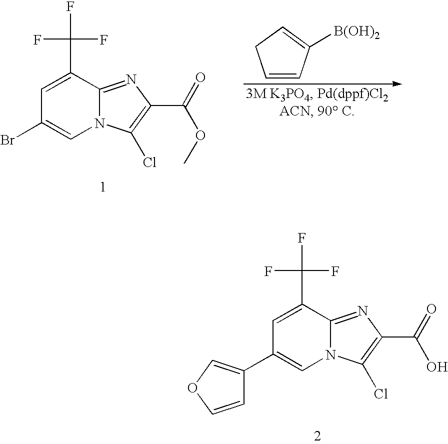

A mixture of 6-bromo-3-chloro-8-trifluoromethyl-imidazo[1,2-a]pyridine-2-carboxylic acid methyl ester (3.50 g, 9.79 mmol), 3-furan boronic acid (1.31 g, 11.75 mmol), Pd(dppf)Cl₂.CH₂Cl₂ (480 mg, 0.59 mmol), and 3M K₃PO₄ (32.63 mL, 97.90 mmol) in ACN (35 mL) was stirred vigorously at 90° C. overnight under N₂(g) atmosphere. The reaction mixture was concentrated, diluted in H₂O (350 mL), cooled to 0° C., acidified to pH ˜1 with conc. HCl, and extracted with EtOAc (2×150 mL). The organic layer was washed with brine (100 mL), dried (MgSO₄), and concentrated to afford a dark brown solid, which was suspended and stirred in ether for 15 min. The precipitate was filtered, the cake was washed with hot ether and dried under vacuum overnight to afford 3-chloro-6-furan-3-yl-8-trifluoromethyl-imidazo[1,2-a]pyridine-2-carboxylic acid as a light brown powder (2.65 g, 82.0%). ¹H NMR (d₆-DMSO, 300 MHz) δ 7.33 (m, 1H), 7.84 (m, 1H), 8.22 (s, 1H), 8.57 (s, 1H), 8.81 (s, 1H), 13.39 (s, 1H); MS (ESI) m/z=331.0 (MH⁺).

### EXAMPLE 2

### (3-Chloro-6-furan-3-yl-8-trifluoromethyl-imidazo[1,2-a]pyridin-2-yl)-(3-fluoro-3′,6′-dihydro-2′H-[2,4′]bipyridinyl-1′-yl)-methanone (hereinafter referred to as “Compound 1”)

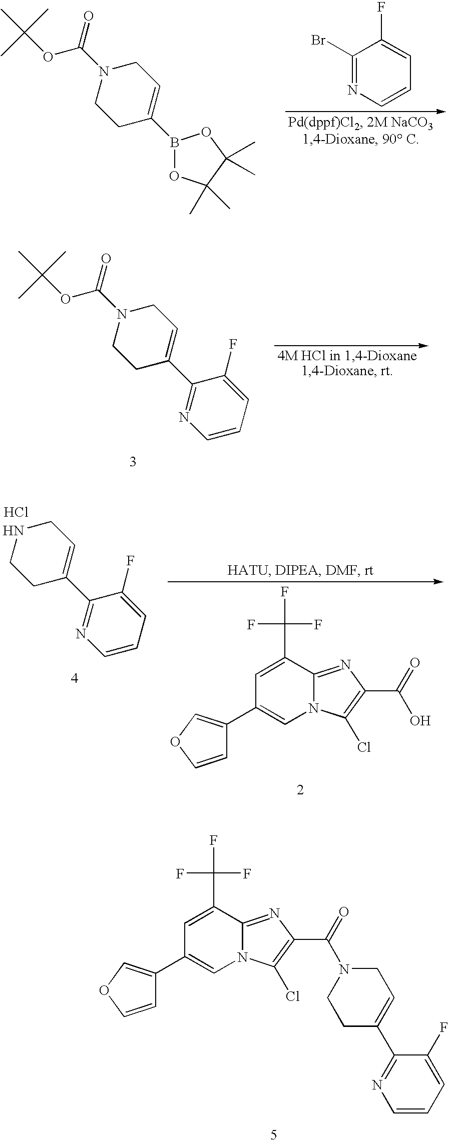

### Step 1: 3-Fluoro-3′,6′-dihydro-2′H-[2,4′]bipyridinyl-1′-carboxylic acid tert-butyl ester

A mixture of 3,6-dihydro-2H-pyridine-1-N-Boc-boronic acid pinacolato ester (8.00 g, 25.9 mmol), 2-bromo-3-fluoropyridine (4.14 g, 23.5 mmol), Pd(dppf)Cl.CH₂Cl₂ (1.15 g, 1.4 mmol) in 2 M Na₂CO₃ (35.28 mL, 70.6 mmol) and 1,4-dioxane (100 mL) was degassed twice and stirred at 90° C. for 2 hours. The mixture was concentrated on silica and flash column chromatography (1:1 EtOAc/hexane) afforded 3-fluoro-3′,6′-dihydro-2′H-[2,4′]bipyridinyl-1′-carboxylic acid tert-butyl ester as a pale yellow oil (5.2 g, 79%). ¹H NMR (CDCl₃, 300 MHz) δ 1.50 (s, 9H), 2.69 (m, 2H), 3.64 (t, 2H, J=5.40 Hz), 4.15 (d, 2H, J=3.00 Hz), 6.53 (m, 1H), 7.18 (m, 1H), 7.39 (m, 1H), 8.39 (m, 1H); MS (ESI) m/z=223.1 (MH⁺-t-Bu).

### Step 2: 3-Fluoro-1′,2′,3′,6′-tetrahydro-[2,4′]bipyridinyl hydrochloride (hereinafter referred to as “Compound 2”)

A 4 M HCl solution in 1,4-dioxane (16.4 mL, 65.8 mmol) was added to a stirring solution of 3-fluoro-3′,6′-dihydro-2′H-[2,4′]bipyridinyl-1′-carboxylic acid tert-butyl ester (3.66 g, 13.2 mmol) in 1,4-dioxane (50 mL). The reaction mixture was stirred at room temperature overnight. The precipitate was filtered, the cake was washed with ether, and dried under vacuum overnight to afford 3-fluoro-1′,2′,3′,6′-tetrahydro-[2,4′]bipyridinyl hydrochloride as a white solid (2.80 g, 99%). ¹H NMR (d₆-DMSO, 300 MHz) δ 2.81 (m, 2H), 3.31 (m, 2H), 3.81 (d, 2H, J=3.00 Hz), 6.56 (m, 1H), 7.43 (m, 1H), 7.78 (m, 1H), 8.45 (m, 1H), 9.24 (s, 2H); MS (ESI) m/z=179.1 (MH⁺).

### Step 3: (3-Chloro-6-furan-3-yl-8-trifluoromethyl-imidazo[1,2-a]pyridin-2-yl)-(3-fluoro-3′,6′-dihydro-2′H-[2,4′]bipyridinyl-1′-yl)-methanone

A solution of 3-chloro-6-furan-3-yl-8-trifluoromethyl-imidazo[1,2-a]pyridine-2-carboxylic acid (616 mg, 1.86 mmol), HATU (779 mg, 2.05 mmol), DIPEA (1.30 mL, 7.45 mmol), and 3-fluoro-1′,2′,3′,6′-tetrahydro-[2,4′]bipyridinyl hydrochloride (480 mg, 2.24 mmol) in DMF (16 mL) was stirred at room temperature for 2 hours. The solvent was evaporated, and the resulting residue was taken up in EtOAc (60 mL), washed with H₂O (2×20 mL), sat. NaHCO₃ (30 mL), brine (30 mL), dried (MgSO₄), concentrated on silica and flash column chromatography (0-30% MeOH/DCM) afforded (3-Chloro-6-furan-3-yl-8-trifluoromethyl-imidazo[1,2-a]pyridin-2-yl)-(3-fluoro-3′,6′-dihydro-2′H-[2,4′]bipyridinyl-1′-yl)-methanone as a white solid (640 mg, 70%). ¹H NMR (d₆-DMSO, 300 MHz) δ 2.73 (m, 2H), 3.85 (m, 2H), 4.42 (d, 2H, J=20.70 Hz), 6.58 (bd, 1H, J=49.50 Hz), 7.32 (m, 1H), 7.42 (m, 1H), 7.75 (m, 1H), 7.84 (t, 1H, J=1.80 Hz), 8.21 (s, 1H), 8.44 (m, 1H), 8.56 (s, 1H), 8.83 (s, 1H), 10.26 (s, 1H); MS (ESI) m/z=491.2 (MH⁺).

### EXAMPLE 3

### (3-Chloro-6-furan-3-yl-8-trifluoromethyl-imidazo[1,2-a]pyridin-2-yl)-[4-(2-fluoro-phenyl)-3-hydroxy-piperidin-1-yl]-methanone (Compound 102)

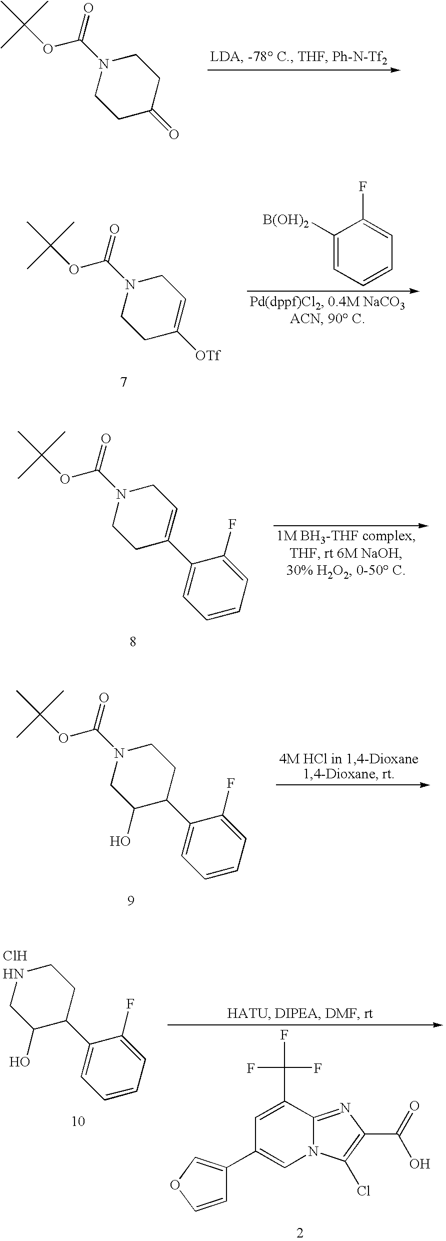

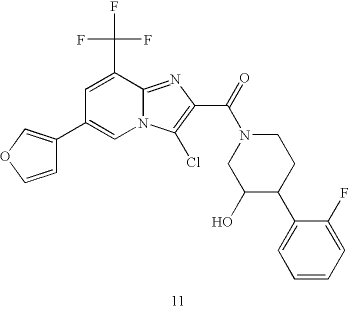

### Step 1: 4-Trifluoromethanesulfonyloxy-3,6-dihydro-2H-pyridine-1-carboxylic acid tert-butyl ester (hereinafter referred to as “Compound 3”)

A solution of t-butoxycarbonyl-4-piperidone (3 g, 15.1 mmol) in THF (10 mL) was slowly added to a stirring 2M solution of LDA (9.0 mL, 18.1 mmol) in THF (10 mL) at −78° C. After 10 min, a solution of N-phenyl bis(trifluoromethanesulfonamide) (5.9 g, 16.6 mmol) in THF (10 mL) was slowly added. After 30 min, the cooling bath was removed and reaction mixture was allowed to warm to room temperature over the course of 1.5 hours. The mixture was cooled to 0° C., quenched with sat. NaHCO₃ (30 mL), and extracted with ether (200 mL). The organic layer was washed with 5% citric acid (40 mL), 1M NaOH (4×40 mL), H₂O (2×40 mL), brine (40 mL), dried (MgSO₄), concentrated on silica and flash column chromatography (15-50% EtOAc/hexane gradient) afforded 4-trifluoromethanesulfonyloxy-3,6-dihydro-2H-pyridine-1-carboxylic acid tert-butyl ester as brown oil (3.4 g, 68%). ¹H NMR (CDCl₃, 300 MHz) δ 1.48 (s, 9H), 2.48 (m, 2H), 3.63 (t, 2H, J=5.70 Hz), 4.07 (m, 2H), 6.10 (t, 1H, J=3.30 Hz); MS (ESI) m/z=276.0 (MH⁺-t-Bu).

### Step 2: 4-(2-Fluoro-phenyl)-3,6-dihydro-2H-pyridine-1-carboxylic acid tert-butyl ester

Prepared similar to Compound 1. ¹H NMR (d₆-DMSO, 300 MHz) δ 1.43 (s, 9H), 2.42 (m, 2H), 3.52 (t, 2H, J=6.00 Hz), 3.99 (m, 2H), 5.98 (m, 1H), 7.19 (m, 2H), 7.30 (m, 2H); MS (ESI) m/z=222.1 (MH⁺-t-Bu).

### Step 3: 4-(2-Fluoro-phenyl)-3-hydroxy-piperidine-1-carboxylic acid tert-butyl ester

A 1 M solution of BH₃ in THF (2.16 mL, 2.16 mmol) was added to a stirring solution of 4-(2-fluoro-phenyl)-3,6-dihydro-2H-pyridine-1-carboxylic acid tert-butyl ester (300 mg, 1.08 mmol) in THF (10 mL) and the mixture stirred for 3 hours at room temperature. The reaction mixture was cooled to 0° C. in ice bath and 6 M NaOH (541 μL, 3.25 mmol) was added dropwise. After 10 min, hydrogen peroxide (30% wt., 368 μL, 3.25 mmol) was added and the resulting mixture was heated to 50° C. and stirred for 90 min. Water (20 mL) was added, and the mixture was extracted with EtOAc (2×20 mL), the organic layer was washed with brine (20 mL), dried (MgSO₄), concentrated on silica and flash column chromatography (0-100% EtOAc/hexane gradient) afforded 4-(2-fluoro-phenyl)-3-hydroxy-piperidine-1-carboxylic acid tert-butyl ester as a white semi-solid (260 mg, 81%). ¹H NMR (d₆-DMSO, 300 MHz) δ 1.42 (s, 9H), 1.62 (m, 2H), 4.12 (m, 1H), 3.55 (m, 1H), 2.80 (m, 2H), 3.96 (m, 2H), 4.91 (d, 1H, J=5.40 Hz), 7.25 (m, 4H); MS (ESI) m/z=240.1 (MH⁺-t-Bu).

### Step 4: Prepared similar to Compound 2

4-(2-Fluoro-phenyl)-piperidin-3-ol hydrochloride ¹H NMR (d₆-DMSO, 300 MHz) δ 1.91 (m, 2H), 2.73 (t, 1H, J=11.70 Hz), 2.98 (m, 2H), 4.00 (m, 1H), 3.21 (m, 2H), 5.31 (d, 1H, J=6.00 Hz), 7.21 (m, 4H), 9.13 (s, 2H); MS (ESI) m/z=196.1 (MH⁺).

### Step 5: Prepared Similar to Compound 1

### (3-Chloro-6-furan-3-yl-8-trifluoromethyl-imidazo[1,2-a]pyridin-2-yl)-[4-(2-fluoro-phenyl)-3-hydroxy-piperidin-1-yl]-methanone

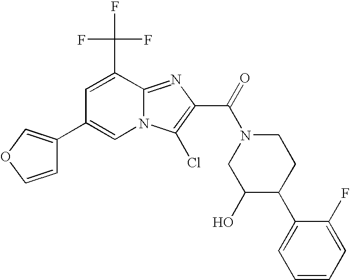

¹H NMR (d₆-DMSO, 300 MHz) δ 1.71 (m, 2H), 2.95 (m, 2H), 3.72 (m, 1H), 4.17 (m, 1H), 4.55 (m, 0.5H), 4.75 (m, 1H), 5.14 (d, 0.5H, J=5.10 Hz), 7.23 (m, 5H), 7.84 (m, 1H), 8.20 (d, 1H, J=6.60 Hz), 8.55 (s, 1H), 8.82 (d, 1H, J=8.70 Hz); MS (ESI) m/z=508.1 (MH⁺).

### EXAMPLE 4

### [3-Chloro-6-(1H-pyrazol-4-yl)-8-trifluoromethyl-imidazo[1,2-a]pyridin-2-yl]-[3-(2-fluoro-phenyl)-2,5-dihydro-pyrrol-1-yl]-methanone (Compound 103)

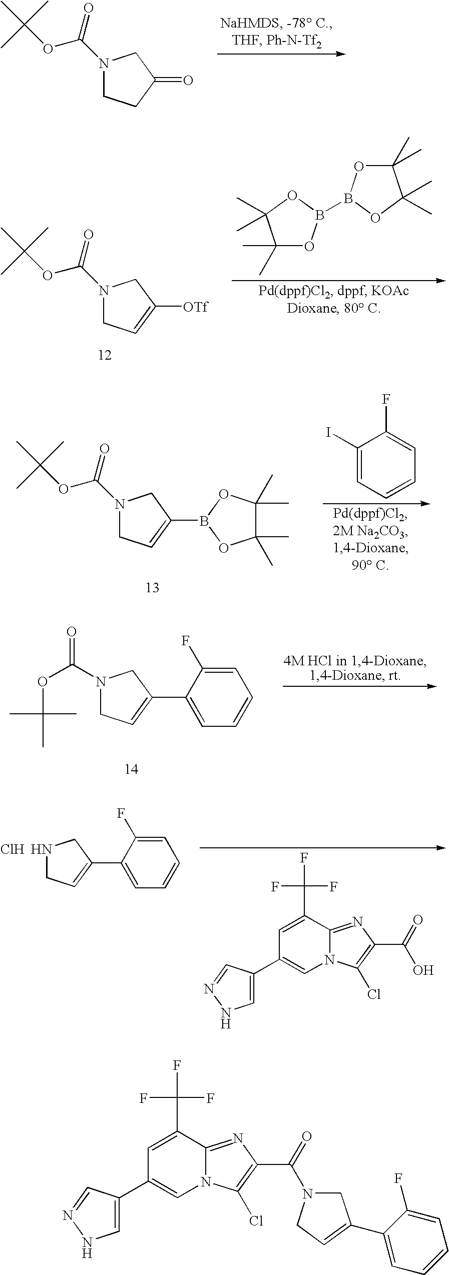

### Step 1: 3-Trifluoromethanesulfonyloxy-2,5-dihydro-pyrrole-1-carboxylic acid tert-butyl ester

Prepared similar to Compound 3, except NaHMDS was used instead of LDA. ¹H NMR (CDCl₃, 300 MHz) δ 1.41 (s, 9H), 4.14 (m, 4H), 5.60 (m, 1H); MS (ESI) m/z=261.9 (MH⁺-t-Bu).

### Step 2: 5-(4,4,5,5-Tetramethyl-[1,3,2]dioxaborolan-2-yl)-2,5-dihydro-pyrrole-1-carboxylic acid tert-butyl ester (hereinafter referred to as “Compound 6”)

3-Trifluoromethanesulfonyloxy-2,5-dihydro-pyrrole-1-carboxylic acid tert-butyl ester (2.00 g, 6.3 mmol) was dissolved in 1,4-dioxane (35 mL) and added under N₂ (g) to a degassed mixture of potassium acetate (1.86 g, 18.9 mmol), Pd(dppf)Cl₂.CH₂Cl₂ (154 mg, 0.19 mmol), dppf (105 mg, 0.19 mmol), bis-pinacolato diborane (1.92 g, 7.56 mmol) and the reaction mixture heated at 80° C. overnight. Concentration of the crude reaction mixture on silica gel followed by flash column chromatography (15-50% EtOAc/hexane gradient) afforded 5-(4,4,5,5-tetramethyl-[1,3,2]dioxaborolan-2-yl)-2,5-dihydro-pyrrole-1-carboxylic acid tert-butyl ester as white semi-solid (1.50 g, 81%). ¹H NMR (CDCl₃, 300 MHz) δ 1.26 (s, 12H), 1.46 (s, 9H), 4.18 (m, 4H), 6.45 (bd, 1H, J=14.10 Hz); MS (ESI) m/z=240.2 (MH⁺-t-Bu).

### Step 3: 3-(2-Fluoro-phenyl)-2,5-dihydro-pyrrole-1-carboxylic acid tert-butyl ester

Prepared similar to Compound 1. ¹H NMR (CDCl₃, 300 MHz) δ 1.51 (s, 9H), 4.34 (m, 2H), 4.53 (m, 2H), 6.34 (m, 1H), 7.10 (m, 2H), 7.52 (m, 2H); MS (ESI) m/z=208.1 (MH⁺-t-Bu).

### Step 4: 3-(2-Fluoro-phenyl)-2,5-dihydro-1H-pyrrole hydrochloride

Prepared similar to Compound 2. ¹H NMR (CDCl₃, 300 MHz) δ 4.39 (m, 2H), 4.57 (m, 2H), 6.45 (bd, 1H, J=95.10 Hz), 7.10 (m, 2H), 7.30 (m, 2H), 10.27 (s, 2H); MS (ESI) m/z=164.1 (MH⁺).

### Step 5: [3-Chloro-6-(1H-pyrazol-4-yl)-8-trifluoromethyl-imidazo[1,2-a]pyridin-2-yl]-[3-(2-fluoro-phenyl)-2,5-dihydro-pyrrol-1-yl]-methanone

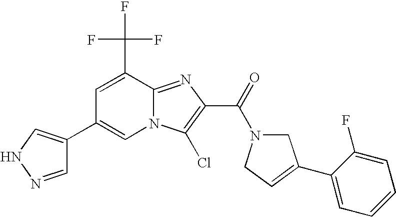

Prepared similar to Compound 1. ¹H NMR (d₆-DMSO, 300 MHz) δ 4.55 (m, 1H), 4.78 (m, 1H), 4.89 (m, 1H), 5.12 (m, 1H), 6.53 (m, 1H), 7.32 (m, 4H), 7.55 (m, 1H), 7.55 (m, 1H), 8.23 (s, 1H), 8.42 (m, 2H), 8.86 (s, 1H); MS (ESI) m/z=476.1 (MH⁺).

### EXAMPLE 5

### [3-Chloro-6-(1H-pyrazol-4-yl)-8-trifluoromethyl-imidazo[1,2-a]pyridin-2-yl]-[3-(1H-imidazol-4-yl)-2,5-dihydro-pyrrol-1-yl]-methanone (Compound 104)

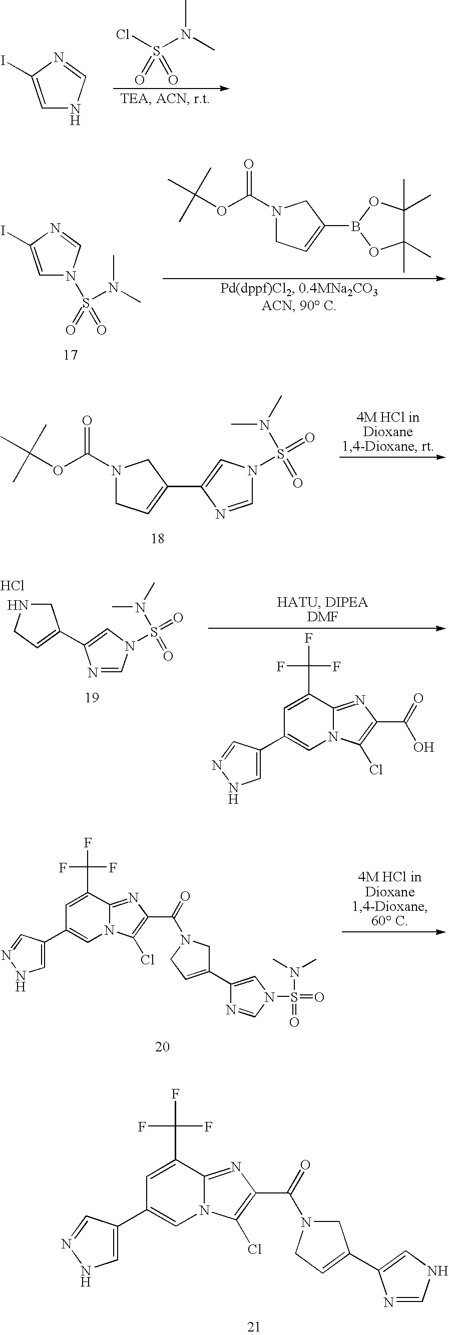

### Step 1: 4-Iodo-imidazole-1-sulfonic acid dimethylamide (hereinafter referred to as “Compound 4”)

N,N′-Dimethylsulfonamide chloride (550 μL, 5.16 mmol) was added to a stirring solution of 4-iodoimidazole (500 mg, 2.58 mmol) and TEA (0.90 mL, 6.44 mmol) in ACN (5 mL) at room temperature. After 2 hours, the mixture was concentrated on silica and subjected to flash column chromatography (10-40% EtOAc/hexane gradient) to afford 4-iodo-imidazole-1-sulfonic acid dimethylamide as white solid (620 mg, 80%). ¹H NMR (CDCl₃, 300 MHz) δ 1.34 (s, 6H), 5.78 (s, 1H), 6.23 (s, 1H); MS (ESI) m/z=301.9 (MH⁺).

### Step 2: 3-(1-Dimethylsulfamoyl-1H-imidazol-4-yl)-2,5-dihydro-pyrrole-1-carboxylic acid tert-butyl ester

Prepared similar to Compound 1. ¹H NMR (CDCl₃, 300 MHz) δ 1.49 (s, 9H), 2.88 (s, 6H), 4.31 (m, 4H), 6.29 (m, 1H), 7.11 (bd, 1H, J=20.40 Hz), 7.88 (m, 1H); S (ESI) m/z=343.2 (MH⁺).

### Step 3: 4-(2,5-Dihydro-1H-pyrrol-3-yl)-imidazole-1-sulfonic acid dimethylamide hydrochloride

Prepared similar to Compound 2. ¹H NMR (d₆-DMSO, 300 MHz) δ 2.84 (s, 6H), 4.11 (m, 2H), 4.21 (m, 2H), 6.26 (s, 1H), 7.92 (s, 1H), 8.24 (s, 1H), 9.44 (s, 2H); MS (ESI) m/z=243.1 (MH⁺).

### Step 4: 4-{1-[3-Chloro-6-(1H-pyrazol-4-yl)-8-trifluoromethyl-imidazo[1,2-a]pyridine-2-carbonyl]-2,5-dihydro-1H-pyrrol-3-yl}-imidazole-1-sulfonic acid dimethylamide

Prepared similar to Compound 1. MS (ESI) m/z=555.1 (MH⁺).

### Step 5: [3-Chloro-6-(1H-pyrazol-4-yl)-8-trifluoromethyl-imidazo[1,2-a]pyridin-2-yl]-[3-(1H-imidazol-4-yl)-2,5-dihydro-pyrrol-1-yl]-methanone

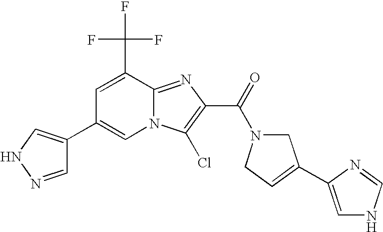

Prepared similar to Compound 2, except the reaction was heated at 60° C. instead of room temperature. ¹H NMR (d₆-DMSO, 300 MHz) δ 4.62 (bd, 2H, J=35.40 Hz), 4.89 (s, 2H), 6.69 (s, 1H), 7.72 (bd, 1H, J=129.60 Hz), 8.24 (s, 1H), 8.43 (s, 2H), 8.88 (d, 1H, J=4.20 Hz), 9.24 (d, 1H, J=6.60 Hz); MS (ESI) m/z=448.1 (MH⁺).

### EXAMPLE 6

### [3-Chloro-6-(1H-pyrazol-4-yl)-8-trifluoromethyl-imidazo[1,2-a]pyridin-2-yl]-(3-thiazol-5-yl-2,5-dihydro-pyrrol-1-yl)-methanone (Compound 105)

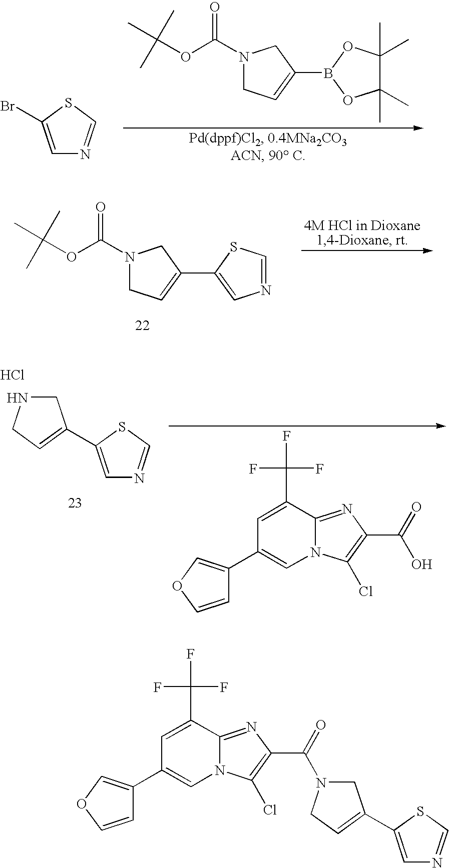

### Step 1: 3-Thiazol-5-yl-2,5-dihydro-pyrrole-1-carboxylic acid tert-butyl ester

Prepared similar to Compound 1. ¹H NMR (CDCl₃, 300 MHz) δ 1.49 (s, 9H), 4.29 (m, 2H), 4.45 (m, 2H), 6.03 (bd, 1H, J=13.80 Hz), 7.73 (s, 1H), 8.71 (s, 1H); MS (ESI) m/z=253.1 (MH⁺).

### Step 2: 5-(2,5-Dihydro-1H-pyrrol-3-yl)-thiazole hydrochloride

Prepared similar to Compound 2. ¹H NMR (d₆-DMSO, 300 MHz) δ 4.11 (m, 2H), 4.33 (m, 2H), 6.26 (m, 1H), 8.03 (s, 1H), 9.13 (s, 1H), 9.83 (s, 2H); S (ESI) m/z=153.0 (MH⁺).

### Step 3: (3-Chloro-6-furan-3-yl-8-trifluoromethyl-imidazo[1,2-a]pyridin-2-yl)-(3-thiazol-5-yl-2,5-dihydro-pyrrol-1-yl)-methanone (Compound 106)

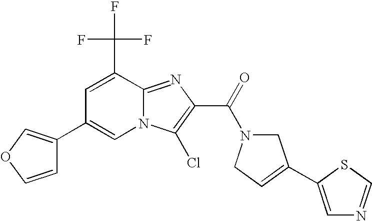

Prepared similar to Compound 1. ¹H NMR (d₆-DMSO, 300 MHz) δ 4.52 (m, 1H), 4.75 (m, 1H), 4.84 (m, 1H), 5.05 (m, 1H), 6.36 (m, 1H), 7.34 (m, 1H), 7.85 (m, 1H), 7.91 (bd, 1H, J=72.30 Hz), 8.24 (d, 1H, J=5.70 Hz), 8.58 (s, 1H), 8.85 (s, 1H), 9.09 (d, 1H, J=2.70 Hz); MS (ESI) m/z=465.1 (MH⁺).

### EXAMPLE 7

### [3-Chloro-6-(1H-pyrazol-4-yl)-8-trifluoromethyl-imidazo[1,2-a]pyridin-2-yl]-(3-thiazol-5-yl-2,5-dihydro-pyrrol-1-yl)-methanone (Compound 105)

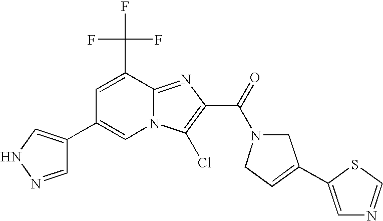

Prepared similar to Compound 1. ¹H NMR (d₆-DMSO, 300 MHz) δ 4.53 (m, 1H), 4.74 (m, 1H), 4.85 (m, 1H), 5.04 (m, 1H), 6.36 (m, 1H), 7.79 (s, 1H), 8.03 (s, 1H), 8.24 (d, 1H, J=5.40 Hz), 8.42 (m, 2H), 8.86 (s, 1H), 9.08 (d, 1H, J=3.00 Hz); MS (ESI) m/z=465.1 (MH⁺).

### EXAMPLE 8

### [3-Chloro-6-(1H-pyrazol-4-yl)-8-trifluoromethyl-imidazo[1,2-a]pyridin-2-yl]-(3-isothiazol-4-yl-2,5-dihydro-pyrrol-1-yl)-methanone (Compound 107)

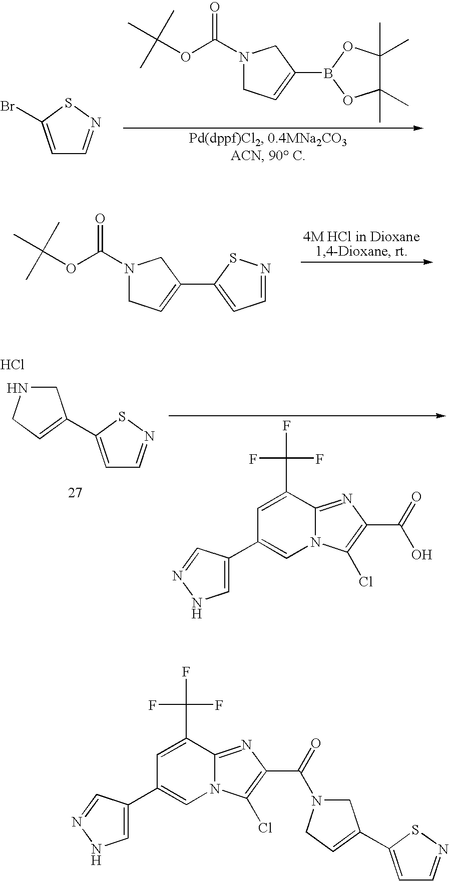

### Step 1: 3-Isothiazol-5-yl-2,5-dihydro-pyrrole-1-carboxylic acid tert-butyl ester

Prepared similar to Compound 1. ¹H NMR (CDCl₃, 300 MHz) δ 1.50 (s, 9H), 4.40 (m, 4H), 6.12 (bd, 1H, J=14.70 Hz), 8.40 (d, 1H, J=7.20 Hz), 8.64 (s, 1H); MS (ESI) m/z=253.1 (MH⁺).

### Step 2: 5-(2,5-Dihydro-1H-pyrrol-3-yl)-isothiazole hydrochloride

Prepared similar to Compound 2. ¹H NMR (d₆-DMSO, 300 MHz) δ 4.11 (m, 2H), 4.29 (m, 2H), 7.29 (m, 1H), 9.13 (s, 1H), 9.85 (s, 2H); MS (ESI) m/z=153.1 (MH⁺).

### Step 3: [3-Chloro-6-(1H-pyrazol-4-yl)-8-trifluoromethyl-imidazo[1,2-a]pyridin-2-yl]-(3-isothiazol-4-yl-2,5-dihydro-pyrrol-1-yl)-methanone

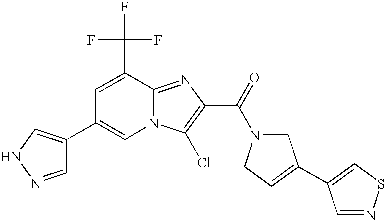

Prepared similar to Compound 1. ¹H NMR (d₆-DMSO, 300 MHz) δ 4.54 (m, 1H), 4.71 (m, 1H), 4.85 (m, 1H), 4.99 (m, 1H), 6.51 (m, 1H), 8.23 (s, 1H), 8.42 (s, 2H), 8.80 (s, 1H), 8.87 (m, 1H), 8.96 (s, 1H), 9.12 (s, 1H); MS (ESI) m/z=465.1 (MH⁺).

### EXAMPLE 9

### (3-Chloro-6-furan-3-yl-8-trifluoromethyl-imidazo[1,2-a]pyridin-2-yl)-(4-methanesulfonyl-piperazin-1-yl)-methanone (Compound 108)

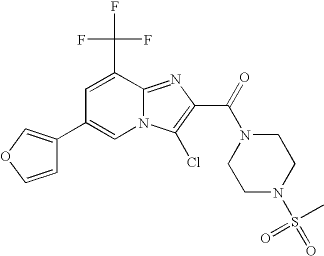

### Prepared Similar to Compound 1.

¹H NMR (d₆-DMSO, 300 MHz) δ 2.94 (s, 3H), 3.16 (m, 2H), 3.25 (m, 2H), 3.81 (m, 4H), 7.33 (d, 1H, J=1.80 Hz), 7.84 (t, 1H, J=1.50 Hz), 8.22 (s, 1H), 8.56 (s, 1H), 8.83 (s, 1H); MS (ESI) m/z=498.8 (MNa⁺).

### EXAMPLE 10

### 2-(6-Furan-3-yl-8-trifluoromethyl-imidazo[1,2-a]pyridin-2-yl)-1-(4-thiazol-2-yl-piperazin-1-yl)-ethanone (Compound 109)

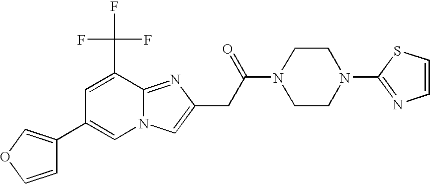

### Prepared Similar to Compound 1.

¹H NMR (d₆-DMSO, 300 MHz) δ 3.45 (m, 2H), 3.51 (m, 2H), 3.67 (m, 2H), 3.80 (m, 2H), 4.00 (m, 2H), 6.93 (dd, 1H, J=0.90, 3.90 Hz), 7.03 (m, 1H), 7.25 (dd, 1H, J=1.20, 4.80 Hz), 7.82 (t, 1H, J=1.80 Hz), 7.98 (s, 1H), 8.03 (s, 1H), 8.40 (s, 1H), 9.14 (s, 1H); MS (ESI) m/z=461.9 (MH⁺).

### EXAMPLE 11

### 2-(6-Furan-3-yl-8-trifluoromethyl-imidazo[1,2-a]pyridin-2-yl)-1-(2-phenyl-piperidin-1-yl)-ethanone (Compound 110)

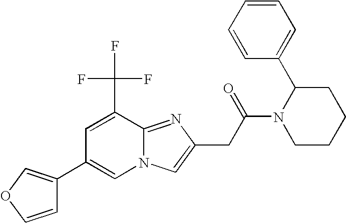

### Prepared Similar to Compound 1.

¹H NMR (d₆-DMSO, 300 MHz) δ 1.35 (m, 2H), 1.56 (m, 2H), 1.82 (m, 1H), 2.42 (m, 1H), 2.96 (m, 1H), 4.10 (m, 2H), 5.65 (bd, 1H, J=80.10 Hz), 7.04 (s, 1H), 7.24 (m, 3H), 7.34 (m, 2H), 7.83 (t, 1H, J=1.50 Hz), 8.03 (m, 2H), 8.41 (s, 1H), 9.16 (s, 1H); MS (ESI) m/z=454.1 (MH⁺).

### EXAMPLE 12

### (3-Chloro-6-furan-3-yl-8-trifluoromethyl-imidazo[1,2-a]pyridin-2-yl)-[4-(1H-imidazol-2-yl)-3,6-dihydro-2H-pyridin-1-yl]-methanone (Compound III)

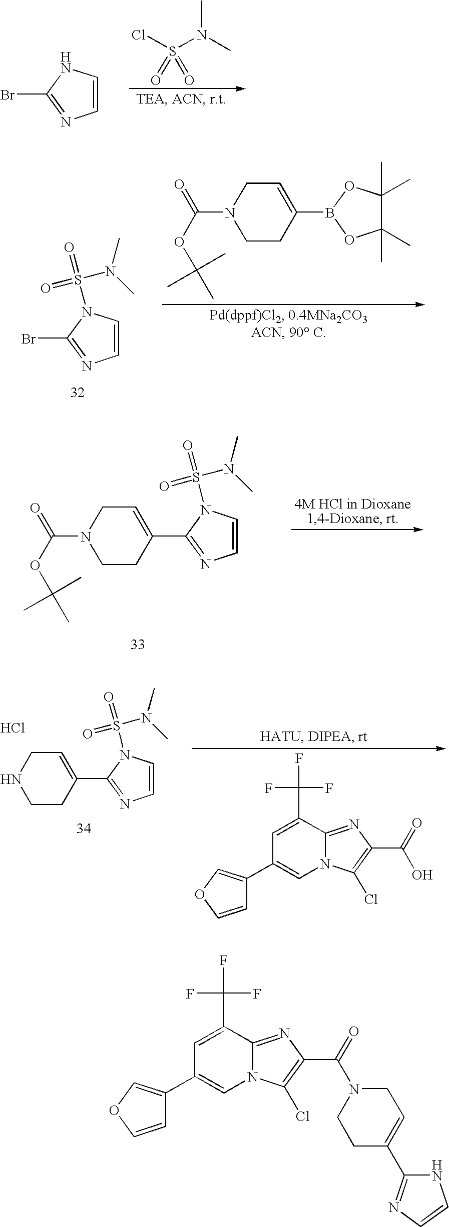

### Step 1: 2-Bromo-imidazole-1-sulfonic acid dimethylamide

Prepared similar to Compound 4. ¹H NMR (CDCl₃, 300 MHz) δ 2.92 (s, 6H), 7.08 (d, 1H, J=1.80 Hz), 7.67 (d, 1H, J=1.80 Hz); MS (ESI) m/z=302 (MH⁺).

### Step 2: 4-(1-Dimethylsulfamoyl-1H-imidazol-2-yl)-3,6-dihydro-2H-pyridine-1-carboxylic acid tert-butyl ester

Prepared similar to Compound 1. ¹H NMR (CDCl₃, 300 MHz) δ 1.48 (s, 9H), 2.54 (m, 2H), 2.82 (s, 6H), 3.62 (m, 2H), 4.09 (m, 2H), 6.17 (s, 1H), 7.01 (d, 1H, J=1.80 Hz), 7.30 (d, 1H, J=1.50 Hz); MS (ESI) m/z=357.2 (MH⁺).

### Step 3: 4-(2,5-Dihydro-1H-pyrrol-3-yl)-imidazole-1-sulfonic acid dimethylamide hydrochloride

Prepared similar to Compound 2. ¹H NMR (d₆-DMSO, 300 MHz) δ 2.78 (m, 2H), 3.33 (m, 2H), 3.46 (m, 6H), 3.87 (m, 2H), 6.94 (s, 1H), 7.71 (m, 2H), 9.45 (s, 2H); MS (ESI) m/z=257.1 (MH⁺).

### Step 4: (3-Chloro-6-furan-3-yl-8-trifluoromethyl-imidazo[1,2-a]pyridin-2-yl)-[4-(1H-imidazol-2-yl)-3,6-dihydro-2H-pyridin-1-yl]-methanone (Compound III)

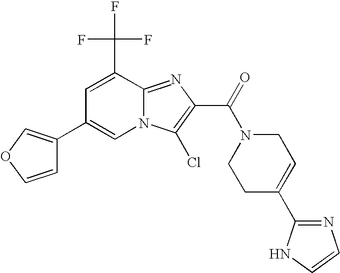

Prepared similar to Compound 1. ¹H NMR (d₆-DMSO, 300 MHz) δ 2.68 (m, 2H), 3.93 (m, 2H), 4.52 (bd, 2H, J=42.30 Hz), 6.85 (bd, 1H, J=43.80 Hz), 7.33 (s, 1H), 7.72 (s, 2H), 7.85 (s, 1H), 8.23 (s, 1H), 8.57 (s, 1H), 8.84 (s, 1H); MS (ESI) m/z=461.9 (MH⁺).

### EXAMPLE 13

### [3-Chloro-6-(1H-pyrazol-4-yl)-8-trifluoromethyl-imidazo[1,2-a]pyridin-2-yl]-[4-(1H-pyrazol-4-yl)-3,6-dihydro-2H-pyridin-1-yl]-methanone (Compound 112)

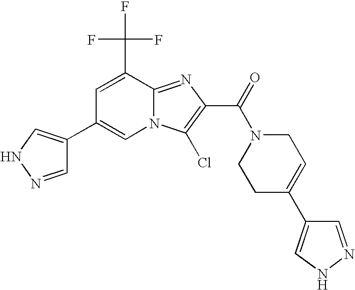

Prepared similar to Compound 1. ¹H NMR (d₆-DMSO, 300 MHz) δ 2.48 (m, 2H), 3.84 (m, 2H), 4.28 (m, 2H), 5.99 (bd, 1H, J=42.60 Hz), 7.74 (d, 2H, J=3.30 Hz), 8.20 (s, 1H), 8.40 (s, 2H), 8.83 (s, 1H); MS (ESI) m/z=461.9 (MH⁺).

### EXAMPLE 14

### 1-(1,3-Dihydro-isoindol-2-yl)-2-(6-furan-3-yl-8-trifluoromethyl-imidazo[1,2-a]pyridin-2-yl)-ethanone (Compound 113)

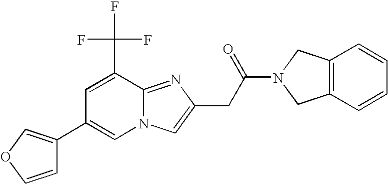

Prepared similar to Compound 1. ¹H NMR (d₆-DMSO, 300 MHz) δ 3.98 (s, 2H), 4.70 (s, 2H), 5.02 (s, 2H), 7.02 (s, 1H), 7.33 (m, 4H), 7.81 (s, 1H), 7.96 (s, 1H), 7.99 (d, 1H, J=3.60 Hz), 8.39 (s, 1H), 9.12 (s, 1H); MS (ESI) m/z=412.1 (MH⁺).

### EXAMPLE 15

### 1′-(3-Chloro-6-furan-3-yl-8-trifluoromethyl-imidazo[1,2-a]pyridine-2-carbonyl)-[1,4′]bipiperidinyl-2-one (Compound 114)

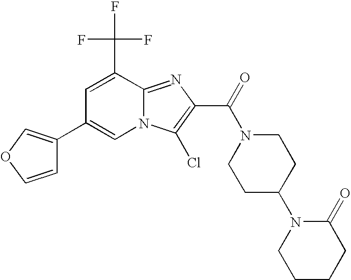

Prepared similar to Compound 1. ¹H NMR (d₆-DMSO, 300 MHz) δ 1.50 (m, 1H), 1.67 (m, 7H), 2.23 (t, 2H, J=6.00 Hz), 2.86 (m, 1H), 3.16 (m, 3H), 4.18 (m, 1H), 4.60 (m, 2H), 7.31 (m, 1H), 7.84 (t, 1H, J=1.20 Hz), 8.19 (s, 1H), 8.55 (s, 1H), 8.81 (s, 1H); MS (ESI) m/z=495.1 (MH⁺).

### EXAMPLE 16

### (3-Chloro-6-furan-3-yl-8-trifluoromethyl-imidazo[1,2-a]pyridin-2-yl)-(3-ethoxy-3′,6′-dihydro-2′H-[2,4′]bipyridinyl-1′-yl)-methanone (Compound 115)

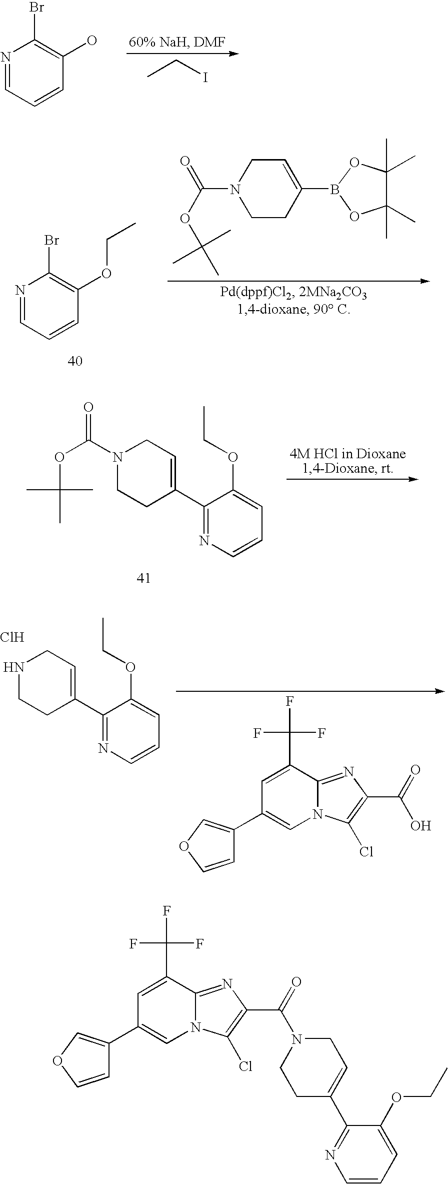

### Step 1: 2-Bromo-3-ethoxy-pyridine

NaH (60% suspension in mineral oil, 345 mg, 8.62 mmol) was added to a solution of 2-bromo-3-hydropyridine (500 mg, 2.87 mmol) in DMF (20 mL) at room temperature. After 15 min, iodoethane (708 μL, 8.62 mmol) was added dropwise and the reaction mixture was stirred overnight. Sat. NH₄Cl was added to quench the reaction at 0° C., and extracted with EtOAc (100 mL), the organic layer was washed with brine (100 mL), dried (MgSO₄), concentrated on silica and flash column chromatography (0-100% EtOAc/hexane gradient) afforded 2-bromo-3-ethoxy-pyridine as yellow oil (300 mg, 52%). ¹H NMR (CDCl₃, 300 MHz) δ 1.51 (t, 3H, J=7.20 Hz), 4.11 (q, 2H, J=7.20 Hz), 7.15 (m, 1H), 7.20 (m, 1H), 7.99 (m, 1H); MS (ESI) m/z=203.9 (MH⁺).

### Step 2: 3-Ethoxy-3′,6′-dihydro-2′H-[2,4′]bipyridinyl-1′-carboxylic acid tert-butyl ester

Prepared similar to Compound 1. ¹H NMR (CDCl₃, 300 MHz) δ 1.49 (s, 11H), 2.67 (m, 2H), 3.62 (t, 2H, J=6.00 Hz), 4.09 (m, 5H), 6.33 (bd, 1H, J=76.50 Hz), 7.16 (m, 2H), 8.17 (m, 1H); MS (ESI) m/z=305.2 (MH⁺).

### Step 3: 3-Ethoxy-1′,2′,3′,6′-tetrahydro-[2,4′]bipyridinyl hydrochloride

Prepared similar to Compound 2. ¹H NMR (d₆-DMSO, 300 MHz) δ 1.38 (t, 3H, J=6.90 Hz), 2.79 (m, 2H), 3.28 (m, 2H), 3.80 (m, 2H), 4.12 (q, 2H, J=6.90 Hz), 6.70 (m, 1H), 7.30 (m, 1H), 7.53 (d, 1H, J=9.00 Hz), 8.16 (d, 1H, J=3.60 Hz), 8.94 (s, 2H); MS (ESI) m/z=205.1 (MH⁺).

### Step 4: (3-Chloro-6-furan-3-yl-8-trifluoromethyl-imidazo[1,2-a]pyridin-2-yl)-(3-ethoxy-3′,6′-dihydro-2′H-[2,4′]bipyridinyl-1′-yl)-methanone

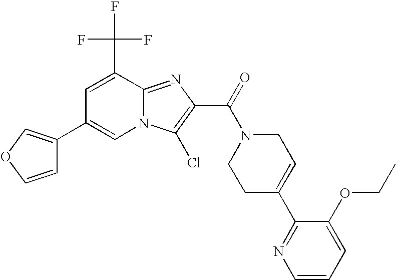

Prepared similar to Compound 1. ¹H NMR (d₆-DMSO, 300 MHz) δ 1.38 (m, 3H), 2.73 (m, 2H), 3.83 (m, 2H), 4.11 (m, 2H), 4.38 (m, 2H), 6.65 (bd, 1H, J=35.10 Hz), 7.26 (m, 1H), 7.33 (m, 1H), 7.44 (m, 1H), 7.85 (t, 1H, J=2.10 Hz), 8.13 (d, 1H, J=3.90 Hz), 8.21 (s, 1H), 8.56 (s, 1H), 8.83 (s, 1H); MS (ESI) m/z=517.1 (MH⁺).

### EXAMPLE 17

### (3-Fluoro-3′,6′-dihydro-2′H-[2,4′]bipyridinyl-1′-yl)-(6-thiophen-2-yl-4-trifluoromethyl-1H-pyrrolo[2,3-b]pyridin-2-yl)-methanone (Compound 116)

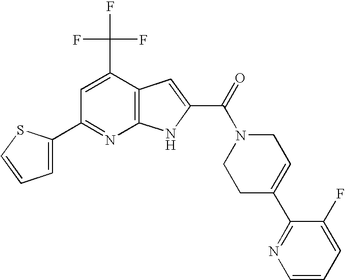

Prepared similar to Compound 1.

¹H NMR (d₆-DMSO, 300 MHz) δ 2.75 (m, 2H), 3.88 (t, 2H, J=6.00 Hz), 4.45 (m, 2H), 6.74 (m, 1H), 6.88 (s, 1H), 7.20 (m, 1H), 7.40 (m, 1H), 7.74 (m, 2H), 8.03 (m, 2H), 8.44 (m, 1H), 12.84 (s, 1H). MS (ESI) m/z=472.9 (MH⁺).

### EXAMPLE 18

### (3-Fluoro-3′,6′-dihydro-2′H-[2,4′]bipyridinyl-1′-yl)-(5-thiophen-2-yl-7-trifluoromethyl-1H-pyrrolo[3,2-b]pyridin-2-yl)-methanone (Compound 117)

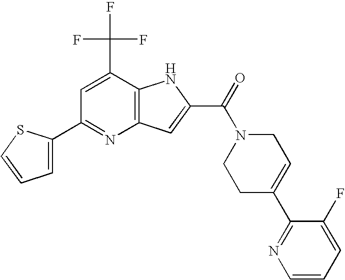

Prepared similar to Compound 1.

¹H NMR (d₆-DMSO, 300 MHz) δ 2.74 (m, 2H), 3.87 (m, 2H), 4.44 (m, 2H), 6.59 (bd, 1H, J=40.80 Hz), 7.10 (s, 1H), 7.19 (m, 1H), 7.38 (m, 1H), 7.64 (m, 1H), 7.75 (m, 1H), 7.96 (m, 1H), 8.06 (m, 1H), 8.44 (m, 1H), 12.39 (s, 1H); MS (ESI) m/z=472.9 (MH⁺).

### EXAMPLE 19

### (6-Boronic acid-3-chloro-8-trifluoromethyl-imidazo[1,2-a]pyridin-2-yl)-(3-fluoro-3′,6′-dihydro-2′H-[2,4′]bipyridinyl-1′-yl)-methanone (Compound 5)

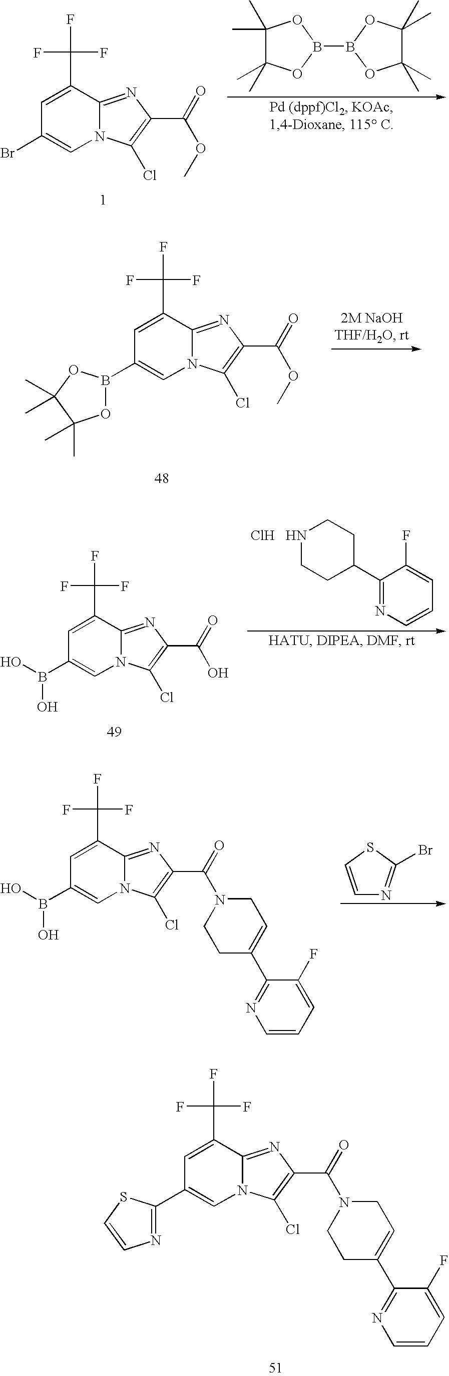

### Step 1: 3-Chloro-6-(4,4,5,5-tetramethyl-[1,3,2]dioxaborolan-2-yl)-8-trifluoromethylimidazo[1,2-a]pyridine-2-carboxylic acid methyl ester

Prepared similar to Compound 6 except the temperature was raised to 115° C. ¹H NMR (CDCl₃, 300 MHz) δ 1.39 (s, 12H), 4.01 (s, 3H), 7.96 (s, 1H), 8.67 (s, 1H); MS (ESI) m/z=323 (MH⁺-pinacol ester).

### Step 2: 6-Boronic acid-3-chloro-8-trifluoromethyl-imidazo[1,2-a]pyridine-2-carboxylic acid

A mixture of 3-chloro-6-(4,4,5,5-tetramethyl-[1,3,2]dioxaborolan-2-yl)-8-trifluoromethylimidazo[1,2-a]pyridine-2-carboxylic acid methyl ester (200 mg, 0.47 mmol) and NaOH (2M, 742 μL, 1.48 mmol) was stirred at room temperature in THF/H₂O (3:1 v/v, 5 mL) for 2 hours. The reaction mixture was concentrated and the residue was acidified with conc. HCl and the precipitates filtered, washed with warm ether to afford 6-boronic acid-3-chloro-8-trifluoromethyl-imidazo[1,2-a]pyridine-2-carboxylic acid as a white powder, which was used without further purification (106 mg, 70%). ¹H NMR (d₆-DMSO, 300 MHz) δ 8.11 (s, 1H), 8.78 (s, 2H), 8.90 (s, 1H). MS (ESI) m/z=309 (MH⁺).

### Step 3: (6-Boronic acid-3-chloro-8-trifluoromethyl-imidazo[1,2-a]pyridin-2-yl)-(3-fluoro-3′,6′-dihydro-2′H-[2,4′]bipyridinyl-1′-yl)-methanone

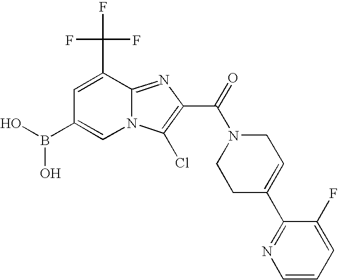

Prepared similar to Compound 1.

¹H NMR (d₆-DMSO, 300 MHz) δ 2.73 (m, 2H), 3.85 (m, 2H), 4.41 (m, 2H), 6.57 (bd, 1H, J=51.30 Hz), 7.38 (m, 1H), 7.74 (m, 1H), 8.12 (s, 1H), 8.44 (m, 1H), 8.78 (s, 2H), 8.91 (s, 1H); MS (ESI) m/z=469.0 (MH⁺).

### Step 4: (3-Chloro-6-thiazol-2-yl-8-trifluoromethyl-imidazo[1,2-a]pyridin-2-yl)-(3-fluoro-3′,6′-dihydro-2′H-[2,4′]bipyridinyl-1′-yl)-methanone (Compound 118)

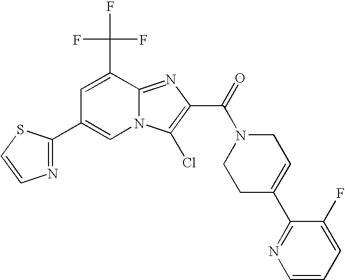

Prepared similar to Compound 1. ¹H NMR (d₆-DMSO, 300 MHz) δ 2.74 (m, 2H), 3.88 (m, 2H), 4.43 (m, 2H), 6.58 (bd, 1H, J=52.50 Hz), 7.40 (m, 1H), 7.75 (m, 1H), 7.98 (d, 1H, J=3.30 Hz), 8.05 (d, 1H, J=3.30 Hz), 8.33 (s, 1H), 8.45 (m, 1H), 9.05 (s, 1H); MS (ESI) m/z=509.1 (MH⁺).

### EXAMPLE 20

### (3-Chloro-6-thiazol-5-yl-8-trifluoromethyl-imidazo[1,2-a]pyridin-2-yl)-(3-fluoro-3′,6′-dihydro-2′H-[2,4′]bipyridinyl-1′-yl)-methanone (Compound 119)

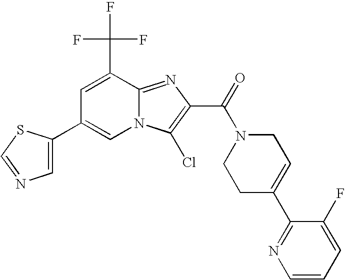

Prepared similar to Compound 1. ¹H NMR (d₆-DMSO, 300 MHz) δ 2.73 (m, 2H), 3.84 (m, 2H), 4.42 (m, 2H), 6.58 (bd, 1H, J=51.00 Hz), 7.39 (m, 1H), 7.75 (m, 1H), 8.22 (s, 1H), 8.44 (m, 1H), 8.61 (s, 1H), 8.85 (s, 1H), 9.23 (s, 1H); MS (ESI) m/z=509 (MH⁺).

### EXAMPLE 21

### (3-Chloro-6-thiazol-4-yl-8-trifluoromethyl-imidazo[1,2-a]pyridin-2-yl)-(3-fluoro-3′,6′-dihydro-2′H-[2,4′]bipyridinyl-1′-yl)-methanone (Compound 120)

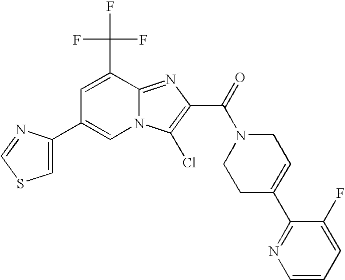

Prepared similar to Compound 1. ¹H NMR (d₆-DMSO, 300 MHz) δ 2.75 (m, 2H), 3.87 (m, 2H), 4.42 (m, 2H), 6.58 (bd, 1H, J=50.40 Hz), 7.39 (m, 1H), 7.75 (m, 1H), 8.43 (m, 1H), 8.52 (s, 1H), 8.66 (s, 1H), 9.11 (s, 1H), 9.32 (s, 1H); MS (ESI) m/z=508.9 (MH⁺).

### EXAMPLE 22

### [3-Chloro-6-(2,5-dihydro-1H-pyrrol-3-yl)-8-trifluoromethyl-imidazo[1,2-a]pyridin-2-yl]-(3-fluoro-3′,6′-dihydro-2′H-[2,4′]bipyridinyl-1′-yl)-methanone (Compound 121)

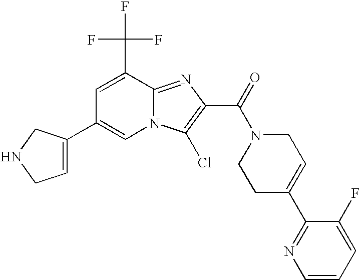

Prepared similar to Compound 1. ¹H NMR (d₆-DMSO, 300 MHz) δ 2.73 (m, 2H), 3.90 (m, 2H), 4.23 (m, 2H), 4.40 (m, 2H), 4.52 (m, 2H), 6.57 (bd, 1H, J=51.30 Hz), 6.89 (s, 1H), 7.39 (m, 1H), 7.75 (m, 1H), 8.25 (s, 1H), 8.44 (m, 1H), 8.53 (s, 1H), 9.57 (s, 1H).; MS (ESI) m/z=492.5 (MH⁺).

### EXAMPLE 23

### [3-Chloro-6-(1H-imidazol-4-yl)-8-trifluoromethyl-imidazo[1,2-a]pyridin-2-yl]-(3-fluoro-3′,6′-dihydro-2′H-[2,4′]bipyridinyl-1′-yl)-methanone (Compound 122)

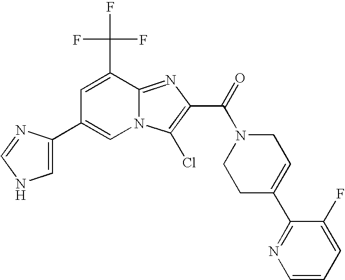

Prepared similar to Compound 1. ¹H NMR (d₆-DMSO, 300 MHz) δ 2.73 (m, 2H), 3.88 (m, 2H), 4.43 (m, 2H), 6.58 (bd, 1H, J=51.30 Hz), 7.40 (m, 1H), 7.75 (m, 1H), 8.36 (s, 1H), 8.43 (s, 1H), 8.44 (m, 1H), 8.97 (s, 1H), 9.11 (s, 1H); MS (ESI) m/z=490.4 (MH⁺).

### EXAMPLE 24

### (3-Chloro-6-thiophen-2-yl-8-trifluoromethyl-imidazo[1,2-a]pyridin-2-yl)-(3-fluoro-3′,6′-dihydro-2′H-[2,4′]bipyridinyl-1′-yl)-methanone (Compound 123)

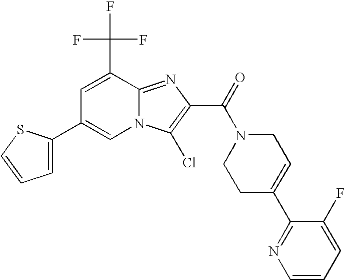

Prepared similar to Compound 1. ¹H NMR (d₆-DMSO, 300 MHz) δ 2.73 (m, 2H), 3.86 (m, 2H), 4.43 (m, 2H), 6.58 (bd, 1H, J=50.70 Hz), 7.24 (m, 1H), 7.40 (m, 1H), 7.73 (m, 2H), 7.86 (m, 1H), 8.15 (s, 1H), 8.44 (m, 1H), 8.74 (s, 1H); MS (ESI) m/z=507.9 (MH⁺).

### EXAMPLE 25

### (3-Chloro-6-phenyl-8-trifluoromethyl-imidazo[1,2-a]pyridin-2-yl)-(3-fluoro-3′,6′-dihydro-2′H-[2,4′]bipyridinyl-1′-yl)-methanone (Compound 124)

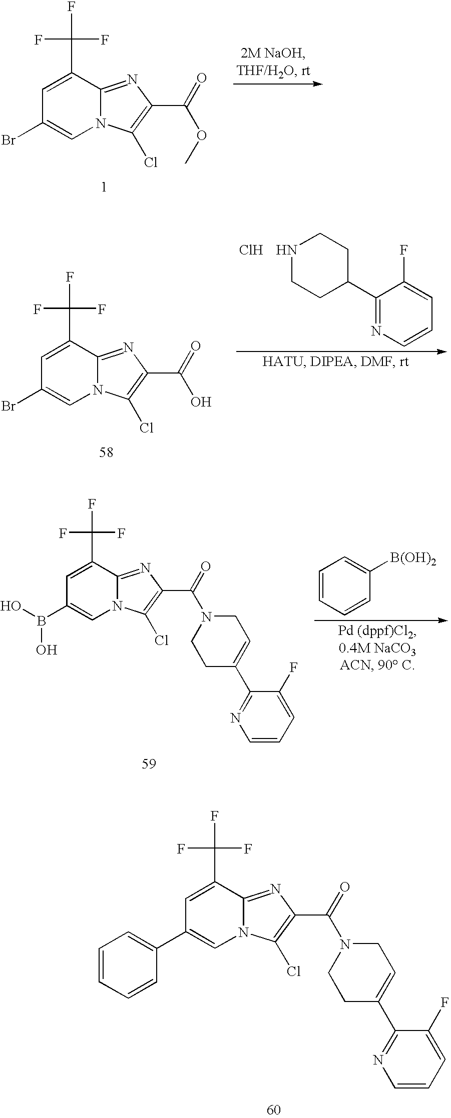

### Step 1: 5-Bromo-3-chloro-7-trifluoromethyl-imidazo[1,2-a]pyridine-2-carboxylic acid

A mixture of 6-bromo-3-chloro-8-trifluoromethyl-imidazo[1,2-a]pyridine-2-carboxylic acid methyl ester (5.00 g, 14 mmol) and NaOH (2M, 21 m F, 42 mmol) was stirred at room temperature in THF/H₂O (3:1 v/v, 100 mL) for 2 hours. The reaction mixture was concentrated and the residue was acidified with 10% HCl and extracted with DCM (2×80 mF). The organic layer was washed with brine (50 mL), dried (MgSO₄), filtered and concentrated to afford 5-bromo-3-chloro-7-trifluoromethyl-imidazo[1,2-a]pyridine-2-carboxylic acid as a light yellow powder, which was used without further purification (4.42 g, 92%). ¹H NMR (d₆-DMSO, 300 MHz) δ 8.09 (s, 1H), 8.98 (d, 1H, J=0.80 Hz), 13.5 (s, 1H); MS (ESI) m/z=344.9, 346.9 (MH⁺).

### Step 2: (6-Bromo-3-chloro-8-trifluoromethyl-imidazo[1,2-a]pyridin-2-yl)-(3-fluoro-3′,6′-dihydro-2′H-[2,4′]bipyridinyl-1′-yl)-methanone (Compound 125)

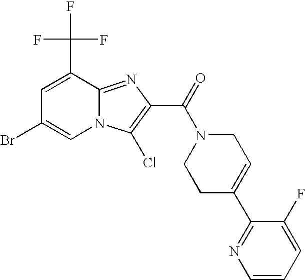

Prepared similar to Compound 1. ¹H NMR (d₆-DMSO, 300 MHz) δ 2.72 (m, 2H), 3.80 (m, 1H), 3.90 (m, 1H), 4.39 (m, 2H), 6.56 (bd, 1H, J=53.40 Hz), 7.38 (m, 1H), 7.74 (m, 1H), 8.09 (s, 1H), 8.44 (m, 1H), 9.01 (s, 1H); MS (ESI) m/z=505.1 (MH⁺).

### Step 3: (3-Chloro-6-phenyl-8-trifluoromethyl-imidazo[1,2-a]pyridin-2-yl)-(3-fluoro-3′,6′-dihydro-2′H-[2,4′]bipyridinyl-1′-yl)-methanone

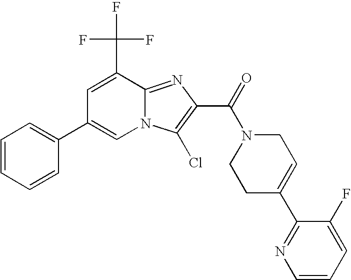

Prepared similar to Compound 1. ¹H NMR (d₆-DMSO, 300 MHz) δ 2.74 (m, 2H), 3.87 (m, 2H), 4.41 (m, 2H), 6.58 (bd, 1H, J=49.50 Hz), 7.39 (m, 1H), 7.56 (m, 3H), 7.73 (m, 1H), 7.89 (m, 2H), 8.19 (s, 1H), 8.44 (m, 1H), 8.81 (s, 1H); MS (ESI) m/z=502 (MH⁺).

### EXAMPLE 26

### (3-Chloro-8-trifluoromethyl-6-vinyl-imidazo[1,2-a]pyridin-2-yl)-(3-fluoro-3′,6′-dihydro-2′H-[2,4′]bipyridinyl-1′-yl)-methanone (Compound 126)

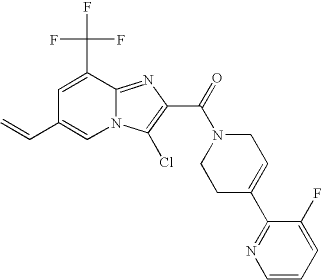

Prepared similar to Compound 1. ¹H NMR (d₆-DMSO, 300 MHz) δ 2.72 (m, 2H), 3.88 (m, 2H), 4.42 (m, 2H), 5.49 (d, 1H, J=11.40 Hz), 6.16 (d, 1H, J=17.40 Hz), 6.56 (bd, 1H, J=50.40 Hz), 6.96 (m, 1H), 7.38 (m, 1H), 7.73 (m, 1H), 8.18 (s, 1H), 8.42 (m, 1H), 8.74 (s, 1H);

MS (ESI) m/z=452 (MH⁺).

### EXAMPLE 27

### [3-Chloro-6-((E)-2-cyclopropyl-vinyl)-8-trifluoromethyl-imidazo[1,2-a]pyridin-2-yl]-(3-fluoro-3′,6′-dihydro-2′H-[2,4′]bipyridinyl-1′-yl)-methanone (Compound 127)

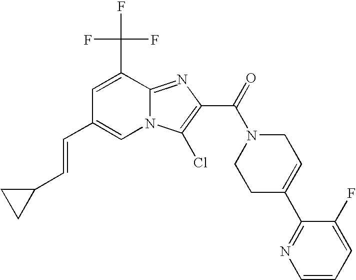

Prepared similar to Compound 1. ¹H NMR (d₆-DMSO, 300 MHz) δ 0.60 (m, 2H), 0.85 (m, 2H), 1.62 (m, 1H), 2.72 (m, 2H), 3.87 (m, 2H), 4.39 (m, 2H), 6.19 (m, 1H), 6.59 (bd, 1H, J=65.70 Hz), 6.65 (s, 1H), 7.38 (m, 1H), 7.73 (m, 1H), 8.07 (s, 1H), 8.43 (m, 1H), 8.56 (s, 1H); MS (ESI) m/z=492.0 (MH⁺).

### EXAMPLE 28

### 2-(3-Fluoro-phenyl)-N-(6-furan-3-yl-8-trifluoromethyl-imidazo[1,2-a]pyidin-2-ylmethyl)-acetamide (Compound 128)

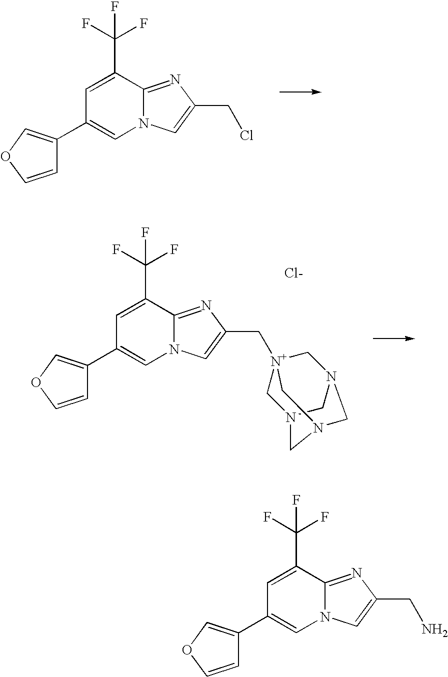

Step 1: C-(6-Furan-3-yl-8-trifluoromethyl-imidazo[1,2-a]pyridin-2-yl)-methylamine hydrochloride

A mixture of 2-chloromethyl-6-furan-3-yl-8-trifluoromethyl-imidazo[1,2-a]pyridine (2.03 g, 6.76 mmol) and hexamethyltetramine (1.895 g, 13.5 mmol) was heated at 100° C. in THF (40 mL) for 9 hours. Upon cooling with ice-water bath, the precipitate was filtered and dried under high vacuum to give 1-(6-furan-3-yl-8-trifluoromethyl-imidazo[1,2-a]pyridin-2-ylmethyl)-3,5,7-triaza-1-azonia-tricyclo[3.3.1.1*3,7*]decane chloride (3.74 g) as a white solid. A solution of 1-(6-furan-3-yl-8-trifluoromethyl-imidazo[1,2-a]pyridin-2-ylmethyl)-3,5,7-triaza-1-azonia-tricyclo[3.3.1.1*3,7*]decane chloride (3.43 g) was heated in EtOH (100 mL) and conc. HCl (20 mL) at 90° C. After 50 min, the mixture was cooled and poured into 500 mL of chilled Et₂O to give a white precipitate which was filtered. The precipitate was digested with saturated aqueous NaHCO₃ (25 mL) and extracted with EtOAc (3×200 mL). The extracts were dried (Na₂SO₄), filtered and concentrated to give a yellow foam. DCM (10 mL) was added and the insoluble material was filtered. To the filtrate was added HCl (2M in Et₂O, 2.5 mL) and the solvent was removed under reduced pressure to give C-(6-furan-3-yl-8-trifluoromethyl-imidazo[1,2-a]pyridin-2-yl)-methylamine hydrochloride (1.29 g, 60%) as a beige solid. ¹H NMR (d₆-DMSO, 300 MHz) δ 4.22 (q, 2H, J=5.6 Hz), 7.07 (brd, 1H, J=1.8 Hz), 7.83 (t, 1H, J=1.8 Hz), 8.08 (s, 1H), 8.17 (s, 1H), 8.43 (s, 1H), 8.46 (brs, 3H), 9.27 (s, 1H); MS (ESI) m/z=282 (MH⁺).

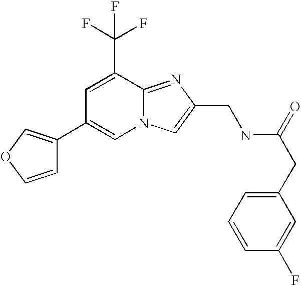

Prepared using standard HATU coupling of C-(6-furan-3-yl-8-trifluoromethyl-imidazo[1,2-a]pyridin-2-yl)-methylamine hydrochloride and (3-fluoro-phenyl)-acetic acid. ¹H NMR (d₆-DMSO, 300 MHz) δ 3.52 (s, 2H), 4.41 (d, 2H, J=6 Hz), 7.12-7.00 (m, 4H), 7.31 (q, 1H, J=8 Hz), 7.80 (s, 1H), 7.87 (s, 1H), 8.01 (s, 1H), 8.37 (s, 1H), 8.72 (t, 1H, J=6 Hz), 9.11 (s, 1H); MS (ESI) m/z=418 (MH⁺).

### EXAMPLE 29

### 2-(2-Fluoro-phenyl)-N-(6-furan-3-yl-8-trifluoromethyl-imidazo[1,2-a]pyidin-2-ylmethyl)-acetamide (Compou8nd 129

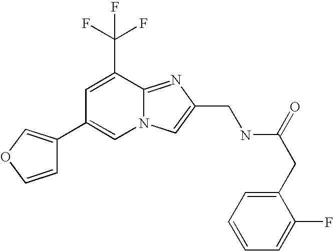

Prepared using standard HATU coupling of C-(6-furan-3-yl-8-trifluoromethyl-imidazo[1,2-a]pyridin-2-yl)-methylamine hydrochloride and (2-fluoro-phenyl)-acetic acid. ¹H NMR (d₆-DMSO, 300 MHz) δ 2.02 (s, 2H), 4.43 (d, 2H, J=6 Hz), 7.01 (s, 1H), 7.12 (m, 2H), 7.30 (m, 2H), 7.80 (s, 1H), 7.87 (s, 1H), 8.02 (s, 1H), 8.38 (s, 1H), 8.71 (t, 1H, J=6 Hz), 9.13 (s, 1H); MS (ESI) m/z=418 (MH⁺).

### EXAMPLE 30

### N-(6-Furan-3-yl-8-trifluoromethyl-imidazo[1,2-a]pyridin-2-ylmethyl)-benzamide (Compound 130

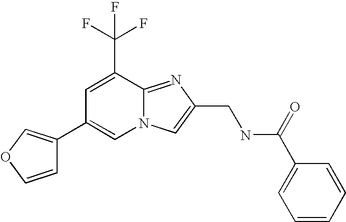

Prepared using standard HATU coupling of C-(6-furan-3-yl-8-trifluoromethyl-imidazo[1,2-a]pyridin-2-yl)-methylamine hydrochloride and phenylacetic acid. ¹H NMR (d₆-DMSO, 300 MHz) δ 4.60 (d, 2H, J=6 Hz), 6.96 (br s, 1H), 7.49 (m, 3H), 7.79 (t, 1H, J=2 Hz), 7.90 (m, 3H), 7.97 (s, 1H), 8.35 (s, 1H), 9.09 (s, 1H), 9.15 (t, 1H, J=6 Hz); MS (ESI) m/z=386 (MH⁺).

### EXAMPLE 31

### (3-Chloro-6-furan-3-yl-8-trifluoromethyl-imidazo[1,2-a]pyridin-2-yl)-(3-thiophen-2-yl-2,5-dihydro-pyrrol-1-yl)-methanone (hereinafter referred to as “Compound 7”)

### Step 1: 3-Trifluoromethanesulfonyloxy-2,5-dihydro-pyrrole-1-carboxylic acid tert-butyl ester

To a solution of lithium diisopropylamide (2M in heptane/THF/ethylbenzene, 6.5 mL, 12.96 mmol) in THF (30 mL) at −78° C. was added a solution of N-Boc-3-pyrrolidinone (2 g, 10.8 mmol) in THF (30 mL) over 10 min. After 40 min, a solution of N-phenylbis(trifluoromethanesulfinimide) (4.24 g, 11.88 mmol) in THF (30 mL) was added. After 3 hours, the mixture was quenched with saturated aqueous solution of NaHCO₃ and diluted with ethyl ether (250 mL). The aqueous phase was discarded and the organic phase was washed with 5% citric acid (2×50 mL), 10% aq NaOH (2×50 mL), water (50 mL), and brine (50 mL). The organic phase was dried (Na₂SO₄), filtered and concentrated. The crude product was absorbed on silica gel followed by column chromatography [n-hex/EtOAc (15:1 v/v) followed by n-hex/EtOAc (9:1 v/v)]gave 3-trifluoromethanesulfonyloxy-2,5-dihydro-pyrrole-1-carboxylic acid tert-butyl ester (1.2 g, 35%) as an oil. ¹H NMR (d₆-DMSO, 300 MHz) δ1.42 (s, 9H), 4.06-4.26 (m, 4H), 6.02-6.18 (m, 1H); MS (ESI) m/z=262 (MH⁺-^{t}Bu).

### Step 2: 3-Thiophen-2-yl-2,5-dihydro-pyrrole-1-carboxylic acid tert-butyl ester

To a solution of 3-trifluoromethanesulfonyloxy-2,5-dihydro-pyrrole-1-carboxylic acid tert-butyl ester (184.5 mg, 0.582 mmol) in THF (3 mL) was added 2-thienylzinc bromide (0.5 M in THF, 1.16 mL, 0.582 mmol) and tetrakis(triphenylphosphine)palladium(0) (67.2 mg, 0.058 mmol). The mixture was heated at 50° C. for 105 min. Upon cooling, the mixture was filtered warm and diluted with EtOAc (50 mL) and washed with brine (20 mL). The organic layer was dried (Na₂SO₄), filtered and concentrated. Column chromatography [n-hex/EtOAc (12:1 v/v)]of the crude gave 3-thiophen-2-yl-2,5-dihydro-pyrrole-1-carboxylic acid tert-butyl ester (49 mg, 33%) as an oil. ¹H NMR (d₆-DMSO, 300 MHz) [symbol] 1.44 (s, 4.5H), 1.45 (s, 4.5H), 4.17 (m, 2H), 4.36 (m, 2H), 6.08 (brd, 1H, J=12.3 Hz), 7.05 (t, 1H, J=3.2 hz), 7.11 (d, 1H, J=3.2 Hz), 7.51 (d, 1H, J=5.3 Hz); MS (ESI) m/z=274 (MNa⁺).

### Step 3: 3-Thiophen-2-yl-2,5-dihydro-1H-pyrrole

A solution of 3-thiophen-2-yl-2,5-dihydro-pyrrole-1-carboxylic acid tert-butyl ester (45.5 mg, 0.181 mmol) was stirred in 30% TFA/DCM solution (10 mL). After 50 min, the solvents were removed and evaporated with toluene (2×3 mL) to give 3-thiophen-2-yl-2,5-dihydro-1H-pyrrole (49 mg) as a brown solid which was used for the next step without further purification. ¹H NMR (d₆-DMSO, 300 MHz).4.12 (brs, 2H), 4.31 (brs, 2H), 6.13 (m, 1H), 7.10 (dd, 1H, J=3.5, 5 Hz), 7.21 (dd, 1H, J=0.6, 5 Hz), 7.60 (dd, 1H, J=0.9, 5 Hz), 9.33 (brs, 2H);

MS (ESI) m/z=152.1 (MH⁺).

### Step 4: (3-Chloro-6-furan-3-yl-8-trifluoromethyl-imidazo[1,2-a]pyridin-2-yl)-(3-thiophen-2-yl-2,5-dihydro-pyrrol-1-yl)-methanone

Prepared using standard HATU coupling of the above amine. ¹H NMR (d₆-DMSO, 300 MHz) [symbol] 4.51 (m, 1H), 4.70 (m, 1H), 4.82 (m, 1H), 6.22 (m, 1H), 5.04 (m, 1H), 7.01 (dd, 0.5H, J=0.9, 2.6 Hz), 7.08 (dd, 0.5H, J=2.6, 3.5 Hz), 7.10 (dd, 0.5H, J=2.5, 3.8 Hz), 7.21 (brd, 0.5H, J=2.5 Hz), 7.32-7.35 (m, 1H), 7.53 (dd, 0.5H, J=1.2, 3.3 Hz), 7.55 (dd, 0.5H, J=0.9, 2.3 Hz), 7.83-7.86 (m, 1H), 8.24-8.26 (brs, 1H), 8.57 (brs, 1H), 8.85 (s, 1H); MS (ESI) m/z=464 (MH⁺).

### EXAMPLE 32

### [3-Chloro-6-(1H-pyrazol-4-yl)-8-trifluoromethyl-imidazo[1,2-a]pyridin-2-yl]-(3-furan-3-yl-2,5-dihydro-pyrrol-1-yl)-methanone (Compound 131)

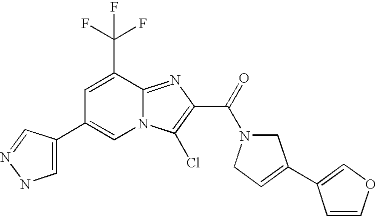

Prepared using experimental procedure described in Example 31 for Compound 7. ¹H NMR (d₆-DMSO, 300 MHz) δ 4.44 (br s, 1H), 4.54 (br s, 1H), 4.75 (br s, 1H), 4.80 (br s, 1H), 6.17 (m, 1H), 6.79 (m, 1H), 7.56 (br s, 0.5H), 7.69 (br s, 1H), 7.90 (s, 0.5H), 8.20 (s, 1H), 8.40 (s, 2H), 8.84 (s, 1H), pyrazole NH not observed; MS (ESI) m/z=448 (MH⁺).

### EXAMPLE 33

### [3-Chloro-6-(1H-pyrazol-4-yl)-8-trifluoromethyl-imidazo[1,2-a]pyridin-2-yl]-(3-thiophen-3-yl-2,5-dihydro-pyrrol-1-yl)-methanone (Compound 132)

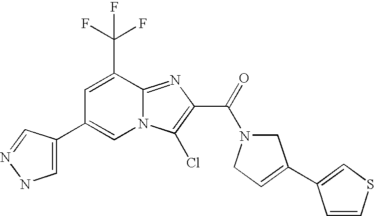

Prepared using experimental procedure described in Example 31 for Compound 7. ¹H NMR (d₆-DMSO, 300 MHz) δ 8.80 (s, 1H), 8.36 (s, 1H), 8.16 (s, 1H), 7.54 (m, 2H), 7.39 (m, 1H), 7.34 (m, 0.5H), 7.22 (m, 0.5H), 6.24 (br s, 1H), 4.89 (br s, 1H), 4.75 (br s, 1H), 4.61 (br s, 1H), 4.44 (br s, 1H), pyrazole H not observed; MS (ESI) m/z=464 (MH⁺).

### EXAMPLE 34

### [3-Chloro-6-(1H-pyrazol-4-yl)-8-trifluoromethyl-imidazo[1,2-a]pyridin-2-yl]-(3-isoxazol-4-1-2,5-dihydro-pyrrol-1-yl)-methanone (Compound 133)

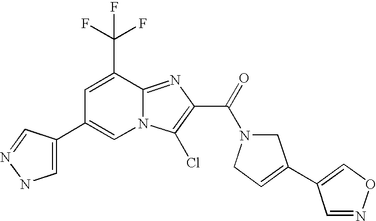

Prepared using experimental procedure described in Example 31 for Compound 7. ¹H NMR (d₆-DMSO, 300 MHz) δ 4.43 (br s, 1H), 4.54 (br s, 2.5H), 4. 8.35 (br s, 2H), 74 (br s, 2H), 6.28 (m, 1H), 8.16 (s, 1H), 8.80 (m, 1H), 8.83 (s, 0.5H), 8.93 (s, 0.5H), 8.98 (s, 0.5H), 9.14 (s, 0.5H), pyrazole NH not observed; MS (ESI) m/z=449 (MH⁺).

### EXAMPLE 35

### [3-Chloro-6-(1H-pyrazol-4-yl)-8-trifluoromethyl-imidazo[1,2-a]pyridin-2-yl]-(3-pyridin-3-yl-2,5-dihydro-pyrrol-1-yl)-methanone (Compound 134)

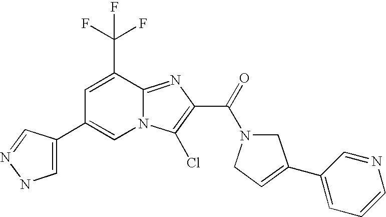

Prepared using experimental procedure described in Example 31 for Compound 7. ¹H NMR (d₆-DMSO, 300 MHz) δ 4.63 (br s, 1H), 4.86 (br s, 1H), 4.97 (br s, 1H), 5.14 (br s, 1H), 6.83 (br s, 1H), 7.70 (m, 1H), 8.08 (dt, 0.5H, J=2, 8 Hz), 8.30 (br s, 1.5H), 8.48 (s, 2H), 8.65 (dd, 0.5H, J=2, 3 Hz), 8.69 (dd, 1H, J=2, 3 Hz), 8.75 (d, 0.5H, J=2 Hz), 8.94 (m, 1.5H), pyrazole NH not observed; MS (ESI) m/z=459 (MH⁺).

### EXAMPLE 36

### [3-Chloro-6-(1H-pyrazol-4-yl)-8-trifluoromethyl-imidazo[1,2-a]pyridin-2-yl]-[3-(1H-pyrazol-4-yl-2,5-dihydro-pyrrol-1-yl]-methanone (Compound 135)

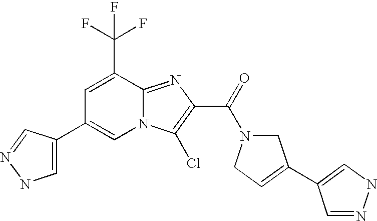

Prepared using experimental procedure described in Example 31 for Compound 7. ¹H NMR (d₆-DMSO, 300 MHz) δ 4.44 (brs, 1H), 4.56 (br s, 1H), 4.73 (br s, 1H), 4.83 (br s, 1H), 6.03 (br s, 1H), 7.68 (s, 1H), 7.85 (s, 1H), 8.20 (s, 1H), 8.40 (br s, 1H), 8.83 (s, 1H), pyrazole NH not observed; MS (ESI) m/z=448 (MH⁺).

### EXAMPLE 37

### [3-Chloro-6-(1H-pyrazol-4-yl)-8-trifluoromethyl-imidazo[1,2-a]pyridin-2-yl]-[3-(1,2,3,6-pyrazol-4-yl-2,5-tetrahydro-pyridin-4-yl)-2,5-dihydro-pyrrol-1-yl]-methanone (Compound 136)

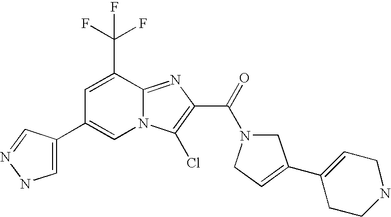

Prepared using experimental procedure described in Example 31 for Compound 7. ¹H NMR (d₆-DMSO, 300 MHz) δ 3.19 (m, 2H), 3.67 (m, 2H), 4.41 (m, 2H), 4.73 (s, 2H), 5.40 (s, 0.5H), 5.72 (s, 0.5H), 6.00 (s, 1H), 8.15 (s, 1H), 8.33 (s, 2H), 8.79 (s, 1H), pyrazole NH not observed; MS (ESI) m/z=463 (MH⁺).

### EXAMPLE 38

### [3-Chloro-6-(1H-pyrazol-4-yl)-8-trifluoromethyl-imidazo[1,2-a]pyridin-2-yl]-[3-(1H-pyrazol-3-yl)-2,5-dihydro-pyrrol-1-yl]-methanone (Compound 137)

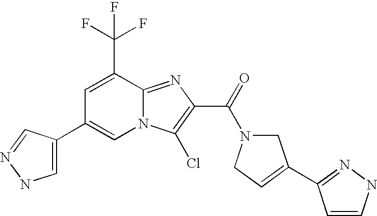

Prepared using experimental procedure described in Example 31 for Compound 7. ¹H NMR (d₆-DMSO, 300 MHz) δ 4.49 (br s, 1H), 4.64 (br s, 1H), 4.80 (br s, 1H), 4.95 (br s, 1H), 6.27 (t, 1H, J=2 Hz), 6.44 (m, 0.5H), 6.52 (d, 0.5H, J=2 Hz), 7.68 (t, 1H, J=2 Hz), 8.21 (s, 1H), 8.40 (s, 2H), 8.84 (s, 1H), pyrazole NH not observed; MS (ESI) m/z=448 (MH⁺).

### EXAMPLE 39

### N-[2-(6-Furan-3-yl-8-trifluoromethyl-imidazo[1,2-a]pyridin-2-yl)-ethyl]-benzamide (Compound 138)

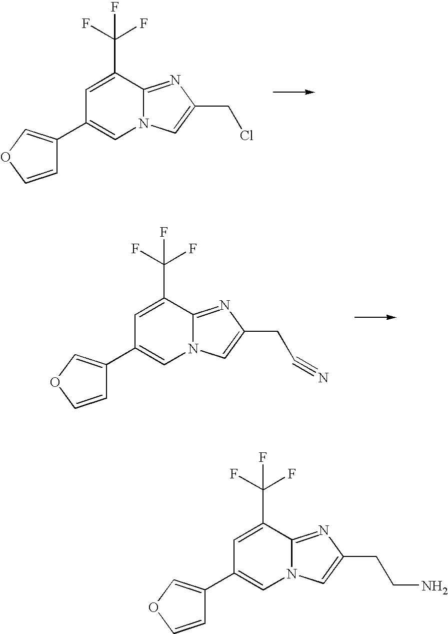

### Step 1: (6-Furan-3-yl-8-trifluoromethyl-imidazo[1,2-a]pyridin-2-yl)-acetonitrile

A mixture of 2-chloromethyl-6-furan-3-yl-8-trifluoromethyl-imidazo[1,2-a]pyridine (1 g, 3.32 mmol) and potassium cyanide (271 mg, 4.16 mmol) was heated in DMF (16 mL) and H₂O (1.6 mL) at 80° C. for 16 hours. Upon cooling, the mixture was diluted with EtOAc (200 mL) and washed with saturated aqueous NaHCO₃ (50 mL), then brine (50 mL). The extracts were filtered through a pad of silica gel and the filtrate concentrated under vacuo. Column chromatography [n-hex/EtOAc (2:1 v/v)]of the crude material gave (6-furan-3-yl-8-trifluoromethyl-imidazo[1,2-a]pyridin-2-yl)-acetonitrile (615 mg, 63%) as a beige solid. ¹H NMR (d₆-DMSO, 300 MHz) δ 4.22 (d, 2H, J=0.9 Hz), 7.02 (dd, 1H, J=0.9, 2 Hz), 7.82 (t, 1H, J=1.8 Hz), 8.04 (s, 2H), 8.40 (t, 1H, J=0.9 Hz), 9.13 (d, 1H, J=0.6 Hz); MS (ESI) m/z=291.9 (MH⁺).

### Step 2: 2-(6-Furan-3-yl-8-trifluoromethyl-imidazo[1,2-a]pyridin-2-yl)-ethylamine

To a solution of (6-furan-3-yl-8-trifluoromethyl-imidazo[1,2-a]pyridin-2-yl)-acetonitrile (610 mg, 2.09 mmol) in MeOH (20 mL) was added nickel(II) chloride hexahydrate (597 mg, 2.51 mmol) and sodium borohydride (396 mg, 10.5 mmol). After 45 min, Boc₂O (914 mg, 4.19 mmol) was added. After 1 hour, the precipitate was filtered and the filtrate was removed under reduced pressure. Column chromatography [h-hex/EtOAc (3:2 v/v)]of the crude gave [2-(6-furan-3-yl-8-trifluoromethyl-imidazo[1,2-a]pyridin-2-yl)-ethyl]-carbamic acid tert-butyl ester (0.5 g). To a stirred solution of Boc-protected amine (0.5 g) in DCM (10 mL) was added HCl (2M in Et₂O, 10 mL). After 5.5 hours, the solvent was concentrated to give 2-(6-furan-3-yl-8-trifluoromethyl-imidazo[1,2-a]pyridin-2-yl)-ethylamine hydrochloride (437 mg, 63%). ¹H NMR (d₆-DMSO, 300 MHz) δ 3.06-3.24 (m, 4H), 7.07 (dd, 1H, J=0.6, 1.8 Hz), 7.83 (t, 1H, J=1.8 Hz), 8.01 (s, 1H), 8.06 (brs, 3H), 8.10 (s, 1H), 8.43 (s, 1H), 9.21 (s, 1H); MS (ESI) m/z=296 (MH⁺).

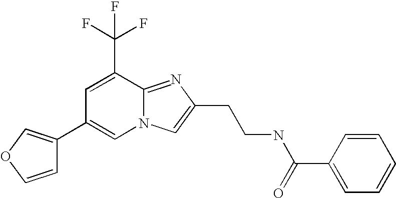

Prepared using standard HATU coupling of 2-(6-furan-3-yl-8-trifluoromethyl-imidazo[1,2-a]pyridin-2-yl)-ethylamine hydrochloride and phenylacetic acid. ¹H NMR (d₆-DMSO, 300 MHz) δ 3.05 (t, 2H, J=7 Hz), 3.64 (q, 2H, J=6 Hz), 7.05 (br s, 1H), 7.46 (m, 3H), 7.81 (br s, 1H), 7.84 (s, 1H), 7.97 (s, 1H), 8.14 (s, 1H), 8.42 (s, 1H), 8.63 (t, 1H, J=6 Hz), 9.18 (s, 1H); MS (ESI) m/z=400 (MH⁺).

### EXAMPLE 40

### N-[1-(3-Chloro-6-furan-2-yl-8-trifluoromethyl-imidazo[1,2-a]pyridine-2-carbonyl)-azetidin-3-yl]-methanesulfonamide (Compound 139)

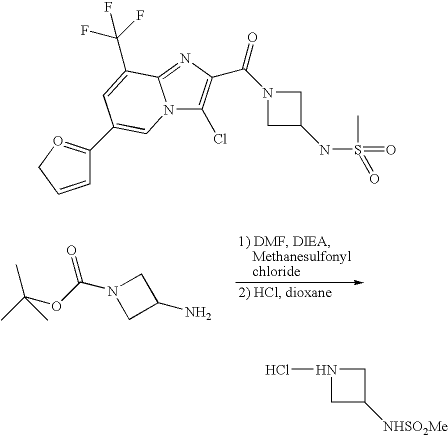

### Step 1: Azetidine-3-N-methanesulfonamide hydrochloride

Methanesulfonyl chloride (758 uL, 9.76 mmol) was added dropwise to a DCM (48 mL) solution of N,N′-diisopropylethylamine (12 mL) and N—BOC-3-amino-azetidine (2.0 g, 9.57 mmol) at 0° C. After 2 hours at 0° C., solvent was removed and to residue was added HCl (4.0 M) in dioxane at room temperature. After 4 hours the solvent was removed and azetidine-3-N-methanesulfonamide hydrochloride was used for the next step without further purification. Azetidine-3-N-methanesulfonamide hydrochloride (1.42 g 99%) was obtained as a pale yellow solid. MS (ESI) m/z=151.2 (MH⁺).

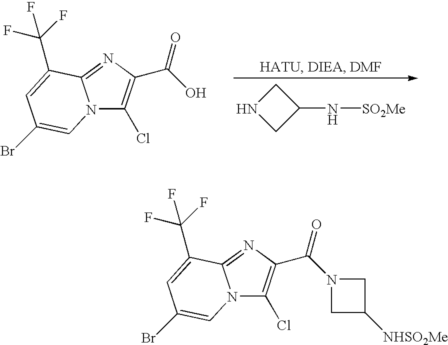

### Step 2: [1-(3-Chloro-6-bromo-8-trifluoromethyl-imidazo[1,2-a]pyridine-2-carbonyl)-azetidin-3-yl]-methanesulfonamide (Compound 140)

Using standard HATU coupling conditions, 3-chloro-bromo-8-trifluoromethyl-imidazo[1,2-a]pyridine-2-carboxylic acid (1.0 g, 2.92 mmol) and azetidine 3-N-methanesulfonamide hydrochloride (1.08 g, 5.84 mmol) gave [1-(3-chloro-6-bromo-8-trifluoromethyl-imidazo[1,2-a]pyridine-2-carbonyl)-azetidin-3-yl]-methanesulfonamide (719 mg, 52%) as a white solid. ¹H NMR (d₆-DMSO, 300 MHz) δ 2.94 (s, 3H), 3.91-3.97 (m, 1H), 4.25-4.43 (m, 3H), 4.83 (dd, 1H, J=7.0, 10.0 Hz), 7.90 (d, 1H, J=7.9 Hz), 8.09 (t, 1H, J=0.9 Hz) 8.99 (d, 1H, J=0.9 Hz); MS (ESI) m/z=477.0 (MH⁺).

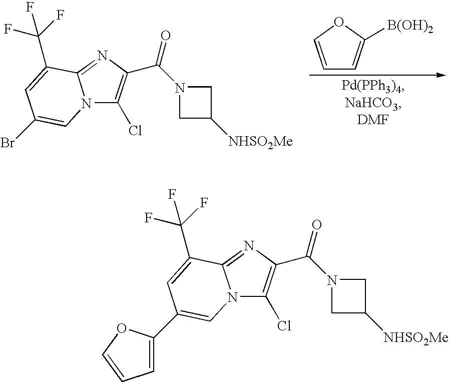

### Step 3: [1-(3-Chloro-6-furan-2-yl-8-trifluoromethyl-imidazo[1,2-a]pyridine-2-carbonyl)-azetidin-3-yl]-methanesulfonamide

DMF (420 uL) was added under argon to a mixture of 6-bromo-3-chloro-8-trifluoromethyl-imidazo[1,2-a]pyridine-1-carboxyl)-azetidin-3-yl-methanesulfonamide (20 mg, 0.042 mmol), 2-furanboronic acid (24 mg, 0.21 mmol), tetrakis(triphenylphosphine)palladium (0) (5 mg, 0.004 mmol), and then saturated aqueous NaHCO₃ (56 uL) was added. Reaction was heated in microwave to 100° C. for 20 min. The solvent was removed in-vacuo, and to the resulting residue was chromatographed to obtain [1-(3-chloro-6-furan-2-yl-8-trifluoromethyl-imidazo[1,2-a]pyridine-2-carbonyl)-azetidin-3-yl]-methanesulfonamide (11 mg, 57%) as a white solid. ¹H NMR (d₆-DMSO, 300 MHz) δ 2.94 (s, 3H), 3.95 (dd, 1H, J=4.1, 9.4 Hz), 4.28-4.45 (m, 3H), 4.86 (dd, 1H, J=7.3, 9.1 Hz), 6.71 (dd, 1H, J=1.8, 3.2 Hz), 7.40 (d, 1H, J=3.2 Hz), 7.89 (t, 1H, J=1.5 Hz), 7.94 (d, 1H, J=7.6 Hz), 8.26 (s, 1H), 8.72 (s, 1H); MS (ESI) m/z=463.1 (MH⁺).

### EXAMPLE 41

The following examples were made by the same method as that used in Example 40 for Compound 139 using the appropriate boronic acid in step 3.

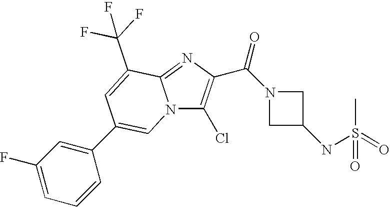

N-{1-[3-Chloro-6-(3-fluoro-phenyl)-8-trifluoromethyl-imidazo[1,2-a]pyridine-2-carbonyl]-azetidin-3-yl}-methanesulfonamide. (Compound 141) (21 mg, 51%) as a white solid. ¹H NMR (d₆-DMSO, 300 MHz) δ 2.95 (s, 3H), 3.96 (dd, 1H, J=5.6, 10.3 Hz), 4.27-4.47 (m, 3H), 4.87 (dd, 1H, J=8.2, 10.0 Hz), 7.32 (td, 1H, J=2.9, 8.5 Hz), 7.55-7.62 (m, 1H), 7.75 (d, 1H, J=8.2 Hz), 7.83 (dt, 1H, J=2.3, 10.3 Hz), 7.92 (d, 1H, J=8.2 Hz) 8.24 (s, 1H), 8.89 (s, 1H); MS (ESI) m/z=491.1 (MH⁺).

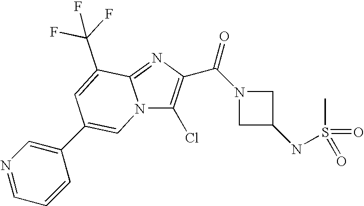

N-[1-(3-Chloro-6-pyridin-3-yl-8-trifluoromethyl-imidazo[1,2-a]pyridine-2-carbonyl)-azetidin-3-yl]-methanesulfonamide. (Compound 142) 35 mg, 88%) as a white solid. ¹H NMR (d₆-DMSO, 300 MHz) δ 2.95 (s, 3H), 3.97 (dd, 1H, J=5.3, 10.0 Hz), 4.28-4.78 (m, 3H), 4.87 (dd, 1H, J=8.2, 10.2 Hz), 7.95-8.02 (m, 2H), 8.38 (s, 1H), 8.82 (d, 1H, J=8.8 Hz), 8.88 (d, 1H, J=5.3 Hz), 9.15 (s, 1H), 9.35 (d, 1H, J=1.8 Hz); MS (ESI) m/z=525.0 (MH⁺).

### EXAMPLE 42

### N-[1-(3-Chloro-6-furan-3-yl-8-trifluoromethyl-imidazo[1,2-a]pyridine-2-carbonyl)-azetidin-3-yl]-N-methyl-methanesulfonamide. (Compound 143)

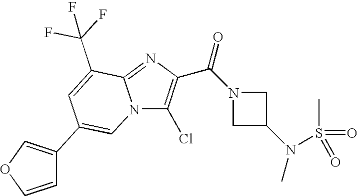

N-[1-(3-chloro-6-furan-3-yl-8-trifluoromethyl-imidazo[1,2-a]pyridine-2-carbonyl)-azetidin-3-yl]-methanesulfonamide (50 mg, 0.11 mmol), methyliodide (77 uL, 0.54 mmol), and cesium carbonate (176 mg, 0.54 mmol), were stirred at room temperature in DMF (550 uL) overnight. Solvent was removed in-vacuo and the residue was chromatographed to obtain N-[1-(3-chloro-6-furan-3-yl-8-trifluoromethyl-imidazo[1,2-a]pyridine-2-carbonyl)-azetidin-3-yl]-N-Methyl-methanesulfonamide (22 mg, 42%) as a white solid. ¹H NMR (d₆-DMSO, 300 MHz) δ 2.87 (s, 3H), 2.93 (s, 3H), 4.19-4.32 (m, 2H), 4.59-4.70 (m, 2H) 4.83 (dd, 1H, J=6.7, 10.5 Hz), 7.34 (dd, 1H, J=0.9, 1.8 Hz), 7.85 (t, 1H, J=1.8 Hz), 8.23 (s, 1H), 8.58 (s, 1H), 8.83 (s, 1H); MS (ESI) m/z=477.1 (MH⁺).

### EXAMPLE 43

The following example was made by the same method as that used in Example 42 for Compound 143 using 2-iodoethanol.

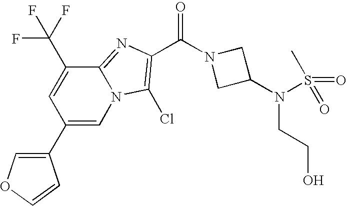

N-[1-(3-Chloro-6-furan-3-yl-8-trifluoromethyl-imidazo[1,2-a]pyridine-2-carbonyl)-azetidin-3-yl]-N-(2-hydroxy-ethyl)-methanesulfonamide. (Compound 144) (12 mg, 11%) as a white solid. ¹H NMR (d₆-DMSO, 300 MHz) δ 2.97 (s, 3H) 3.52-3.58 (m, 2H), 4.19-4.45 (m, 3H), 4.60-4.73 (m, 2H), 4.84-4.94 (m, 2H), 7.32 (dd, 1H, J=0.9, 2.1 Hz), 7.84 (t, 1H, J=1.8 Hz), 8.22 (s, 1H), 8.56 (s, 1H), 8.82 (s, 1H); MS (ESI) m/z=507.1 (MH⁺).

### EXAMPLE 44

### N-{1-[6-(5-Bromo-furan-3-yl)-8-trifluoromethyl-imidazo[1,2-a]pyridine-2-carbonyl]-azetidin-3-yl}-methanesulfonamide (Compound 145)

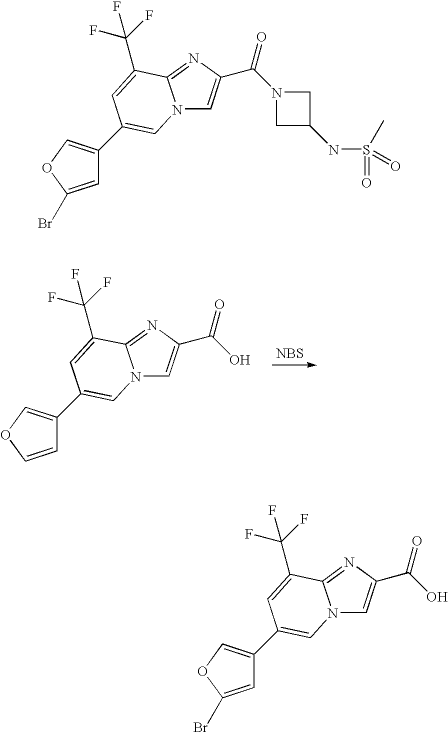

### Step 1: 6-(5-Bromo-furan-3-yl)-8-trifluoromethyl-imidazo[1,2-a]pyridine-2-carboxylic acid

NBS (661 mg, 3.72 mmol) was added to a stirring solution of 1-(3-chloro-6-furan-3-yl-8-trifluoromethyl-imidazo[1,2-a]pyridine-2-carboxylic acid (1 g, 3.38 mmol) in DMF (17 mL) at 0° C. The reaction was allowed to warm to room temperature overnight. Additional NBS (500 mg) was added and the reaction was stirred for 3 hours. Reaction was quenched with 5% aq. NaHSO₃, and the aqueous mixture was extracted with ether. The ether extracts were dried (MgSO₄) and solvent was removed to obtain 1-(3-chloro-6-(2-bromofuran)-4-yl-8-trifluoromethyl-imidazo[1,2-a]pyridine-2-carboxylic acid (332 mg 26%) as a light yellow solid. MS (ESI) m/z=374.9 (MH⁺).

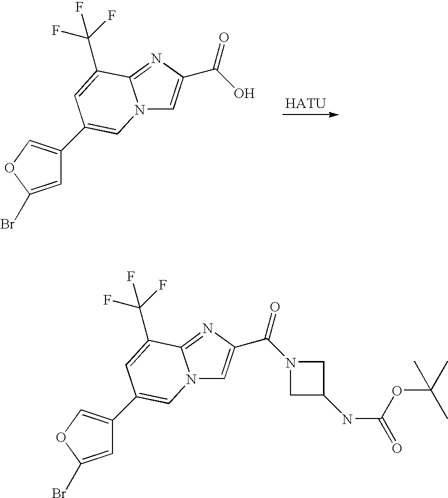

### Step 2: {1-[6-(5-Bromo-furan-3-yl)-8-trifluoromethyl-imidazo[1,2-a]pyridine-2-carbonyl]-azetidin-3-yl}-carbamic acid tert-butyl ester

HATU (673 mg, 1.77 mmol) was added to a DMF (4 mL) solution of 6-(5-Bromo-furan-3-yl)-8-trifluoromethyl-imidazo[1,2-a]pyridine-2-carboxylic acid (332 mg, 0.885 mmol), DIEA (570 uL, 4.42 mmol), and 3-tert-butoxycarbonyl aminoazetadine at room temperature. After 18 hours, water was added and the precipitate was filtered. Column chromatography of the crude gave {1-[6-(5-bromo-furan-3-yl)-8-trifluoromethyl-imidazo[1,2-a]pyridine-2-carbonyl]-azetidin-3-yl}-carbamic acid tert-butyl ester (168 mg 36%) as a white solid. MS (ESI) m/z=529.0 (MH⁺).

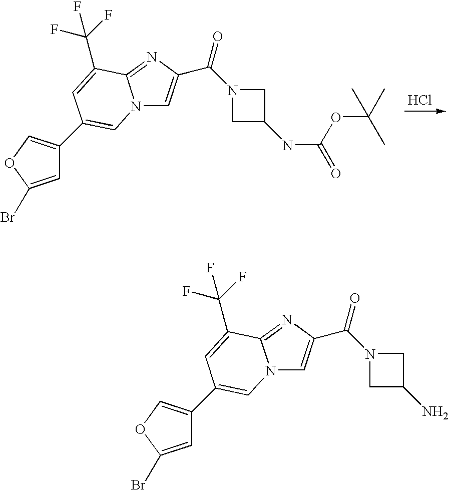

### Step 3: (3-Amino-azetidin-1-yl)-[6-(5-bromo-furan-3-yl)-8-trifluoromethyl-imidazo[1,2-a]pyridin-2-yl]-methanone-hydrochloride

A solution of hydrogen chloride in dioxane (4M, 2 mL) was added to {1-[6-(5-bromo-furan-3-yl)-8-trifluoromethyl-imidazo[1,2-a]pyridine-2-carbonyl]-azetidin-3-yl}-carbamic acid tert-butyl ester (168 mg, 0.32 mmol) and after 2 hours, the solvent was removed to obtain (3-amino-azetidin-1-yl)-[6-(5-bromo-furan-3-yl)-8-trifluoromethyl-imidazo[1,2-a]pyridin-2-yl]-methanone-hydrochloride (172 mg, 100%) as an off white solid. MS (ESI) m/z=429.3 (MH⁺).

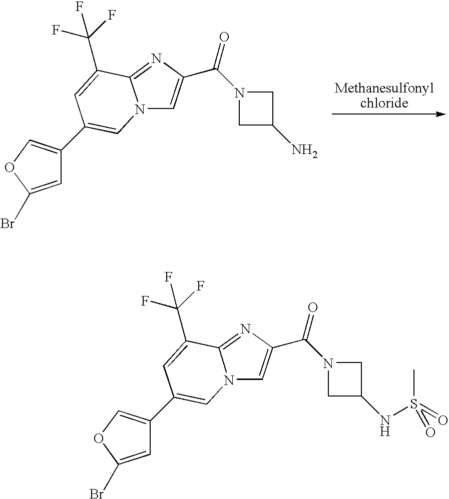

### Step 4: N-{1-[6-(5-Bromo-furan-3-yl)-8-trifluoromethyl-imidazo[1,2-a]pyridine-2-carbonyl]-azetidin-3-yl}-methanesulfonamide

Methanesulfonyl chloride (10 uL, 0.13 mmol) was added to a DMF (0.72 mL) solution of N,N′-diisopropylethylamine (184 uL) and (3-amino-azetidin-1-yl)-[6-(5-bromo-furan-3-yl)-8-trifluoromethyl-imidazo[1,2-a]pyridin-2-yl]-methanone-hydrochloride (60 mg, 0.14 mmol). After 2 hours, water was added and the precipitate was filtered and subjected to silica chromatography to give N-{1-[6-(5-bromo-furan-3-yl)-8-trifluoromethyl-imidazo[1,2-a]pyridine-2-carbonyl]-azetidin-3-yl}-methanesulfonamide (34 mg, 39%) as a white solid. ¹H NMR (d₆-DMSO, 300 MHz) δ 2.95 (s, 3H), 3.95 (dd, 1H, J=4.7, 10.0 Hz), 4.28-4.41 (m, 2H), 4.47 (dd, 1H, J=5.3, 10.5 Hz), 4.93 (dd, 1H, J=4.9, 9.4 Hz), 7.10 (d, 1H, J=2.1 Hz), 7.91 (d, 1H, J=7.9 Hz), 8.00 (d, 1H, J=2.1 Hz), 8.03 (s, 1H), 8.58 (s, 1H), 9.16 (d, 1H, J=0.9 Hz); MS (ESI) m/z=508.0 (MH⁺).

### EXAMPLE 45

The following examples were made by the same method as that used in Example 44 for Compound 145 using the appropriate sulfonyl chloride in step 4.

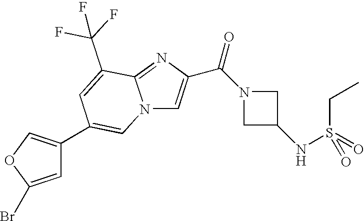

Ethanesulfonic acid {1-[6-(5-bromo-furan-3-yl)-8-trifluoromethyl-imidazo[1,2-a]pyridine-2-carbonyl]-azetidin-3-yl}-amide. (Compound 146) (31 mg, 61%) as a white solid. ¹H NMR (d₆-DMSO, 300 MHz) δ 1.21 (t, 3H, J=7.3 Hz), 3.03 (q, 2H, J=7.3 Hz), 3.93 (dd, 1H, J=4.7, 10.0 Hz), 4.25-4.40 (m, 2H), 4.48 (dd, 1H, J=5.0, 10.5 Hz), 4.92 (dd, 1H, J=8.2, 9.4 Hz), 7.10 (d, 1H, J=2.3 Hz), 7.94 (d, 1H, J=8.5 Hz), 8.00 (d, 1H, J=2.3 Hz), 8.03 (s, 1H), 8.58 (s, 1H), 9.16 (s, 1H); MS (ESI) m/z=522.0 (MH⁺).

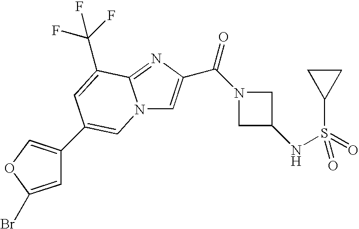

Cyclopropanesulfonic acid {1-[6-(5-bromo-furan-3-yl)-8-trifluoromethyl-imidazo[1,2-a]pyridine-2-carbonyl]-azetidin-3-yl}-amide. (Compound 147) (27 mg, 52%) as a white solid. ¹H NMR (d₆-DMSO, 300 MHz) δ 0.90-1.01 (m, 4H), 2.52-2.61 (m, 1H), 3.97 (dd, 1H, J=4.4, 9.4 Hz), 4.26-4.41 (m, 2H), 4.51 (dd, 1H, J=5.6, 10.8 Hz), 4.93 (dd, 1H, J=7.6, 9.4 Hz), 7.10 (d, 1H, J=2.3 Hz), 7.97 (d, 1H, J=8.5 Hz), 8.00 (d, 1H, J=2.1 Hz), 8.03 (s, 1H), 8.58 (s, 1H), 9.16 (d, 1H, J=1.2 Hz); MS (ESI) m/z=534.0 (MH⁺).

### EXAMPLE 46

### (3-Cyclopropyl-6-furan-3-yl-8-trifluoromethyl-imidazo[1,2-a]pyridin-2-yl)-(3-thiazol-2-yl-2,5-dihydro-pyrrol-1-yl)-methanone (Compound 148)

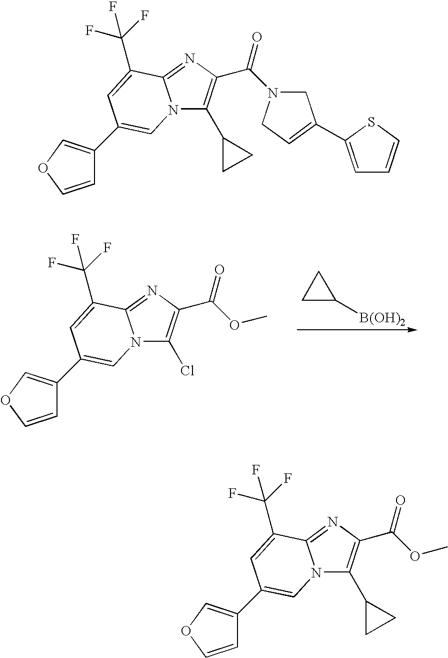

### Step 1: 3-Cyclopropyl-6-furan-3-yl-8-trifluoromethyl-imidazo[1,2-a]pyridine-2-carboxylic acid methyl ester

DMF (12 mL) was added under argon to a mixture of 3-chloro-6-furan-3-yl-8-trifluoromethyl-imidazo[1,2-a]pyridine-2-carboxylic acid methyl ester (500 mg, 1.45 mmol), cyclopropyl-boronic acid (625 mg, 7.26 mmol), and tetrakis(triphenylphosphine)palladium (0) (251 mg, 0.022 mmol) and then saturated aqueous NaHCO₃ (2 mL) was added. The reaction was heated in microwave to 120° C. for 40 min. The solvent was removed in-vacuo, and the resulting residue was chromatographed to obtain 3-cyclopropyl-6-furan-3-yl-8-trifluoromethyl-imidazo[1,2-a]pyridine-2-carboxylic acid methyl ester (420 mg, 83%) as a white solid. MS (ESI) m/z=351.1 (MH⁺).

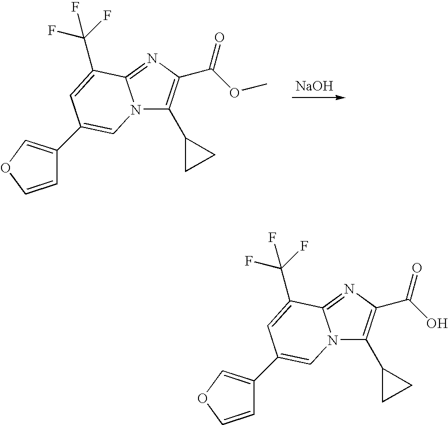

### Step 2: 3-Cyclopropyl-6-furan-3-yl-8-trifluoromethyl-imidazo[1,2-a]pyridine-2-carboxylic acid

3-Cyclopropyl-6-furan-3-yl-8-trifluoromethyl-imidazo[1,2-a]pyridine-2-carboxylic acid methyl ester was dissolved in THF (1.7 mL) and DMF (1 mL) and 3N NaOH (2.2 mL) was added. After 4 hours, the reaction mixture was adjusted to pH 4 with 1M citric acid. The resulting mixture was extracted with ethyl acetate. The collected organics were dried with MgSO₄ and removed in-vacuo to obtain 3-cyclopropyl-6-furan-3-yl-8-trifluoromethyl-imidazo[1,2-a]pyridine-2-carboxylic acid (290 mg, 72%) as a tan solid. MS (ESI) m/z=337.1 (MH⁺).

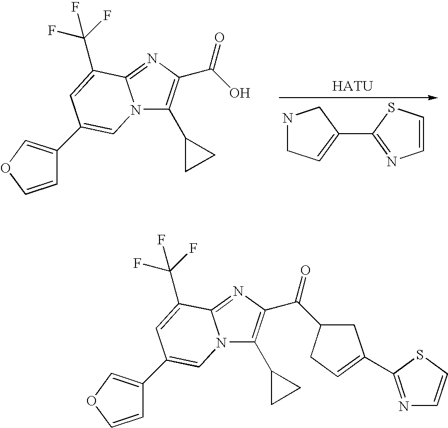

### Step 3: (3-Cyclopropyl-6-furan-3-yl-8-trifluoromethyl-imidazo[1,2-a]pyridin-2-yl)-(3-thiazol-2-yl-2,5-dihydro-pyrrol-1-yl)-methanone

Using standard HATU coupling conditions, 3-cyclopropyl-6-furan-3-yl-8-trifluoromethyl-imidazo[1,2-a]pyridine-2-carboxylic acid (100 mg, 0.29 mmol) and 2-(2,5-dihydro-1H-pyrrol-3-yl)-thiazole trifluororacetate (87 mg, 0.33 mmol) gave (3-cyclopropyl-6-furan-3-yl-8-trifluoromethyl-imidazo[1,2-a]pyridin-2-yl)-(3-thiazol-2-yl-2,5-dihydro-pyrrol-1-yl)-methanone (11 mg, 8%) as a white solid. ¹H NMR (d₆-DMSO, 300 MHz) δ 0.77-0.82 (m, 2H), 1.10-1.15 (m, 2H), 2.09-2.15 (m, 1H), 4.54-4.60 (m, 2H), 4.74-4.78 (m, 2H), 6.67 (d, 1H, J=19.9 Hz), 7.25 (s, 1H), 7.73-7.91 (m, 3H), 8.11 (s, 1H), 8.50 (s, 1H), 8.90 (s, 1H); MS (ESI) m/z=471.2 (MH⁺).

The following example was made by the same method as that used in Example 45 for Compound 148 using the appropriate amine in step 3.

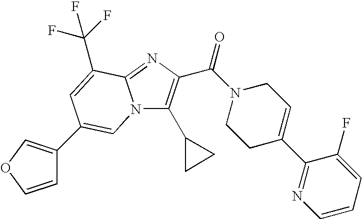

(3-Cyclopropyl-6-furan-3-yl-8-trifluoromethyl-imidazo[1,2-a]pyridin-2-yl)-(3-fluoro-3′,6′-dihydro-2′H-[2,4′]bipyridinyl-1′-yl)-methanone (Compound 149) Using standard HATU coupling conditions, 3-cyclopropyl-6-furan-3-yl-8-trifluoromethyl-imidazo[1,2-a]pyridine-2-carboxylic acid (70 mg, 0.21 mmol) and 3-fluoro-1′,2′,3′,6′-tetrahydro-[2,4′]bipyridinyl-hydrochloride (49 mg, 0.23 mmol) gave (3-cyclopropyl-6-furan-3-yl-8-trifluoromethyl-imidazo[1,2-a]pyridin-2-yl)-(3-fluoro-3′,6′-dihydro-2′H-[2,4′]bipyridinyl-1′-yl)-methanone (12 mg, 12%) as a white solid. ¹H NMR (d₆-DMSO, 300 MHz) δ 0.67-0.76 (m, 2H), 1.07-1.11 (m, 2H), 2.08-2.14 (m, 1H), 2.68 (d, 2H, J=26.7 Hz), 3.58 (t, 1H, J=5.6 Hz), 3.61 (t, 1H, J=5.9 Hz), 4.14 (s, 1H), 4.40 (s, 1H), 6.54 (d, 1H, J=66.8 Hz), 7.25 (s, 1H), 7.35-7.41 (m, 1H), 7.68-7.77 (m, 1H), 7.85 (s, 1H), 8.09 (s, 1H), 8.43 (s, 1H), 8.49 (s, 1H) 8.88 (d, 1H, J=4.1 Hz); MS (ESI) m/z=497.2 (MH⁺).

### EXAMPLE 47

### (3-Chloro-6-cyclopent-1-enyl-8-trifluoromethyl-imidazo[1,2-a]pyridin-2-yl)-(3-thiazol-2-yl-2,5-dihydro-pyrrol-1-yl)-methanone (Compound 150)

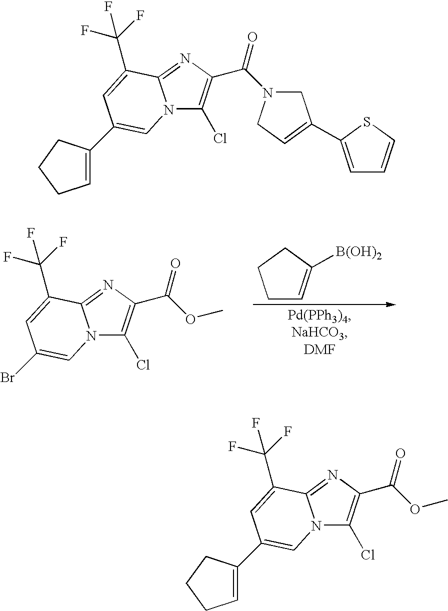

### Step 1: 3-Chloro-6-cyclopent-1-enyl-8-trifluoromethyl-imidazo[1,2-a]pyridine-2-carboxylic acid methyl ester

DMF (16 mL) was added under argon to a mixture of 6-bromo-3-chloro-8-trifluoromethyl-imidazo[1,2-a]pyridine-2-carboxylic acid methyl ester (500 mg, 1.4 mmol), cyclopent-1-enyl-boronic acid (470 mg, 4.2 mmol), and tetrakis(triphenylphosphine)palladium (0) (162 mg, 0.14 mmol), and then saturated aqueous NaHCO₃ (3 mL) was added. Reaction was heated in microwave to 100° C. for 20 min. The solvent was removed in-vacuo, and to the resulting residue was chromatographed to obtain 3-chloro-6-cyclopent-1-enyl-8-trifluoromethyl-imidazo[1,2-a]pyridine-2-carboxylic acid methyl ester (152 mg, 31%) as a white solid. MS (ESI) m/z=345.0 (MH⁺).

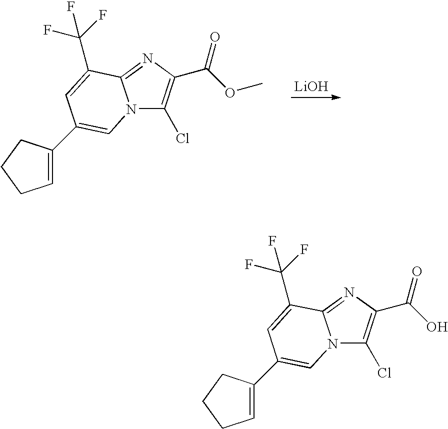

### Step 2: 3-Chloro-6-cyclopent-1-enyl-8-trifluoromethyl-imidazo[1,2-a]pyridine-2-carboxylic acid

LiOH was added to a suspension of 3-chloro-6-cyclopent-1-enyl-8-trifluoromethyl-imidazo[1,2-a]pyridine-2-carboxylic acid methyl ester (152 mg, 0.44 mmol) in ACN (10 mL) and DMF (10 mL) and mixture was stirred at room temperature for 1 hour. The reaction was then neutralized with 4 N HCl and white precipitate was filtered and dried to obtain 3-chloro-6-cyclopent-1-enyl-8-trifluoromethyl-imidazo[1,2-a]pyridine-2-carboxylic acid as a white solid. MS (ESI) m/z=331.0 (MH⁺).

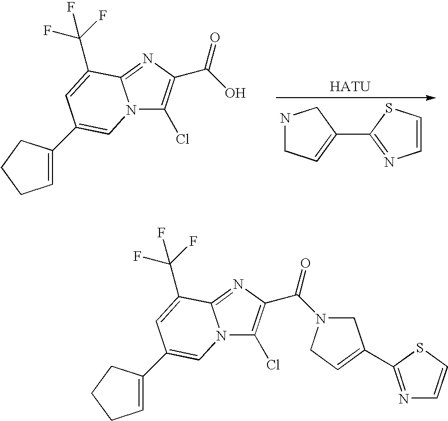

### Step 3: (3-Chloro-6-cyclopent-1-enyl-8-trifluoromethyl-imidazo[1,2-a]pyridin-2-yl)-(3-thiazol-2-yl-2,5-dihydro-pyrrol-1-yl)-methanone

Using standard HATU coupling conditions, 3-chloro-6-cyclopent-1-enyl-8-trifluoromethyl-imidazo[1,2-a]pyridine-2-carboxylic acid (85 mg, 0.25 mmol) and 2-(2,5-dihydro-1H-pyrrol-3-yl)-thiazole hydrochloride (72 mg, 0.39 mmol) gave (3-chloro-6-cyclopent-1-enyl-8-trifluoromethyl-imidazo[1,2-a]pyridin-2-yl)-(3-thiazol-2-yl-2,5-dihydro-pyrrol-1-yl)-methanone (60 mg, 52%) as a white solid. ¹H NMR (d₆-DMSO, 300 MHz) δ 1.98-2.08 (m, 2H), 2.51-2.59 (m, 2H), 2.75-2.81 (m, 2H), 4.55-4.59 (m, 1H), 4.75-4.78 (m, 1H), 4.91-4.93 (m, 1H), 5.09-5.12 (m, 1H), 6.69-6.72 (m, 2H), 7.78 (dd, 1H, J=3.5, 9.1 Hz), 7.87 (dd, 1H, J=3.5, 15.6 Hz), 8.20 (s, 1H), 8.27 (s, 1H); MS (ESI) m/z=464.9 (MH⁺).

### EXAMPLE 48

The following example was made by the same method as that used in Example 48 for Compound 150 using the appropriate amine in step 3.

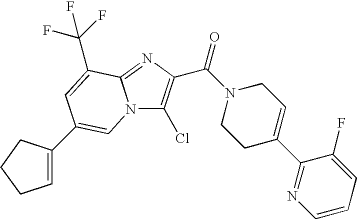

(3-Chloro-6-cyclopent-1-enyl-8-trifluoromethyl-imidazo[1,2-a]pyridin-2-yl)-(3-fluoro-3′,6′-dihydro-2′H-[2,4′]bipyridinyl-1′-yl)-methanone. (Compound 151) (71 mg, 50%) as a white solid. ¹H NMR (d₆-DMSO, 300 MHz) δ 1.98-2.07 (m, 2H), 2.54-2.57 (m, 2H), 2.69-2.80 (m, 4H), 3.83-3.92 (m, 2H), 4.38-4.46 (m, 2H), 6.49-6.70 (m, 2H), 7.35-7.41 (m, 1H), 7.68-7.77 (m, 1H), 8.16 (s, 1H), 8.25 (s, 1H), 8.42-8.45 (m, 1H); MS (ESI) m/z=490.9 (MH⁺).

### EXAMPLE 49

### (3-Chloro-6-furan-3-yl-8-trifluoromethyl-imidazo[1,2-a]pyridin-2-yl)-[4-(cyclopropylmethyl-amino)-piperidin-1-yl]-methanone (Compound 152)

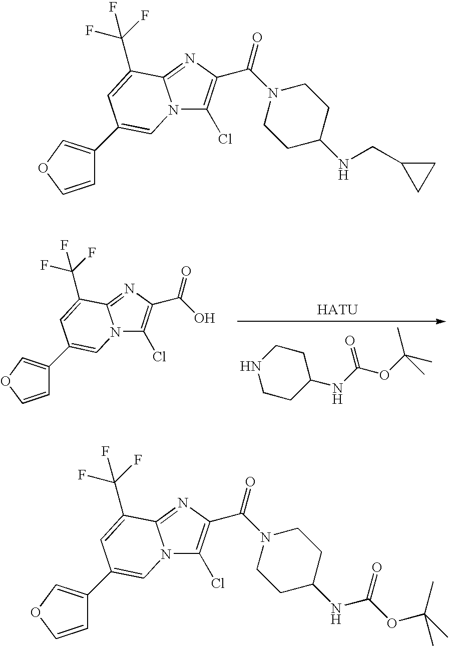

### Step 1: [1-(3-Chloro-6-furan-3-yl-8-trifluoromethyl-imidazo[1,2-a]pyridine-2-carbonyl)-piperidin-4-yl]-carbamic acid tert-butyl ester

Using standard HATU coupling conditions, 3-chloro-6-furan-3-yl-8-trifluoromethyl-imidazo[1,2-a]pyridine-2-carboxylic acid (1.33 g, 4.04 mmol) and piperidin-4-yl-carbamic acid tert-butyl ester (969 mg, 4.85 mmol) gave [1-(3-chloro-6-furan-3-yl-8-trifluoromethyl-imidazo[1,2-a]pyridine-2-carbonyl)-piperidin-4-yl]-carbamic acid tert-butyl ester (1.473 g, 71%) as a white solid. MS (ESI) m/z=513.0 (MH⁺).

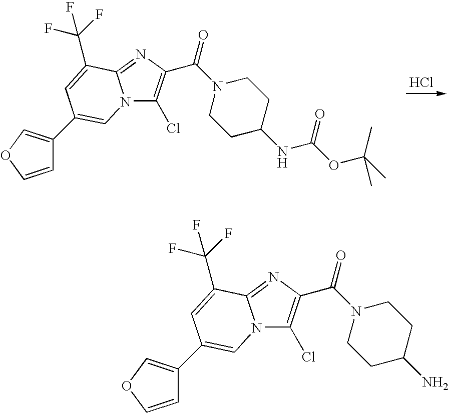

### Step 2: (4-Amino-piperidin-1-yl)-(3-chloro-6-furan-3-yl-8-trifluoromethyl-imidazo[1,2-a]pyridin-2-yl)-methanone

A solution of hydrogen chloride in dioxane (4M, 2 mL) was added to [1-(3-chloro-6-furan-3-yl-8-trifluoromethyl-imidazo[1,2-a]pyridine-2-carbonyl)-piperidin-4-yl]-carbamic acid tert-butyl ester (1.0 g, 1.95 mmol) and after 0.5 hour, solution was neutralized with 3 N NaOH. Water was added and the mixture was extracted with ethyl acetate. Collected organics were dried (MgSO₄) and removed to obtain (4-amino-piperidin-1-yl)-(3-chloro-6-furan-3-yl-8-trifluoromethyl-imidazo[1,2-a]pyridin-2-yl)-methanone (200 mg, 25%) as an off white solid. MS (ESI) m/z=413.3 (MH⁺).

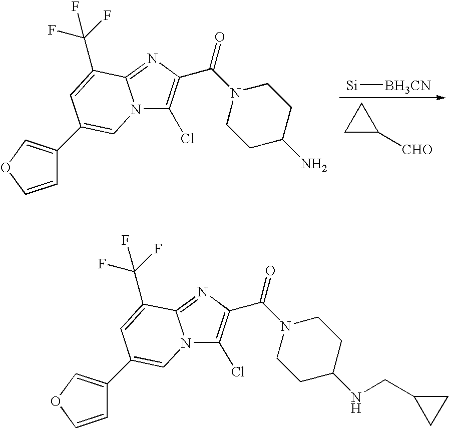

### Step 3: (3-Chloro-6-furan-3-yl-8-trifluoromethyl-imidazo[1,2-a]pyridin-2-yl)-[4-(cyclopropylmethyl-amino)-piperidin-1-yl]-methanone

Silica supported borohydride (256 mg, 0.238 mmol) was added to (4-amino-piperidin-1-yl)-(3-chloro-6-furan-3-yl-8-trifluoromethyl-imidazo[1,2-a]pyridin-2-yl)-methanone (98 mg, 0.238 mmol), and cyclopropanecarbaldehyde (18 uL, 0.238 mmol) in methanol (1 mL). After 3 hours at room temperature solvent was removed and sample was chromatographed to obtain (3-chloro-6-furan-3-yl-8-trifluoromethyl-imidazo[1,2-a]pyridin-2-yl)-[4-(cyclopropylmethyl-amino)-piperidin-1-yl]-methanone (37 mg, 33%) as a white solid. ¹H NMR (d₆-DMSO, 300 MHz) δ 0.82 (d, 2H, J=4.7 Hz), 0.45-0.50 (m, 2H), 0.88-0.95 (m, 1H), 1.31-1.44 (m, 2H), 1.95 (dd, 2H, J=13.5, 38.1 Hz), 2.60 (d, 2H, J=6.2 Hz), 2.92-2.99 (m, 2H), 3.13-3.20 (m, 2H), 4.06 (d, 1H, J=13.2 Hz), 4.42 (d, 1H, J=12.6 Hz), 7.32 (d, 1H, J=0.9 Hz), 7.84 (t, 1H, J=1.8 Hz), 8.20 (s, 1H), 8.55 (s, 1H), 8.81 (s, 1H); MS (ESI) m/z=467.2 (MH⁺).

### EXAMPLE 50

### (S)-6-tert-Butoxycarbonylamino-2-[(6-furan-3-yl-8-trifluoromethyl-imidazo[1,2-a]pyridine-2-carbonyl)-amino]-hexanoic acid methyl ester (Compound 153)

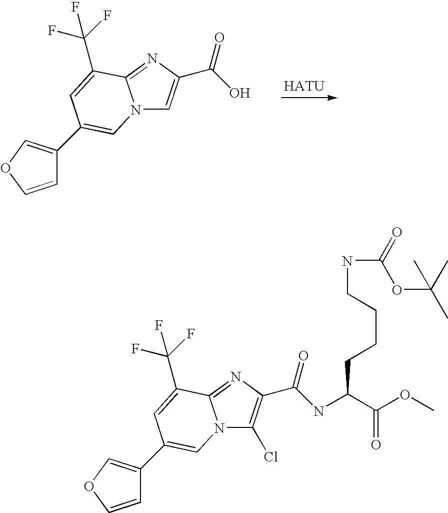

(S)-6-tert-Butoxycarbonylamino-2-[(6-furan-3-yl-8-trifluoromethyl-imidazo[1,2-a]pyridine-2-carbonyl)-amino]-hexanoic acid methyl ester. Using standard HATU coupling conditions, 3-chloro-6-furan-3-yl-8-trifluoromethyl-imidazo[1,2-a]pyridine-2-carboxylic acid (100 mg, 0.34 mmol) and H-L-Lys(Boc)-OMe hydrochloride (100 mg, 0.34 mmol) gave (S)-6-tert-butoxycarbonylamino-2-[(6-furan-3-yl-8-trifluoromethyl-imidazo[1,2-a]pyridine-2-carbonyl)-amino]-hexanoic acid methyl ester (98 mg, 54%) as a white solid. MS (ESI) m/z=539.0 (MH⁺).

### EXAMPLE 51

The following examples were made by the same method as that used in Example 50 for Compound 153 using the appropriate amino acid ester.

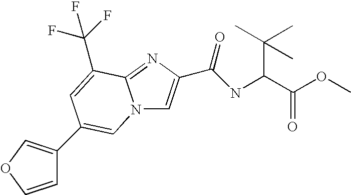

2-[(6-Furan-3-yl-8-trifluoromethyl-imidazo[1,2-a]pyridine-2-carbonyl)-amino]-3,3-dimethyl-butyric acid methyl ester. (Compound 154). Using standard HATU coupling conditions (70 mg, 49%) as a white solid. MS (ESI) m/z=424.0 (MH⁺).

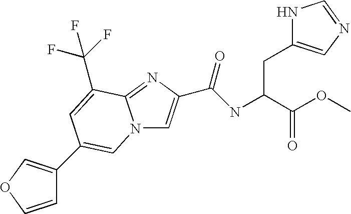

2-[(6-Furan-3-yl-8-trifluoromethyl-imidazo[1,2-a]pyridine-2-carbonyl)-amino]-3-(3H-imidazol-4-yl)-propionic acid methyl ester. (Compound 155). Using standard HATU coupling conditions (64 mg, 42%) as a white solid. MS (ESI) m/z=447.9 (MH⁺).

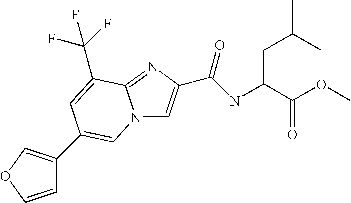

2-[(6-Furan-3-yl-8-trifluoromethyl-imidazo[1,2-a]pyridine-2-carbonyl)-amino]-4-methyl-pentanoic acid methyl ester. (Compound 156) Using standard HATU coupling conditions (66 mg, 46%) as a white solid. MS (ESI) m/z=424.0 (MH⁺).

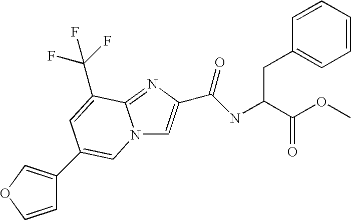

2-[(6-Furan-3-yl-8-trifluoromethyl-imidazo[1,2-a]pyridine-2-carbonyl)-amino]-3-phenyl-propionic acid methyl ester. (Compound 157). Using standard HATU coupling conditions (129 mg, 83%) as a white solid. MS (ESI) m/z=458.1 (MH⁺).

### EXAMPLE 52

### (3-Chloro-6-furan-3-yl-8-trifluoromethyl-imidazo[1,2-a]pyridin-2-yl)-[4-(3-methyl-butoxy)-piperidin-1-yl]-methanone (Compound 158)

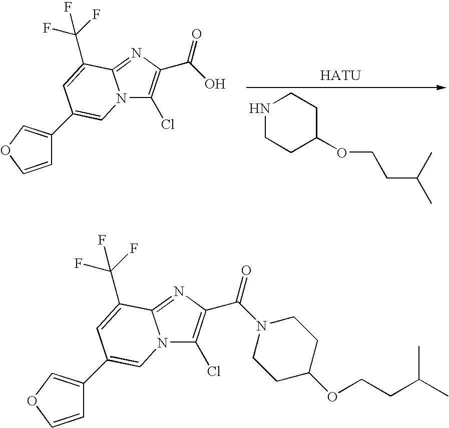

(3-Chloro-6-furan-3-yl-8-trifluoromethyl-imidazo[1,2-a]pyridin-2-yl)-[4-(3-methyl-butoxy)-piperidin-1-yl]-methanone. Using standard HATU coupling conditions, 3-chloro-6-furan-3-yl-8-trifluoromethyl-imidazo[1,2-a]pyridine-2-carboxylic acid (100 mg, 0.30 mmol) and 4-(3-methyl-butoxy)-piperidine (63 mg, 0.30 mmol) gave (3-chloro-6-furan-3-yl-8-trifluoromethyl-imidazo[1,2-a]pyridin-2-yl)-[4-(3-methyl-butoxy)-piperidin-1-yl]-methanone (13 mg, 9% yield) as a yellow solid. ¹H NMR (d₆-DMSO, 300 MHz) δ 0.88 (d, 6H, J=6.4 Hz), 1.23-1.27 (m, 2H), 1.36-1.50 (m, 4H), 1.61-1.72 (m, 1H), 1.80-1.96 (m, 2H), 3.35-3.58 (m, 3H), 3.75-3.83 (m, 1H), 3.95-34.02 (m, 1H), 7.32 (d, 1H, J=0.9 Hz), 7.84 (t, 1H, J=1.8 Hz), 8.19 (s, 1H), 8.55 (s, 1H), 8.81 (s, 1H); MS (ESI) m/z=483.8 (MH⁺).

### EXAMPLE 53

### 2-[1-(3-Chloro-6-furan-3-yl-8-trifluoromethyl-imidazo[1,2-a]pyridine-2-carbonyl)-1,2,3,6-tetrahydro-pyridin-4-yl]-cyclopent-2-enone (Compound 159)

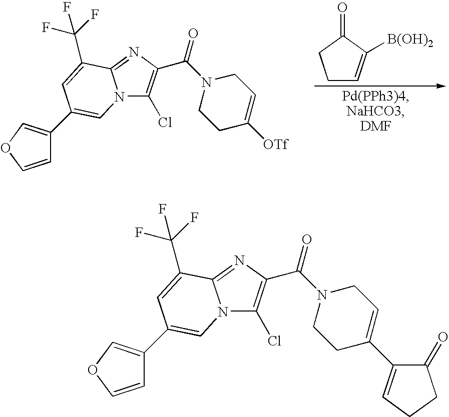

Step 1: 2-[1-(3-Chloro-6-furan-3-yl-8-trifluoromethyl-imidazo[1,2-a]pyridine-2-carbonyl)-1,2,3,6-tetrahydro-pyridin-4-yl]-cyclopent-2-enone. DMF (6.6 mL) was added under argon to a mixture of trifluoro-methanesulfonic acid 1-(3-chloro-6-furan-3-yl-8-trifluoromethyl-imidazo[1,2-a]pyridine-2-carbonyl)-1,2,3,6-tetrahydro-pyridin-4-yl ester (359 mg, 0.66 mmol), 2-cyclopentenone-2-boronic acid (250 mg, 1.98 mmol), and tetrakis(triphenylphosphine)palladium(0) (76 mg, 0.066 mmol), and then saturated aqueous NaHCO₃ (1.3 mL) was added. The reaction was heated in microwave to 100° C. for 20 min. The solvent was removed in-vacuo, and the resulting residue was chromatographed to obtain 2-[1-(3-chloro-6-furan-3-yl-8-trifluoromethyl-imidazo[1,2-a]pyridine-2-carbonyl)-1,2,3,6-tetrahydro-pyridin-4-yl]-cyclopent-2-enone (226 mg, 72%) as a yellow solid. ¹H NMR (d₆-DMSO, 300 MHz) δ 2.40-2.44 (m, 4H), 2.56 (s, 2H), 3.77-3.85 (m, 2H), 4.27-4.33 (m, 2H), 6.74-6.89 (m, 1H), 7.32 (s, 1H), 7.84-7.95 (m, 1H), 8.20 (s, 1H) 8.56 (s, 1H) 8.82 (s, 1H); MS (ESI) m/z=475.9 (MH⁺).

### EXAMPLE 56

### N—[(R)-1-(3-Chloro-6-furan-3-yl-8-trifluoromethyl-imidazo[1,2-a]pyridine-2-carbonyl)-pyrrolidin-3-yl]-2,2,2-trifluoro-acetamide (Compound 162)

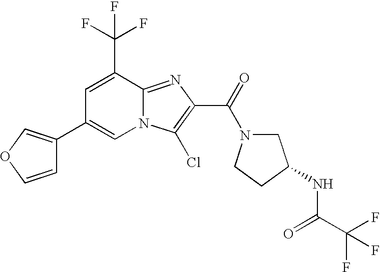

### Step 1: (R)-1-(3-Chloro-6-furan-3-yl-8-trifluoromethyl-imidazo[1,2-a]pyridine-2-carbonyl)-pyrrolidin-3-yl]-carbamic acid tert-butyl ester

(R)-1-(3-Chloro-6-furan-3-yl-8-trifluoromethyl-imidazo[1,2-a]pyridine-2-carbonyl)-pyrrolidin-3-yl]-carbamic acid tert-butyl ester was prepared by standard HATU coupling of 3-chloro-6-furan-3-yl-8-trifluoromethyl-imidazo[1,2-a]pyridine-2-carboxylic acid and (R)-(−)-3-(Boc-amino)pyrrolidine. ¹H NMR (d₆-DMSO, 300 MHz) δ 1.36 (s, 4.5H), 1.41 (s, 4.5H), 2.00-2.12 (m, 1H), 1.76-1.90 (m, 1H), 3.36-4.10 (m, 5H), 7.24 (m, 1H), 7.32 (m, 1H), 7.84 (t, 1H, J=1.7 Hz), 8.20 (s, 1H), 8.55 (s, 1H), 8.81 (s, 1H); MS (ESI) m/z=499.1 (MH⁺).

### Step 2: ((R)-3-Amino-pyrrolidin-1-yl)-(3-chloro-6-furan-3-yl-8-trifluoromethyl-imidazo[1,2-a]pyridin-2-yl)-methanone

To a solution of (R)-1-(3-chloro-6-furan-3-yl-8-trifluoromethyl-imidazo[1,2-a]pyridine-2-carbonyl)-pyrrolidin-3-yl]-carbamic acid tert-butyl ester (203.2 mg) in DCM (4 mL) was added trifluoroacetic acid (2 mL). After 45 min, the solvent was concentrated under reduced pressure to give ((R)-3-amino-pyrrolidin-1-yl)-(3-chloro-6-furan-3-yl-8-trifluoromethyl-imidazo[1,2-a]pyridin-2-yl)-methanone (425 mg) as a TFA salt which was used for the next step without further purification. ¹H NMR (d₆-DMSO, 300 MHz) δ 2.18-2.32 (m, 1H), 1.94-2.12 (m, 1H), 3.60-4.21 (m, 5H), 7.33 (m, 1H), 7.85 (t, 1H, J=1.8 Hz), 8.22 (brs, 4H), 8.56 (d, 1H, J=0.9 Hz), 8.84 (s, 1H); MS (ESI) m/z=399 (MH⁺).

### Step 3: N—[(R)-1-(3-Chloro-6-furan-3-yl-8-trifluoromethyl-imidazo[1,2-a]pyridine-2-carbonyl)-pyrrolidin-3-yl]-2,2,2-trifluoro-acetamide (Compound 162)

To a solution of ((R)-3-amino-pyrrolidin-1-yl)-(3-chloro-6-furan-3-yl-8-trifluoromethyl-imidazo[1,2-a]pyridin-2-yl)-methanone (425 mg, 0.407 mmol) in DMF (2 mL) was added di-isopropylethylamine (0.384 mL). Upon cooling with ice-water bath, 4-chlorobutyryl chloride (50.3 μL, 0.448 mmol) was added. After 30 min, additional di-isopropylethylamine (0.2 mL) and 4-chlorobutyryl chloride (30 μL) were added. After 15 min, the mixture was diluted with EtOAc (20 mL) and washed with saturated aqueous NaHCO₃ (10 mL), then brine (10 mL). The extracts were dried (Na₂SO₄), filtered and concentrated. Column chromatography [gradient elution with n-hex/EtOAc (1:1 v/v) to (1:6 v/v)]gave N—[(R)-1-(3-chloro-6-furan-3-yl-8-trifluoromethyl-imidazo[1,2-a]pyridine-2-carbonyl)-pyrrolidin-3-yl]-2,2,2-trifluoro-acetamide (77.2 mg) as a side product. ¹H NMR (d₆-DMSO, 300 MHz) δ 1.90-2.25 (m, 2H), 3.50-4.11 (m, 4H), 4.43 (m, 1H), 7.32 (brs, 1H), 7.84 (t, 1H, J=1.5 Hz), 8.21 (s, 1H), 8.56 (s, 1H), 8.83 (s, 1H), 9.70 (d, 0.5H, J=6.7 Hz), 9.76 (d, 0.5H, J=6.4 Hz).

### EXAMPLE 58

### 6-Furan-3-yl-2-(3-thiazol-2-yl-2,5-dihydro-pyrrol-1-ylmethyl)-8-trifluoromethyl-imidazo[1,2-a]pyridine (Compound 163)

### Step 1: 5-Furan-3-yl-3-trifluoromethyl-pyridin-2-ylamine

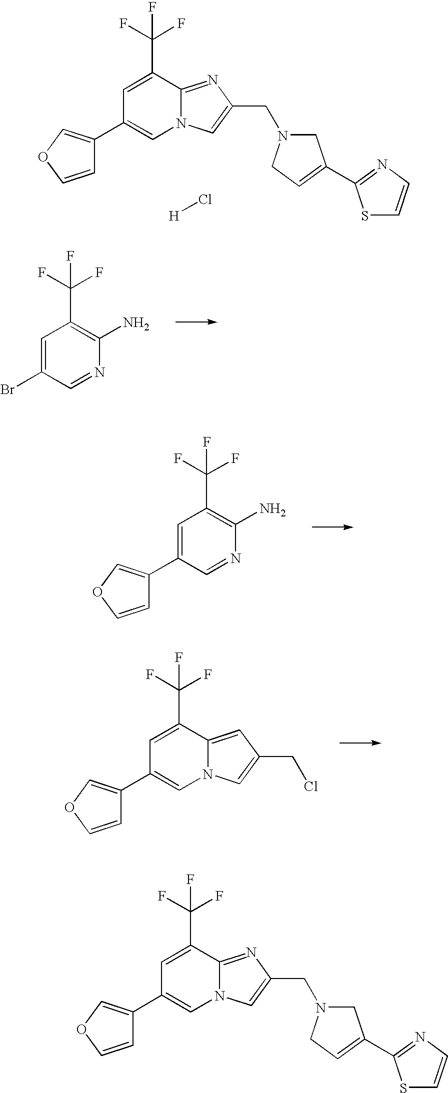

A mixture of 5-bromo-3-trifluoromethyl-pyridin-2-ylamine (4.82 g, 20 mmol), 3-furanboronic acid (4.48 g, 40 mmol) and tetrakis(triphenylphosphine) palladium(0) (693 mg, 0.6 mmol) was stirred at 80° C. in 1,4-dioxane (100 mL) and 1M K₃PO₄ (25 mL) for 2.5 hours. Upon cooling, the organic solvent was removed under reduced pressure. The crude was diluted with EtOAc (300 mL) and washed with saturated NaHCO₃, (2×100 mL), then brine (100 mL). The extracts were filtered through a pad of silica gel and washed with EtOAc (150 mL). Column chromatographed [n-hex/EtOAc (5:1 v/v) to (3:1 v/v)]of the crude gave 5-furan-3-yl-3-trifluoromethyl-pyridin-2-ylamine (3.82 g) as a solid. ¹H NMR (d₆-DMSO, 300 MHz) δ 6.47 (brs, 2H), 6.99 (dd, 1H, J=0.9, 2 Hz), 7.72 (t, 1H, J=1.8 Hz), 7.95 (d, 1H, J=2 Hz), 8.17 (dd, 1H, J=0.9, 1.8 Hz), 8.50 (dd, 1H, J=0.6, 2 Hz); MS (ESI) m/z=229 (MH⁺).

### Step 2: 2-Chloromethyl-6-furan-3-yl-8-trifluoromethyl-imidazo[1,2-a]pyridine

A mixture of 5-furan-3-yl-3-trifluoromethyl-pyridin-2-ylamine (3.41 g, 14.94 mmol), and 1,3-dichloroacetone (5.69 g, 44.81 mmol) was heated in THF (3.7 mL) at 80° C. for 39 hours. Upon cooling, the product was filtered, washed with THF (2×3 mL), and dried under high vacuum to give 2-chloromethyl-6-furan-3-yl-8-trifluoromethyl-imidazo[1,2-a]pyridine (4.97 g) as a white powder. ¹H NMR (d₆-DMSO, 300 MHz) δ 4.93 (s, 2H), 7.04 (dd, 1H, J=0.9, 2 Hz), 7.82 (t, 1H, J=1.8 Hz), 8.06 (brs, 1H), 8.14 (s, 1H), 8.41 (t, 1H, J=1.2 Hz), 9.16 (d, 1H, J=0.9 Hz); MS (ESI) m/z=301.1 (MH⁺).

### Step 3: 6-Furan-3-yl-2-(3-thiazol-2-yl-2,5-dihydro-pyrrol-1-ylmethyl)-8-trifluoromethyl-imidazo[1,2-a]pyridine

A suspension of 2-chloromethyl-6-furan-3-yl-8-trifluoromethyl-imidazo[1,2-a]pyridine (78.5 mg, 0.261 mmol), 2-pyrrolidin-3-yl-thiazole trifluoroacetate (139 mg, 0.522 mmol), and potassium carbonate (180 mg, 1.31 mmol) was stirred in DMF (2.5 mL) at 80° C. for 4 hours. The mixture was then stirred at room temperature overnight. The precipitate was filtered and subjected to preparative HPLC purification (20-99% ACN gradient) to give 6-furan-3-yl-2-(3-thiazol-2-yl-2,5-dihydro-pyrrol-1-ylmethyl)-8-trifluoromethyl-imidazo[1,2-a]pyridine chloride (20 mg).

Data for 6-furan-3-yl-2-(3-thiazol-2-yl-2,5-dihydro-pyrrol-1-ylmethyl)-8-trifluoromethyl-imidazo[1,2-a]pyridine chloride

¹H NMR (d₆-DMSO, 300 MHz) δ 4.42 (brs, 2H), 4.64 (brs, 2H), 4.78 (brs, 2H), 6.63 (brs, 1H), 7.06 (dd, 1H, J=0.6, 1.8 Hz), 7.82-7.90 (m, 3H), 8.10 (s, 1H), 8.31 (s, 1H), 8.44 (s, 1H), 9.25 (s, 1H); MS (ESI) m/z=417.1 (MH⁺).

### EXAMPLE 61

### N—[(R)-1-(3-Chloro-6-furan-3-yl-8-trifluoromethyl-imidazo[1,2-a]pyridine-2-carbonyl)-pyrrolidin-3-yl]-acrylamide (Compound 167)

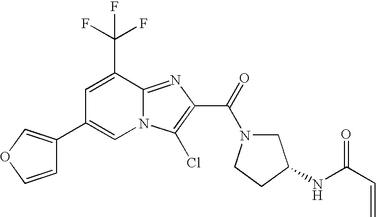

A mixture of ((R)-3-amino-pyrrolidin-1-yl)-(3-chloro-6-furan-3-yl-8-trifluoromethyl-imidazo[1,2-a]pyridin-2-yl)-methanone (174 mg, 0.30 mmol), 3-chloropropionic acid (35.8 mg, 0.33 mmol), HATU (125.5 mg, 0.33 mmol) and di-isopropylethylamine (0.26 mL, 1.5 mmol) was stirred in DMF (1.5 mL) for 35 min. The mixture was diluted with EtOAc (75 mL) and washed with saturated aqueous NaHCO₃ (30 mL), then brine (30 mL). The extracts were dried (Na₂SO₄), filtered and concentrated to give 3-chloro-N—[(R)-1-(3-chloro-6-furan-3-yl-8-trifluoromethyl-imidazo[1,2-a]pyridine-2-carbonyl)-pyrrolidin-3-yl]-propionamide (189 mg) as yellow solid which was used for the next step without further purification. To a stirred solution of 3-chloro-N—[(R)-1-(3-chloro-6-furan-3-yl-8-trifluoromethyl-imidazo[1,2-a]pyridine-2-carbonyl)-pyrrolidin-3-yl]-propionamide (154 mg) in DMF (2 mL) was added NaH (95%, 12 mg). After 30 min, the mixture was diluted with EtOAc (75 mL) and washed with saturated aqueous NaHCO₃ (30 mL), then brine (30 mL). The extracts were dried (Na₂SO₄), filtered and concentrated. Column chromatography [DCM/MeOH (95:5 v/v)]of the crude material followed by precipitation with EtOAc/n-hex gave N—[(R)-1-(3-chloro-6-furan-3-yl-8-trifluoromethyl-imidazo[1,2-a]pyridine-2-carbonyl)-pyrrolidin-3-yl]-acrylamide (34.3 mg) as yellow solid. ¹H NMR (d₆-DMSO, 300 MHz) δ 1.80-1.96 (m, 1H), 2.06-2.20 (m, 1H), 3.40-4.08 (m, 4H), 4.38 (brs, 1H), 5.66 (dt, 1H, J=2.6, 9.7 Hz), 6.02-6.30 (m, 2H), 7.32 (brs, 1H), 7.82-7.86 (m, 1H), 8.20 (brs, 1H), 8.38 (d, 0.5H, J=7 Hz), 8.45 (d, 0.5H, J=6 Hz), 8.56 (s, 1H), 8.82 (d, 1H, J=4.7 Hz); MS (ESI) m/z=453.4 (MH⁺).

### EXAMPLE 63

### 3-Chloro-8-difluoromethyl-6-furan-3-yl-2-(3-thiazol-2-yl-2,5-dihydro-pyrrole-1-carbonyl)-imidazo[1,2-a]pyridine-5-carbonitrile (Compound 169)

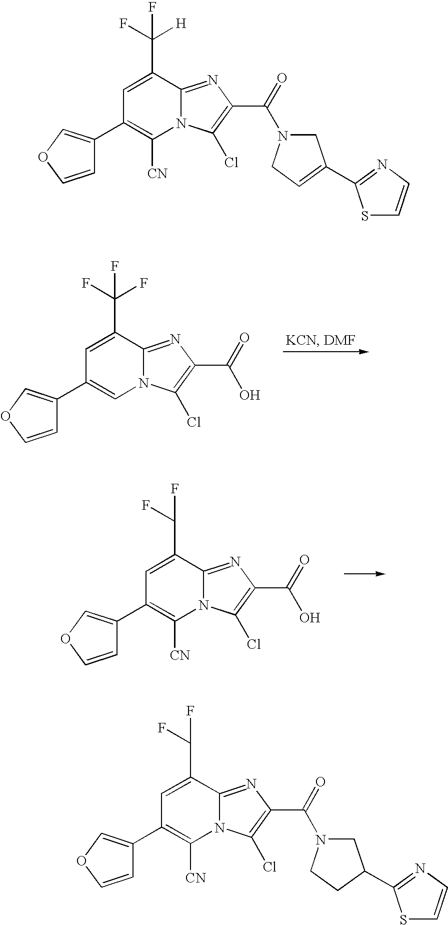

### Step 1: 3-Chloro-5-cyano-8-difluoromethyl-6-furan-3-yl-imidazo[1,2-a]pyridine-2-carboxylic acid

A mixture of 3-chloro-6-furan-3-yl-8-trifluoromethyl-imidazo[1,2-a]pyridine-2-carboxylic acid (331 mg, 1 mmol) and potassium cyanide (195 mg, 3 mmol) was stirred in DMF (5 mL) and water (1 mL) at 80° C. for 100 min. Upon cooling, the solvent was removed under reduced pressure. Water (5 mL) was added followed by careful addition of 2N HCl in which any gas evolved was passed through an aqueous KOH solution. The reddish precipitate was centrifuged. EtOAc was added to dissolve the product, and the solution was dried (Na₂SO₄), filtered and concentrated to give crude 3-chloro-5-cyano-8-difluoromethyl-6-furan-3-yl-imidazo[1,2-a]pyridine-2-carboxylic acid (267 mg) as a dark purple solid which was used for the next step without further purification. ¹H NMR (d₆-DMSO, 300 MHz) δ 7.14 (m, 1H), 7.50 (t, 1H, J=53.6 Hz), 7.89 (s, 1H), 7.95 (t, 1H, J=1.8 Hz), 8.48 (s, 1H); MS (ESI) m/z=338 (MH⁺).

### Step 2: 3-Chloro-8-difluoromethyl-6-furan-3-yl-2-(3-thiazol-2-yl-pyrrolidine-1-carbonyl)-imidazo[1,2-a]pyridine-5-carbonitrile

To a solution of 3-chloro-5-cyano-8-difluoromethyl-6-furan-3-yl-imidazo[1,2-a]pyridine-2-carboxylic acid (267 mg, 0.79 mmol), 2-pyrrolidin-3-yl-thiazole hydrochloride (164 mg, 0.87 mmol) in DMF (4 mL) was added di-isopropylethylamine (0.689 mL, 3.95 mmol) and HATU (331 mg, 0.87 mmol). After 30 min, the mixture was diluted with EtOAc (100 mL) and washed with saturated aqueous NaHCO₃ (30 mL), then brine (30 mL). The organic later was filtered through a pad of silica gel and the solvent removed under reduced pressure. The crude material was purified by flash chromatography [n-hex:EtOAc (5:4 v/v)]to give 3-chloro-8-difluoromethyl-6-furan-3-yl-2-(3-thiazol-2-yl-2,5-dihydro-pyrrole-1-carbonyl)-imidazo[1,2-a]pyridine-5-carbonitrile as a yellow solid (186 mg). ¹H NMR (d₆-DMSO, 300 MHz) δ 4.58 (m, 1H), 4.78 (m, 1H), 4.82 (m, 1H), 4.96 (m, 1H), 6.70 (m, 1H), 7.14 (m, 1H), 7.53 (dt, 1H, J=8.5, 53 Hz), 7.74-7.96 (m, 4H), 8.47 (d, 1H, J=0.9 Hz); MS (ESI) m/z=472 (MH⁺).

### EXAMPLE 64

### (6-Furan-3-yl-3-hydroxymethyl-8-trifluoromethyl-imidazo[1,2-a]pyridin-2-yl)-(3-thiazol-2-yl-2,5-dihydro-pyrrol-1-yl)-methanone (Compound 170)

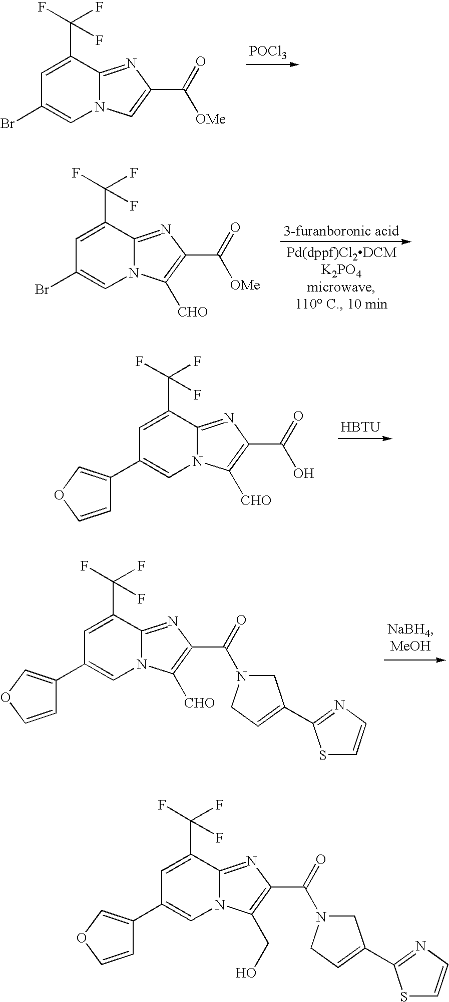

### Step 1: 6-Bromo-3-formyl-8-trifluoromethyl-imidazo[1,2-a]pyridine-2-carboxylicacid methyl ester

6-Bromo-8-trifluoromethyl-imidazo[1,2-a]pyridine-2-carboxylic acid methyl ester (1.02 g, 3.16 mmol) was dissolved in DMF (2.5 mL), POCl₃ (3 mL) was added and the mixture microwaved at 110° C. for 5 minutes. The reaction mixture was poured into conc. HCl and stirred for 15 minutes. The reaction mixture was basified with aqueous NaOH (20% w/v) and an extractive work up with ethyl acetate afforded the crude product. Purification by flash chromatography afforded 6-bromo-3-formyl-8-trifluoromethyl-imidazo[1,2-a]pyridine-2-carboxylicacid methyl ester (0.29 g, 26%). MS (ESI) m/z=353.7 (M+2).

### Step 2: 3-Formyl-6-furan-3-yl-8-trifluoromethyl-imidazo[1,2-a]pyridine-2-carboxylic acid

6-Bromo-3-formyl-8-trifluoromethyl-imidazo[1,2-a]pyridine-2-carboxylic acid methyl ester (0.12 g, 0.34 mmol) and 3-furanboronic acid (0.05 g, 0.44 mmol) were dissolved in 1,4-dioxane (3 mL). Potassioum phosphate solution (3M, 1.1 mL) was added and Argon gas was bubbled through the mixture. Catalyst (Pd(dppf)Cl₂.DCM, 0.017 g, 0.02 mmol) was added and the mixture microwaved at 110° C. for 10 minutes. The procedure was repeated with another batch of 0.17 g of 6-bromo-3-formyl-8-trifluoromethyl-imidazo[1,2-a]pyridine-2-carboxylicacid methyl ester and the 2 batches were combined. The solvents were evaporated and redissolved in EtOAc (30 mL) and water. The aqueous layer was acidified to pH-2 with 1N HCl. An extractive work-up afforded crude 3-formyl-6-furan-3-yl-8-trifluoromethyl-imidazo[1,2-a]pyridine-2-carboxylic acid (0.29 g) which was used for the next step without further purification. MS (ESI) m/z=324.5 (MH⁺).

### Step 3: (6-Furan-3-yl-3-hydroxymethyl-8-trifluoromethyl-imidazo[1,2-a]pyridin-2-yl)-(3-thiazol-2-yl-2,5-dihydro-pyrrol-1-yl)-methanone

3-Formyl-6-furan-3-yl-8-trifluoromethyl-imidazo[1,2-a]pyridine-2-carboxylic acid (0.29 g, 0.89 mmol) and 2-(2,5-dihydro-1H-pyrrol-3-yl)-thiazole were coupled using standard amide coupling procedures with HBTU as the coupling agent. Purification using flash chromatography afforded 6-furan-3-yl-2-(3-thiazol-2-yl-2,5-dihydro-pyrrole-1-carbonyl)-8-trifluoromethyl-imidazo[1,2-a]pyridine-3-carbaldehyde (0.042 g, 10%). MS (ESI) m/z=459.1 (MH⁺).

6-Furan-3-yl-2-(3-thiazol-2-yl-2,5-dihydro-pyrrole-1-carbonyl)-8-trifluoromethyl-imidazo[1,2-a]pyridine-3-carbaldehyde (0.042 g, 0.09 mmol) was suspended in MeOH (5 mL) and the mixture was cooled to 0° C. NaBH₄ (0.01 g) was added and the mixture refluxed for 10 min. An additional portion of NaBH₄ (0.02 g) and the mixture refluxed for an additional 15 minutes. The mixture was cooled to room temperature and dried under vacuum. The solid residue obtained was washed with 1N HCl, aqueous NaHCO₃, water and finally ethyl acetate to afford (6-furan-3-yl-3-hydroxymethyl-8-trifluoromethyl-imidazo[1,2-a]pyridin-2-yl)-(3-thiazol-2-yl-2,5-dihydro-pyrrol-1-yl)-methanone (0.02 g, quantitative) as a white solid. ¹H NMR (d₆-DMSO, 300 MHz) δ 4.57 (m, 1H), 4.76 (m, 1H), 4.93 (m, 1H), 5.10 (m, 1H), 5.11 (d, 2H, J=8.1 Hz), 6.70 (brdt, 1H), 7.17 (brs, 1H), 7.76 (dd, 1H, J=3, 8.4 Hz), 7.83-7.89 (m, 2H), 8.15 (s, 1H), 8.48 (s, 1H), 8.92 (s, 1H); MS (ESI) m/z=461.1 (MH⁺).

### EXAMPLE 65

### (3-fluoro-3′,6′-dihydro-2′H-[2,4′]bipyridinyl-1′-yl)-(6-furan-3-yl-3-hydroxymethyl-8-trifluoromethyl-imidazo[1,2-a]pyridin-2-yl)-methanone (Compound 171)

The synthesis of (3-fluoro-3′,6′-dihydro-2′H-[2,4′]bipyridinyl-1′-yl)-(6-furan-3-yl-3-hydroxymethyl-8-trifluoromethyl-imidazo[1,2-a]pyridin-2-yl)-methanone followed procedures similar to those described for (6-furan-3-yl-3-hydroxymethyl-8-trifluoromethyl-imidazo[1,2-a]pyridin-2-yl)-(3-thiazol-2-yl-2,5-dihydro-pyrrol-1-yl)-methanone (Compound 170) above, except that 3-fluoro-1′,2′,3′,6′-tetrahydro-[2,4′]bipyridinyl amine was used in the amide coupling step. ¹H NMR (d₆-DMSO, 300 MHz) δ 2.75 (s, 2H), 3.92 (s, 2H), 4.39 (s, 1.3H), 4.53 (s, 0.7H), 5.03 (d, 2H, J=5.7 Hz), 5.45 (t, 1H, J=5.7 Hz), 6.51 (s, 0.5H), 6.65 (s, 0.5H), 7.38 (m, 1H), 7.74 (m, 1H), 7.85 (brs, 1H), 8.14 (s, 1H), 8.44 (d, 1H, J=4.5 Hz), 8.48 (s, 1H), 8.91 (s, 1H); MS (ESI) m/z=487.2 (MH⁺).

### EXAMPLE 66

### (3-fluoro-3′,6′-dihydro-2′H-[2,4′]bipyridinyl-1′-yl)-[6-furan-3-yl-3-(1-hydroxy-ethyl)-8-trifluoromethyl-imidazo[1,2-a]pyridin-2-yl]-methanone (Compound 172)

(3-Fluoro-3′,6′-dihydro-2′H-[2,4′]bipyridinyl-1′-yl)-(6-furan-3-yl-3-hydroxymethyl-8-trifluoromethyl-imidazo[1,2-a]pyridin-2-yl)-methanone was dissolved in THF (4 mL) and cooled to 0° C. Methyl magnesium bromide (0.24 mL, 1.4M in THF, toluene) was added and the mixture was warmed to room temperature and stirred for 1 hour. The procedure was repeated with another identical batch and the batches pooled and purified by preparative TLC to afford (3-fluoro-3′,6′-dihydro-2′H-[2,4′]bipyridinyl-1′-yl)-[6-furan-3-yl-3-(1-hydroxy-ethyl)-8-trifluoromethyl-imidazo[1,2-a]pyridin-2-yl]-methanone (0.01 g, 17%). ¹H NMR (d₆-DMSO, 300 MHz) δ 1.58 (d, 3H, J=6.6 Hz), 2.72 (m, 1H), 3.80 (m, 2H), 4.40 (m, 2H), 5.50 (m, 1H), 5.78 (m, 1H), 6.48 (s, 0.5H), 6.65 (s, 0.5H), 7.12 (s, 1H), 7.40 (m, 1H), 7.75 (m, 1H), 7.84 (s, 1H), 8.09 (s, 1H), 8.43 (d, 1H, J=4.5 Hz), 8.46 (s, 1H), 9.00 (s, 1H); MS (ESI) m/z=501.2 (MH⁺).

### EXAMPLE 67

### [3-Hydroxymethyl-6-(1H-pyrazol-4-yl)-8-trifluoromethyl-imidazo[1,2-a]pyridin-2-yl]-(3-thiazol-2-yl-2,5-dihydro-pyrrol-1-yl)-methanone (Compound 172)

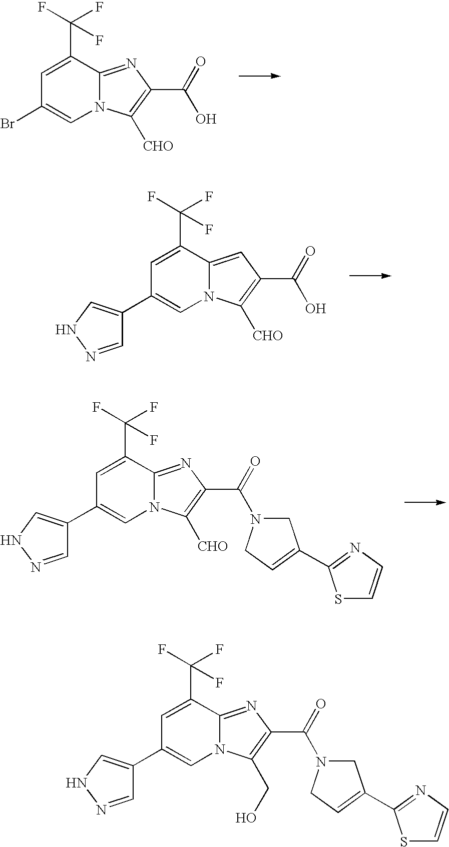

6-Bromo-3-formyl-8-trifluoromethyl-imidazo[1,2-a]pyridine-2-carboxylic acid methyl ester (0.72 g, 2.05 mmol) and 4-(4,4,5,5-tetramethyl-[1,3,2]dioxaborolan-2-yl)-pyrazole-1-carboxylic acid tert-butyl ester (0.91 g, 3.08 mmol) were dissolved in 1,4-dioxane (8 mL) and Argon gas bubbled through. Catalyst (Pd(PPh₃)₄, 0.12 g, 0.1 mmol) was added followed by saturated aqueous NaHCO₃ (1.5 mL). The mixture was microwaved at 120° C. for 50 minutes. The reaction mixture was left standing overnight. Water (15 mL) and citric acid (5%, 15 mL) were added and the solids were filtered, washed with water and dried to afford a crude mixture of 3-formyl-6-(1H-pyrazol-4-yl)-8-trifluoromethyl-imidazo[1,2-a]pyridine-2-carboxylic acid and 3-formyl-6-(1H-pyrazol-4-yl)-8-trifluoromethyl-imidazo[1,2-a]pyridine-2-carboxylic acid methyl ester. This mixture was suspended in MeOH (15 mL) and NaOH (5%, 3 mL) was added and stirred for 2 hours. The MeOH was evaporated and the mixture acidified to pH 2 with 1N HCl. Additional water (15 mL) was added and the solids were filtered, washed (water) and dried to afford 3-formyl-6-(1H-pyrazol-4-yl)-8-trifluoromethyl-imidazo[1,2-a]pyridine-2-carboxylic acid (0.13 g, 20%). Amide coupling and aldehyde reduction steps followed procedures described above for Compound 170.

Compound 172 ¹H NMR (d₆-DMSO, 300 MHz) δ 4.60 (m, 1H), 4.75 (m, 1H), 4.95 (m, 1H), 5.05-5.30 (m, 3H), 6.71 (m, 1H), 7.77 (dd, 1H, J=3.3, 9 Hz), 7.87 (dd, 1H, J=3.3, 11.4 Hz), 8.15 (s, 1H), 8.34-8.40 (m, 2H), 9.03 (s, 1H); MS (ESI) m/z=461.2 (MH⁺).

### EXAMPLE 68

The following Compounds were synthesized using standard amide coupling procedures with HBTU as the coupling agent.

Compound 174 ¹H NMR (d₆-DMSO, 300 MHz) δ 0.20 (m, 2H), 0.45 (m, 2H), 1.00 (m, 1H), 1.45 (m, 2H), 1.80 (m, 2H), 3.04-4.05 (m, 7H), 8.18 (s, 1H), 8.24 (s, 1H), 8.55 (s, 1H), 8.81 (s, 1H), 13.13 (s, 1H); MS (ESI) m/z=468.2 (MH⁺).

Compound 175 ¹H NMR (d₆-DMSO, 300 MHz) δ 0.15 (m, 2H), 0.45 (m, 2H), 1.00 (m, 1H), 1.45 (m, 2H), 1.70 (m, 2H), 3.30-4.20 (m, 9H), 7.30 (s, 1H), 7.83 (s, 1H), 8.18 (s, 1H), 8.54 (s, 1H), 8.80 (s, 1H); MS (ESI) m/z=468.2 (MH⁺).

Compound 176 ¹H NMR (d₆-DMSO, 300 MHz) δ 0.88 (t, 3H, J=7.5 Hz), 1.40-1.60 (m, 4H), 1.85 (m, 2H), 3.30-3.45 (m, 4H), 3.56 (m, 1H), 3.80 (m, 1H), 4.00 (m, 1H), 8.18 (s, 1H), 8.39 (brs, 2H), 8.81 (s, 1H); MS (ESI) m/z=455.9 (MH⁺).

Compound 177 ¹H NMR (d₆-DMSO, 300 MHz) δ 1.12 (t, 3H, J=7 Hz), 1.45 (m, 2H), 1.90 (m, 2H), 3.30-3.60 (m, 5H), 3.82 (m, 1H), 4.02 (m, 1H), 8.18 (s, 1H), 8.24 (s, 1H), 8.55 (s, 1H), 8.81 (s, 1H), 13.1 (s, 1H); MS (ESI) m/z=442.2 (MH⁺).

Compound 178 ¹H NMR (d₆-DMSO, 300 MHz) δ 2.00 (m, 1H), 2.20 (m, 1H), 3.30-4.20 (m, 5H), 6.30-6.50 (m, 3H), 7.06 (t, 0.5H, J=8.7 Hz), 7.12 (t, 0.5H, J=8.7 Hz), 7.29 (s, 0.5H), 7.31 (s, 0.5H), 7.83 (s, 1H), 8.18 (s, 0.5H), 8.20 (s, 0.5H), 8.54 (s, 0.5H), 8.55 (s, 0.5H), 8.80 (s, 0.5H), 8.82 (s, 0.5H); MS (ESI) m/z=505.1 (MH⁺).

Compound 179 ¹H NMR (d₆-DMSO, 300 MHz) δ 1.80 (m, 1H), 2.40 (m, 1H), 3.30-4.40 (m, 5H), 5.86 (d, 0.5H, J=7 Hz), 5.90 (d, 0.5H, J=6 Hz), 6.50-6.54 (m, 3H) 7.00-7.25 (m, 2H), 8.18 (s, 0.5H), 8.20 (s, 0.5H), 8.23 (brs, 1H), 8.53 (brs, 1H), 8.80 (s, 0.5H), 8.83 (s, 0.5H); MS (ESI) m/z=475.1 (MH⁺).

Compound 180 ¹H NMR (d₆-DMSO, 300 MHz) δ 1.82 (m, 1H), 2.20 (m, 1H), 3.64 (s, 1.5H), 3.68 (s, 1.5H), 3.50-4.40 (m, 6H), 6.60-6.80 (m, 4H), 7.32 (brs, 1H), 7.84 (brs, 1H), 8.19 (s, 0.5H), 8.21 (s, 0.5H), 8.55 (s, 0.5H), 8.56 (s, 0.5H), 8.81 (s, 0.5H), 8.83 (s, 0.5H); MS (ESI) m/z=505.1 (MH⁺).

Compound 181 ¹H NMR (d₆-DMSO, 300 MHz) δ 1.09 (t, 1.5H, J=7 Hz), 1.14 (s, 1.5H, J=7 Hz), 2.00 (m, 2H), 3.30-4.40 (m, 7H), 8.20 (s, 1H), 8.40 (brs, 2H), 8.80 (s, 1H), 13.15 (s, 1H); MS (ESI) m/z=428.2 (MH⁺).

Compound 182 ¹H NMR (d₆-DMSO, 300 MHz) δ 1.56 (m, 2H), 1.80 (m, 2H), 3.84 (s, 2H), 3.30-4.00 (m, 5H), 7.17 (s, 1H), 7.24 (s, 1H), 8.17 (s, 1H), 8.22 (brs, 1H), 8.52 (brs, 1H), 8.80 (s, 1H); MS (ESI) m/z=471 (MH⁺).

Compound 183 ¹H NMR (d₆-DMSO, 300 MHz) δ 1.80-2.30 (m, 2H), 3.40-4.55 (m, 6H), 6.69 (t, 0.5H, J=5.1 Hz), 6.75 (t, 0.5H, J=5.1 Hz), 7.31 (m, 1H), 7.84 (m, 1H), 8.18 (s, 0.5H), 8.20 (s, 0.5H), 8.36 (d, 1H, J=5.1 Hz), 8.43 (d, 1H, J=5.1 Hz), 8.55 (s, 0.5H), 8.56 (s, 0.5H), 8.80 (s, 0.5H), 8.82 (s, 0.5H); MS (ESI) m/z=477 (MH⁺).

### EXAMPLE 69

### 2-[6-(furan-3-yl)-8-(trifluoromethyl)imidazo[1,2-a]pyridin-2-yl]-N-(thiophen-2-ylmethyl)acetamide (hereinafter referred to as “Compound 8”)

### Step 1: (6-Bromo-8-trifluoromethyl-imidazo[1,2-a]pyridin-2-yl)-acetic acid ethyl ester

(6-Bromo-8-trifluoromethyl-imidazo[1,2-a]pyridin-2-yl)-acetic acid ethyl ester was prepared by reacting 5-bromo-3-trifluoromethyl-pyridin-2-ylamine with 4-chloro-3-oxo-butyric acid ethyl. MS (ESI) m/z=352 (MH⁺).

### Step 2: (6-Bromo-8-trifluoromethyl-imidazo[1,2-a]pyridin-2-yl)-acetic acid

(6-Bromo-8-trifluoromethyl-imidazo[1,2-a]pyridin-2-yl)-acetic acid ethyl ester was saponified using lithium hydroxide to give (6-bromo-8-trifluoromethyl-imidazo[1,2-a]pyridin-2-yl)-acetic acid. MS (ESI) m/z=324 (MH⁺).

### Step 3: (6-Furan-3-yl-8-trifluoromethyl-imidazo[1,2-a]pyridin-2-yl)-acetic acid

Prepared using standard Suzuki reaction of the above acid. MS (ESI) m/z=311 (MH⁺).

### Step 4: 2-[6-(furan-3-yl)-8-(trifluoromethyl)imidazo[1,2-a]pyridin-2-yl]-N-(thiophen-2-ylmethyl)acetamide

Using standard HATU coupling of the above acid. ¹H NMR (d₆-DMSO, 300 MHz) δ 3.62 (s, 2H), 4.39 (d, 1H, J=6.0 Hz), 6.87 (m, 1H), 6.93 (m, 1H), 6.97 (m, 1H), 7.32 (m, 1H), 7.75 (m, 1H), 7.87 (s, 1H), 7.91 (s, 1H), 8.33 (s, 1H), 8.64 (m, 1H), 9.07 (s, 1H); MS (ESI) m/z=406 (MH⁺).

### EXAMPLE 70

### 2-(6-Furan-3-yl-8-trifluoromethyl-imidazo[1,2-a]pyridin-2-yl)-N-thiazol-2-yl-acetamide (Compound 184)

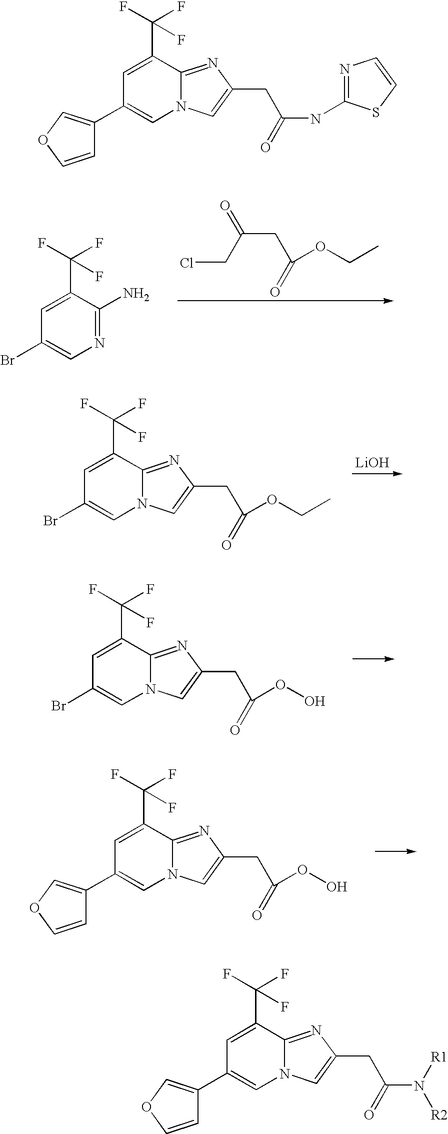

### Step 1-3: Described Before for the Synthesis of Compound 8

### Step 4: Prepared using standard HATU coupling of (6-furan-3-yl-8-trifluoromethyl-imidazo[1,2-a]pyridin-2-yl)-acetic acid

¹H NMR (d₆-DMSO, 300 MHz) δ 2.70 (s, 2H), 7.06 (s, 1H), 7.23 (d, 1H, J=3.3 Hz), 7.48 (d, 1H, J=3.6 Hz), 7.84 (s, 1H), 8.18 (s, 1H), 8.29 (s, 1H), 8.47 (s, 1H), 9.35 (s, 1H); MS (ESI) m/z=393 (MH⁺).

### EXAMPLE 71

### N-Benzyl-2-(6-furan-3-yl-8-trifluoromethyl-imidazo[1,2-a]pyridin-2-yl)acetamide (Compound 185)

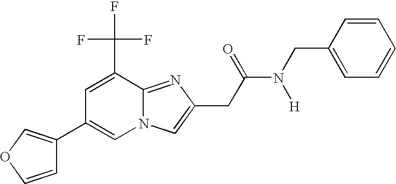

Using standard HATU coupling of the above acid.

¹H NMR (d₆-DMSO, 300 MHz) δ 4.29 (dd, 1H, J=5.7, 5.1 Hz), 7.28 (m, 15), 7.83 (m, 1H), 7.98 (d, 1H, J=3.0 Hz), 8.09 (s, 0.5H), 8.41 (s, 0.5H), 8.60 (s, 1H), 9.19 (s, 1H), MS (ESI) m/z=400 (MH⁺).

### EXAMPLE 72

### 2-(6-Furan-3-yl-8-trifluoromethyl-imidazo[1,2-a]pyridin-2-yl)-1-(3-thiazol-2-yl-2,5-dihydro-pyrrol-1-yl)ethanone (Compound 186)

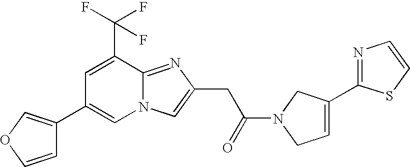

Using standard HATU coupling of the above acid.

¹H NMR (d₆-DMSO, 300 MHz) δ 3.88 (d, 1H, J=12.9 Hz), 4.28 (s, 1H), 4.48 (s, 1H), 4.61 (s, 1H), 4.81 (s, 1H), 6.60 (m, 1H), 6.96 (s, 1H), 7.72 (d, 1H, J=3.3 Hz), 7.75 (s, 1H), 7.80 (d, 1H, J=3.3 Hz), 7.89 (s, 1H), 7.91 (d, 1H, J=4.2 Hz), 8.32 (s, 1H), 9.06 (s, 1H), MS (ESI) m/z=445 (MH⁺).

### EXAMPLE 73

### 2-(6-Furan-3-yl-8-trifluoromethyl-imidazo[1,2-a]pyridin-2-yl)-1-(3-thiophen-2-yl-pyrrolidin-1-yl)-ethanone (Compound 187)

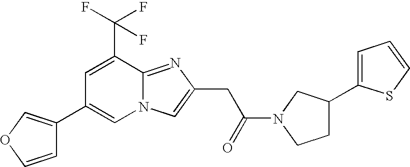

Using standard HATU coupling of the above acid.

¹H NMR (d₆-DMSO, 300 MHz) δ 1.95 (m, 1H), 2.35 (m, 1H), 3.31 (m, 2H), 3.61 (m, 5H), 6.97 (m, 2H), 7.02 (s, 1H), 7.38 (m, 1H), 7.82 (s, 1H), 7.99 (d, 1H, J=3.90 Hz), 8.08 (d, 1H, J=4.2 Hz), 8.60 (s, 1H), 9.16 (s, 1H); MS (ESI) m/z=446 (MH⁺).

### EXAMPLE 74

### 2-(6-Furan-3-yl-8-trifluoromethyl-imidazo[1,2-a]pyridin-2-yl)-1-(2-thiophen-2-yl-pyrrolidin-1-yl)-ethanone (Compound 188)

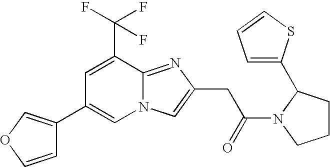

Using standard HATU coupling of the above acid.

MS (ESI) m/z=446 (MH⁺).

### EXAMPLE 75

### Thiophene-2-sulfonic acid [2-(6-furan-3-yl-8-trifluoromethyl-imidazo[1,2-a]pyridin-2-yl)-acetyl]-amide (Compound 189)

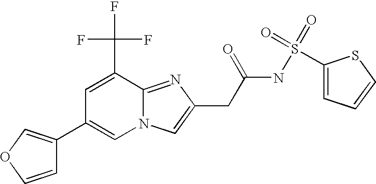

Using standard EDC coupling of the above acid.

MS (ESI) m/z=456 (MH⁺).

### EXAMPLE 76

### N-{1-[2-(6-Furan-3-yl-8-trifluoromethyl-imidazo[1,2-a]pyridin-2-yl)-acetyl]-azetidin-3-yl}-methanesulfonamide (Compound 190)

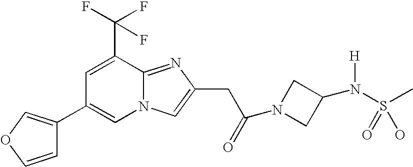

Using standard HATU coupling of the above acid. MS (ESI) m/z=444 (MH⁺).

### EXAMPLE 77

### 2-(6-Furan-3-yl-8-trifluoromethyl-imidazo[1,2-a]pyridin-2-yl)-1-(4-thiazol-2-yl-3,6-dihydro-2H-pyridin-1-yl)-ethanone (Compound 191)

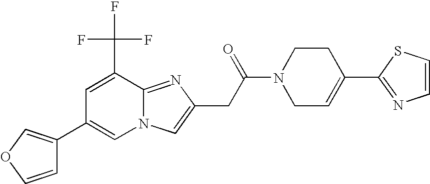

Using standard HATU coupling of the above acid. MS (ESI) m/z=459 (MH⁺).

### EXAMPLE 78

### 2-(6-Furan-3-yl-8-trifluoromethyl-imidazo[1,2-a]pyridin-2-yl)-N-phenethyl-acetamide (Compound 192)

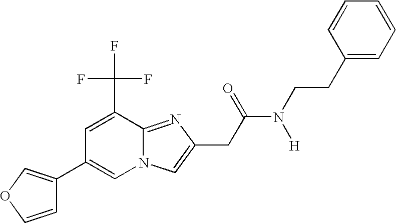

Using standard HATU coupling of the above acid. MS (ESI) m/z=415 (MH⁺).

### EXAMPLE 79

### 3-Chloro-6-furan-3-yl-8-trifluoromethyl-imidazo[1,2-a]pyridine-2-carboxylic acid 4-phenyl-cyclohexyl)-amide (Compound 193)

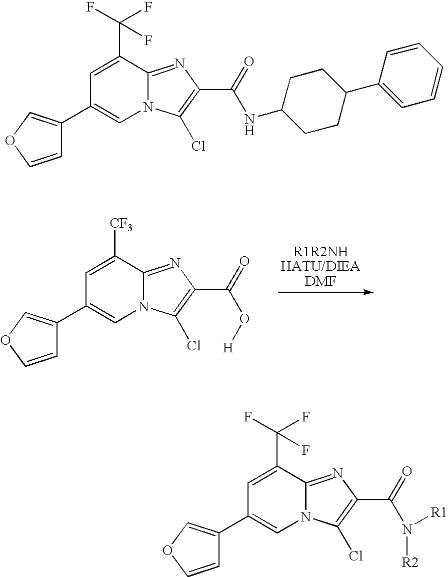

A mixture of 3-chloro-6-furan-2-yl-8-trifluoromethyl-imidazo[1,2-a]pyridine-2-carboxylic acid (40 mg, 0.12 mmol), amine (0.14 mmol), HATU (69 mg, 0.18 mmol), and di-isopropylethylamine (0.08 mL, 0.42 mmol) in DMF (0.8 mL) was stirred at room temperature. After 1.5 hours, the mixture was diluted with EtOAc (20 mL) and washed with saturated aqueous NaHCO₃ (10 mL), then brine (10 mL). The filtrate was dried (Na₂SO₄), filtered and concentrated. Column chromatography [n-hex/EtOAc (5:4 v/v)]of the crude material gave Compound 193 (51 mg, 74%) as a white powder. ¹H NMR (d₆-DMSO, 300 MHz) δ 1.61 (m, 5H), 1.87 (m, 4H), 3.85 (m, 1H), 7.12 (m, 1H), 7.22 (m, 5H), 7.78 (m, 1H), 7.91 (m, 1H), 8.17 (m, 1H), 8.51 (m, 1H), 8.78 (m, 1H); MS (ESI) m/z=489 (MH⁺).

### EXAMPLE 80

### (4-Benzoyl-piperidin-1-yl)-(3-chloro-6-furan-3-yl-8-trifluoromethyl-imidazo[1,2-a]pyridin-2-yl)-methanone (Compound 194)

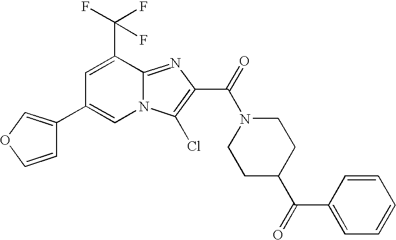

Using standard HATU coupling of the above acid. ¹H NMR (d₆-DMSO, 300 MHz) δ 1.55 (m, 2H), 1.65 (m, 1H), 1.95 (m, 1H), 3.05 (s, 1H), 3.30 (m, 1H), 3.80 (m, 1H), 4.15 (m, 1H), 4.51 (m, 1H), 7.31 (m, 1H), 7.54 (m, 2H), 7.63 (s, 1H), 7.83 (m, 1H), 8.00 (s, 1H), 8.03 (s, 1H), 8.18 (s, 1H), 8.55 (s, 1H), 8.80 (m, 1H); MS (ESI) m/z=503 (MH⁺). (4-Benzenesulfonyl-piperidin-1-yl)-(3-chloro-6-furan-3-yl-8-trifluoromethyl-imidazo[1,2-a]pyridin-2-yl)-methanone (Compound 268)

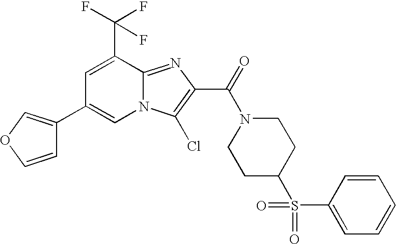

Using standard HATU coupling of the above acid. MS (ESI) m/z=539 (MH⁺).

### EXAMPLE 81

### (3-Chloro-6-furan-3-yl-8-trifluoromethyl-imidazo[1,2-a]pyridin-2-yl)-(4-pyrrolidin-1-yl-piperidin-1-yl)-methanone (Compound 195)

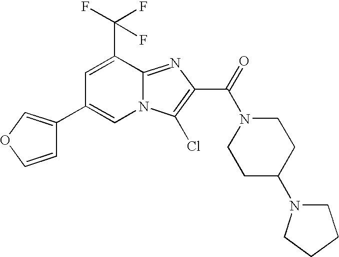

Using standard HATU coupling of the above acid. MS (ESI) m/z=468 (MH⁺).

### EXAMPLE 82

### [3-Chloro-6-(1H-pyrazol-3-yl)-8-trifluoromethyl-imidazo[1,2-a]pyridin-2-yl]-(4-thiazol-2-yl-3,6-dihydro-2H-pyridin-1-yl)-methanone (Compound 196)

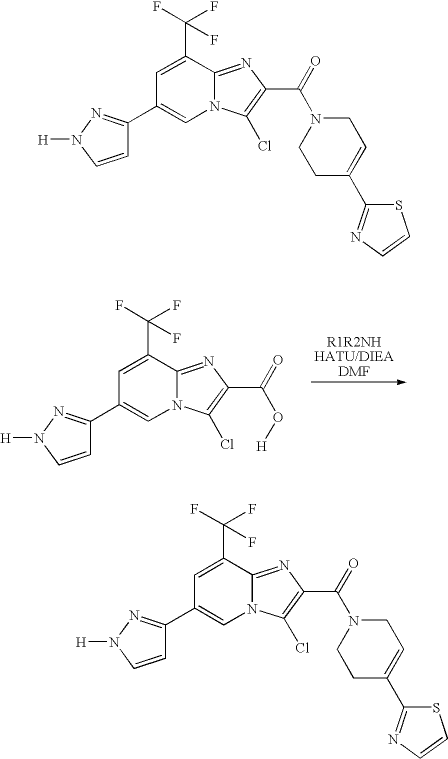

Prepared using standard HATU coupling of the above acid. Yield: 20%

¹H NMR (d₆-DMSO, 300 MHz) δ 2.71 (m, 2H), 3.86 (m, 2H), 4.39 (m, 2H), 6.63 (bd, 1H), 7.66 (m, 1H), 7.79 (d, 1H, J=3.0 Hz), 8.20 (s, 1H), 8.40 (m, 2H), 8.83 (s, 1H), 10.11 (s, 1H); MS (ESI) m/z=480 (MH⁺).

### EXAMPLE 83

### (3-Chloro-6-furan-3-yl-8-trifluoromethyl-imidazo[1,2-a]pyridin-2-yl)-(4-imidazol-1-yl-piperidin-1-yl)-methanone (Compound 197)

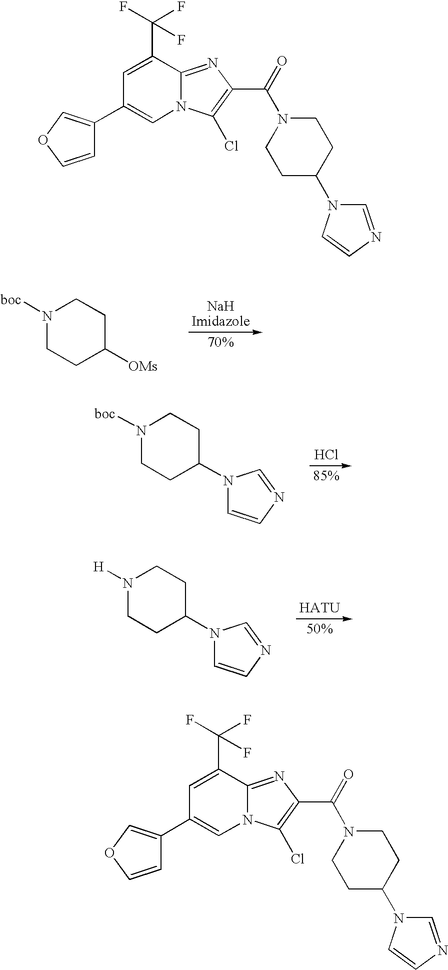

### Step 1: 4-Imidazol-1-yl-piperidine-1-carboxylic acid tert-butyl ester

A solution of 4-methanesulfonyloxy-piperidine-1-carboxylic acid tert-butyl ester

(250 mg, 1.25 mmol) in DMF (2 mL) was added to a stirred solution of NaH (86 mg, 2.50 mmol) and imidazole (170 mg, 2.50 mmol) in DMF (2 mL) under nitrogen atmosphere. The mixture was stirred at 80° C. for 16 hours. DMF was evaporated in vacuo. The resulting crude product was extracted with EtOAc and the extracts were successively washed with water and brine, and the organic layer was evaporated to leave the title residue which was used without further purification. MS (ESI) m/z=252 (MH⁺).

### Step 2: 4-Imidazol-1-yl-piperidine

A solution of 4-imidazol-1-yl-piperidine-1-carboxylic acid tert-butyl ester from step 1 (200 mg, 0.202 mmol) in THF (2 mL) and 4M HCl in dioxanes (2 mL) was stirred at room temperature for 12 hours, and concentrated in vacuo to afford 4-imidazol-1-yl-piperidine (66.8 mg, 73%) as a pale yellow solid. MS (ESI) m/z=152.0 (MH⁺).

### Step 3: Title Compound was Prepared Using Standard HATU Coupling

Data: δ 1.95 (m, 1H), 2.10 (m, 1H), 2.24 (m, 1H), 2.98 (s, 1H), 3.30 (m, 2H), 4.40 (m, 1H), 4.65 (m, 2H), 7.31 (m, 1H), 7.71 (dd, 1H, J=1.2, 1.8 Hz), 7.83 (t, 1H, J=1.8 Hz), 7.96 (t, 1H, J=1.5 Hz), 8.20 (s, 1H), 8.55 (s, 1H), 8.81 (s, 1H), 9.21 (s, 1H); MS (ESI) m/z=465 (MH⁺).

### EXAMPLE 84

### Trifluoro-methanesulfonic acid 1-(3-chloro-6-furan-3-yl-8-trifluoromethyl-imidazo[1,2-a]pyridine-2-carbonyl)-1,2,3,6-tetrahydro-pyridin-4-yl ester (hereinafter referred to as “Compound 9”)

### Step 1: 4-Trifluoromethanesulfonyloxy-3,6-dihydro-2H-pyridine-1-carboxylic acid tert-butyl ester

A solution of t-butoxycarbonyl-4-piperidone (3 g, 15.06 mmol) in THF (10 mL) was slowly added to a stirring 2M solution of LDA (9.03 mL, 18.07 mmol) in THF (10 mL) at −78° C. After 10 min, a solution of N-phenyl bis(trifluoromethanesulfonimide) (5.92 g, 16.56 mmol) in THF (10 mL) was slowly added. After 30 min, the cooling bath was removed and the mixture was allowed to warm to room temperature over the course of 1.5 hours. The mixture was cooled to 0° C., quenched with saturated aqueous NaHCO₃ (30 mL), and extracted with ether (200 mL). The organic layer was washed with 5% citric acid (40 mL), aqueous NaOH (1M, 4×40 mL), H₂O (2×40 mL), brine (40 mL), dried (MgSO₄), the filtrate was concentrated on silica and subjected to flash column chromatography (15-50% EtOAc/hexane gradient) to afford 4-trifluoromethanesulfonyloxy-3,6-dihydro-2H-pyridine-1-carboxylic acid tert-butyl ester (3.40 g, 68.2%) as a brown oil. ¹H NMR (CDCl₃, 300 MHz) δ 6.10 (t, J=3.30 Hz, 1H), 4.07 (m, 2H), 3.63 (t, J=5.70 Hz, 2H), 2.48 (m, 2H), 1.48 (s, 9H); MS (ESI) m/z=276 (MH⁺-^{t}Bu).

### Step 2: Trifluoro-methanesulfonic acid 1,2,3,6-tetrahydro-pyridin-4-yl-ester hydrochloride

4-Trifluoromethanesulfonyloxy-3,6-dihydro-2H-pyridine-1-carboxylic acid tert-butyl ester underwent HCl deprotection to give trifluoro-methanesulfonic acid 1,2,3,6-tetrahydro-pyridin-4-yl-ester hydrochloride. ¹H NMR (CDCl₃, 300 MHz) δ 9.76 (s, 1H), 7.32 (m, 1H), 3.94 (m, 2H), 2.79 (m, 2H), 2.11 (m, 2H). MS (ESI) m/z=232.0 (MH⁺).

### Step 3: Trifluoro-methanesulfonic acid 1-(3-chloro-6-furan-3-yl-8-trifluoromethyl-imidazo[1,2-a]pyridine-2-carbonyl)-1,2,3,6-tetrahydro-pyridin-4-yl ester

Prepared using standard HATU coupling of the above amine. ¹H NMR (d₆-DMSO, 300 MHz) δ 8.83 (s, 1H), 8.56 (s, 1H), 8.22 (s, 1H), 7.84 (t, J=2.10 Hz, 1H), 7.32 (m, 1H), 6.15 (s, 0.5H), 6.04 (s, 0.5H), 4.37 (d, J=31.20 Hz, 2H), 3.87 (m, 2H), 2.60 (m, 2H). MS (ESI) m/z=544 (MH⁺).

### EXAMPLE 85

### (3-Chloro-6-furan-3-yl-8-trifluoromethyl-imidazo[1,2-a]pyridin-2-yl)-(4-pyridin-3-ylethynyl-3,6-dihydro-2H-pyridin-1-yl)-methanone (Compound 198)

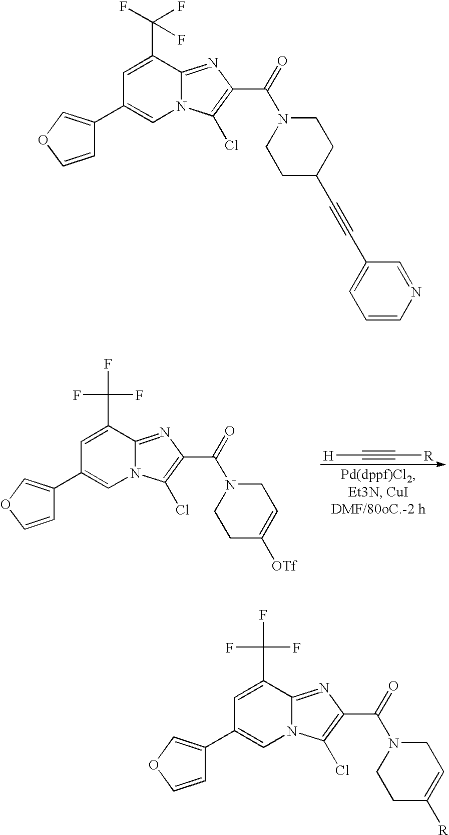

A mixture of trifluoro-methanesulfonic acid 1-(3-chloro-6-furan-3-yl-8-trifluoromethyl-imidazo[1,2-a]pyridine-2-carbonyl)-1,2,3,6-tetrahydro-pyridin-4-yl ester (Compound 9) (50 mg, 0.10 mmol), alkyne (0.20 mmol), Et₃N (0.20 mmol), CuI (5 mol %) and Pd(dppf)Cl₂*CH₂Cl₂ (4 mg, 0.005 mmol) in DMF (1.5 mL) was heated at 80° C. for 12 hours. The mixture was diluted with EtOAc (25 mL) and washed with saturated aqueous NaHCO₃ (10 mL), and brine (10 mL). The extracts were dried (Na₂SO₄), filtered and concentrated. Preparative HPLC purification (30-100% ACN gradient) of the crude product gave the final product (˜70% yield) as a white powder. ¹H NMR (d₆-DMSO, 300 MHz) δ 2.37 (m, 2H), 3.74 (m, 2H), 4.30 (m, 2H), 6.24 (bd, 1H), 7.27 (s, 1H), 7.42 (m, 1H), 7.78 (m, 1H), 7.88 (m, 1H), 8.15 (s, 1H), 8.52 (m, 2H), 8.63 (s, 1H), 8.77 (s, 1H); MS (ESI) m/z=498 (MH⁺).

### EXAMPLE 86

### (3-Chloro-6-furan-3-yl-8-trifluoromethyl-imidazo[1,2-a]pyridin-2-yl)-[4-(3,3-dimethyl-but-1-ynyl)-3,6-dihydro-2H-pyridin-1-yl]-methanone (Compound 199)

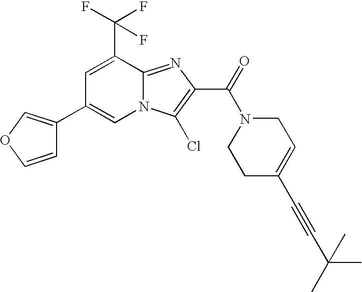

Prepared using similar procedure as in Example 85 for Compound 198. Yield: 60%.

¹H NMR (d₆-DMSO, 300 MHz) δ 1.28 (s, 9H), 2.21 (m 2H), 3.66 (m, 2H), 4.17 (m, 2H), 5.91 (bd, 1H), 7.31 (s, 1H), 7.82 (s, 2H), 8.19 (s, 1H), 8.54 (s, 1H), 8.80 (s, 1H); MS (ESI) m/z=477 (MH⁺).

### EXAMPLE 87

### (3-Chloro-6-furan-3-yl-8-trifluoromethyl-imidazo[1,2-a]pyridin-2-yl)-(4-thiophen-2-yl-3,6-dihydro-2H-pyridin-1-yl)-methanone (hereinafter referred to as “Compound 10”)

A mixture of (3-chloro-6-furan-3-yl-8-trifluoromethyl-imidazo[1,2-a]pyridin-2-yl)-[4-(4,4,5,5-tetramethyl-[1,3,2]dioxaborolan-2-yl)-3,6-dihydro-2H-pyridin-1-yl]-methanone

(52 mg, 0.10 mmol), R—Br (0.25 mmol) and Pd(dppf)Cl₂*CH₂Cl₂ (4 mg, 0.005 mmol) in 2M Na₂CO₃ (0.5 mL) and 1,4-dioxane (1.2 mL) was heated at 100° C. for 12 hours. The mixture was diluted with EtOAc (25 mL) and washed with saturated aqueous NaHCO₃ (10 mL), and brine (10 mL). The extracts were dried (Na₂SO₄), filtered and concentrated. Preparative HPLC purification (30-100% ACN gradient) of the crude product gave the final product (˜25% Yield) as a white powder.

¹H NMR (d₆-DMSO, 300 MHz) δ 2.54 (bs, 2H), 3.80 (m, 2H), 4.26 (m, 2H), 5.98 & 6.14 (bs, 1H), 6.97 (m, 1H), 7.07 (m, 1H), 7.27 (m, 1H), 7.36 (t, 1H, J=4.5), 7.78 (m, 1H), 8.16 (m, 1H), 8.50 (s, 1H), 8.77 (s, 1H), MS (ESI) m/z=479 (MH⁺).

### EXAMPLE 88

### (3-Chloro-6-furan-3-yl-8-trifluoromethyl-imidazo[1,2-a]pyridin-2-yl)-(4-cyclopentylethynyl-3,6-dihydro-2H-pyridin-1-yl)-methanone

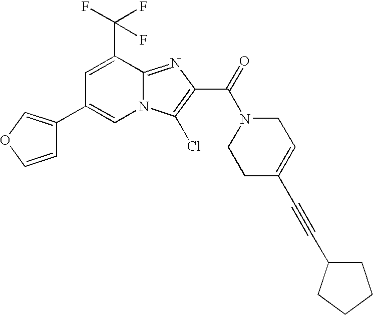

Prepared using similar procedure as in Example 87 for Compound 10. Yield: 65%.

¹H NMR (d₆-DMSO, 300 MHz) δ 1.48 (m, 3H), 1.63 (m, 2H), 1.86 (m, 2H), 2.21 (m 2H), 2.71 (m, 1H), 3.30 (m, 1H), 3.72 (m, 2H), 4.19 (m, 2H), 5.90 (bd, 1H), 7.30 s, 1H), 7.82 (s, 1H), 8.18 (s, 1H), 8.53 (s, 1H), 8.79 (s, 2H); MS (ESI) m/z=489 (MH⁺).

### EXAMPLE 89

### (3-Chloro-6-furan-3-yl-8-trifluoromethyl-imidazo[1,2-a]pyridin-2-yl)-(3-hydroxy-3′,6′-dihydro-2′H-[2,4′]bipyridinyl-1′-yl)-methanone (Compound 201)

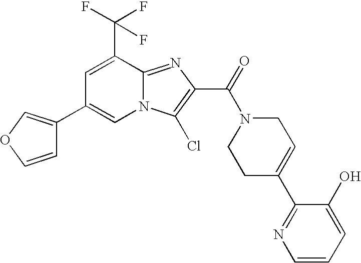

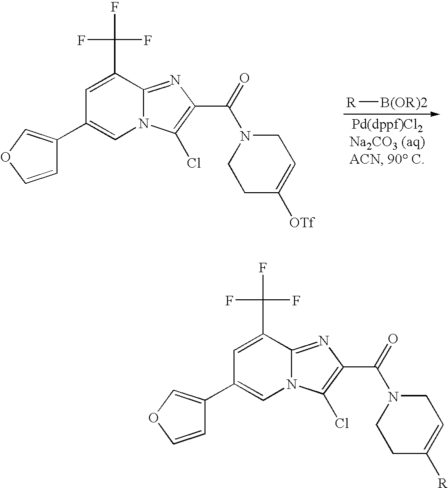

A mixture of trifluoro-methanesulfonic acid 1-(3-chloro-6-furan-3-yl-8-trifluoromethyl-imidazo[1,2-a]pyridine-2-carbonyl)-1,2,3,6-tetrahydro-pyridin-4-yl ester (Compound 9) (50 mg, 0.10 mmol), R—B(OR)₂ (0.25 mmol) and Pd(dppf)Cl₂.CH₂Cl₂ (4 mg, 0.005 mmol) in 2M Na₂CO₃ (0.5 mL) and ACN (1.2 mL) was heated at 100° C. for 12 hours. The mixture was diluted with EtOAc (25 mL) and washed with saturated aqueous NaHCO₃ (10 mL), and brine (10 mL). The extracts were dried (Na₂SO₄), filtered and concentrated. Preparative HPLC purification (30-100% ACN gradient) of the crude product gave the final product (˜45% yield) as a white powder.

¹H NMR (d₆-DMSO, 300 MHz) δ 2.64 (bs, 2H), 3.77 (m, 2H), 4.34 (m, 2H), 6.66 (bd, 1H), 7.08 (m, 1H), 7.21 (m, 1H), 7.27 (m, 1H), 7.78 (t, 1H, J=1.5 Hz), 7.98 (m, 1H), 8.15 (s, 1H), 8.50 (s, 1H), 8.77 (s, 1H), 10.20 (bs, 1H); MS (ESI) m/z=490 (MH⁺).

### EXAMPLE 90

### (3-Chloro-3′,6′-dihydro-2′H-[2,4′]bipyridinyl-1′-yl)-(3-chloro-6-furan-3-yl-8-trifluoromethyl-imidazo[1,2-a]pyridin-2-yl)-methanone (Compound 202)

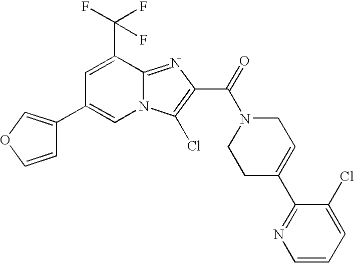

Prepared using similar procedure as in Example 87 for Compound 10.

¹H NMR (d₆-DMSO, 300 MHz) δ 2.62 (m, 2H), 3.84 (m, 2H), 4.36 (m, 2H), 6.22 (bd, 1H), 7.34 (m, 2H), 7.82 (t, 1H, J=1.8 Hz), 7.94 (m, 1H), 8.20 (s, 1H), 8.51 (m, 1H), 8.55 (s, 1H), 8.82 (s, 1H); MS (ESI) m/z=508 (MH⁺).

### EXAMPLE 91

### (3-Chloro-6-furan-3-yl-8-trifluoromethyl-imidazo[1,2-a]pyridin-2-yl)-(5-fluoro-3′,6′-dihydro-2′H-[3,4′]bipyridinyl-1′-yl)-methanone (Compound 203)

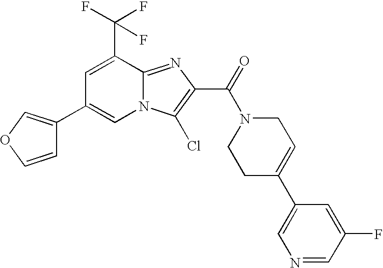

Prepared using similar procedure as in Example 87 for Compound 10.

¹H NMR (d₆-DMSO, 300 MHz) δ 2.62 (m, 2H), 3.89 (m, 2H), 4.38 (m, 2H), 6.44 (bd, 1H), 7.33 (m, 1H), 7.84 (m, 2H), 8.21 (s, 1H), 8.48 (m, 1H), 8.57 (m, 2H), 8.83 (s, 1H); MS (ESI) m/z=492 (MH⁺).

### EXAMPLE 92

### (3-Chloro-6-furan-3-yl-8-trifluoromethyl-imidazo[1,2-a]pyridin-2-yl)-(6-fluoro-3′,6′-dihydro-2′H-[2,4′]bipyridinyl-1′-yl)-methanone (Compound 204)

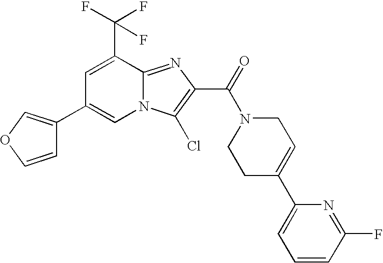

Prepared using similar procedure as in Example 87 for Compound 10.

¹H NMR (d₆-DMSO, 300 MHz) δ 2.58 (m, 2H), 3.79 (m, 2H), 4.35 (m, 2H), 6.73 (bd, 1H), 7.00 (m, 1H), 7.28 (s, 1H), 7.44 (m, 1H), 7.79 (s, 1H), 7.93 (m, 1H), 8.16 (s, 1H), 8.51 (s, 1H), 8.78 (s, 1H); MS (ESI) m/z=492 (MH⁺).

### EXAMPLE 93

### (3-Chloro-6-furan-3-yl-8-trifluoromethyl-imidazo[1,2-a]pyridin-2-yl)-(3-trifluoromethyl-3′,6′-dihydro-2′H-[2,4′]bipyridinyl-1′-yl)-methanone (Compound 205)

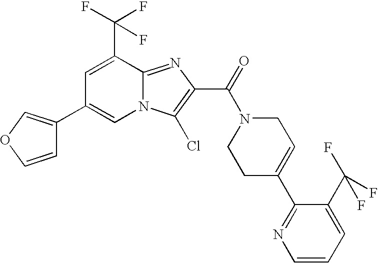

Prepared using similar procedure as in Example 87 for Compound 10.

¹H NMR (d₆-DMSO, 300 MHz) δ 2.54 (m, 2H), 3.90 (m, 2H), 4.33 (m, 2H), 5.77 (bd, 1H), 7.31 (s, 1H), 7.53 (m, 1H), 7.83 (t, 1H, J=1.8 Hz), 8.20 (m, 2H), 8.55 (s, 1H), 8.82 (m, 2H); MS (ESI) m/z=542 (MH⁺).

### EXAMPLE 94

### (3-Chloro-6-furan-3-yl-8-trifluoromethyl-imidazo[1,2-a]pyridin-2-yl)-(3-fluoro-4-methyl-3′,6′-dihydro-2′H-[2,4′]bipyridinyl-1′-yl)-methanone (Compound 206)

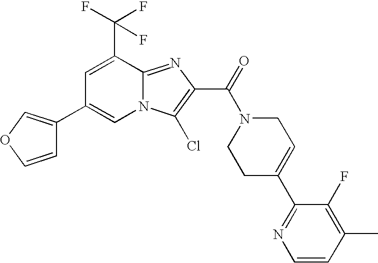

Prepared using similar procedure as in Example 87 for Compound 10.

¹H NMR (d₆-DMSO, 300 MHz) δ 2.28 (m, 3H), 2.69 (m, 2H), 3.86 (m, 2H), 4.35 (m, 2H), 6.49 (bd, 1H), 7.23 (m, 1H), 7.31 (s, 1H), 7.82 (m, 1H), 8.19 (s, 1H), 8.26 (d, 1H, J=4.5 Hz), 8.55 (s, 1H), 8.82 (s, 1H); MS (ESI) m/z=506 (MH⁺).

### EXAMPLE 95

### (3-Chloro-6-furan-3-yl-8-trifluoromethyl-imidazo[1,2-a]pyridin-2-yl)-(3-fluoro-6-methyl-3′,6′-dihydro-2′H-[2,4′]bipyridinyl-1′-yl)-methanone (Compound 207)

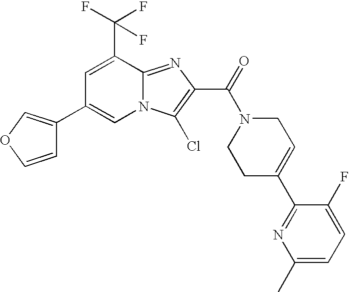

Prepared using similar procedure as in Example 87 for Compound 10.

¹H NMR (d₆-DMSO, 300 MHz) δ 2.44 (m, 3H), 2.69 (m, 2H), 3.83 (m, 2H), 4.39 (m, 2H), 6.48 (bd, 1H), 7.19 (m, 1H), 7.30 (s, 1H), 7.59 (m, 1H), 7.82 (s, 1H), 8.19 (s, 1H), 8.54 (s, 1H), 8.81 (s, 1H); MS (ESI) m/z=506 (MH⁺).

### EXAMPLE 96

### (3-Chloro-6-furan-3-yl-8-trifluoromethyl-imidazo[1,2-a]pyridin-2-yl)-[4-(5-fluoro-pyrimidin-4-yl)-3,6-dihydro-2H-pyridin-1-yl]-methanone (Compound 208)

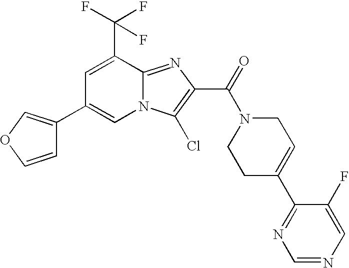

Prepared using similar procedure as in Example 87 for Compound 10.

¹H NMR (d₆-DMSO, 300 MHz) δ 2.68 (m, 2H), 3.81 (m, 2H), 4.47 (m, 2H), 6.84 (bd, 1H), 7.27 (m, 1H), 7.78 (s, 1H), 8.16 (s, 1H), 8.51 (s, 1H), 8.79 (m, 2H), 8.96 (d, 1H, J=2.71 Hz); MS (ESI) m/z=493 (MH⁺).

### EXAMPLE 97

### (3-Chloro-6-furan-3-yl-8-trifluoromethyl-imidazo[1,2-a]pyridin-2-yl)-(4-fluoro-3′,6′-dihydro-2′H-[2,4′]bipyridinyl-1′-yl)-methanone (Compound 209)

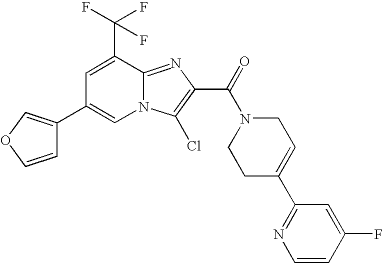

Prepared using similar procedure as in Example 87 for Compound 10.

¹H NMR (d₆-DMSO, 300 MHz) δ 2.63 (m, 2H), 3.82 (m, 2H), 4.32 (m, 2H), 6.84 (bd, 1H), 7.16 (s, 1H), 7.27 (s, 2H), 7.52 (m, 1H), 7.78 (s, 1H), 8.16 (s, 1H), 8.50 (m, 1H), 8.77 (s, 1H); MS (ESI) m/z=492 (MH⁺).

### EXAMPLE 98

### (3-Chloro-6-furan-3-yl-8-trifluoromethyl-imidazo[1,2-a]pyridin-2-yl)-(3′,6′-dihydro-2′H-[2,4′]bipyridinyl-1′-yl)-methanone (Compound 210)

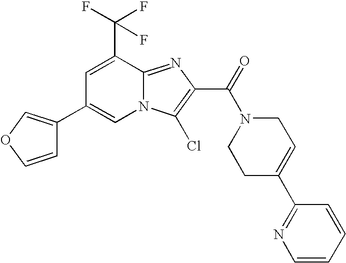

Prepared using similar procedure as in Example 87 for Compound 10.

¹H NMR (d₆-DMSO, 300 MHz) δ 2.71 (m, 2H), 3.88 (m, 2H), 4.43 (m, 2H), 6.78 (bd, 1H), 7.32 (m, 1H), 7.44 (m, 1H), 7.77 (m, 1H), 7.83 (s, 1H), 8.00 (m, 1H), 8.20 (s, 1H), 8.55 (m, 1H), 8.62 (m, 1H), 8.82 (s, 1H); MS (ESI) m/z=474 (MH⁺).

### EXAMPLE 99

### 1′-(3-Chloro-6-furan-3-yl-8-trifluoromethyl-imidazo[1,2-a]pyridine-2-carbonyl)-1′,2′,3′,6′-tetrahydro-[2,4′]bipyridinyl-3-carbonitrile (Compound 211)

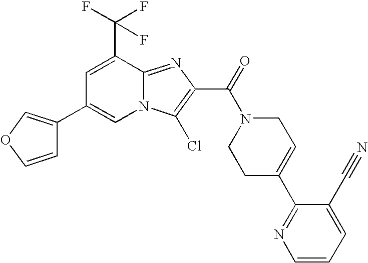

Prepared using similar procedure as in Example 87 for Compound 10.

¹H NMR (d₆-DMSO, 300 MHz) δ 2.69 (m, 2H), 3.83 (m, 2H), 4.39 (m, 2H), 6.9 (bd, 1H), 7.27 (m, 1H), 7.46 (m, 1H), 7.78 (m, 1H), 8.16 (s, 1H), 8.28 (m, 1H), 8.51 (s, 1H), 8.76 (m, 2H); MS (ESI) m/z=499 (MH⁺).

### EXAMPLE 100

### (3-Chloro-6-furan-3-yl-8-trifluoromethyl-imidazo[1,2-a]pyridin-2-yl)-[4-((E)-2-cyclopropyl-vinyl)-3,6-dihydro-2H-pyridin-1-yl]-methanone (Compound 212)

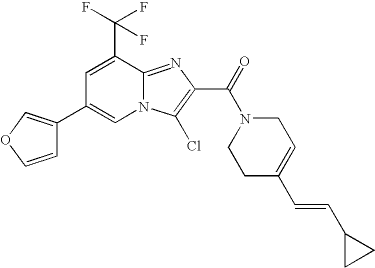

Prepared using similar procedure as in Example 87 for Compound 10.

¹H NMR (d₆-DMSO, 300 MHz) δ 0.02 (m, 2H), 0.32 (m, 2H), 1.04 (m, 1H), 1.84 (m, 2H), 3.33 (m, 2H), 3.84 (m, 2H), 4.77 (m, 1H), 5.20 (bd, 1H), 5.79 (m, 1H), 6.93 (m, 1H), 7.44 (s, 1H), 7.80 (s, 1H), 8.16 (s, 1H), 8.42 (s, 1H); MS (ESI) m/z=463 (MH⁺).

### EXAMPLE 101

### (3-Chloro-6-furan-3-yl-8-trifluoromethyl-imidazo[1,2-a]pyridin-2-yl)-[4-(2,5-dihydro-1H-pyrrol-3-yl)-3,6-dihydro-2H-pyridin-1-yl]-methanone (Compound 213)

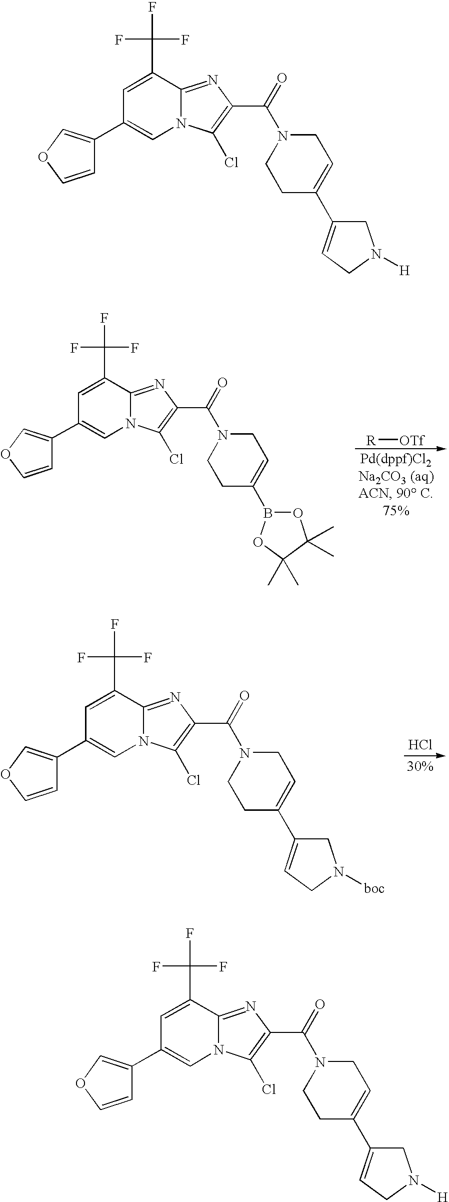

### Step 1: 3-[1-(3-Chloro-6-furan-3-yl-8-trifluoromethyl-imidazo[1,2-a]pyridine-2-carbonyl)-1,2,3,6-tetrahydro-pyridin-4-yl]-2,5-dihydro-pyrrole-1-carboxylic acid tert-butyl ester

Prepared using similar Suzuki procedure as in Example 87 for Compound 10 and the crude was used in the next step without further purification.

### Step 2

Title Compound: 4M HCl in Dioxane (1.0 mmol) was added to a solution of 3-[1-(3-chloro-6-furan-3-yl-8-trifluoromethyl-imidazo[1,2-a]pyridine-2-carbonyl)-1,2,3,6-tetrahydro-pyridin-4-yl]-2,5-dihydro-pyrrole-1-carboxylic acid tert-butyl ester (0.2 mmol) in THF (1 mL) and was stirred at room temperature for 12 hours. The reaction mixture was concentrated and the crude material was subjected to preparative TLC (6% MeOH/DCM) to give the title Compound (65%) as a light brown solid. ¹H NMR (d₆-DMSO, 300 MHz) δ 2.44 (m, 2H), 3.78 (m, 2H), 4.05 (m, 4H), 4.30 (m, 2H), 5.78 (m, 2H), 7.31 (s, 1H), 7.82 (s, 1H), 8.19 (s, 1H), 8.55 (s, 1H), 8.80 (s, 1H); MS (ESI) m/z=464 (MH⁺).

### EXAMPLE 102

### (3-Chloro-6-furan-3-yl-8-trifluoromethyl-imidazo[1,2-a]pyridin-2-yl)-(3-fluoro-1-hydroxy-3′,6′-dihydro-2′H-[2,4′]bipyridinyl-1′-yl)-methanone (Compound 214)

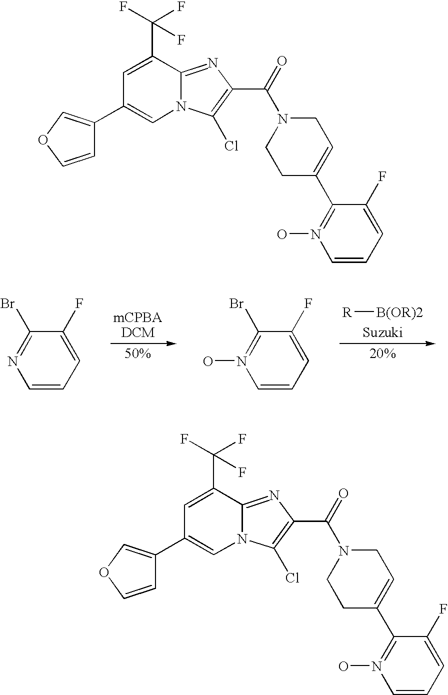

### Step 1: 2-Bromo-3-fluoro-2H-pyridin-1-ol

To a solution of 2 bromo-3-fluoro pyrdine (300 mg, 1.67 mmol) in CH₂Cl₂ (40 mL) was added m-chloroperbenzoic acid (520 mg, 3.5 mmol) at 0° C. After the reaction mixture was stirred for 30 min, sat. NaHCO₃ was added. The aqueous layer was extracted with CH₂Cl₂. The combined organic layers were dried (Na₂SO₄), filtered and concentrated. Purification of the residue by column chromatography [CH₂Cl₂/MeOH (10:1 v/v)]gave 2-bromo-3-fluoro-2H-pyridin-1-ol (50%) as colorless oil which was used without further purification. MS (ESI) m/z=195 (MH⁺).

### Step 2

Title Compound, Yield: 20%

Prepared using similar Suzuki procedure as in Example 87 for Compound 10.

¹H NMR (d₆-DMSO, 300 MHz) δ 2.49 (m 2H), 3.85 (m, 2H), 4.36 (m, 2H), 6.03 (bd, 1H), 7.31 (m, 1H), 7.39 (m, 2H) 7.82 (m, 1H), 8.16 (m, 2H), 8.55 (s, 1H), 8.82 (s, 1H); MS (ESI) m/z=509 (MH⁺).

### EXAMPLE 103

### [3-(3-Fluoro-phenyl)-pyrrolidin-1-yl]-(8-trifluoromethyl-imidazo[1,2-a]pyridin-2-yl)-methanone (hereinafter referred to as “Compound 11”)

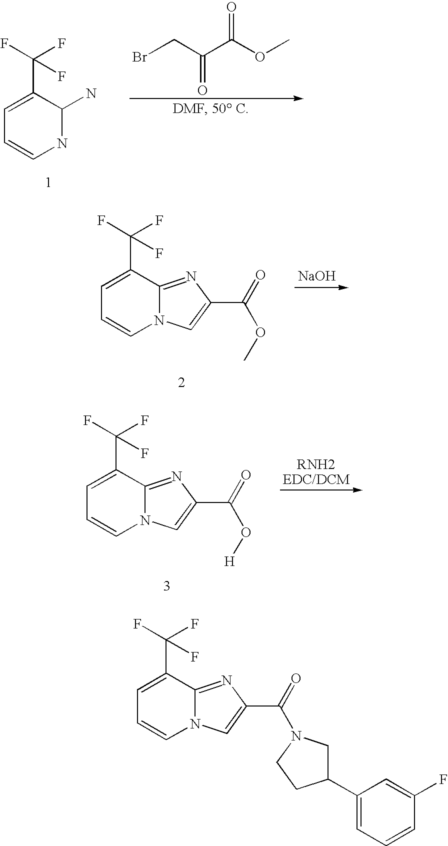

### Step 1: 8-Trifluoromethyl-imidazo[1,2-a]pyridine-2-carboxylic acid methyl ester

A solution of 3-trifluoromethyl-1,2-dihydro-pyridin-2-ylamine (2.5 g, 15.2 mmol) and methyl-3-bromopyruvate (5.5 g, 30.4 mmol) in DMF (50 mL) was heated at 60° C. for 1 hour. The mixture was concentrated, ice H₂O was added with rapid stirring, and the resulting precipitate filtered, washed with H₂O (4×300 mL), dried under vacuum overnight to give 8-trifluoromethyl-imidazo[1,2-a]pyridine-2-carboxylic acid methyl ester (80%) as a yellow powder. MS (ESI) m/z=245 (MH⁺).

### Step 2: 8-Trifluoromethyl-imidazo[1,2-a]pyridine-2-carboxylic acid

A mixture of 8-trifluoromethyl-imidazo[1,2-a]pyridine-2-carboxylic acid methyl ester (0.5 g, 2 mmol) and NaOH (2M, 4 mmol) was stirred at room temperature in THF/H₂O (3:1 v/v, 100 mL) for 2 hours. The reaction mixture was concentrated and the residue was acidified with 10% HCl and extracted with DCM (2×80 mL). The organic layer was washed with brine (50 mL), dried (MgSO₄), filtered and concentrated to afford 8-trifluoromethyl-imidazo[1,2-a]pyridine-2-carboxylic acid as a light yellow powder (60%) which was used without further purification. MS (ESI) m/z=231 (MH⁺).

### Step 3: [3-(3-Fluoro-phenyl)-pyrrolidin-1-yl]-(8-trifluoromethyl-imidazo[1,2-a]pyridin-2-yl)-methanone

A solution of 8-trifluoromethyl-imidazo[1,2-a]pyridine-2-carboxylic acid (75 mg, 0.3 mmol), EDC (0.12 g, 0.6 mmol), and 3-(3-fluoro-phenyl)-pyrrolidine. HCl salt (0.12 g, 0.6 mmol in DCM (2 mL) was stirred at room temperature for 2 hours. The reaction mixture was washed with H₂O (30 mL), saturated aqueous NaHCO₃ (30 mL), brine (30 mL), dried (MgSO₄). The crude material was purified by preparative TLC (50% EtOAc/hexane) to give [3-(3-fluoro-phenyl)-pyrrolidin-1-yl]-(8-trifluoromethyl-imidazo[1,2-a]pyridin-2-yl)-methanone (30%) as a white powder. MS (ESI) m/z=378 (MH⁺).

### EXAMPLE 104

### (3-Chloro-6-phenyl-8-trifluoromethyl-imidazo[1,2-a]pyridin-2-yl)-(3-thiazol-2-yl-2,5-dihydro-pyrrol-1-yl)-methanone (Compound 215)

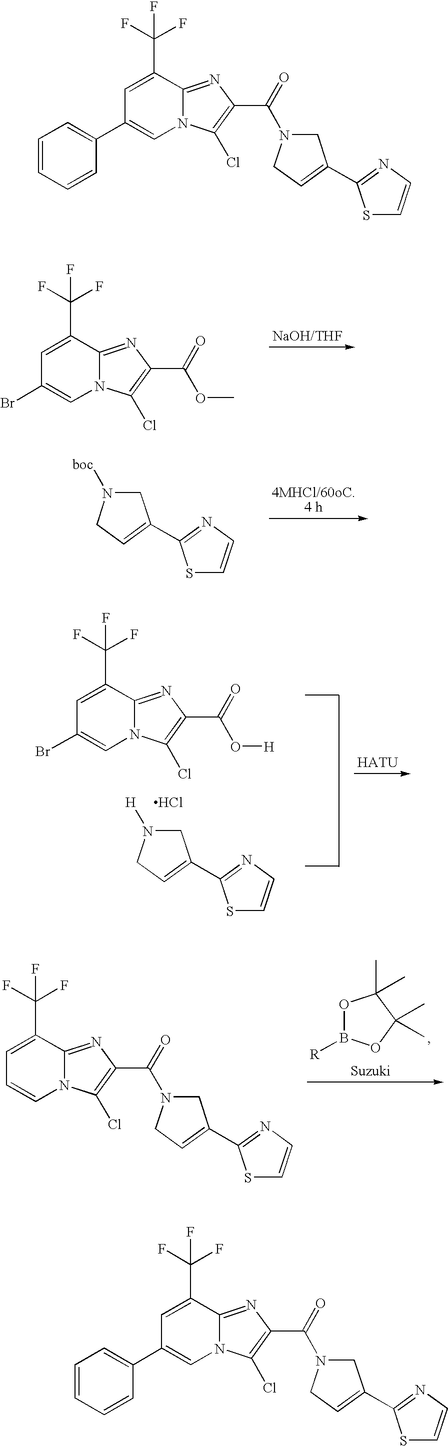

### Step 1: (6-Bromo-3-chloro-8-trifluoromethyl-imidazo[1,2-a]pyridin-2-yl)-(3-thiazol-2-yl-2,5-dihydro-pyrrol-1-yl)-methanone

Following similar procedure as in Example 103 for Compound 11. MS (ESI) m/z=478 (MH⁺).

### Step 2

Title Compound: Prepared using standard Suzuki condition of the bromo Compound from step 1.

¹H NMR (d₆-DMSO, 300 MHz) δ 4.57 (m, 1H), 4.70 (m, 1H), 4.92 (m, 1H), 5.10 (m, 1H), 6.71 (m, 1H), 7.51 (m, 3H), 7.78 (m, 1H), 7.86 (m, 3H), 8.20 (s, 1H), 8.82 (s, 1H); MS (ESI) m/z=476 (MH⁺).

### EXAMPLE 105

### (3-Chloro-6-pyridin-3-yl-8-trifluoromethyl-imidazo[1,2-a]pyridin-2-yl)-(3-thiazol-2-yl-2,5-dihydro-pyrrol-1-yl)-methanone (Compound 216)

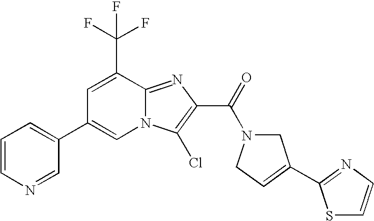

Title Compound: Prepared using standard Suzuki condition of the bromo Compound from step 1.

¹H NMR (d₆-DMSO, 300 MHz) δ 4.57 (bs, 1H), 4.77 (bs, 1H), 4.91 (bs, 1H), 5.10 (bs, 1H), 6.70 (m, 1H), 7.56 (m, 1H), 7.77 (m, 1H), 7.86 (m, 1H), 8.29 (s, 2H), 8.66 (m, 1H), 8.99 (s, 1H), 9.08 (s, 1H); MS (ESI) m/z=477 (MH⁺).

### EXAMPLE 106

### (3-Chloro-6-cyclohex-1-enyl-8-trifluoromethyl-imidazo[1,2-a]pyridin-2-yl)-(3-thiazol-2-yl-2,5-dihydro-pyrrol-1-yl)-methanone (Compound 217)

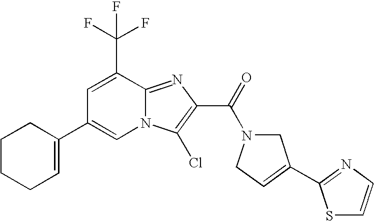

Title Compound: Prepared using standard Suzuki condition of the bromo Compound from step 1.

¹H NMR (d₆-DMSO, 300 MHz) δ 1.62 (m, 2H), 1.74 (m, 2H), 2.22 (m, 2H), 4.54 (m, 2H), 4.75 (bs, 2H), 4.89 (bs, 1H), 5.08 (bs, 1H), 6.49 (m, 1H), 6.70 (m, 1H), 7.76 (m, 1H), 7.86 (m, 1H), 8.05 (s, 1H), 8.35 (s, 1H); MS (ESI) m/z=480 (MH⁺).

### EXAMPLE 107

### [3-Chloro-6-(1,2,3,6-tetrahydro-pyridin-4-yl)-8-trifluoromethyl-imidazo[1,2-a]pyridin-2-yl]-(3-thiazol-2-yl-2,5-dihydro-pyrrol-1-yl)-methanone (Compound 218)

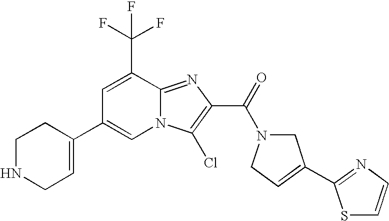

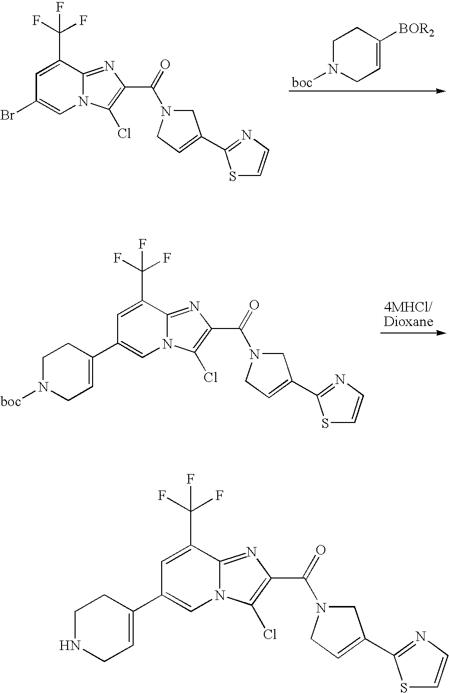

### Step 1: 4-[3-Chloro-2-(3-thiazol-2-yl-2,5-dihydro-pyrrole-1-carbonyl)-8-trifluoromethyl-imidazo[1,2-a]pyridin-6-yl]-3,6-dihydro-2H-pyridine-1-carboxylic acid tert-butyl ester

Prepared using similar Suzuki procedure as in Example 87 for Compound 10 and the crude was used in the next step without further purification. Yield: 50%.

### Step 2

Title Compound: 4M HCl in dioxane (1.0 mmol) was added to a solution of 4-[3-chloro-2-(3-thiazol-2-yl-2,5-dihydro-pyrrole-1-carbonyl)-8-trifluoromethyl-imidazo[1,2-a]pyridin-6-yl]-3,6-dihydro-2H-pyridine-1-carboxylic acid tert-butyl ester (70 mg, 0.12 mmol) in THF (1 mL) and was stirred at room temperature for 12 hours. The reaction mixture was concentrated and the crude material was subjected to preparative TLC (6% MeOH/DCM) to give the title Compound (45%) as a light brown solid.

¹H NMR (d₆-DMSO, 300 MHz) δ 2.81 (m, 2H), 3.33 (m, 2H), 3.74 (m, 2H), 4.55 (bs, 1H), 4.75 (bs, 1H), 4.89 (bs, 1H), 5.07 (bs, 1H), 6.56 (m, 1H), 6.69 (m, 1H), 7.76 (m, 1H), 7.84 (m, 1H), 8.10 (s, 1H), 8.48 (s, 1H), 9.20 (bs, 1H); MS (ESI) m/z=481 (MH⁺).

### EXAMPLE 108

### (3-Chloro-6-furan-3-yl-8-trifluoromethyl-imidazo[1,2-a]pyridin-2-yl)-(3-fluoro-3′,4′,5′,6′-tetrahydro-2′H-[2,4′]bipyridinyl-1′-yl)-methanone (Compound 219)

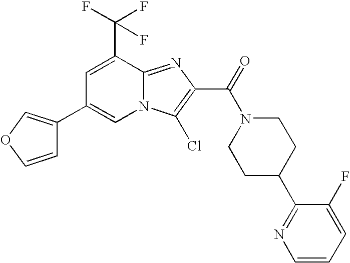

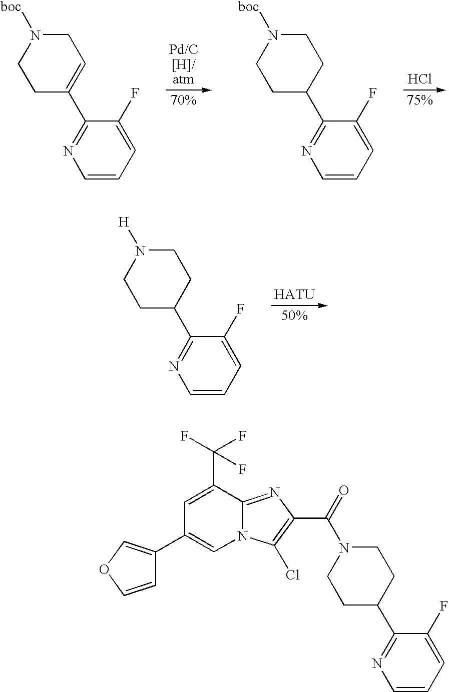

### Step 1: 3-Fluoro-3′,6′-dihydro-2′H-[2,4′]bipyridinyl-1′-carboxylic acid tert-butyl ester

A suspension of 3-fluoro-3′,6′-dihydro-2′H-[2,4′]bipyridinyl-1′-carboxylic acid tert-butyl ester (200 mg, 0.71 mmol) and 10% Pd/C (100 mg) was stirred under H₂ at atm pressure in EtOH. After 72 hours, the catalyst was filtered through Celite and the solvent was concentrated under reduced pressure to give the title compound (70%) which was used for the next step without further purification. ¹H NMR (d₆-DMSO, 300 MHz) δ 1.46 (m, 9H), 1.84 (m, 4H), 2.84 (m, 2H), 3.18 (m, 1H), 4.23 (m, 2H), 7.15 (m, 1H), 7.32 (m, 1H), 8.34 (m, 1H); MS (ESI) m/z=281 (MH⁺).

### Step 2: 3-Fluoro-1′,2′,3′,4′,5′,6′-hexahydro-[2,4′]bipyridinyl

Prepared using standard Boc removal procedure using 4M HCl in dioxane (5 eq) to give the title Compound as a light brown solid. MS (ESI) m/z=454 (MH⁺).

### Step 3: Title Compound was Prepared Using Standard HATU Coupling of the Above Amine with the Acid Described Before

¹H NMR (d₆-DMSO, 300 MHz) δ 1.81 (m, 4H), 2.99 (m, 1H), 3.33 (m, 2H), 4.13 (m, 1H), 4.65 (m, 1H), 7.31 (m, 1H), 7.34 (m, 1H), 7.66 (m, 1H), 7.82 (t, 1H, J=4.2 Hz), 8.18 (s, 1H), 8.39 (m, 1H), 8.54 (s, 1H), 8.77 (s, 1H); MS (ESI) m/z=494 (MH⁺).

### EXAMPLE 109

### 1′-(3-Chloro-6-furan-3-yl-8-trifluoromethyl-imidazo[1,2-a]pyridine-2-carbonyl)-3-fluoro-2′,3′,5′,6′-tetrahydro-1′H-[2,4′]bipyridinyl-4′-carbonitrile (Compound 220)

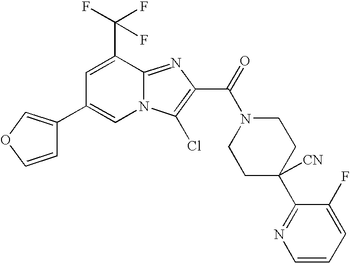

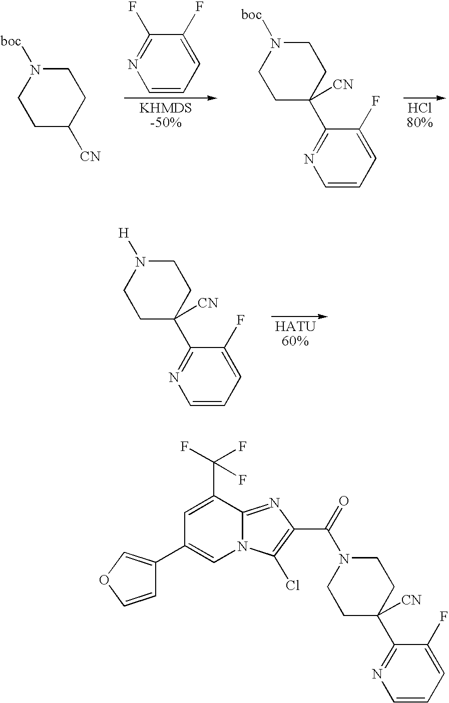

### Step 1: 4′-Cyano-3-fluoro-3′,4′,5′,6′-tetrahydro-2′H-[2,4′]bipyridinyl-1′-carboxylic acid tert-butyl ester

A solution of 4-cyano-piperidine-1-carboxylic acid tert-butyl ester (200 mg, 0.95 mmol) in DMF (2 mL) was added to a stirred solution of KHMDS (0.5 M in hexane, 1.9 mmol). After 5 min, a solution of 2,3-difluoropyridine (0.13 g, 1.14 mmol) in DMF (2 mL) was added and stirred for additional 2 h under nitrogen atmosphere. DMF was evaporated in vacuo. The resulting crude product was extracted with EtOAc and the extracts were successively washed with water and brine, and the organic layer was evaporated and subjected to column chromatography (50% Ethyl acetate/hexane) to provide the title Compound. ¹H NMR (d₆-DMSO, 300 MHz) δ 1.40 (m, 9H), 2.18 (m, 4H), 3.20 (m, 2H), 4.15 (m, 2H), 7.28 (m, 1H), 7.42 (m, 1H), 8.34 (m, 1H); MS (ESI) m/z=307 (MH⁺).

### Step 2: 3-Fluoro-2′,3′,5′,6′-tetrahydro-1′H-[2,4′]bipyridinyl-4′-carbonitrile

A solution of 4′-cyano-3-fluoro-3′,4′,5′,6′-tetrahydro-2′H-[2,4′]bipyridinyl-1′-carboxylic acid tert-butyl ester from step 1 (200 mg, 0.202 mmol) in THF (2 mL) and 4M HCl in dioxane (2 mL) was stirred at room temperature for 12 hours, and concentrated in vacuo to afford the title Compound (66.8 mg, 73%) as a pale yellow solid. MS (ESI) m/z=207 (MH⁺).

### Step 3: Title Compound was Prepared Using Standard HATU Coupling

¹H NMR (d₆-DMSO, 300 MHz) 2.22 (m, 3H), 2.37 (m, 1H), 3.18 (m, 1H), 3.44 (m, 1H), 4.34 (m, 1H), 4.61 (m, 1H), 7.27 (m, 1H), 7.53 (m, 1H), 7.77 (m, 1H), 7.84 (m, 1H), 8.15 (s, 1H), 8.43 (m, 1H), 8.50 (s, 1H), 8.77 (s, 1H); MS (ESI) m/z=494 (MH⁺).

### EXAMPLE 110

### 1-(3-Chloro-6-furan-3-yl-8-trifluoromethyl-imidazo[1,2-a]pyridine-2-carbonyl)-4-thiophen-2-yl-piperidine-4-carbonitrile (Compound 221)

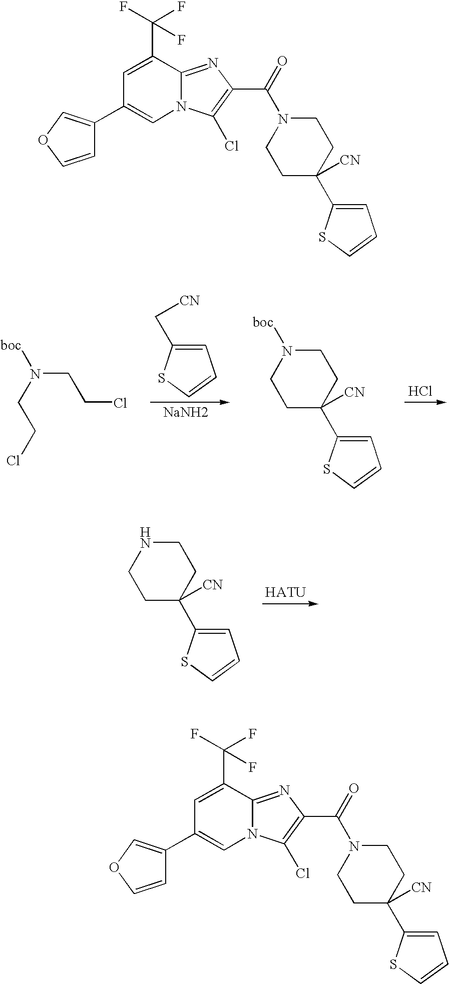

### Step 1: 4-Cyano-4-thiophen-2-yl-piperidine-1-carboxylic acid tert-butyl ester

To a solution of thiophen-2-yl-acetonitrile (700 mg, 5.69 mmol) at 0° C. in DMF (30 mL) was added NaNH₂ (665 mg, 17.0 mmol) carefully over 10 min and N-tert-butoxycarbonyl-bis(2-chloroethyl)amine (4.12 g, 10 mmol) in DMF was added slowly for 20 min. The resulting suspension was stirred at room temperature for 96 hours. The reaction mixture was cooled and quenched with ice-water (100 mL) and the mixture was extracted with EtOAc (3×50 mL). The combined organic layers were dried (MgSO₄), filtered and concentrated. The residue was purified by column chromatography on silica gel (20% EtOAc/hexanes) to yield the BOC-protected piperidine (31% yield). ¹H NMR (d₆-DMSO, 300 MHz) δ 1.47 (m, 9H), 1.96 (m, 2H), 2.24 (m, 2H), 3.20 (m, 2H), 4.25 (m, 2H), 7.01 (m, 1H), 7.15 (m, 1H), 7.30 (m, 1H); MS (ESI) m/z=293 (MH⁺).

### Step 2: 4-Thiophen-2-yl-piperidine-4-carbonitrile

Prepared using standard Boc removal procedure using 4M HCl in dioxane (5 eq) to give the title Compound as a light brown solid (90% yield). MS (ESI) m/z=194 (MH⁺).

### Step 3: Title Compound was Prepared Using Standard HATU Coupling of the Above Amine with the Acid Described Before

¹H NMR (d₆-DMSO, 300 MHz) 2.05 (m, 2H), 2.37 (m, 1H), 3.18 (m, 1H), 3.30 (m, 1H), 3.38 (m, 1H), 4.34 (m, 1H), 4.62 (m, 1H), 7.08 (m, 1H), 7.26 (m, 1H), 7.32 (m, 1H), 7.59 (m, 1H), 7.83 (m, 1H), 8.30 (s, 1H), 8.43 (m, 1H), 8.55 (s, 1H), 8.82 (s, 1H); MS (ESI) m/z=506 (MH⁺).

### EXAMPLE 111

### (3-Chloro-6-furan-3-yl-8-trifluoromethyl-imidazo[1,2-a]pyridin-2-yl)-(3-fluoro-4′-hydroxy-3′,4′,5′,6′-tetrahydro-2′H-[2,4′]bipyridinyl-1′-yl)-methanone (Compound 222)

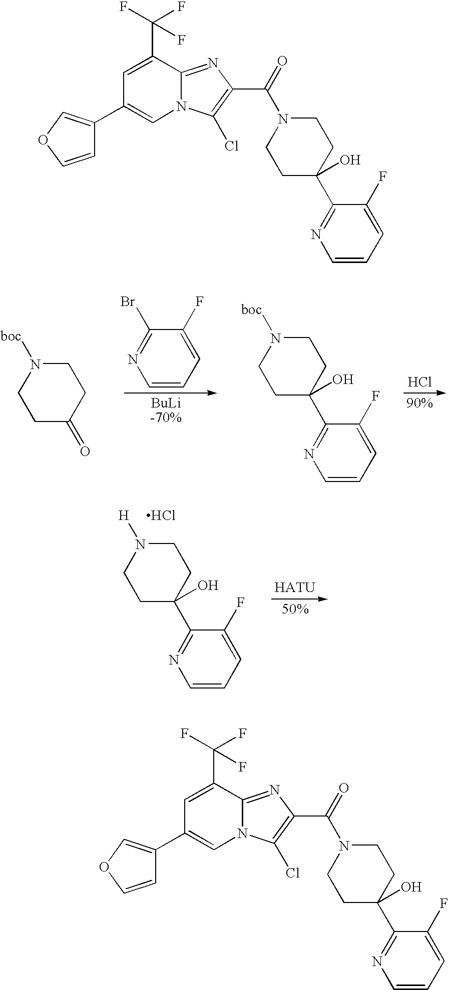

### Step 1: 3-Fluoro-4′-hydroxy-3′,4′,5′,6′-tetrahydro-2′H-[2,4′]bipyridinyl-1′-carboxylic acid tert-butyl ester

To a solution of 2-bromo-3-fluoropyrdine (0.52 g, 2.97 mmol) in THF at −50° C. was added n-BuLi (2.5M solution in THF, 5.60 mmol). After 10 min 4-oxo-piperidine-1-carboxylic acid tert-butyl ester (450 mg, 2.25 mmol) in THF (30 mL). The resulting suspension was stirred at room temperature for 12 hours. The reaction mixture was cooled and quenched with ice-water (100 mL) and the mixture was extracted with EtOAc (50 mL). The combined organic layers were dried (MgSO₄), filtered and concentrated. The residue was purified by column chromatography on silica gel (20% EtOAc/hexanes) to yield the title Compound (65%). ¹H NMR (d₆-DMSO, 300 MHz) δ 1.47 (s, 9H), 2.27 (m, 2H), 2.45 (dd, 1H, J=6.6, 6.0 Hz), 3.30 (m, 2H), 3.72 (t, 1H, J=6.4 Hz), 4.09 (m, 2H), 7.29 (m, 1H), 7.46 (m, 1H), 8.38 (m, 1H); MS (ESI) m/z=297 (MH⁺).

### Step 2: 3-Fluoro-2′,3′,5′,6′-tetrahydro-1′H-[2,4′]bipyridinyl-4′-ol

Prepared using standard Boc removal procedure using 4M HCl in dioxane (5 eq) to give the title Compound as a light brown solid. MS (ESI) m/z=197 (MH⁺).

### Step 3: Title Compound was Prepared Using Standard HATU Coupling of the Above Amine with the Acid Described Before

¹H NMR (d₆-DMSO, 300 MHz) δ 1.80 (m, 1H), 1.85 (m, 1H), 2.15 (m, 2H), 3.45 (m, 1H), 3.60 (m, 1H), 4.35 (m, 3H), 7.30 (s, 1H), 7.43 (m, 1H), 7.70 (m, 1H), 7.81 (m, 1H), 8.17 (s, 1H), 8.37 (m, 1H), 8.53 (s, 1H), 8.79 (s, 1H); MS (ESI) m/z=510 (MH⁺).

### EXAMPLE 112

### (3-Chloro-6-furan-3-yl-8-trifluoromethyl-imidazo[1,2-a]pyridin-2-yl)-(3,4′-difluoro-3′,4′,5′,6′-tetrahydro-2′H-[2,4′]bipyridinyl-1′-yl)-methanone (Compound 223)

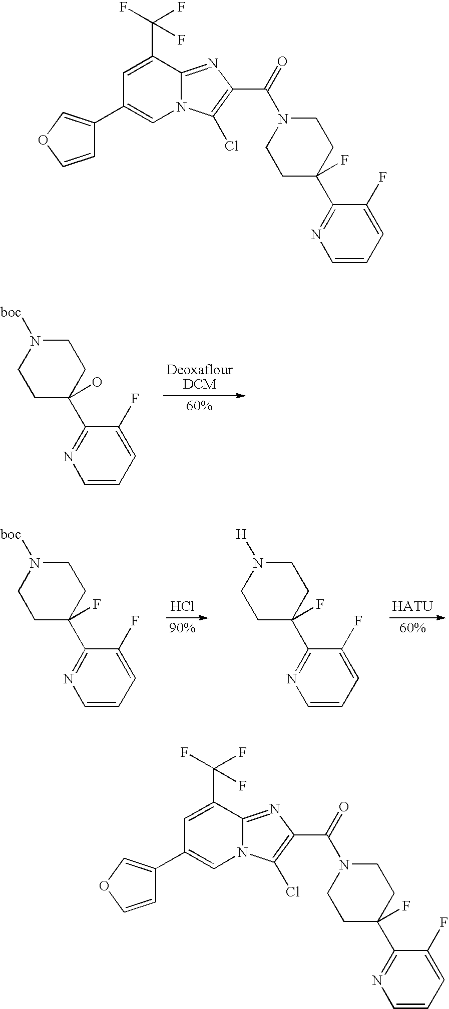

### Step 1: 3,4′-Difluoro-3′,4′,5′,6′-tetrahydro-2′H-[2,4′]bipyridinyl-1′-carboxylic acid tert-butyl ester

3-Fluoro-2′,3′,5′,6′-tetrahydro-1′H-[2,4′]bipyridinyl-4′-ol (200 mg, 0.67 mmol) was suspended in dichloromethane (10 mL) and cooled to −78° C. Deoxoflour (1.34 mmol) was added and the reaction mixture was stirred at −78° C. for 2 hours and then at room temperature for 12 hours. When the reaction was complete, the reaction mixture was extracted with cold water (20 mL), the aqueous phase was subsequently re-extracted with CH₂Cl₂ (3×15 mL). The combined organic phases were dried (Na₂SO₄), filtered and then evaporated at reduced pressure to yield the crude product. Column chromatography [n-hex/EtOAc (5:4 v/v)]of the crude material gave 3,4′-difluoro-3′,4′,5′,6′-tetrahydro-2′H-[2,4′]bipyridinyl-1′-carboxylic acid tert-butyl ester as viscous oil (60%). MS (ESI) m/z=299 (MH⁺).

### Step 2: 3,4′-Difluoro-1′,2′,3′,4′,5′,6′-hexahydro-[2,4′]bipyridinyl

Prepared using standard Boc removal procedure using 4M HCl in dioxane (5 eq) to give the title Compound as a light brown solid. MS (ESI) m/z=199 (MH⁺).

### Step 3: Title Compound was Prepared Using Standard HATU Coupling of the Above Amine with the Acid Described Before

¹H NMR (d₆-DMSO, 300 MHz) 2.22 (m, 4H), 3.30 (m, 1H), 3.55 (m, 1H), 4.15 (m, 1H), 4.45 (m, 1H), 7.32 (m, 1H), 7.54 (m, 1H), 7.82 (m, 2H), 8.19 (s, 1H), 8.44 (m, 1H), 8.55 (s, 1H), 8.80 (s, 1H); MS (ESI) m/z=512 (MH⁺).

### EXAMPLE 113

### (3-Chloro-6-furan-3-yl-8-trifluoromethyl-imidazo[1,2-a]pyridin-2-yl)-(4-hydroxy-4-thiazol-2-yl-piperidin-1-yl)-methanone (Compound 224)

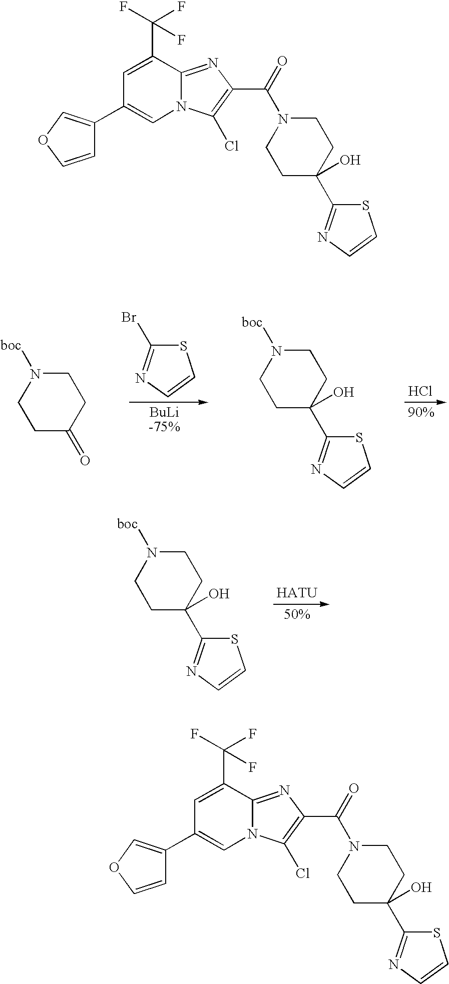

### Steps 1-4: Prepared Using Similar Procedure Described for the Synthesis of Compound 223

¹H NMR (d₆-DMSO, 300 MHz) δ 1.67 (m, 1H), 1.80 (m, 1H), 2.08 (m, 2H), 3.21 (m, 1H), 3.43 (m, 1H), 3.98 (bs, 1H), 4.36 (m, 2H), 7.26 (m, 1H), 7.56 (d, 1H, J=3.3 Hz), 7.67 (d, 1H, J=3.6 Hz), 7.77 (m, 1H), 8.13 (s, 1H), 8.20 (m, 1H, 8.76 (s, 1H); MS (ESI) m/z=510 (MH⁺).

### EXAMPLE 114

### (3-Chloro-6-furan-3-yl-8-trifluoromethyl-imidazo[1,2-a]pyridin-2-yl)-[4-(3-methyl-butyl)-piperidin-1-yl]-methanone (Compound 225)

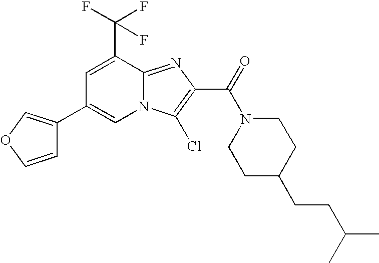

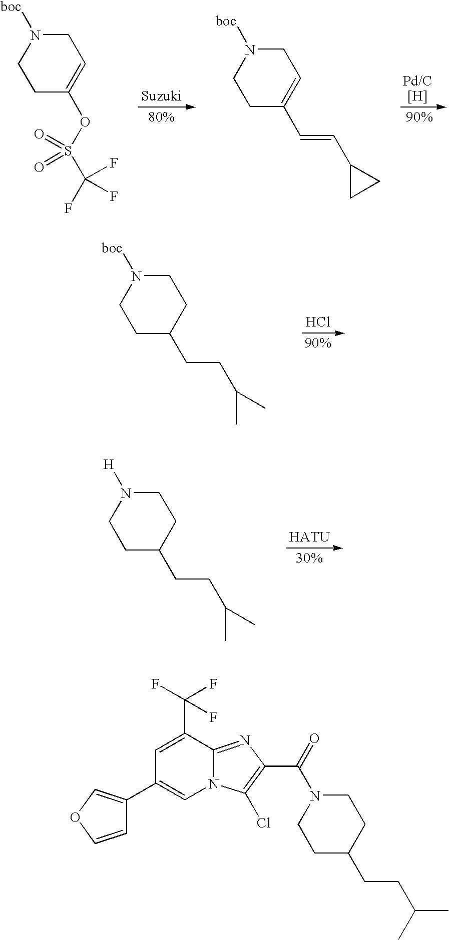

### Step 1: 4-((E)-2-Cyclopropyl-vinyl)-3,6-dihydro-2H-pyridine-1-carboxylic acid tert-butyl ester

Standard Suzuki conditions utilizing 4-trifluoromethanesulfonyloxy-3,6-dihydro-2H-pyridine-1-carboxylic acid tert-butyl ester with vinyl boronate yielded the title Compound in 80% yield. ¹H NMR (d₆-DMSO, 300 MHz) δ 0.40 (m, 2H), 0.74 (m, 2H), 1.25 (m, 1H), 1.37 (m, 9H), 2.18 (m, 2H), 2.51 (m, 2H), 3.96 (m, 2H), 5.16 (m, 1H), 5.52 (m, 1H), 6.18 (d, 1H, J=15.9 Hz); MS (ESI) m/z=250 (MH⁺).

### Step 2: 4-(3-Methyl-butyl)-piperidine-1-carboxylic acid tert-butyl ester

Procedure similar to the synthesis reported for Step 1 of Compound 219

MS (ESI) m/z=256 (MH⁺).

### Step 3: 4-(3-Methyl-butyl)-piperidine

Prepared using standard Boc removal procedure using 4M HCl in dioxane (5 eq) to give the title Compound as a light brown solid. MS (ESI) m/z=156 (MH⁺).

### Step 4: Title Compound was Prepared Using Standard HATU Coupling of the Above Amine with the Acid Described as Before

¹H NMR (d₆-DMSO, 300 MHz) δ 0.85 (m, 2H), 1.08 (m, 2H), 1.24 (m, 8H), 1.51 (m, 1H), 1.63 (m, 1H), 1.78 (m, 1H), 2.77 (m, 1H), 3.05 (m, 1H), 3.98 (m, 2H), 4.48 (m, 1H), 7.30 (m, 1H), 7.83 (t, 1H, J=1.8 Hz), 8.17 (s, 1H), 8.54 (s, 1H), 8.79 (s, 1H); MS (ESI) m/z=469 (MH⁺).

A further elaboration of processes suitable for preparing compounds of Formula 1, Formula 1a, Formula (I), Formula (II), Formula (III), Formula (IV), and Formula (V) are disclosed in Schmitz et al, U.S. patent application Ser. No. 12/228,139, filed on Aug. 8, 2008, which claims the benefit under 35 U.S.C. §119(e) of U.S. Provisional Patent Application Nos. 60/041,084, filed on Mar. 31, 2008, and 60/964,223, filed on Aug. 10, 2007, all of which applications are incorporated herein in their entirety by reference.

### BIOLOGICAL EXAMPLES

### Example 1

### Anti-Hepatitis C Activity

Compounds can exhibit anti-hepatitis C activity by inhibiting HCV polymerase, by inhibiting other enzymes needed in the replication cycle, or by other pathways. A number of assays have been published to assess these activities. A general method that assesses the gross increase of HCV virus in culture was disclosed in U.S. Pat. No. 5,738,985 to Miles et al. In vitro assays have been reported in Ferrari et al. Jnl. of Vir., 73:1649-1654, 1999; Ishii et al., Hepatology, 29:1227-1235, 1999; Lohmann et al., Jnl of Bio. Chem., 274:10807-10815, 1999; and Yamashita et al., Jnl. of Bio. Chem., 273:15479-15486, 1998.

### Example 2

### Replicon Assay

A cell line, ET (Huh-lucubineo-ET) is used for screening of compounds for inhibiting HCV RNA dependent RNA polymerase. The ET cell line is stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T1280I; K1846T) (Krieger at al, 2001 and unpublished). The ET cells are grown in DMEM (Dulbeco's Modified Eagle's Medium), supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They are all available through Life Technologies (Bethesda, Md.). The cells are plated at 0.5−1.0×10⁴ cells/well in the 96 well plates and incubated for 24 hrs before adding test compound. The compounds are added to the cells to achieve a final concentration of 0.1 nM to 50 μm and a final DMSO (dimethylsulfoxide) concentration of 0.5%. Luciferase activity is measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication data is plotted relative to no compound control. Under the same condition, cytotoxicity of the compounds are determined using cell proliferation reagent, WST-1 (Roche, Germany). The compounds showing antiviral activities, but no significant cytotoxicities are chosen to determine EC₅₀ and TC₅₀. For these determinations, a 10 point, 2-fold serial dilution for each compound was used, which spans a concentration range of 1000 fold. EC₅₀ and similarly TC₅₀ values were calculated by fitting % inhibition at each concentration to the following equation:

% inhibition=100%/[(EC₅₀/[I])^{b}+1]

where b is Hill's coefficient.

When tested, certain compounds of Tables 1 to 6 were found to have EC₅₀ values listed in Table 7.

TABLE 7

| Compound Number | EC50 (μM) |
|---|---|
| 101 | 7.008 |
| 102 | 0.04689 |
| 103 | 0.038917 |
| 104 | 5.964 |
| 105 | 0.12995 |
| 106 | 0.0886 |
| 107 | 0.24052 |
| 108 | 24.07 |
| 109 | 50 |
| 110 | 49 |
| 111 | 50 |
| 112 | 0.36816 |
| 113 | 8.628 |
| 114 | 1.332 |
| 115 | 22.47 |
| 116 | 24.43 |
| 117 | 23.92 |
| 118 | 0.655167 |
| 119 | 0.35425 |
| 120 | 3.042 |
| 121 | 2.538 |
| 122 | 5.889 |
| 123 | 0.14665 |
| 124 | 2.475 |
| 125 | 0.02929 |
| 126 | 0.027533 |
| 127 | 0.42845 |
| 128 | 4.826 |
| 129 | 20.26 |
| 130 | 49.76 |
| 131 | 0.2682 |
| 132 | 0.107357 |
| 133 | 3.47 |
| 134 | 0.1877 |
| 135 | 0.59586 |
| 136 | 5.37 |
| 137 | 50 |
| 138 | 35.27 |
| 139 | 0.112093 |
| 140 | 13.795 |
| 141 | 0.47585 |
| 142 | 1.937 |
| 143 | 0.901 |
| 144 | 7.549 |
| 145 | 3.496 |
| 146 | 2.244 |
| 147 | 3.989 |
| 149 | 5.08 |
| 150 | 0.00686 |
| 151 | 0.733933 |
| 152 | 19 |
| 153 | 13.93 |
| 154 | 27.69 |
| 155 | 44.5 |
| 156 | 46.84 |
| 157 | 50 |
| 158 | 8.112 |
| 159 | 0.2475 |
| 162 | 43.54 |
| 163 | 42.36 |
| 167 | 1.68 |
| 169 | 0.280275 |
| 170 | 0.025969 |
| 171 | 1.4135 |
| 172 | 3.1105 |
| 173 | 0.4443 |
| 174 | 0.31025 |
| 175 | 0.7902 |
| 176 | 0.8829 |
| 177 | 1.08 |
| 178 | 2.405 |
| 179 | 10.16 |
| 180 | 12.07 |
| 181 | 13.48 |
| 182 | 54.61 |
| 183 | 55 |
| 184 | 50 |
| 185 | 4.8 |
| 186 | 5.976 |
| 187 | 2.0016 |
| 188 | 50 |
| 189 | 50 |
| 190 | 50 |
| 191 | 50 |
| 192 | 50 |
| 193 | 2.504 |
| 194 | 0.8888 |
| 195 | 50 |
| 196 | 0.293667 |
| 197 | 3.499 |
| 198 | 14.59 |
| 199 | 50 |
| 200 | 50 |
| 201 | 15.29 |
| 202 | 0.329167 |
| 203 | 0.9745 |
| 204 | 3.714 |
| 205 | 18 |
| 206 | 5.953 |
| 207 | 17.85333 |
| 208 | 0.311717 |
| 209 | 0.3987 |
| 210 | 0.480733 |
| 211 | 1.481 |
| 212 | 0.3382 |
| 213 | 0.942 |
| 214 | 1.443 |
| 215 | 0.032183 |
| 216 | 0.11085 |
| 217 | 0.05055 |
| 218 | 2.001 |
| 219 | 0.065085 |
| 220 | 0.776525 |
| 221 | 0.04546 |
| 222 | 0.6235 |
| 223 | 0.657167 |
| 224 | 3.496 |
| 225 | 2.295 |
| 226 | 50 |
| 227 | 50 |
| 228 | 50 |
| 229 | 50 |
| 230 | 50 |
| 231 | 50 |
| 232 | 50 |
| 233 | 50 |
| 234 | 50 |
| 235 | 40.235 |
| 236 | 50 |
| 237 | 50 |
| 238 | 50 |
| 239 | 50 |
| 240 | 50 |
| 241 | 50 |
| 242 | 50 |
| 243 | 50 |
| 244 | 50 |
| 245 | 50 |
| 246 | 50 |
| 247 | 50 |
| 248 | 50 |
| 249 | 50 |
| 250 | 50 |
| 251 | 50 |
| 252 | 50 |
| 253 | 50 |
| 254 | 50 |
| 255 | 50 |
| 256 | 50 |
| 257 | 50 |
| 258 | 50 |
| 259 | 50 |
| 260 | 50 |
| 261 | 50 |
| 262 | 50 |
| 263 | 50 |
| 264 | 50 |
| 265 | 50 |
| 266 | 50 |
| 267 | 50 |
| 268 | 50 |
| 269 | 50 |
| 270 | 50 |
| 271 | 50 |
| 272 | 50 |
| 273 | 50 |
| 274 | 50 |
| 275 | 50 |
| 276 | 50 |
| 277 | 50 |
| 278 | 50 |
| 279 | 50 |
| 280 | 50 |
| 281 | 50 |
| 282 | 50 |
| 283 | 50 |
| 284 | 50 |
| 285 | 50 |
| 286 | 50 |
| 287 | 50 |
| 288 | 50 |
| 289 | 50 |
| 290 | 50 |
| 291 | 50 |
| 292 | 50 |
| 293 | 50 |
| 294 | 50 |
| 295 | 50 |
| 296 | 50 |
| 297 | 50 |
| 298 | 50 |
| 299 | 50 |
| 300 | 50 |
| 301 | 50 |
| 302 | 50 |
| 303 | 50 |
| 304 | 50 |
| 305 | 50 |
| 306 | 50 |
| 307 | 50 |
| 309 | 50 |
| 310 | 50 |
| 311 | 50 |
| 312 | 50 |
| 313 | 50 |
| 314 | 50 |
| 321 | 50 |
| 322 | 0.2661 |
| 323 | 10.7 |
| 324 | 50 |
| 325 | 50 |
| 326 | 11.42 |
| 327 | 50 |
| 330 | 50 |
| 331 | 26.76 |

### FORMULATION EXAMPLES

The following are representative pharmaceutical formulations containing a compound of Formula (I).

### Formulation Example 1

### Tablet Formulation

The following ingredients are mixed intimately and pressed into single scored tablets.

| | Quantity per |
|---|---|
| Ingredient | tablet, mg |
| compound | 400 |
| cornstarch | 50 |
| croscarmellose sodium | 25 |
| lactose | 120 |
| magnesium stearate | 5 |

### Formulation Example 2

### Capsule Formulation

The following ingredients are mixed intimately and loaded into a hard-shell gelatin capsule.

| | Quantity per |
|---|---|
| Ingredient | capsule, mg |
| compound | 200 |
| Lactose, spray-dried | 148 |
| magnesium stearate | 2 |

### Formulation Example 3

### Suspension Formulation

The following ingredients are mixed to form a suspension for oral administration.

| Ingredient | Amount | |
|---|---|---|
| compound | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |
| distilled water | q.s. (quantity sufficient) to 100 | mL |

### Formulation Example 4

### Injectable Formulation

The following ingredients are mixed to form an injectable formulation.

| Ingredient | Amount |
|---|---|
| compound | 0.2 mg-20 mg |
| sodium acetate buffer solution, | 0.4 M 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g is prepared by mixing the compound with Witepsol® H-15 (triglycerides of saturated vegetable fatty acid; Riches-Nelson, Inc., New York), and has the following composition:

| Ingredient | Amount |
|---|---|
| compound | 500 mg |
| Witepsol ® H-15 | balance |

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

1. A compound of Formula (I) 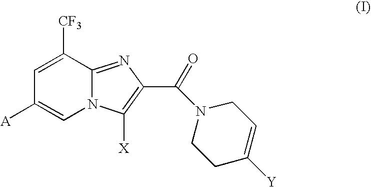
or a pharmaceutically acceptable salt thereof, wherein:
A is selected from the group consisting of furanyl, thiazolyl, imidazolyl, thienyl, dihydropyrrolyl, cyclopentenyl, phenyl, ethenyl, cyclopropylvinyl, and halo;
X is selected from the group consisting of hydrogen, halo, cyclopropyl, hydroxymethyl, hydroxyethyl, and hydroxy; and
Y is selected from the group consisting of aryl, heteroaryl, and heteroaryl substituted with 1 to 3 groups independently selected from the group consisting of halo, hydroxy, trifluoromethyl, methyl, cyano, methoxy, and ethoxy.

2. A compound of claim 1 or a pharmaceutically acceptable salt thereof selected from the group consisting of

| Compound | |
|---|---|
| Number | Name |
| 111 | (3-Chloro-6-furan-3-yl-8-trifluoromethyl-imidazo[1,2-a]pyridin-2-yl)-[4- |
| | (1H-imidazol-2-yl)-3,6-dihydro-2H-pyridin-1-yl]-methanone |
| 115 | (3-Chloro-6-furan-3-yl-8-trifluoromethyl-imidazo[1,2-a]pyridin-2-yl)-(3- |
| | ethoxy-3′,6′-dihydro-2′H-[2,4′]bipyridinyl-1′-yl)-methanone |
| 118 | (3-Chloro-6-thiazol-2-yl-8-trifluoromethyl-imidazo[1,2-a]pyridin-2-yl)-(3- |
| | fluoro-3′,6′-dihydro-2′H-[2,4′]bipyridinyl-1′-yl)-methanone |
| 119 | (3-Chloro-6-thiazol-5-yl-8-trifluoromethyl-imidazo[1,2-a]pyridin-2-yl)-(3- |
| | fluoro-3′,6′-dihydro-2′H-[2,4′]bipyridinyl-1′-yl)-methanone |
| 120 | (3-Chloro-6-thiazol-4-yl-8-trifluoromethyl-imidazo[1,2-a]pyridin-2-yl)-(3- |
| | fluoro-3′,6′-dihydro-2′H-[2,4′]bipyridinyl-1′-yl)-methanone |
| 121 | [3-Chloro-6-(2,5-dihydro-1H-pyrrol-3-yl)-8-trifluoromethyl-imidazo[1,2- |
| | a]pyridin-2-yl]-(3-fluoro-3′,6′-dihydro-2′H-[2,4′]bipyridinyl-1′-yl)- |
| | methanone |
| 122 | [3-Chloro-6-(1H-imidazol-4-yl)-8-trifluoromethyl-imidazo[1,2-a]pyridin-2- |
| | yl]-(3-fluoro-3′,6′-dihydro-2′H-[2,4′]bipyridinyl-1′-yl)-methanone |
| 123 | (3-Chloro-6-thiophen-2-yl-8-trifluoromethyl-imidazo[1,2-a]pyridin-2-yl)-(3- |
| | fluoro-3′,6′-dihydro-2′H-[2,4′]bipyridinyl-1′-yl)-methanone |
| 124 | (3-Chloro-6-phenyl-8-trifluoromethyl-imidazo[1,2-a]pyridin-2-yl)-(3- |
| | fluoro-3′,6′-dihydro-2′H-[2,4′]bipyridinyl-1′-yl)-methanone |
| 125 | (6-Bromo-3-chloro-8-trifluoromethyl-imidazo[1,2-a]pyridin-2-yl)-(3-fluoro- |
| | 3′,6′-dihydro-2′H-[2,4′]bipyridinyl-1′-yl)-methanone |
| 126 | (3-Chloro-8-trifluoromethyl-6-vinyl-imidazo[1,2-a]pyridin-2-yl)-(3-fluoro- |
| | 3′,6′-dihydro-2′H-[2,4′]bipyridinyl-1′-yl)-methanone |
| 127 | [3-Chloro-6-(2-cyclopropyl-vinyl)-8-trifluoromethyl-imidazo[1,2-a]pyridin- |
| | 2-yl]-(3-fluoro-3′,6′-dihydro-2′H-[2,4′]bipyridinyl-1′-yl)-methanone |
| 149 | (3-Cyclopropyl-6-furan-3-yl-8-trifluoromethyl-imidazo[1,2-a]pyridin-2-yl)- |
| | (3-fluoro-3′,6′-dihydro-2′H-[2,4′]bipyridinyl-1′-yl)-methanone |
| 151 | (3-Chloro-6-cyclopent-1-enyl-8-trifluoromethyl-imidazo[1,2-a]pyridin-2- |
| | yl)-(3-fluoro-3′,6′-dihydro-2′H-[2,4′]bipyridinyl-1′-yl)-methanone |
| 171 | (3-Fluoro-3′,6′-dihydro-2′H-[2,4′]bipyridinyl-1′-yl)-(6-furan-3-yl-3- |
| | hydroxymethyl-8-trifluoromethyl-imidazo[1,2-a]pyridin-2-yl)-methanone |
| 172 | (3-Fluoro-3′,6′-dihydro-2′H-[2,4′]bipyridinyl-1′-yl)-[6-furan-3-yl-3-(1- |
| | hydroxy-ethyl)-8-trifluoromethyl-imidazo[1,2-a]pyridin-2-yl]-methanone |
| 201 | (3-Chloro-6-furan-3-yl-8-trifluoromethyl-imidazo[1,2-a]pyridin-2-yl)-(3- |
| | hydroxy-3′,6′-dihydro-2′H-[2,4′]bipyridinyl-1′-yl)-methanone |
| 202 | (3-Chloro-3′,6′-dihydro-2′H-[2,4′]bipyridinyl-1′-yl)-(3-chloro-6-furan-3-yl-8- |
| | trifluoromethyl-imidazo[1,2-a]pyridin-2-yl)-methanone |
| 203 | (3-Chloro-6-furan-3-yl-8-trifluoromethyl-imidazo[1,2-a]pyridin-2-yl)-(5- |
| | fluoro-3′,6′-dihydro-2′H-[3,4′]bipyridinyl-1′-yl)-methanone |
| 204 | (3-Chloro-6-furan-3-yl-8-trifluoromethyl-imidazo[1,2-a]pyridin-2-yl)-(6- |
| | fluoro-3′,6′-dihydro-2′H-[2,4′]bipyridinyl-1′-yl)-methanone |
| 205 | (3-Chloro-6-furan-3-yl-8-trifluoromethyl-imidazo[1,2-a]pyridin-2-yl)-(3- |
| | trifluoromethyl-3′,6′-dihydro-2′H-[2,4′]bipyridinyl-1′-yl)-methanone |
| 206 | (3-Chloro-6-furan-3-yl-8-trifluoromethyl-imidazo[1,2-a]pyridin-2-yl)-(3- |
| | fluoro-4-methyl-3′,6′-dihydro-2′H-[2,4′]bipyridinyl-1′-yl)-methanone |
| 207 | (3-Chloro-6-furan-3-yl-8-trifluoromethyl-imidazo[1,2-a]pyridin-2-yl)-(3- |
| | fluoro-6-methyl-3′,6′-dihydro-2′H-[2,4′]bipyridinyl-1′-yl)-methanone |
| 208 | (3-Chloro-6-furan-3-yl-8-trifluoromethyl-imidazo[1,2-a]pyridin-2-yl)-[4-(5- |
| | fluoro-pyrimidin-4-yl)-3,6-dihydro-2H-pyridin-1-yl]-methanone |
| 209 | (3-Chloro-6-furan-3-yl-8-trifluoromethyl-imidazo[1,2-a]pyridin-2-yl)-(4- |
| | fluoro-3′,6′-dihydro-2′H-[2,4′]bipyridinyl-1′-yl)-methanone |
| 210 | (3-Chloro-6-furan-3-yl-8-trifluoromethyl-imidazo[1,2-a]pyridin-2-yl)-(3′,6′- |
| | dihydro-2′H-[2,4′]bipyridinyl-1′-yl)-methanone |
| 211 | 1′-(3-Chloro-6-furan-3-yl-8-trifluoromethyl-imidazo[1,2-a]pyridine-2- |
| | carbonyl)-1′,2′,3′,6′-tetrahydro-[2,4′]bipyridinyl-3-carbonitrile |
| 214 | (3-Chloro-6-furan-3-yl-8-trifluoromethyl-imidazo[1,2-a]pyridin-2-yl)-(3- |
| | fluoro-1-hydroxy-3′,6′-dihydro-2′H-[2,4′]bipyridinyl-1′-yl)-methanone |

3. A compound of Formula (II) 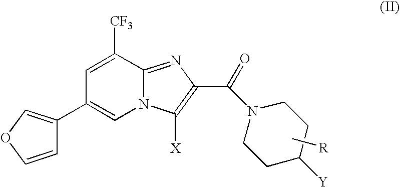
or a pharmaceutically acceptable salt thereof, wherein:
X is selected from the group consisting of hydrogen, halo, hydroxymethyl, and hydroxy;
R is selected from the group consisting of hydrogen, cyano, hydroxy, and halo; and
Y is selected from the group consisting of aryl, aryl substituted with halo, heteroaryl, and heteroaryl substituted with halo.

4. A compound of claim 3 or a pharmaceutically acceptable salt thereof selected from the group consisting of

| Compound | |
|---|---|
| Number | Name |
| 102 | (3-Chloro-6-furan-3-yl-8-trifluoromethyl-imidazo[1,2-a]pyridin-2-yl)-[4-(2- |
| | fluoro-phenyl)-3-hydroxy-piperidin-1-yl]-methanone |
| 197 | (3-Chloro-6-furan-3-yl-8-trifluoromethyl-imidazo[1,2-a]pyridin-2-yl)-(4- |
| | imidazol-1-yl-piperidin-1-yl)-methanone |
| 219 | (3-Chloro-6-furan-3-yl-8-trifluoromethyl-imidazo[1,2-a]pyridin-2-yl)-(3- |
| | fluoro-3′,4′,5′,6′-tetrahydro-2′H-[2,4′]bipyridinyl-1′-yl)-methanone |
| 220 | 1′-(3-Chloro-6-furan-3-yl-8-trifluoromethyl-imidazo[1,2-a]pyridine-2- |
| | carbonyl)-3-fluoro-2′,3′,5′,6′-tetrahydro-1′H-[2,4′]bipyridinyl-4′-carbonitrile |
| 221 | 1-(3-Chloro-6-furan-3-yl-8-trifluoromethyl-imidazo[1,2-a]pyridine-2- |
| | carbonyl)-4-thiophen-2-yl-piperidine-4-carbonitrile |
| 222 | (3-Chloro-6-furan-3-yl-8-trifluoromethyl-imidazo[1,2-a]pyridin-2-yl)-(3- |
| | fluoro-4′-hydroxy-3′,4′,5′,6′-tetrahydro-2′H-[2,4′]bipyridinyl-1′-yl)-methanone |
| 223 | (3-Chloro-6-furan-3-yl-8-trifluoromethyl-imidazo[1,2-a]pyridin-2-yl)-(3,4′- |
| | difluoro-3′,4′,5′,6′-tetrahydro-2′H-[2,4′]bipyridinyl-1′-yl)-methanone |
| 224 | (3-Chloro-6-furan-3-yl-8-trifluoromethyl-imidazo[1,2-a]pyridin-2-yl)-(4- |
| | hydroxy-4-thiazol-2-yl-piperidin-1-yl)-methanone |

5. A compound of Formula (III) 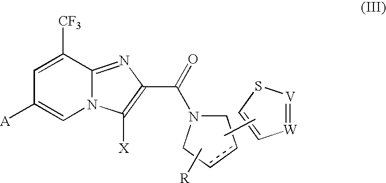
or a pharmaceutically acceptable salt thereof, wherein: [symbol]
represents a single or a double bond;
X is selected from the group consisting of hydrogen, halo, cyclopropyl, hydroxymethyl, and hydroxy;
R is selected from the group consisting of hydrogen, halo, cyano, and hydroxy;
one of W and V is CH and the other is N; and
A is selected from the group consisting of furanyl, thiazolyl, imidazolyl, thienyl, pyrazolyl, pyridyl, dihydropyridyl, dihydropyrrolyl, cyclopentenyl, cyclohexenyl, and phenyl.

6. A compound of claim 5 or a pharmaceutically acceptable salt thereof selected from the group consisting of

| Compound | |
|---|---|
| Number | Name |
| 105 | [3-Chloro-6-(1H-pyrazol-4-yl)-8-trifluoromethyl-imidazo[1,2-a]pyridin- |
| | 2-yl]-(3-thiazol-5-yl-2,5-dihydro-pyrrol-1-yl)-methanone |
| 106 | (3-Chloro-6-furan-3-yl-8-trifluoromethyl-imidazo[1,2-a]pyridin-2-yl)-(3- |
| | thiazol-5-yl-2,5-dihydro-pyrrol-1-yl)-methanone |
| 107 | [3-Chloro-6-(1H-pyrazol-4-yl)-8-trifluoromethyl-imidazo[1,2-a]pyridin- |
| | 2-yl]-(3-isothiazol-4-yl-2,5-dihydro-pyrrol-1-yl)-methanone |
| 148 | (3-Cyclopropyl-6-furan-3-yl-8-trifluoromethyl-imidazo[1,2-a]pyridin-2- |
| | yl)-(3-thiazol-2-yl-2,5-dihydro-pyrrol-1-yl)-methanone |
| 150 | (3-Chloro-6-cyclopent-1-enyl-8-trifluoromethyl-imidazo[1,2-a]pyridin-2- |
| | yl)-(3-thiazol-2-yl-2,5-dihydro-pyrrol-1-yl)-methanone |
| 170 | (6-Furan-3-yl-3-hydroxymethyl-8-trifluoromethyl-imidazo[1,2-a]pyridin- |
| | 2-yl)-(3-thiazol-2-yl-2,5-dihydro-pyrrol-1-yl)-methanone |
| 173 | [3-Hydroxymethyl-6-(1H-pyrazol-4-yl)-8-trifluoromethyl-imidazo[1,2- |
| | a]pyridin-2-yl]-(3-thiazol-2-yl-2,5-dihydro-pyrrol-1-yl)-methanone |
| 215 | (3-Chloro-6-phenyl-8-trifluoromethyl-imidazo[1,2-a]pyridin-2-yl)-(3- |
| | thiazol-2-yl-2,5-dihydro-pyrrol-1-yl)-methanone |
| 216 | (3-Chloro-6-pyridin-3-yl-8-trifluoromethyl-imidazo[1,2-a]pyridin-2-yl)- |
| | (3-thiazol-2-yl-2,5-dihydro-pyrrol-1-yl)-methanone |
| 217 | (3-Chloro-6-cyclohex-1-enyl-8-trifluoromethyl-imidazo[1,2-a]pyridin-2- |
| | yl)-(3-thiazol-2-yl-2,5-dihydro-pyrrol-1-yl)-methanone |
| 218 | [3-Chloro-6-(1,2,3,6-tetrahydro-pyridin-4-yl)-8-trifluoromethyl- |
| | imidazo[1,2-a]pyridin-2-yl]-(3-thiazol-2-yl-2,5-dihydro-pyrrol-1-yl)- |
| | methanone |

7. A compound of Formula (IV) 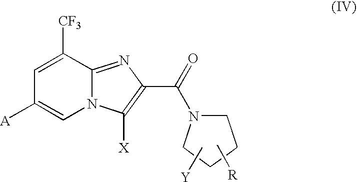
A is heteroaryl;
X is selected from the group consisting of hydrogen, halo, hydroxymethyl, and hydroxy;
R is selected from the group consisting of hydrogen, halo, cyano, and hydroxy; and
Y is selected from the group consisting of ethoxy, —O(CH₂)₂OCH₂CH₃, —NHR¹, and —NHC(O)R², wherein R¹ is selected from the group consisting of aryl, heteroaryl, and aryl substituted with alkoxy, and R² is selected from the group consisting of ethenyl, C₁-C₃ alkoxy substituted with halo, and C₁-C₃ alkyl substituted with 1 to 3 halo.

8. A compound of claim 7 or a pharmaceutically acceptable salt thereof selected from the group consisting of

| Compound | |
|---|---|
| Number | Name |
| 162 | N-[1-(3-Chloro-6-furan-3-yl-8-trifluoromethyl-imidazo[1,2-a]pyridine-2- |
| | carbonyl)-pyrrolidin-3-yl]-2,2,2-trifluoro-acetamide |
| 167 | N-[1-(3-Chloro-6-furan-3-yl-8-trifluoromethyl-imidazo[1,2-a]pyridine-2- |
| | carbonyl)-pyrrolidin-3-yl]-acrylamide |
| 178 | (3-Chloro-6-furan-3-yl-8-trifluoromethyl-imidazo[1,2-a]pyridin-2-yl)-[3- |
| | (3-methoxy-phenylamino)-pyrrolidin-1-yl]-methanone |
| 179 | [3-Chloro-6-(1H-pyrazol-4-yl)-8-trifluoromethyl-imidazo[1,2-a]pyridin-2- |
| | yl]-(3-phenylamino-pyrrolidin-1-yl)-methanone |
| 180 | (3-Chloro-6-furan-3-yl-8-trifluoromethyl-imidazo[1,2-a]pyridin-2-yl)-[3- |
| | (4-methoxy-phenylamino)-pyrrolidin-1-yl]-methanone |
| 181 | [3-Chloro-6-(1H-pyrazol-4-yl)-8-trifluoromethyl-imidazo[1,2-a]pyridin-2- |
| | yl]-(3-ethoxy-pyrrolidin-1-yl)-methanone |
| 183 | (3-Chloro-6-furan-3-yl-8-trifluoromethyl-imidazo[1,2-a]pyridin-2-yl)-[3- |
| | (pyrimidin-2-ylamino)-pyrrolidin-1-yl]-methanone |
| 233 | [3-Chloro-6-(1H-pyrazol-4-yl)-8-trifluoromethyl-imidazo[1,2-a]pyridin-2- |
| | yl]-[3-(2-ethoxy-ethoxy)-pyrrolidin-1-yl]-methanone |
| 321 | [1-(3-Chloro-6-furan-3-yl-8-trifluoromethyl-imidazo[1,2-a]pyridine-2- |
| | carbonyl)-pyrrolidin-3-yl]-carbamic acid 2-chloro-ethyl ester |

9. A compound of Formula (V) 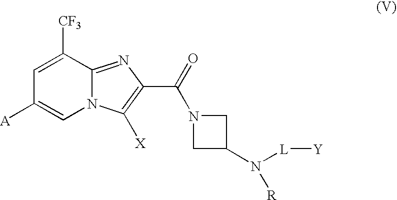
A is selected from the group consisting of halo, aryl, aryl substituted with halo, heteroaryl, and heteroaryl substituted with halo;
X is selected from the group consisting of hydrogen, halo, hydroxymethyl, and hydroxy;
R is selected from the group consisting of hydrogen, C₁-C₃ alkyl, and C₁-C₃ alkyl substituted with hydroxy;
L is —C(O)— or —S(O)₂—; and
Y is C₁-C₃ alkyl or cyclopropyl.

10. A compound of claim 9 or a pharmaceutically acceptable salt thereof selected from the group consisting of

| Compound | |
|---|---|
| Number | Name |
| 139 | N-[1-(3-Chloro-6-furan-2-yl-8-trifluoromethyl-imidazo[1,2-a]pyridine-2- |
| | carbonyl)-azetidin-3-yl]-methanesulfonamide |
| 140 | N-[1-(6-Bromo-3-chloro-8-trifluoromethyl-imidazo[1,2-a]pyridine-2- |
| | carbonyl)-azetidin-3-yl]-methanesulfonamide |
| 141 | N-{1-[3-Chloro-6-(3-fluoro-phenyl)-8-trifluoromethyl-imidazo[1,2- |
| | a]pyridine-2-carbonyl]-azetidin-3-yl}-methanesulfonamide |
| 142 | N-[1-(3-Chloro-6-pyridin-3-yl-8-trifluoromethyl-imidazo[1,2-a]pyridine-2- |
| | carbonyl)-azetidin-3-yl]-methanesulfonamide |
| 143 | N-[1-(3-Chloro-6-furan-3-yl-8-trifluoromethyl-imidazo[1,2-a]pyridine-2- |
| | carbonyl)-azetidin-3-yl]-N-methyl-methanesulfonamide |
| 144 | N-[1-(3-Chloro-6-furan-3-yl-8-trifluoromethyl-imidazo[1,2-a]pyridine-2- |
| | carbonyl)-azetidin-3-yl]-N-(2-hydroxy-ethyl)-methanesulfonamide |
| 145 | N-{1-[6-(5-Bromo-furan-3-yl)-8-trifluoromethyl-imidazo[1,2-a]pyridine-2- |
| | carbonyl]-azetidin-3-yl}-methanesulfonamide |
| 146 | Ethanesulfonic acid {1-[6-(5-bromo-furan-3-yl)-8-trifluoromethyl- |
| | imidazo[1,2-a]pyridine-2-carbonyl]-azetidin-3-yl}-amide |
| 147 | Cyclopropanesulfonic acid {1-[6-(5-bromo-furan-3-yl)-8-trifluoromethyl- |
| | imidazo[1,2-a]pyridine-2-carbonyl]-azetidin-3-yl}-amide |

11. A compound or a pharmaceutically acceptable salt thereof selected from the group consisting of

| Compound | |
|---|---|
| Number | Name |
| 101 | 3-Chloro-6-furan-3-yl-8-trifluoromethyl-imidazo[1,2-a]pyridine-2- |
| | carboxylic acid |
| 103 | [3-Chloro-6-(1H-pyrazol-4-yl)-8-trifluoromethyl-imidazo[1,2-a]pyridin-2- |
| | yl]-[3-(2-fluoro-phenyl)-2,5-dihydro-pyrrol-1-yl]-methanone |
| 104 | [3-Chloro-6-(1H-pyrazol-4-yl)-8-trifluoromethyl-imidazo[1,2-a]pyridin-2- |
| | yl]-[3-(1H-imidazol-4-yl)-2,5-dihydro-pyrrol-1-yl]-methanone |
| 108 | (3-Chloro-6-furan-3-yl-8-trifluoromethyl-imidazo[1,2-a]pyridin-2-yl)-(4- |
| | methanesulfonyl-piperazin-1-yl)-methanone |
| 109 | 2-(6-Furan-3-yl-8-trifluoromethyl-imidazo[1,2-a]pyridin-2-yl)-1-(4- |
| | thiazol-2-yl-piperazin-1-yl)-ethanone |
| 110 | 2-(6-Furan-3-yl-8-trifluoromethyl-imidazo[1,2-a]pyridin-2-yl)-1-(2- |
| | phenyl-piperidin-1-yl)-ethanone |
| 112 | [3-Chloro-6-(1H-pyrazol-4-yl)-8-trifluoromethyl-imidazo[1,2-a]pyridin-2- |
| | yl]-[4-(1H-pyrazol-4-yl)-3,6-dihydro-2H-pyridin-1-yl]-methanone |
| 113 | 1-(1,3-Dihydro-isoindol-2-yl)-2-(6-furan-3-yl-8-trifluoromethyl- |
| | imidazo[1,2-a]pyridin-2-yl)-ethanone |
| 114 | 1′-(3-Chloro-6-furan-3-yl-8-trifluoromethyl-imidazo[1,2-a]pyridine-2- |
| | carbonyl)-[1,4′]bipiperidinyl-2-one |
| 116 | (3-Fluoro-3′,6′-dihydro-2′H-[2,4′]bipyridinyl-1′-yl)-(6-thiophen-2-yl-4- |
| | trifluoromethyl-1H-pyrrolo[2,3-b]pyridin-2-yl)-methanone |
| 117 | (3-Fluoro-3′,6′-dihydro-2′H-[2,4′]bipyridinyl-1′-yl)-(5-thiophen-2-yl-7- |
| | trifluoromethyl-1H-pyrrolo[3,2-b]pyridin-2-yl)-methanone |
| 128 | 2-(3-Fluoro-phenyl)-N-(6-furan-3-yl-8-trifluoromethyl-imidazo[1,2- |
| | a]pyridin-2-ylmethyl)-acetamide |
| 129 | 2-(2-Fluoro-phenyl)-N-(6-furan-3-yl-8-trifluoromethyl-imidazo[1,2- |
| | a]pyridin-2-ylmethyl)-acetamide |
| 130 | N-(6-Furan-3-yl-8-trifluoromethyl-imidazo[1,2-a]pyridin-2-ylmethyl)- |
| | benzamide |
| 131 | [3-Chloro-6-(1H-pyrazol-4-yl)-8-trifluoromethyl-imidazo[1,2-a]pyridin-2- |
| | yl]-(3-furan-3-yl-2,5-dihydro-pyrrol-1-yl)-methanone |
| 132 | [3-Chloro-6-(1H-pyrazol-4-yl)-8-trifluoromethyl-imidazo[1,2-a]pyridin-2- |
| | yl]-(3-thiophen-3-yl-2,5-dihydro-pyrrol-1-yl)-methanone |
| 133 | [3-Chloro-6-(1H-pyrazol-4-yl)-8-trifluoromethyl-imidazo[1,2-a]pyridin-2- |
| | yl]-(3-isoxazol-4-yl-2,5-dihydro-pyrrol-1-yl)-methanone |
| 134 | [3-Chloro-6-(1H-pyrazol-4-yl)-8-trifluoromethyl-imidazo[1,2-a]pyridin-2- |
| | yl]-(3-pyridin-3-yl-2,5-dihydro-pyrrol-1-yl)-methanone |
| 135 | [3-Chloro-6-(1H-pyrazol-4-yl)-8-trifluoromethyl-imidazo[1,2-a]pyridin-2- |
| | yl]-[3-(1H-pyrazol-4-yl)-2,5-dihydro-pyrrol-1-yl]-methanone |
| 136 | [3-Chloro-6-(1H-pyrazol-4-yl)-8-trifluoromethyl-imidazo[1,2-a]pyridin-2- |
| | yl]-[3-(1,2,3,6-tetrahydro-pyridin-4-yl)-2,5-dihydro-pyrrol-1-yl]- |
| | methanone |
| 137 | [3-Chloro-6-(1H-pyrazol-4-yl)-8-trifluoromethyl-imidazo[1,2-a]pyridin-2- |
| | yl]-[3-(1H-pyrazol-3-yl)-2,5-dihydro-pyrrol-1-yl]-methanone |
| 138 | N-[2-(6-Furan-3-yl-8-trifluoromethyl-imidazo[1,2-a]pyridin-2-yl)-ethyl]- |
| | benzamide |
| 152 | (3-Chloro-6-furan-3-yl-8-trifluoromethyl-imidazo[1,2-a]pyridin-2-yl)-[4- |
| | (cyclopropylmethyl-amino)-piperidin-1-yl]-methanone |
| 153 | 6-tert-Butoxycarbonylamino-2-[2-(6-furan-3-yl-8-trifluoromethyl- |
| | imidazo[1,2-a]pyridin-2-yl)-acetylamino]-hexanoic acid methyl ester |
| 154 | 2-[(6-Furan-3-yl-8-trifluoromethyl-imidazo[1,2-a]pyridine-2-carbonyl)- |
| | amino]-3,3-dimethyl-butyric acid methyl ester |
| 155 | 2-[(6-Furan-3-yl-8-trifluoromethyl-imidazo[1,2-a]pyridine-2-carbonyl)- |
| | amino]-3-(3H-imidazol-4-yl)-propionic acid methyl ester |
| 156 | 2-[(6-Furan-3-yl-8-trifluoromethyl-imidazo[1,2-a]pyridine-2-carbonyl)- |
| | amino]-4-methyl-pentanoic acid methyl ester |
| 157 | 2-[(6-Furan-3-yl-8-trifluoromethyl-imidazo[1,2-a]pyridine-2-carbonyl)- |
| | amino]-3-phenyl-propionic acid methyl ester |
| 158 | (3-Chloro-6-furan-3-yl-8-trifluoromethyl-imidazo[1,2-a]pyridin-2-yl)-[4- |
| | (3-methyl-butoxy)-piperidin-1-yl]-methanone |
| 159 | 2-[1-(3-Chloro-6-furan-3-yl-8-trifluoromethyl-imidazo[1,2-a]pyridine-2- |
| | carbonyl)-1,2,3,6-tetrahydro-pyridin-4-yl]-cyclopent-2-enone |
| 163 | 6-Furan-3-yl-2-(3-thiazol-2-yl-2,5-dihydro-pyrrol-1-ylmethyl)-8- |
| | trifluoromethyl-imidazo[1,2-a]pyridine |
| 169 | 3-Chloro-8-difluoromethyl-6-furan-3-yl-2-(3-thiazol-2-yl-2,5-dihydro- |
| | pyrrole-1-carbonyl)-imidazo[1,2-a]pyridine-5-carbonitrile |
| 174 | [3-Chloro-6-(1H-pyrazol-4-yl)-8-trifluoromethyl-imidazo[1,2-a]pyridin-2- |
| | yl]-(4-cyclopropylmethoxy-piperidin-1-yl)-methanone |
| 175 | (3-Chloro-6-furan-3-yl-8-trifluoromethyl-imidazo[1,2-a]pyridin-2-yl)-(4- |
| | cyclopropylmethoxy-piperidin-1-yl)-methanone |
| 176 | [3-Chloro-6-(1H-pyrazol-4-yl)-8-trifluoromethyl-imidazo[1,2-a]pyridin-2- |
| | yl]-(4-propoxy-piperidin-1-yl)-methanone |
| 177 | [3-Chloro-6-(1H-pyrazol-4-yl)-8-trifluoromethyl-imidazo[1,2-a]pyridin-2- |
| | yl]-(4-ethoxy-piperidin-1-yl)-methanone |
| 182 | 2-{1-[3-Chloro-6-(1H-pyrazol-4-yl)-8-trifluoromethyl-imidazo[1,2- |
| | a]pyridine-2-carbonyl]-piperidin-4-yloxy}-acetamide |
| 184 | 2-(6-Furan-3-yl-8-trifluoromethyl-imidazo[1,2-a]pyridin-2-yl)-N-thiazol- |
| | 2-yl-acetamide |
| 185 | N-Benzyl-2-(6-furan-3-yl-8-trifluoromethyl-imidazo[1,2-a]pyridin-2-yl)- |
| | acetamide |
| 186 | 2-(6-Furan-3-yl-8-trifluoromethyl-imidazo[1,2-a]pyridin-2-yl)-1-(3- |
| | thiazol-2-yl-2,5-dihydro-pyrrol-1-yl)-ethanone |
| 187 | 2-(6-Furan-3-yl-8-trifluoromethyl-imidazo[1,2-a]pyridin-2-yl)-1-(3- |
| | thiophen-2-yl-pyrrolidin-1-yl)-ethanone |
| 188 | 2-(6-Furan-3-yl-8-trifluoromethyl-imidazo[1,2-a]pyridin-2-yl)-1-(2- |
| | thiophen-2-yl-pyrrolidin-1-yl)-ethanone |
| 189 | Thiophene-2-sulfonic acid [2-(6-furan-3-yl-8-trifluoromethyl- |
| | imidazo[1,2-a]pyridin-2-yl)-acetyl]-amide |
| 190 | N-{1-[2-(6-Furan-3-yl-8-trifluoromethyl-imidazo[1,2-a]pyridin-2-yl)- |
| | acetyl]-azetidin-3-yl}-methanesulfonamide |
| 191 | 2-(6-Furan-3-yl-8-trifluoromethyl-imidazo[1,2-a]pyridin-2-yl)-1-(4- |
| | thiazol-2-yl-3,6-dihydro-2H-pyridin-1-yl)-ethanone |
| 192 | 2-(6-Furan-3-yl-8-trifluoromethyl-imidazo[1,2-a]pyridin-2-yl)-N- |
| | phenethyl-acetamide |
| 193 | 3-Chloro-6-furan-3-yl-8-trifluoromethyl-imidazo[1,2-a]pyridine-2- |
| | carboxylic acid (4-phenyl-cyclohexyl)-amide |
| 194 | (4-Benzoyl-piperidin-1-yl)-(3-chloro-6-furan-3-yl-8-trifluoromethyl- |
| | imidazo[1,2-a]pyridin-2-yl)-methanone |
| 195 | (3-Chloro-6-furan-3-yl-8-trifluoromethyl-imidazo[1,2-a]pyridin-2-yl)-(4- |
| | pyrrolidin-1-yl-piperidin-1-yl)-methanone |
| 196 | [3-Chloro-6-(1H-pyrazol-4-yl)-8-trifluoromethyl-imidazo[1,2-a]pyridin-2- |
| | yl]-(4-thiazol-2-yl-3,6-dihydro-2H-pyridin-1-yl)-methanone |
| 198 | (3-Chloro-6-furan-3-yl-8-trifluoromethyl-imidazo[1,2-a]pyridin-2-yl)-(4- |
| | pyridin-3-ylethynyl-3,6-dihydro-2H-pyridin-1-yl)-methanone |
| 199 | (3-Chloro-6-furan-3-yl-8-trifluoromethyl-imidazo[1,2-a]pyridin-2-yl)-[4- |
| | (3,3-dimethyl-but-1-ynyl)-3,6-dihydro-2H-pyridin-1-yl]-methanone |
| 200 | (3-Chloro-6-furan-3-yl-8-trifluoromethyl-imidazo[1,2-a]pyridin-2-yl)-(4- |
| | cyclopentylethynyl-3,6-dihydro-2H-pyridin-1-yl)-methanone |
| 212 | (3-Chloro-6-furan-3-yl-8-trifluoromethyl-imidazo[1,2-a]pyridin-2-yl)-[4- |
| | (2-cyclopropyl-vinyl)-3,6-dihydro-2H-pyridin-1-yl]-methanone |
| 213 | (3-Chloro-6-furan-3-yl-8-trifluoromethyl-imidazo[1,2-a]pyridin-2-yl)-[4- |
| | (2,5-dihydro-1H-pyrrol-3-yl)-3,6-dihydro-2H-pyridin-1-yl]-methanone |
| 225 | (3-Chloro-6-furan-3-yl-8-trifluoromethyl-imidazo[1,2-a]pyridin-2-yl)-[4- |
| | (3-methyl-butyl)-piperidin-1-yl]-methanone |
| 226 | Cyclopropanecarboxylic acid (6-furan-3-yl-8-trifluoromethyl- |
| | imidazo[1,2-a]pyridin-2-ylmethyl)-amide |
| 227 | Cyclopropanesulfonic acid (6-furan-3-yl-8-trifluoromethyl-imidazo[1,2- |
| | a]pyridin-2-ylmethyl)-amide |
| 228 | 6-Furan-3-yl-2-(4-phenyl-imidazol-1-ylmethyl)-8-trifluoromethyl- |
| | imidazo[1,2-a]pyridine |
| 229 | 6-Furan-3-yl-2-(3-phenyl-[1,2,4]oxadiazol-5-ylmethyl)-8-trifluoromethyl- |
| | imidazo[1,2-a]pyridine |
| 230 | 2-(3-Benzyl-[1,2,4]oxadiazol-5-ylmethyl)-6-furan-3-yl-8-trifluoromethyl- |
| | imidazo[1,2-a]pyridine |
| 231 | 6-Furan-3-yl-2-(3-phenoxymethyl-[1,2,4]oxadiazol-5-ylmethyl)-8- |
| | trifluoromethyl-imidazo[1,2-a]pyridine |
| 232 | 2-Methyl-propane-1-sulfonic acid (6-furan-3-yl-8-trifluoromethyl- |
| | imidazo[1,2-a]pyridin-2-ylmethyl)-amide |
| 234 | 2-(6-Furan-3-yl-8-trifluoromethyl-imidazo[1,2-a]pyridin-2-ylmethyl)- |
| | isoindole-1,3-dione |
| 235 | 1-(4-Chloro-benzyl)-2-pyrrolidin-1-ylmethyl-1H-benzoimidazole |
| 236 | (6-Furan-3-yl-8-trifluoromethyl-imidazo[1,2-a]pyridin-2-yl)-acetonitrile |
| 237 | 1-(3-Chloro-6-furan-3-yl-8-trifluoromethyl-imidazo[1,2-a]pyridine-2- |
| | carbonyl)-pyrrolidine-2-carboxylic acid amide |
| 238 | [3-Chloro-6-(1H-pyrazol-4-yl)-8-trifluoromethyl-imidazo[1,2-a]pyridin-2- |
| | yl]-(3-dimethylamino-pyrrolidin-1-yl)-methanone |
| 239 | 2-Dimethylamino-N-(6-furan-3-yl-8-trifluoromethyl-imidazo[1,2- |
| | a]pyridin-2-ylmethyl)-acetamide |
| 240 | 2-Acetylamino-N-(6-furan-3-yl-8-trifluoromethyl-imidazo[1,2-a]pyridin- |
| | 2-ylmethyl)-acetamide |
| 241 | 2-Amino-4-methyl-pentanoic acid (6-furan-3-yl-8-trifluoromethyl- |
| | imidazo[1,2-a]pyridin-2-ylmethyl)-amide |
| 242 | [2-(6-Furan-3-yl-8-trifluoromethyl-imidazo[1,2-a]pyridin-2-yl)-ethyl]- |
| | carbamic acid tert-butyl ester |
| 243 | 2-Acetylamino-4-methyl-pentanoic acid (6-furan-3-yl-8-trifluoromethyl- |
| | imidazo[1,2-a]pyridin-2-ylmethyl)-amide |
| 244 | 2-Acetylamino-N-(6-furan-3-yl-8-trifluoromethyl-imidazo[1,2-a]pyridin- |
| | 2-ylmethyl)-propionamide |
| 245 | 2-Acetylamino-N-(6-furan-3-yl-8-trifluoromethyl-imidazo[1,2-a]pyridin- |
| | 2-ylmethyl)-3-(1H-indol-3-yl)-propionamide |
| 246 | [(6-Furan-3-yl-8-trifluoromethyl-imidazo[1,2-a]pyridine-2-carbonyl)- |
| | amino]-acetic acid methyl ester |
| 247 | 6-Furan-3-yl-8-trifluoromethyl-imidazo[1,2-a]pyridine-2-carboxylic acid |
| | carbamoylmethyl-amide |
| 248 | 6-Furan-3-yl-8-trifluoromethyl-imidazo[1,2-a]pyridine-2-carboxylic acid |
| | methylcarbamoylmethyl-amide |
| 249 | 6-Furan-3-yl-8-trifluoromethyl-imidazo[1,2-a]pyridine-2-carboxylic acid |
| | dimethylcarbamoylmethyl-amide |
| 250 | 2-[(6-Furan-3-yl-8-trifluoromethyl-imidazo[1,2-a]pyridine-2-carbonyl)- |
| | amino]-propionic acid methyl ester |
| 251 | 2-[(6-Furan-3-yl-8-trifluoromethyl-imidazo[1,2-a]pyridine-2-carbonyl)- |
| | amino]-4-methylsulfanyl-butyric acid methyl ester |
| 252 | 2-[(6-Furan-3-yl-8-trifluoromethyl-imidazo[1,2-a]pyridine-2-carbonyl)- |
| | amino]-3-hydroxy-propionic acid methyl ester |
| 253 | 2-[(6-Furan-3-yl-8-trifluoromethyl-imidazo[1,2-a]pyridine-2-carbonyl)- |
| | amino]-3-hydroxy-butyric acid methyl ester |
| 254 | 1-(6-Furan-3-yl-8-trifluoromethyl-imidazo[1,2-a]pyridine-2-carbonyl)- |
| | pyrrolidine-2-carboxylic acid methyl ester |
| 255 | [(6-Furan-3-yl-8-trifluoromethyl-imidazo[1,2-a]pyridine-2-carbonyl)- |
| | amino]-phenyl-acetic acid methyl ester |
| 256 | 2-[(6-Furan-3-yl-8-trifluoromethyl-imidazo[1,2-a]pyridine-2-carbonyl)- |
| | amino]-3-methyl-butyric acid methyl ester |
| 257 | 6-tert-Butoxycarbonylamino-2-[(6-furan-3-yl-8-trifluoromethyl- |
| | imidazo[1,2-a]pyridine-2-carbonyl)-amino]-hexanoic acid methyl ester |
| 258 | 2-(6-Furan-3-yl-8-trifluoromethyl-imidazo[1,2-a]pyridin-2-yl)-ethylamine |
| 259 | 2-Acetylamino-N-(6-furan-3-yl-8-trifluoromethyl-imidazo[1,2-a]pyridin- |
| | 2-ylmethyl)-3-hydroxy-propionamide |
| 260 | 2-Acetylamino-N-(6-furan-3-yl-8-trifluoromethyl-imidazo[1,2-a]pyridin- |
| | 2-ylmethyl)-3-phenyl-propionamide |
| 261 | 2-Acetylamino-N-(6-furan-3-yl-8-trifluoromethyl-imidazo[1,2-a]pyridin- |
| | 2-ylmethyl)-3-methyl-butyramide |
| 262 | 2-Dimethylamino-N-(6-furan-3-yl-8-trifluoromethyl-imidazo[1,2- |
| | a]pyridin-2-ylmethyl)-propionamide |
| 263 | Thiophene-2-carboxylic acid [2-(6-furan-3-yl-8-trifluoromethyl- |
| | imidazo[1,2-a]pyridin-2-yl)-ethyl]-amide |
| 264 | [(6-Furan-3-yl-8-trifluoromethyl-imidazo[1,2-a]pyridine-2-carbonyl)- |
| | amino]-acetic acid |
| 265 | 2-[(6-Furan-3-yl-8-trifluoromethyl-imidazo[1,2-a]pyridine-2-carbonyl)- |
| | amino]-propionic acid |
| 266 | 2-[(6-Furan-3-yl-8-trifluoromethyl-imidazo[1,2-a]pyridine-2-carbonyl)- |
| | amino]-4-methylsulfanyl-butyric acid |
| 267 | 2-[(6-Furan-3-yl-8-trifluoromethyl-imidazo[1,2-a]pyridine-2-carbonyl)- |
| | amino]-3-hydroxy-propionic acid |
| 268 | (4-Benzenesulfonyl-piperidin-1-yl)-(3-chloro-6-furan-3-yl-8- |
| | trifluoromethyl-imidazo[1,2-a]pyridin-2-yl)-methanone |
| 269 | 2-[(6-Furan-3-yl-8-trifluoromethyl-imidazo[1,2-a]pyridine-2-carbonyl)- |
| | amino]-3-hydroxy-butyric acid |
| 270 | 2-[(6-Furan-3-yl-8-trifluoromethyl-imidazo[1,2-a]pyridine-2-carbonyl)- |
| | amino]-4-methyl-pentanoic acid |
| 271 | [(6-Furan-3-yl-8-trifluoromethyl-imidazo[1,2-a]pyridine-2-carbonyl)- |
| | amino]-phenyl-acetic acid |
| 272 | 2-[(6-Furan-3-yl-8-trifluoromethyl-imidazo[1,2-a]pyridine-2-carbonyl)- |
| | amino]-3-methyl-butyric acid |
| 273 | 2-[(6-Furan-3-yl-8-trifluoromethyl-imidazo[1,2-a]pyridine-2-carbonyl)- |
| | amino]-3,3-dimethyl-butyric acid |
| 274 | N-[2-(6-Furan-3-yl-8-trifluoromethyl-imidazo[1,2-a]pyridin-2-yl)-ethyl]- |
| | 2-thiophen-2-yl-acetamide |
| 275 | Cyclopropanecarboxylic acid [2-(6-furan-3-yl-8-trifluoromethyl- |
| | imidazo[1,2-a]pyridin-2-yl)-ethyl]-amide |
| 276 | 3-[(6-Furan-3-yl-8-trifluoromethyl-imidazo[1,2-a]pyridine-2-carbonyl)- |
| | amino]-propionic acid methyl ester |
| 277 | 6-Furan-3-yl-8-trifluoromethyl-imidazo[1,2-a]pyridine-2-carboxylic acid |
| | (1-carbamoyl-ethyl)-amide |
| 278 | 6-Furan-3-yl-8-trifluoromethyl-imidazo[1,2-a]pyridine-2-carboxylic acid |
| | (1-carbamoyl-2-methyl-propyl)-amide |
| 279 | 6-Furan-3-yl-8-trifluoromethyl-imidazo[1,2-a]pyridine-2-carboxylic acid |
| | (1-carbamoyl-2-hydroxy-ethyl)-amide |
| 280 | 1-(6-Furan-3-yl-8-trifluoromethyl-imidazo[1,2-a]pyridine-2-carbonyl)- |
| | pyrrolidine-2-carboxylic acid amide |
| 281 | 1-(6-Furan-3-yl-8-trifluoromethyl-imidazo[1,2-a]pyridine-2-carbonyl)- |
| | pyrrolidine-2-carboxylic acid dimethylamide |
| 282 | 6-Furan-3-yl-8-trifluoromethyl-imidazo[1,2-a]pyridine-2-carboxylic acid |
| | (1-carbamoyl-2-hydroxy-propyl)-amide |
| 283 | 1-[2-(6-Furan-3-yl-8-trifluoromethyl-imidazo[1,2-a]pyridin-2-yl)-ethyl]-3- |
| | phenyl-urea |
| 284 | 1-Acetyl-pyrrolidine-2-carboxylic acid (6-furan-3-yl-8-trifluoromethyl- |
| | imidazo[1,2-a]pyridin-2-ylmethyl)-amide |
| 285 | 3-[(6-Furan-3-yl-8-trifluoromethyl-imidazo[1,2-a]pyridine-2-carbonyl)- |
| | amino]-propionic acid |
| 286 | 2-[(6-Furan-3-yl-8-trifluoromethyl-imidazo[1,2-a]pyridine-2-carbonyl)- |
| | amino]-3-phenyl-propionic acid |
| 287 | 2-Acetylamino-N-(6-furan-3-yl-8-trifluoromethyl-imidazo[1,2-a]pyridin- |
| | 2-ylmethyl)-4-methylsulfanyl-butyramide |
| 288 | 2-Acetylamino-N-(6-furan-3-yl-8-trifluoromethyl-imidazo[1,2-a]pyridin- |
| | 2-ylmethyl)-3-methyl-butyramide |
| 289 | 2-Acetylamino-N-(6-furan-3-yl-8-trifluoromethyl-imidazo[1,2-a]pyridin- |
| | 2-ylmethyl)-3-hydroxy-butyramide |
| 290 | [2-(6-Furan-3-yl-8-trifluoromethyl-imidazo[1,2-a]pyridin-2-yl)- |
| | acetylamino]-acetic acid methyl ester |
| 291 | 2-[2-(6-Furan-3-yl-8-trifluoromethyl-imidazo[1,2-a]pyridin-2-yl)- |
| | acetylamino]-4-methylsulfanyl-butyric acid methyl ester |
| 292 | 2-[2-(6-Furan-3-yl-8-trifluoromethyl-imidazo[1,2-a]pyridin-2-yl)- |
| | acetylamino]-3-hydroxy-propionic acid methyl ester |
| 293 | 2-[2-(6-Furan-3-yl-8-trifluoromethyl-imidazo[1,2-a]pyridin-2-yl)- |
| | acetylamino]-3-(3H-imidazol-4-yl)-propionic acid methyl ester |
| 294 | 2-[2-(6-Furan-3-yl-8-trifluoromethyl-imidazo[1,2-a]pyridin-2-yl)- |
| | acetylamino]-3,3-dimethyl-butyric acid methyl ester |
| 295 | 3-[2-(6-Furan-3-yl-8-trifluoromethyl-imidazo[1,2-a]pyridin-2-yl)- |
| | acetylamino]-propionic acid methyl ester |
| 296 | 2-[2-(6-Furan-3-yl-8-trifluoromethyl-imidazo[1,2-a]pyridin-2-yl)- |
| | acetylamino]-propionamide |
| 297 | 2-[2-(6-Furan-3-yl-8-trifluoromethyl-imidazo[1,2-a]pyridin-2-yl)- |
| | acetylamino]-3-methyl-butyramide |
| 298 | 2-[2-(6-Furan-3-yl-8-trifluoromethyl-imidazo[1,2-a]pyridin-2-yl)- |
| | acetylamino]-3-hydroxy-propionamide |
| 299 | 2-[2-(6-Furan-3-yl-8-trifluoromethyl-imidazo[1,2-a]pyridin-2-yl)- |
| | acetylamino]-3-hydroxy-butyramide |
| 300 | N-Carbamoylmethyl-2-(6-furan-3-yl-8-trifluoromethyl-imidazo[1,2- |
| | a]pyridin-2-yl)-acetamide |
| 301 | 2-(6-Furan-3-yl-8-trifluoromethyl-imidazo[1,2-a]pyridin-2-yl)-N- |
| | methylcarbamoylmethyl-acetamide |
| 302 | N-Dimethylcarbamoylmethyl-2-(6-furan-3-yl-8-trifluoromethyl- |
| | imidazo[1,2-a]pyridin-2-yl)-acetamide |
| 303 | 2-[2-(6-Furan-3-yl-8-trifluoromethyl-imidazo[1,2-a]pyridin-2-yl)- |
| | acetylamino]-3-(1H-indol-3-yl)-propionamide |
| 304 | 2-[2-(6-Furan-3-yl-8-trifluoromethyl-imidazo[1,2-a]pyridin-2-yl)- |
| | acetylamino]-propionic acid methyl ester |
| 305 | 2-[2-(6-Furan-3-yl-8-trifluoromethyl-imidazo[1,2-a]pyridin-2-yl)- |
| | acetylamino]-3-hydroxy-butyric acid methyl ester |
| 306 | 2-[2-(6-Furan-3-yl-8-trifluoromethyl-imidazo[1,2-a]pyridin-2-yl)- |
| | acetylamino]-4-methyl-pentanoic acid methyl ester |
| 307 | 1-[2-(6-Furan-3-yl-8-trifluoromethyl-imidazo[1,2-a]pyridin-2-yl)-acetyl]- |
| | pyrrolidine-2-carboxylic acid methyl ester |
| 309 | [2-(6-Furan-3-yl-8-trifluoromethyl-imidazo[1,2-a]pyridin-2-yl)- |
| | acetylamino]-phenyl-acetic acid methyl ester |
| 310 | 2-[2-(6-Furan-3-yl-8-trifluoromethyl-imidazo[1,2-a]pyridin-2-yl)- |
| | acetylamino]-3-methyl-butyric acid methyl ester |
| 311 | 2-[2-(6-Furan-3-yl-8-trifluoromethyl-imidazo[1,2-a]pyridin-2-yl)- |
| | acetylamino]-3-(1H-indol-3-yl)-propionic acid methyl ester |
| 312 | 2-[2-(6-Furan-3-yl-8-trifluoromethyl-imidazo[1,2-a]pyridin-2-yl)- |
| | acetylamino]-3-phenyl-propionic acid methyl ester |
| 313 | 1-[2-(6-Furan-3-yl-8-trifluoromethyl-imidazo[1,2-a]pyridin-2-yl)-acetyl]- |
| | pyrrolidine-2-carboxylic acid amide |
| 314 | 1-[2-(6-Furan-3-yl-8-trifluoromethyl-imidazo[1,2-a]pyridin-2-yl)-acetyl]- |
| | pyrrolidine-2-carboxylic acid dimethylamide |
| 322 | N-[1-(3-Chloro-6-furan-3-yl-8-trifluoromethyl-imidazo[1,2-a]pyridine-2- |
| | carbonyl)-piperidin-4-yl]-methanesulfonamide |
| 323 | N-[1-(3-Chloro-6-furan-3-yl-8-trifluoromethyl-imidazo[1,2-a]pyridine-2- |
| | carbonyl)-piperidin-4-yl]-acetamide |
| 324 | 9-(3-Chloro-6-furan-3-yl-8-trifluoromethyl-imidazo[1,2-a]pyridine-2- |
| | carbonyl)-1,9-diaza-spiro[5.5]undecan-2-one |
| 325 | 1-[4-(3-Chloro-6-furan-3-yl-8-trifluoromethyl-imidazo[1,2-a]pyridine-2- |
| | carbonyl)-[1,4]diazepan-1-yl]-ethanone |
| 326 | (3-Chloro-6-furan-3-yl-8-trifluoromethyl-imidazo[1,2-a]pyridin-2-yl)-(4- |
| | methanesulfonyl-[1,4]diazepan-1-yl)-methanone |
| 327 | 1-[8-(3-Chloro-6-furan-3-yl-8-trifluoromethyl-imidazo[1,2-a]pyridine-2- |
| | carbonyl)-1-thia-4,8-diaza-spiro[4.5]dec-4-yl]-ethanone |
| 330 | 8-(3-Chloro-6-furan-3-yl-8-trifluoromethyl-imidazo[1,2-a]pyridine-2- |
| | carbonyl)-1,3,8-triaza-spiro[4.5]decane-2,4-dione |
| 331 | 4-(3-Chloro-6-furan-3-yl-8-trifluoromethyl-imidazo[1,2-a]pyridine-2- |
| | carbonyl)-[1,4]diazepane-1-carboxylic acid methyl ester |

12. A pharmaceutical composition comprising a pharmaceutically acceptable diluent and a therapeutically effective amount of a compound of claim 1.

13. A method for treating a viral infection in a mammal mediated at least in part by a virus in the Flaviviridae family of viruses which method comprises administering to a mammal, that has been diagnosed with said viral infection or is at risk of developing said viral infection, a compound of claim 1.

14. The method of claim 13, wherein said virus is hepatitis C virus.

15. The method of claim 14, further comprising administration of a therapeutically effective amount of one or more agents active against hepatitis C virus.

16. The method of claim 15, wherein said agent active against hepatitis C virus is an inhibitor of HCV proteases, HCV polymerase, HCV helicase, HCV NS4B protein, HCV entry, HCV assembly, HCV egress, HCV replicase, HCV NS5A protein, or inosine 5′-monophosphate dehydrogenase.

17. The method of claim 14, wherein said agent active against hepatitis C virus is interferon.
